SEQUENCE LISTING (3 sequences)

SEQ ID NO: 1 - protein, 4 aa, synthetic construct
DEVD

SEQ ID NO: 2 - protein, 7 aa, synthetic construct
KDEVDAP

SEQ ID NO: 3 - protein, 8 aa, synthetic construct
GKDEVDAP

(19) United States
(12) Patent Application Publication
(10) Pub. No.: US 2024/0382628 A1
(43) Pub. Date: Nov. 21, 2024

(54) COMPOSITIONS AND METHODS RELATING TO CELLS WITH ADHERED PARTICLES

(71) Applicant: PRESIDENT AND FELLOWS OF HARVARD COLLEGE, Cambridge, MA (US)

(72) Inventors: Samir MITRAGOTRI, Cambridge, MA (US); Li-Wen WANG, Cambridge, MA (US); Yongsheng GAO, Cambridge, MA (US); Neha KAPATE, Cambridge, MA (US); Supriya Suraj PRAKASH, Cambridge, MA (US); Ninad KUMBHOJKAR, Cambridge, MA (US)

(73) Assignee: PRESIDENT AND FELLOWS OF HARVARD COLLEGE, Cambridge, MA (US)

(21) Appl. No.: 18/577,453

(22) PCT Filed: Jul. 15, 2022
(86) PCT No.: PCT/US2022/037261

§ 371 Date: Jan. 8, 2024

Related U.S. Application Data
(60) Provisional application No. 63/222,120, filed on Jul. 15, 2021.

(51) Int. Cl. A61K 49/18; A61K 49/14

(52) U.S. Cl. CPC A61K 49/1896; A61K 49/14; A61K 49/1821

(57) ABSTRACT

Provided herein are polymeric particles and compositions (i.e., “backpacks”) that can adhere to cells and provide delivery of payload agents to those cells, and/or direct therapeutic activity of those cells.

### CROSS-REFERENCE TO RELATED APPLICATIONS

This application claims benefit under 35 U.S.C. § 119(e) of U.S. Provisional Application No. 63/222,120 filed Jul. 15, 2021, the contents of which are incorporated herein by reference in their entirety.

### GOVERNMENT SUPPORT

This invention was made with government support under W81XWH-19-2-0011 awarded by the U.S. Army. The government has certain rights in the invention.

### TECHNICAL FIELD

The technology described herein relates to methods and compositions relating to cells with particles adhered to their cell surface.

### BACKGROUND

Particles adhered to cells have shown promise in targeted delivery of therapeutics or diagnostics in patients. But existing technology for such particles has been limited both in binding efficacy and the types of cells to which the particles can be adhered.

### SUMMARY

Described herein are improved polymeric particles that provide improved binding efficacy, e.g., strong enough to permit direct injection of polymeric particles which will adhere in vivo, as well as polymeric particles that can bind effectively to a wider range of cell types. Furthermore, described herein are polymeric particles capable of successfully delivery of imagining agents, e.g. MRI contrast agents.

In one aspect of any of the embodiments, described herein is a polymeric particle comprising at least one imaging reagent. In some embodiments of any of the aspects, the at least one imaging reagent is at least one MRI contrast reagent. In some embodiments of any of the aspects, the polymeric particle further comprises at least one binding reagent selected from the group consisting of: a CD11b binding reagent; a CD3 binding reagent; a CD19 binding reagent; a CD49b binding reagent; a CD56 binding reagent; a CD11a binding reagent; a CD27 binding reagent; a CD44 binding reagent; a CD45 binding reagent; a NKG2D binding reagent; a NKp30 binding reagent; a NKp46 binding reagent; and an ICAM1 binding reagent. In some embodiments of any of the aspects, the polymeric particle further comprises at least one binding reagent selected from the group consisting of: a CD11b binding reagent; a CD3 binding reagent; a CD19 binding reagent; a CD49b binding reagent; and a CD56 binding reagent.

In one aspect of any of the embodiments, described herein is a polymeric particle comprising at least one binding reagent selected from the group consisting of: a CD11b binding reagent; a CD3 binding reagent; a CD19 binding reagent; a CD49b binding reagent; and a CD56 binding reagent. In some embodiments of any of the aspects, the binding reagent is a CD11b binding reagent; a CD3 binding reagent; or a CD19 binding reagent. In one aspect of any of the embodiments, described herein is a polymeric particle comprising at least one binding reagent selected from the group consisting of: a CD11b binding reagent; a CD3 binding reagent; a CD19 binding reagent; a CD49b binding reagent; a CD56 binding reagent; a CD11a binding reagent; a CD27 binding reagent; a CD44 binding reagent; a CD45 binding reagent; a NKG2D binding reagent; a NKp30 binding reagent; a NKp46 binding reagent; and an ICAM1 binding reagent.

In some embodiments of any of the aspects, the binding reagent is a CD11b binding reagent. In some embodiments of any of the aspects, the binding reagent is a CD45 binding reagent.

In some embodiments of any of the aspects, the binding reagent is an antibody or an antibody reagent. In some embodiments of any of the aspects, the antibody or antibody reagent is an anti-CD11b antibody or anti-CD11b antibody reagent. In some embodiments of any of the aspects, the antibody or antibody reagent is an anti-CD45 antibody or anti-CD45 antibody reagent. In some embodiments of any of the aspects, the antibody or the antibody reagent comprises one or more CDRs of an antibody or antibody reagent selected from Table 2. In some embodiments of any of the aspects, the antibody or the antibody reagent comprises the six CDRs of an antibody or antibody reagent selected from Table 2. In some embodiments of any of the aspects, the binding reagent further comprises a streptavidin molecule or biotin molecule.

In one aspect of any of the embodiments, described herein is a polymeric particle as described herein and further comprising ICAM, NKp30, and/or NKp46. In one aspect of any of the embodiments, described herein is a polymeric particle comprising ICAM, NKp30, and/or NKp46. In some embodiments of any of the aspects, the polymeric particle comprises ICAM and NKp30.

In some embodiments of any of the aspects, the polymeric particle further comprises one or more cell adhesive molecules (e.g., polyelectrolytes). In some embodiments of any of the aspects, the polymeric particle comprises a single region comprising a hydrogel of one or more cell adhesive molecules (e.g., polyelectrolytes). In some embodiments of any of the aspects, the polymeric particle further comprises one or more structural polymers. In some embodiments of any of the aspects, the polymeric particle comprises a single region comprising a hydrogel of one or more structural polymers.

In some embodiments of any of the aspects, the polymeric particle comprises:
a. a first region comprising a first selection of one or more cell adhesive molecules (e.g., polyelectrolytes) and optionally the one or more binding reagents;
b. a second region comprising a second selection of one or more cell adhesive molecules (e.g., polyelectrolytes); and
c. a third region comprising one or more structural polymers and optionally the one or more binding reagents.

In some embodiments of any of the aspects, the first selection of one or more cell adhesive molecules comprises one or more of: hyaluronic acid (HA) and bovine serum albumin (BSA); the second selection of one or more cell adhesive molecules is poly(allylamine) hydrochloride (PAH); and the one or more structural polymers is one or more of: poly(lactic-co-glycolic) acid (PLGA), PLGA-PEG, PLGA-PEG-maleimide, and PLGA-PEG-biotin.

In some embodiments of any of the aspects, the polymeric particle comprises:
a. a first region comprising a first selection of one or more structural polymers and optionally the one or more binding reagents; and
b. a second region comprising a second selection of one or more structural polymers.

In some embodiments of any of the aspects, the polymeric particle comprises:
a. a first region comprising a first selection of one or more structural polymers and optionally the one or more binding reagents;
b. a second region comprising a second selection of one or more structural polymers; and
c. a third region comprising a third selection of one or more structural polymers and optionally the one or more binding reagents.

In some embodiments of any of the aspects, the second region is located between the first region and third region and/or the second region separates the first region and third region from each other.

In some embodiments of any of the aspects, the cell adhesive molecules comprise one or more of cell adhesive polyelectrolytes, immunoglobulins, or ligands for receptors on cell surfaces. In some embodiments of any of the aspects, the cell adhesive polyelectrolytes comprise one or more of: hyaluronic acid (HA), methylacrylate HA, hyaluronic acid-aldehyde, bovine serum albumin (BSA), PEG, PEG dimethylacrylate, and/or poly(allylamine) hydrochloride (PAH). In some embodiments of any of the aspects, the cell adhesive polyelectrolytes comprise one or more of: hyaluronic acid (HA), methylacrylated HA, and hyaluronic acid-aldehyde; and one or more of PEG and PEG dimethylacrylate.

In some embodiments of any of the aspects, the structural polymer comprises one or more of poly(lactic-co-glycolic) acid (PLGA); a combination of PLGA and poly (D,L-lactide-co-glycolide); a combination of PLGA and poly (D,L-lactide-co-glycolide) with acid ends; a combination of PLGA and poly (D,L-lactide-co-glycolide) at a 50:50 molar ratio; -polyvinyl alcohol (PVA); hyaluronic acid (HA); gelatin; collagen;PLGA-PEG; or poly(glycerol sebacate) (PGS).

In some embodiments of any of the aspects, the second region further comprises poly(lactic-co-caprolactone) (PLCL). In some embodiments of any of the aspects, the second region comprises or further comprises a near-infrared degradable polymer or polymer linker.

In some embodiments of any of the aspects, the polymeric particle comprises a binding reagent comprising a streptavidin molecule bound to a structural polymer further comprising biotin. In some embodiments of any of the aspects, the polymeric particle comprises a binding reagent comprising a streptavidin molecule bound to PLGA-PEG-biotin. In some embodiments of any of the aspects, the polymeric particle comprises a binding reagent bound to PLGA-PEG-maleimide via DTT catalyzed-reaction. In some embodiments of any of the aspects, the polymeric particle comprises a binding reagent comprising a biotin molecule bound to a structural polymer further comprising streptavidin.

In some embodiments of any of the aspects, the polymeric particle further comprises one or more imaging agents. In some embodiments of any of the aspects, the one or more imaging agents are selected from the group consisting of: one or more contrast agents, one or more MRI contrast agents, one or more microbubbles, one or more metal ions, one or more radioactive isotopes, one or more optical imaging agents, one or more SPECT imaging agents, and one or more PET imaging agents. In some embodiments of any of the aspects, the one or more MRI contrast agents comprise one or more of: a gadoliunium-based contrast agent, superparamagnetic iron oxide, ultrasmall superparamagnetic iron oxide, superparamagnetic iron-platinum, manganese chelates, iron salts, and perflubron. In some embodiments of any of the aspects, the gadoliunium-based contrast agent comprises one or more of: gadolinium, gadoxetate, gadobutrol, gadoterate, gadoteridol, gadopentetate, gadobenate, gadopentetic acid dimegulmine, gadoxentate, gadoversetamide, gadodiamide, gadofosveset, gadocoletic acid, gadomelitol, gadomer 17, gadoxetic acid. In some embodiments of any of the aspects, the one or more imaging agents further comprise methylacrylate. In some embodiments of any of the aspects, the one or more imaging agents are methylacrylate cross-linked to or more of methylacrylated HA and PEG dimethylacrylate.

In some embodiments of any of the aspects, the polymeric particle is substantially discoidal in shape. In some embodiments of any of the aspects, the polymeric particle is discoidal in shape. In some embodiments of any of the aspects, the diameter of the polymeric particle is from about 100 nm to about 10 μm. In some embodiments of any of the aspects, the diameter of the polymeric particle is from about 100 nm to about 1 μm. In some embodiments of any of the aspects, the polymeric particle is about 6 μm×500 nm in size. In some embodiments of any of the aspects, the polymeric particle is about 6 μm×250 nm in size. In some embodiments of any of the aspects, the polymeric particle is 1-2 μm×7-9 μm in size. In some embodiments of any of the aspects, the polymeric particle has a volume of 0.5×10⁻¹¹ cm³ to 10×10⁻¹¹ cm³. In some embodiments of any of the aspects, the polymeric particle has a volume of 1.25×10⁻¹¹ cm³ to 5×10⁻¹¹ cm³. In some embodiments of any of the aspects, the polymeric particle has a shape which is a rod, a cylinder, a cube, a cuboid, a hexahedron, or a pyramid. In some embodiments of any of the aspects, a region is a layer.

In some embodiments of any of the aspects, the polymeric particle further comprises one or more cell-targeting ligands. In some embodiments of any of the aspects, the cell-targeting ligand is located in a region comprising cell adhesive molecules (e.g., polyelectrolytes). In some embodiments of any of the aspects, the cell-targeting ligand is IgG, an antibody, a polypeptide, or an aptamer.

In some embodiments of any of the aspects, the polymeric particle further comprises one or more payload reagents. In some embodiments of any of the aspects, the payload reagent is a therapeutic molecule. In some embodiments of any of the aspects, the payload reagent is a small molecule or polypeptide. In some embodiments of any of the aspects, the payload reagent is present in admixture with the structural polymer. In some embodiments of any of the aspects, the payload reagent is present in the second region.

In some embodiments of any of the aspects, the polymeric particle further comprises an echogenic liposome. In some embodiments of any of the aspects, the polymeric particle further comprises a magnetic nanoparticle. In some embodiments of any of the aspects, the polymeric particle further comprises a gold nanoparticle.

In some embodiments of any of the aspects, the polymeric particle further comprises at least one polarizing agent. In some embodiments of any of the aspects, the polarizing agent is an N1/M1-polarizing agent. In some embodiments of any of the aspects, the polarizing agent is an N1/M2-polarizing agent. In some embodiments of any of the aspects, the N1/M1-polarizing agent is selected from the group consisting of: IFN-γ; TNF; TNF-alpha; a Toll-like receptor agonist (e.g., LPS, muramyl dipeptide, or lipoteichoic acid); GM-CSF; IL-1β; IL-6; IL-12; IL-23, and CD11b. In some embodiments of any of the aspects, the N2/M2-polarizing agent is selected from the group consisting of: IL-4; IL-10; glucocortoids (e.g., cortisol, cortisone, prednisone, prednisolone, methylprednisonolone, dexamethasone, betamethasone, triamcinolone, fludrocortisone acetate, and deoxycorticosterone acetate); M-CSF, TGF-beta, IL-6; and IL-13. In some embodiments of any of the aspects, the release of one or more of the polarizing agents is triggered by contacting the particle with a small molecule or nucleic acid. In some embodiments of any of the aspects, the phenotype of a macrophage is regulated by the release of the one or more polarizing agents.

In one aspect of any of the embodiments, described herein is an engineered cellular composition comprising:
a. a cell; and
b. a polymeric particle of any of the preceding claims, wherein the particle is located on the cell surface of the cell.
   In some embodiments of any of the aspects, the cell is a monocyte, macrophage, natural killer cell, or neutrophil. In some embodiments of any of the aspects, the macrophage is an M0 macrophage. In some embodiments of any of the aspects, the macrophage is an M1-polarized macrophage. In some embodiments of any of the aspects, the macrophage is an M2-polarized macrophage. In some embodiments of any of the aspects, the macrophage is substantially driven to an M1 or M2 phenotype.

In one aspect of any of the embodiments, described herein is a method of obtaining images of a subject, the method comprising administering to the subject the polymeric particle or the engineered cellular composition described herein, wherein the polymeric particle comprises an imaging reagent, and then subjecting the subject to an imaging scan that can detect the imaging reagent. In some embodiments of any of the aspects, the cell is a macrophage, monocyte, or T cell.

In one aspect of any of the embodiments, described herein is a method of treating cancer and/or a tumor in a subject in need thereof, the method comprising administering to the subject the polymeric particle or the engineered cellular composition described herein. In some embodiments of any of the aspects, the polymeric particle comprises a payload reagent that is a chemotherapeutic. In some embodiments of any of the aspects, the method further comprises administering radiation or at least one chemotherapy to the subject. In some embodiments of any of the aspects, the cell is a macrophage, NK cell, or T cell.

In one aspect of any of the embodiments, described herein is a method of treating a fracture, wound, injury (e.g. TBI), or infection in a subject in need thereof, the method comprising administering to the subject the polymeric particle or the engineered cellular composition described herein. In some embodiments of any of the aspects, the polymeric particle comprises a payload reagent that is an antibiotic, antiviral, antimicrobial, a hemostatic agent, an anti-inflammatory agent, or an analgesic.

In one aspect of any of the embodiments, described herein is a method of treating inflammation in a subject in need thereof, the method comprising administering to the subject the polymeric particle or the engineered cellular composition described herein. In some embodiments of any of the aspects, the polymeric particle comprises a payload reagent that is an anti-inflammatory agent. In some embodiments of any of the aspects, the inflammation is in the lungs, joints, or skin. In some embodiments of any of the aspects, the polymeric particle comprises IL-4. In some embodiments of any of the aspects, the cell is a neutrophil.

In one aspect of any of the embodiments, described herein is a method of treating an autoimmune condition in a subject in need thereof, the method comprising administering to the subject the polymeric particle or the engineered cellular composition described herein. In some embodiments of any of the aspects, the polymeric particle comprises a payload reagent that is an immunosuppressive agent. In some embodiments of any of the aspects, the autoimmune condition is multiple scelarosis, diabetes, or arthritis. In some embodiments of any of the aspects, the cell is a macrophage or T cell.

In one aspect of any of the embodiments, described herein is a method of providing hemostatic treatment to a subject in need thereof, the method comprising administering to the subject the polymeric particle or the engineered cellular composition described herein. In some embodiments of any of the aspects, the polymeric particle comprises a payload reagent that is a hemostatic agent.

In one aspect of any of the embodiments, described herein is a method of treating a neurodegenerative disorder, a central nervous system disorder, or a peripheral nervous system disorder in a subject in need thereof, the method comprising administering to the subject the polymeric particle or the engineered cellular composition described herein. In some embodiments of any of the aspects, the polymeric particle comprises a payload reagent that is therapeutic for the neurodegenerative disorder, the central nervous system disorder, or the peripheral nervous system disorder.

In one aspect of any of the embodiments, described herein is a method of providing delivering a payload reagent to the central nervous system of a subject in need thereof, the method comprising administering to the subject the polymeric particle or the engineered cellular composition described herein, wherein the cell is a monocyte and/or the binding reagent is an anti-CD11b binding reagent.

In one aspect of any of the embodiments, described herein is a method of providing a gene therapy vector to a subject in need thereof, the method comprising administering to the subject the polymeric particle or the engineered cellular composition described herein, wherein the polymeric particle comprises a payload reagent that is a gene therapy vector. In some embodiments of any of the aspects, the gene therapy vector is an AAV.

In one aspect of any of the embodiments, described herein is a method of vaccinating a subject or inducing an immune response in a subject in need thereof, the method comprising administering to the subject the polymeric particle or the engineered cellular composition described herein, wherein the polymeric particle comprises a payload reagent that is an antigen. In some embodiments of any of the aspects, the cell is a B cell.

In some embodiments of any of the aspects, the cell is autologous to the subject. In some embodiments of any of the aspects, the cell is heterologous to the subject. In some embodiments of any of the aspects, the method further comprises a first step of obtaining the cell from a donor and/or the subject and contacting the cell with the polymeric particle ex vivo. In some embodiments of any of the aspects, a therapeutically effective dose of the polymeric particle or engineered cellular composition is administered.

In one aspect of any of the embodiments, described herein is a polymeric particle or engineered cellular composition as described herein for use in a method as described herein.

### BRIEF DESCRIPTION OF THE DRAWINGS

FIG. 1 depicts the adherence of exemplary anti-CD11b backpack designs to monocytes.

FIG. 2 depicts the design and assembly of exemplary backpack designs.

FIG. 3 depicts the release kinetics of exemplary backpacks.

FIGS. 4-5 depict the design and assembly of exemplary backpack designs.

FIG. 6 depicts flow cytometry measurement of backpack adherence.

FIG. 7 depicts adherence of anti-CD11b backpacks to neutrophils.

FIG. 8 depicts the adherence of BSA-PAH backpacks to NK cells.

FIG. 9 depicts backpack adherence rates under different conditions.

FIG. 10 depicts the design and assembly of several exemplary backpack designs.

FIG. 11 depicts the neutrophil adhesion rate for several exemplary backpack designs.

FIG. 12 depicts the adhesion rate for several exemplary backpack designs.

FIG. 13 demonstrates the biodistribution of backpacks.

FIG. 14 demonstrates the biodistribution of neutrophils.

FIGS. 15-16 demonstrates the loading and release profiles of several backpack designs.

FIGS. 17-18 depict an exemplary protocol for preparing gadolinium-loaded backpacks.

FIG. 19 demonstrate the relaxivity of gadolinium-loaded backpacks.

FIG. 20 demonstrates the stability of gadolinium-loaded backpack adherence to macrophages.

FIG. 21 depicts MRI imaging of gadolinium-loaded backpacks injected into ex vivo rat brain tissue.

FIG. 22 depicts exemplary backpack designs.

FIGS. 23-24 depict exemplary procedures for priting backpacks.

FIG. 25 depicts the binding of HA backpacks to macrophages.

FIG. 26 depicts the stability of HA backpacks adhered to macrophages.

FIG. 27 depicts the release kinetics of IFN-γ from backpacks.

FIG. 28 depicts the stability of IFN-γ in backpacks.

FIG. 29 depicts release of IL-4 from the backpacks.

FIGS. 30A-30C demonstrate the relaxivity of gadolinium-loaded backpacks.

FIG. 31 depicts the adherence of exemplary backpack designs.

FIG. 32 depicts the influence of backpacks loaded with IFN-γ on macrophage phenotypes.

FIG. 33 depicts the influence of administering backpacks loaded with IFN-γ to mice with cancer.

FIG. 34 depicts schematics of exemplary polymeric particle architectures.

FIGS. 35A-35B depict exemplary methods of modifying backpacks.

FIGS. 36A-36B depict the rate of attachment of unmodified backpacks to neutrophils. FIG. 36A provides a schematic of the backpack structure used. FIG. 36B depicts a graph of adhesion rates of backpacks using different types of PLGA based on termination (acid or ester) or L:G Ratio (50:50 or 65:35). FIG. 36C depicts a graph of adhesion rates of backpacks using different cell:BP ratios and cell concentrations.

FIGS. 37A-37D demonstrate that backpacks modified with antibodies demonstrate increased attachment to neutrophils. FIG. 37A shows a schematic of the method of preparing antibody reagents for use in backpacks. FIG. 37B depicts a schematic of the modified backpacks used in FIGS. 37C-37D. FIG. 37C depicts a graph of adhesion rate for backpacks modified with combinations of BSA, PAH, and HA. FIG. 37D depicts a graph of adhesion rate for backpacks modified with anti-CD11b.

FIG. 38A, from Miralda et al. 2017, depicts a list of priming agents. FIG. 38B depicts a graph of adhesion rates of backpacks in the presence or absence of IFNg and GMCSF. FIG. 38C depicts the effect of GCSF concentration on backpack adherence. FIG. 38D depicts the effect of GMCSF concentration on backpack adherence. For FIGS. 38C-38D, cells were treated with respective concentrations of cytokines for ˜30 minutes prior to addition of backpacks. In general, higher concentration of cytokines leads to higher attachment to neutrophils up to a certain threshold concentration (highlighted in boxes).

FIGS. 39A-39E depict scaling up of the backpack attachment to neutrophils. FIG. 39A depicts adhesion rates of 1 million (M) cells+1 million (M) backpacks in a 96 well plate. FIG. 39B depicts adhesion rates in a 15 mL centrifuge tuble. Changing the reactor vessel significantly impacts the attachment, possibly because of lower area for settling in a 15 mL tube. FIGS. 39C-39D depict the rate of attachment in the indicated volumes and reactors. FIG. 39E depicts the rate of attachment when incubated in 96 well plates and then collected in a 15 mL tube.

FIG. 40 depicts microscopy images showing backpacks are retained on the surface of the neutrophils.

FIGS. 41A-41G depict neutrophil viability under different conditions. FIG. 41A depicts the effect of medium formulations on neutrophil viability. FIGS. 41B-41E depict adhesion rates and viability overtime. FIGS. 41F-41G depict the amount of neutrophil degranulationa upon backpack attachment.

FIG. 42 depicts graphs of N1 polarization marker levels after backpack attachment.

FIGS. 43A-43B depict the level of neutrophil activation markers after backpack attachment.

FIGS. 44A-44B depict the level of neutrophil polarization markers after backpack attachment. FIG. 44C depicts the in vitro toxicity of neutrophils against 4T1 cells. FIG. 44D depicts the level of MPO release after nanoparticle treatment of neutrophils. FIG. 44E depicts the level of MPO release after attachment to different surfaces.

FIGS. 45A-45K demonstrate that neutrophils with backpacks reach tumors within 4 hours. FIG. 45A depicts a schematic of the experiment. FIG. 45B depicts a graph of the amount of neutrophils in the tumors over time. FIG. 45C depicts images of the amount of neutrophils in the tumors over time. FIGS. 45D-45E depict the accumulation of cells and backpacks in organs. In FIG. 45D, cells were stained with VivoTrack680. In FIG. 45E backpacks were stained with Rhodamine B. Organs were excised at 4 and 24 hours and imaged using IVIS. FIGS. 45F-45H depict graphs of neutrophil accumulation in the 4T1 tumors and other tissues over time. FIGS. 41F-45K depict graphs of backpack accumulation in the 4T1 tumors and other tissues over time.

FIGS. 46A-46G depict the immune response to neutrophils+backpacks after intraumoral injection. FIG. 46A depicts a schematic of the experiment. FIGS. 46B-46G depict graphs demonstrating that neutrophil backpacks alter the tumor microenvironment (TME).

FIGS. 47A-47B depict the efficacy of neutrophil backpacks in vivo.

FIGS. 48A-48H. FIG. 48A depicts a schematic of antibody-mediated coupling of backpacks to cells. FIG. 48B depicts images of backpacks of on murine and human NK cells. FIG. 48C depicts the effect of media composition when backpacks are adhered to C57BL/6 Spleen derived mouse NK cells in a 2h incubation. FIG. 48D depicts the effect of backpack:cell ratios when backpacks are adhered to C57BL/6 Spleen derived mouse NK cells in a 2h incubation. FIG. 48E depicts the rate of adhesion with different ligands in a 2:1 backpacks to cell ratio with complete media. FIG. 48F depicts the rate of adhesion with different ligands and time in a 2:1 backpacks to cell ratio with complete media. FIGS. 48G-FIG. 48H depicts the adhesion of backpacks to primary NKs and NK-92 cells using ICAM1 as a ligand.

FIGS. 49A-49K. FIG. 49A depicts the level of degranulation surface marker expressed on NK-92 cells after adherence of the indicated backpacks. FIG. 49B depicts the level of IFN-gamma secretion in NK-92 cells after adherence of the indicated backpacks at a 2:1 backpack to cell ratio.

FIG. 49C depicts the level of IFN-gamma secretion in NK-92 cells after adherence of the indicated backpacks at a 2:1 backpack to cell ratio. FIGS. 49D-49I depict graphs of gene expression measured by RT-PCR in human primary NK cells after backpack adherence. FIG. 49J depicts a graph of IFN-gamma secretion in mouse NK cells after backpack adherence. FIG. 49K depicts a graph of IFN-gamma secretion in IL-15 expanded mouse NK cells after backpack adherence.

FIGS. 50A-50D. FIGS. 50A-50B depict IFN-gamma production on NK-92 cells after adherence of the indicated backpacks. A 2:1 BP to cell ratio was used and free cytokines at a concentration of 100 ng/ml were used. The cells received 3 washes after incubation with cytokine.

FIGS. 50C-50D depict degranulation and surface marker expression on NK-92 cells after adherence of the indicated backpacks. A 2:1 BP to cell ratio was used and free cytokines at a concentration of 100 ng/ml were used. The cells received 3 washes after incubation with cytokine.

FIGS. 51A-51E. FIGS. 51A-51B depict the biodistribution of NK-92 in A375 melanoma tumor bearing mice. Mice were J:Nu nude mice with a subcutaneous A375 tumor on flank. FIGS. 51C-51E depict the biodistribution of NK-92 bound BPs in A375 melanoma tumor bearing mice. Mice were J:Nu nude mice with a subcutaneous A375 tumor on flank. The BPs (Rhodamine signal) overlaps with organ autofluorescence but BPs are detectable in single cells suspension.

FIGS. 52A-52G. FIG. 52A depicts a schematic summary of monocyte/backpack adhesion methods. FIG. 52B depicts a graph of adhesion to monocytes for a number of backpack formulations. FIG. 52C depicts a graph of adhesion to primary human monocytes for the indicated backpack formulations. FIG. 52D depicts a graph of adhesion to primary murine monocytes for the indicated backpack formulations. FIGS. 52E-52F depict images of backpacks adhered to monocytes. FIG. 52G depicts a graph of antibody-mediated backpack adherence to monocytes under different shear conditions.

FIGS. 53A-53C. FIG. 53A depicts a schematic of backpacks loaded with cytokines or small molecules. FIG. 53B depicts a graph showing IL-4 loading into backpacks. FIG. 53C depicts graphs showing dex loading into backpacks.

FIGS. 54A-54N. FIG. 54A depicts the viability of monocytes after backpacks adherence.

FIG. 54B depicts the level of CD11b expression in monocytes 1 hour after backpack adherence. FIG. 54C depicts the level of Ly6C expression in monocyptes 1 hour after backpack adherence. FIG. 54D depicts the level of CCR2 expression in monocyptes 1 hour after backpack adherence. FIG. 54E depicts the level of CX3CR1 expression in monocyptes 1 hour after backpack adherence. FIG. 54F depicts the viability of monocytes after backpacks adherence. FIGS. 54G-54H depict CD11b expression at 1 hour and 24 hours after backpack adherence. FIGS. 54I-54J depict Ly6C expression at 1 hour and 24 hours after backpack adherence. FIGS. 54K-54L depict CCR2 expression at 1 hour and 24 hours after backpack adherence. FIGS. 54M-54N depict CX3CR1 expression at 1 hour and 24 hours after backpack adherence.

FIGS. 55A-55I. FIG. 55A depicts the level of M1 marker gene expression 72 hours after adherence of the indicated backpacks to monocytes. FIG. 55B depicts the level of M2 marker gene expression 72 hours after adherence of the indicated backpacks to monocytes. FIGS. 55C-55H depict the level of phenotypic markers with different backpacks adhered, as compared to monocytes alone.

FIG. 551 shows a graph demonstrating that drug-loaded backpacks allow carrier monocyte viability to be maintained after 72 hours.

FIGS. 56A-56Q. FIG. 56A depicts a 1 dose regimen treatment of chronic EAE in vivo. FIG. 56B depicts a 2 dose regimen treatment of chronic EAE in vivo. FIGS. 56C-56D depict an EAE biodistribution study. FIGS. 56E-56H depict the level of immune cells/markers in the blood after a 2 dose regimen after treatment of acute EAE. FIGS. 56I-56K depict the level of the indicated cell types in the brain after a 2 dose regimen after treatment of acute EAE. FIGS. 56L-56M depict the level of the indicated cell types in the spinal cord after a 2 dose regimen after treatment of acute EAE. FIG. 56N depict the level of the indicated cell types in the spinal cord after a 2 dose regimen after treatment of acute EAE. FIGS. 56O-56P depict the level of IFN-gamma production in the CNS after a 2 dose regimen after treatment of acute EAE. FIG. 56Q depicts the level of IL-17A production in serum after a 2 dose regimen after treatment of acute EAE. In FIGS. 56E-56Q, “drugs” or “drug-loaded” refer to IL-4 and dexamethasone.

FIG. 57A depicts a schematic of a TBI backpack therapy. FIG. 57B depicts adherence of these backpacks to macrophages.

FIGS. 58A-58R. FIGS. 58A-58D depict adherence of backpacks to different cells times. Adherence was conducted ex vivo in whole blood for 1 hour, under static conditions. FIGS. 58E-58I depicts binding to different cell types over time in vivo. FIGS. 58J-58N depict biodistribution of backpacks with and without CD11b inclusion, overtime. FIGS. 580-58P depict the binding of backpacks to liver-associated immune cells in vivo. FIGS. 58Q-58R depict the binding of backpacks to lung-associated immune cells in vivo.

FIGS. 59A-59H depict the organ distribution of different backpack types. The backpacks were injected intravenously to healthy mice at a dose of 2 million backpacks/mouse.

FIGS. 60A-60D. FIG. 60A depicts a schematic of the structure of Gd backpacks. FIGS. 60B-60C depict the preparation of Gd-loaded hydrogel backpacks. FIG. 60D depicts a graph of backpack yield.

FIGS. 61A-61E. FIG. 61A depicts a summary of the paramagnetic properties of Gd BPs.

FIGS. 61B-61E depict the analysis of the paramagnetic properties of Gd BPs and free gadovist.

FIGS. 62A-62E depict the preparation of a cellular backpack system. FIGS. 62A-62B depict images of cellular backpacks attached to cells. FIG. 62C depicts a schematic of the process. FIGS. 62D and 62E depict the rate of attachment.

FIGS. 63A-63N. FIG. 63A depicts different methods of antibody modification of backpacks. FIGS. 63B-63C depict cBP stability under shear stress (FIG. 63B) and freezing (FIG. 63C).

FIGS. 63D-63E depict MR images of Gd BPs in rat brain tissue. FIGS. 63F-63G depict graphs of biodistribution of Gd BPs in healthy mice. FIGS. 63H-63J depict biodistribution of Gd BPs in mice with lung metastases. FIGS. 63K-63L depict graphs of the lung to liver accumulation ratio. FIGS. 63M-63N depict the Brain Distribution of BPs and cBPs.

FIGS. 64A-64G. FIG. 64A-64C depict the level of M1 and M2 macrophage markers in BP-laden macrophages and all macrophages. FIG. 64D-64G depict the level of cytokine release in BP-laden macrophages and all macrophages. Macrophage were porcine macrophage. LPS was used at 100 ng/mL and IL4 at 20 ng/mL.

### DETAILED DESCRIPTION

The methods and compositions described herein relate to polymeric particles which can be adhered to a cell surface, e.g., the surface of a monocyte or macrophage. When the polymeric particles described herein are adhered to a cell surface, they are resistant to phagocytosis. These polymeric particles are also referred to herein as “backpacks.”

Adhering such polymeric particles to certain cell types, e.g, natural killer cells, monocytes, and neutrophils, is particularly difficult. The inventors have demonstrated herein that including binding reagents specific for certain cell surface targets provides surprising increases in the ability of the polymeric particles to adhere to cells, particularly natural killer cells, monocytes, and neutrophils. The increase in binding efficiency is great enough to permit direct injection of polymeric particles into subjects, where the polymeric particles will then adhere to in vivo cells, instead of requiring adherence be conducted ex vivo and followed by injection of cells with adhered polymeric particles.

Accordingly, in one aspect of any of the embodiments, described herein is a polymeric particle comprising at least one binding reagent selected from the group consisting of: a CD11b binding reagent; a CD3 binding reagent; a CD19 binding reagent; a CD49b binding reagent; and a CD56 binding reagent. In one aspect of any of the embodiments, described herein is a polymeric particle comprising at least one binding reagent selected from the group consisting of: a CD11b binding reagent; a CD3 binding reagent; a CD19 binding reagent; a CD49b binding reagent; a CD56 binding reagent; a CD11a binding reagent; a CD27 binding reagent; a CD44 binding reagent; a CD45 binding reagent; a NKG2D binding reagent; a NKp30 binding reagent; a NKp46 binding reagent; and an ICAM1 binding reagent. In some embodiments of any of the aspects, the binding reagent is a CD11b binding reagent; a CD3 binding reagent; or a CD19 binding reagent. In some embodiments of any of the aspects, the binding reagent is a CD11b binding reagent.

As used herein, “binding reagent” refers to an agent that binds specifically to a target molecule. Exemplary binding reagents can include antibodies, antibody reagents, aptamers, ligands, intrabodies, and the like.

As used herein, “integrin subunit alpha M”, “ITGAM”, “Cluster of Differentiation 11b”, or “CD11b” refers to an integrin chain that combines with ITGB2 to form the CR3 or Mac-1 leukocyte-specific receptor. Sequences are known for CD11b genes and polypeptides for a number of species, e.g., human CD11b (NCBI Gene ID No: 3684) mRNA (e.g., NCBI Ref Seq: NM_000632.4 and NM_001145808.2) and polypeptide (e.g., NCBI Ref Seq: NP_001139280.1 and NP_000623.2).

As used herein, “CD3” or “Cluster of Differentiation 3” is a protein complex that functions as a T cell co-receptor. As used herein, binding to CD3 can refer to binding to the entire complex or any one or more of the 4 chains comprised by CD3. The four chains of CD3 are a CD3γ, a CD36, and two CD3E's. Sequences are known for CD3γ genes and polypeptides for a number of species, e.g., human CD3γ (NCBI Gene ID No: 917) mRNA (e.g., NCBI Ref Seq: NM_000073.3) and polypeptide (e.g., NCBI Ref Seq: NP_00064.1). Sequences are known for CD36 genes and polypeptides for a number of species, e.g., human CD36 (NCBI Gene ID No: 915) mRNA (e.g., NCBI Ref Seq: NM_000732.6 and NM_001040651.2) and polypeptide (e.g., NCBI Ref Seq: NP_000723.1 and NP_001035741.1). Sequences are known for CD3E genes and polypeptides for a number of species, e.g., human CD3E (NCBI Gene ID No: 916) mRNA (e.g., NCBI Ref Seq: NM_000733.4) and polypeptide (e.g., NCBI Ref Seq: NP_000724.1).

As used herein, “Cluster of Differentiation 19”, or “CD19” refers to a transmembrane protein typically found on B cells. Sequences are known for CD19 genes and polypeptides for a number of species, e.g., human CD19 (NCBI Gene ID No: 930) mRNA (e.g., NCBI Ref Seq: NM_001178098.2, NM_001385732.1, and NM_001770.6) and polypeptide (e.g., NCBI Ref Seq: NP_001171569.1, NP_001372661.1, and NP_001761.3).

As used herein, “Cluster of Differentiation 49b”, or “CD49” refers to an integrin alpha subunit protein. Sequences are known for CD49b genes and polypeptides for a number of species, e.g., human CD49b (NCBI Gene ID No: 3673) mRNA (e.g., NCBI Ref Seq: NM_002203.4) and polypeptide (e.g., NCBI Ref Seq: NP_002194.2).

As used herein, “Cluster of Differentiation 56”, or “CD56” refers to a glycoprotein often found on NK cells. Sequences are known for CD56 genes and polypeptides for a number of species, e.g., human CD56 (NCBI Gene ID No: 4684) mRNA (e.g., NCBI Ref Seq: NM_000615.7) and polypeptide (e.g., NCBI Ref Seq: NP_000606.3).

In some embodiments of any of the aspects, the binding reagent is an antibody or an antibody reagent.

In some embodiments of any of the aspects, the antibody or antibody reagent is an anti-CD11b antibody or anti-CD11b antibody reagent. In some embodiments of any of the aspects, the antibody or antibody reagent is an anti-CD3 antibody or anti-CD3 antibody reagent. In some embodiments of any of the aspects, the antibody or antibody reagent is an anti-CD19 antibody or anti-CD19 antibody reagent.

In some embodiments of any of the aspects, the antibody or antibody reagent is an anti-CD49b antibody or anti-CD49b antibody reagent. In some embodiments of any of the aspects, the antibody or antibody reagent is an anti-CD56 antibody or anti-CD56 antibody reagent.

Antibodies and antibody reagents specific for the targets/antigens provided herein are known in the art and available commercially. For example, Table 2 provides exemplary commercially-available species of antibodies specific to the targets/antigens described herein. Additional species are known to those of ordinary skill in the art, e.g., are published and available in the scientific literature.

TABLE 2

| Target/Antigen | Exemplary Antibodies |
|---|---|
| CD11b | M1/70 (BioLegend Cat. No. 101207) |
| | ICRF44 (ThermoFisher Cat. No. 11-0118-42) |
| | CBRM1/5 (ThermoFisher Cat. No. 11-0113-42) |
| | M1/70.15 (ThermoFisher Cat. No. MA1-80091) |
| | C67F154 (ThermoFisher Cat. No. 14-0196-82) |
| | LM2/1 (ThermoFisher Cat. No. BMS104BT) |
| | OTI2D11 (ThermoFisher Cat. No. CF807953) |
| | OTI12C10 (ThermoFisher Cat. No. CF807952) |
| | VIM12 (ThermoFisher Cat. No. CD11B01) |
| | 5C6 (ThermoFisher Cat. No. MA5-16527) |
| | ARC0414 (ThermoFisher MA5-35199) |
| | MEM-174 (ThermoFisher MA1-19004) |
| | EPR19387 (AbCam Cat. No. ab184308) |
| | EP1345Y (AbCam Cat. No. ab52478) |
| | EPR19373 (AbCam Cat. No. ab184307) |
| | SP300 (AbCam Cat. No. ab224805) |
| CD3 | SP7 (AbCam Cat. No. ab16669) |
| | SP162 (AbCam Cat. No. ab135372) |
| | CAL57 (AbCam Cat. No. ab237721) |
| | CD3-12 (AbCam Cat. No. ab11089) |
| | OKT3 (ThermoFisher Cat. No. 48-0037-42) |
| | UCHT1 (ThermoFisher Cat. No. 58-0038-42) |
| | 145-2C11 (ThermoFisher Cat. No. 47-0031-82) |
| | 17A2 (ThermoFisher Cat. No. 50-0032-82) |
| CD19 | HIB19 (ThermoFisher Cat. No. 12-0199-42) |
| | 1D3 (ThermoFisher Cat. No. 25-0193-82) |
| | 6OMP31 (ThermoFisher Cat. No. 14-0194-82) |
| | SJ25C1 (ThermoFisher Cat. No. 47-0198-42) |
| | LC1 (ThermoFisher Cat. No. 14-0190-82) |
| | 771404 (ThermoFisher Cat. No. MA5-24372) |
| | EPR5906 (AbCam Cat. No. ab134114) |
| | EPR23174-145 (AbCam Cat. No. ab245235) |
| | SP291 (AbCam Cat. No. ab227688) |
| | SP110 (AbCam Cat. No. ab225717) |
| | BU12 (AbCam Cat. No. ab254170) |
| | CD19/3117 (AbCam Cat. No. ab270715) |
| CD49b | HMa2 (AbCam Cat. No. ab253088) |
| | Y418 (ThermoFisher Cat. No. 14-0498-80) |
| | DX5 (ThermoFisher Cat. No. 48-5971-82) |
| | SN0752 (ThermoFisher Cat. No. MA5-32306) |
| | 31H4 (ThermoFisher Cat. No. MA1-26714) |
| | ARC0457 (ThermoFisher Cat. No. MA5-35243) |
| | AK7 (ThermoFisher Cat. No. MA5-38698) |
| CD56 | 123C3 (ThermoFisher Cat. No. MA1-06801) |
| | MEM-188 (ThermoFisher Cat. No. MA1-19129) |
| | CMSSB (ThermoFisher Cat. No. 12-0567-42) |
| | TULY56 (ThermoFisher Cat. No. 48-0566-42) |
| | NCAM1/1496 (AbCam Cat. No. ab218467) |
| | NCAM1/784 (AbCam Cat. No. ab216019) |
| | EPR21827 (AbCam Cat. No. ab220360) |
| | CAL53 (AbCam Cat. No. ab237708) |
| CD11a | EP1258Y (AbCam Cat. No. ab52895) |
| | EPR22578-312 (AbCam Cat. No. ab228964) |
| | Hu1124 (AbCam Cat. No. ab275978) |
| | I21/7 (AbCam Cat. No. ab25383) |
| | M17/4 (AbCam Cat. No. ab119878) |
| | MEM-25 (AbCam Cat. No. ab269335) |
| CD27 | EPR8569 (AbCam Cat. No. ab131254) |
| | EPR8568 (AbCam Cat. No. ab133761) |
| | BLR083G (AbCam Cat. No. ab265589) |
| | EPR21209 (AbCam Cat. No. ab213318) |
| | EPR20585 (AbCam Cat. No. ab214043) |
| | LPRS2/1611 (AbCam Cat. No. ab268144) |
| CD44 | BLR038F (AbCam Cat. No. ab243894) |
| | Hermes-3 (AbCam Cat. No. ab254530) |
| | EPR18668 (AbCam Cat. No. ab189524) |
| | EPR1013Y (AbCam Cat. No. ab51037) |
| | CD44Mab-5 (AbCam Cat. No. ab264539) |
| | SP37 (AbCam Cat. No. ab101531) |
| CD45 | EP322Y (AbCam Cat. No. ab40763) |
| | EPR20033 (AbCam Cat. No. ab208022) |
| | BL-178-12C7 (AbCam Cat. No. ab243869) |
| | RM1007 (AbCam Cat. No. ab281586) |
| | MEM-28 (AbCam Cat. No. ab8216) |
| | F10-89-4 (AbCam Cat. No. ab30470) |
| NKG2D | EPR22935-131 (AbCam Cat. No. ab233557) |
| | MM0489-10R27 (AbCam Cat. No. ab89807) |
| | 1D11 (AbCam Cat. No. ab35033) |
| | A10 (AbCam Cat No. ab171243) |
| | CX5 (AbCam Cat. No. ab95795) |
| | EPR23366-2693 (AbCam Cat. No. ab281162) |
| NKp30 | EPR14509 (AbCam Cat. No. ab186425) |
| | EPR23058-137 (AbCam Cat. No. ab243649) |
| | EPR14509 (AbCam Cat. No. ab250880) |
| | AF29-4D12 (ThermoFisher Cat. No. 25-3379-42) |
| | 210845 (R&D Systems Cat. No. MAB1849) |
| | P30-15 (BioLegend Cat. No. 325207) |
| NKp46 | EPR23097-35 (AbCam Cat. No. ab233558) |
| | mNCR1.05 (AbCam Cat. No. ab283505) |
| | EPR22403-57 (AbCam Cat. No. ab224703) |
| | EPR22403-40 (AbCam Cat. No. ab244208) |
| | EPR26322-23 (AbCam Cat. No. ab300044) |
| | MM0491-8F24 (AbCam Cat. No. ab89877) |
| ICAM1 | EPR24639-3 (AbCam Cat. No. ab282575) |
| | EPR22161-284 (AbCam Cat. No. ab222736) |
| | EPR4776 (AbCam Cat. No. ab109361) |
| | 1A29 (AbCam Cat. No. ab171123) |
| | EPR16608 (AbCam Cat. No. ab179707) |
| | EP1442Y (AbCam Cat. No. ab53013) |

In some embodiments of any of the aspects, the antibody or the antibody reagent comprises one or more CDRs of an antibody or antibody reagent selected from Table 2. In some embodiments of any of the aspects, the antibody or the antibody reagent comprises the six CDRs of an antibody or antibody reagent selected from Table 2.

In one aspect of any of the embodiments, described herien is a polymeric particle as described herein and further comprising ICAM1, NKp30, and/or NKp46. In one aspect of any of the embodiments, described herien is a polymeric particle comprising ICAM1, NKp30, and/or NKp46. The sequences of ICAM1, NKp30, and NKp46 are known in the art, e.g., human ICAM1 (NCBI Gene ID 3383), human NKp46 (NCBI LGene ID: 9437) and human NKp30 (NCBI Gene ID: 259197). In some embodiments of any of the aspects, the ICAM1, NKp46, or NKp30 is a polypeptide associated with the foregoing NCBI Gene ID Nos as of the filing date of this application. In some embodiments of any of the aspects, the polymeric particle comprises ICAM and NKp30.

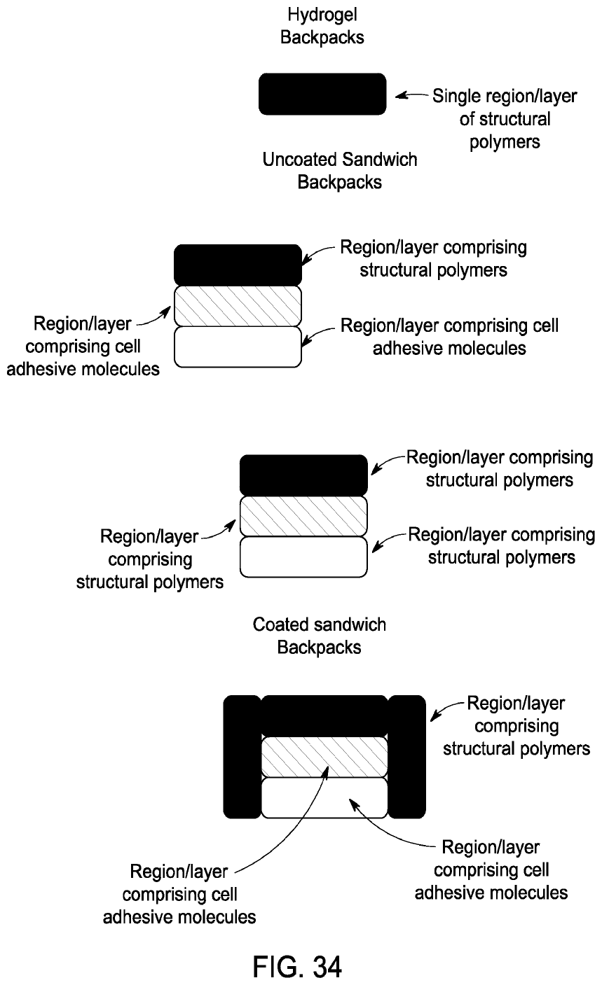

Several polymeric particle architectures are provided herein. These architectures are termed herein, 1) hydrogel backpack; 2) an uncoated sandwich backpack or layer-by-layer (LbL) backpack; and 3) a coated sandwich backpack or layer-by-layer (LbL) backpack. Exemplary embodiments of each are depicted in FIG. 34.

A hydrogel backpack comprises a single layer or region of structural polymers or cell adhesive molecules, e.g., present as a hydrogel.

In some embodiments of any of the aspects, a polymeric particle described herein further comprises one or more structural polymers. In some embodiments of any of the aspects, a polymeric particle described herein further comprises a single polymer region, the region comprising one or more structural polymers. In some embodiments of any of the aspects, a polymeric particle described herein further comprises a single polymer region or layer, the region or layer comprising a hydrogel comprising one or more structural polymers.

As used herein, the term “gel” refers to the state of matter between liquid and solid. As such, a “gel” has some of the properties of a liquid (i.e., the shape is resilient and deformable) and some of the properties of a solid (i.e., the shape is discrete enough to maintain three dimensions on a two dimensional surface.). A gel may be provided in pharmaceutical acceptable carriers known to those skilled in the art, such as saline or phosphate buffered saline. Such carriers may routinely contain pharmaceutically acceptable concentrations of salt, buffering agents, preservatives, compatible carriers and optionally other therapeutic agents. A non-limiting example of a gel is a hydrogel. A hydrogel is a substance that is formed when an organic polymer (natural or synthetic) is crosslinked via covalent, ionic, or hydrogen bonds to create a three-dimensional open-lattice structure which entraps water molecules to form a gel.

Structural polymers are those which are suitable for forming into a thin disk. Exemplary structural polymers can include, by way of non-limiting example polylactide (PLA): polyglycolide (PGA); poly-(ε-caprolactone) (PCL); polyphosphazenes; polyorthoesters; polyanhydrides; poly(α-hydroxy esters); poly(ether esters); copolymers comprising lactide of glycolide and ε-caprolactone or trimethylene carbonate; poly(polyol sebacate) elastomers; elastomers; poly(polyol citrate); polyesters; poly(glycolic acid); poly(lactic acid); poly(caprolactone); poly(lactic-co-glycolic acid); poly(butylene succinate); poly(trimethylene carbonate); poly(p-dioxanone); poly(butylene terephthalate); poly(ester amide)s; Hybrane™ S1200; DegraPol™; polyurethanes; polyanhydrides; poly[(caboxyphenoxy) propane-sebacic acid]; polyphsophoesters; poly[bis(hydroxyethyl) terephthalate-ethyl orthophosphorylate/terephthaloyl chloride]; poly(ortho esters); poly(alkyl cyanoacrylates); poly(butyl cyanoacrylate); polyethers; poly(ethylene glycol); poly(amino acids); tyrosine derived polycarbonate; microbial polyesters; poly([3-hydroxyalkanoate); poly(hydroxybutyrate); poly(hydroxybutyrate-co-hydroxyvalerate); collagen; albumin; gluten; chitosan; hyaluronate; cellulose; alginate; and starch. Suitable structural polymers are discussed in more detail at, e.g., Bat et al. Regen. Med. 9:385-398 (2014) and Marin et al. Int. J Nanomedicine 8:3071-3091 (2013); which are incorporated by reference herein in their entireties. In some embodiments of any of the aspects, the structural polymer comprises poly(lactic-co-glycolic) acid (PLGA), polyvinyl alcohol (PVA), hyaluronic acid (HA), gelatin, collagen and/or poly(glycerol sebacate) (PGS).

In some embodiments of any of the aspects, a region comprising one or more structural polymers comprises a single structural polymer. In some embodiments of any of the aspects a region comprising one or more structural polymers comprises two or more structural polymers.

In some embodiments of any of the aspects, a region comprising a structural polymer comprises poly(lactic-co-caprolactone) (PLCL). In some embodiments of any of the aspects, a region comprising a structural polymer comprises a) poly(lactic-co-glycolic) acid (PLGA) and/or poly(glycerol sebacate) (PGS) and b) poly(lactic-co-caprolactone) (PLCL).

In some embodiments of any of the aspects, the one or more structural polymers comprises poly(lactic-co-glycolic) acid (PLGA); a combination of PLGA and poly (D,L-lactide-co-glycolide); a combination of PLGA and poly (D,L-lactide-co-glycolide) with acid ends; a combination of PLGA and poly (D,L-lactide-co-glycolide) at a 50:50 molar ratio; -polyvinyl alcohol (PVA); hyaluronic acid (HA); gelatin; collagen; PLGA-PEG; or poly(glycerol sebacate) (PGS). In some embodiments of any of the aspects, the one or more structural polymers comprises poly(lactic-co-glycolic) acid (PLGA); a combination of PLGA and poly (D,L-lactide-co-glycolide); a combination of PLGA and poly (D,L-lactide-co-glycolide) with acid ends; a combination of PLGA and poly (D,L-lactide-co-glycolide) at a 50:50 molar ratio; -polyvinyl alcohol (PVA); gelatin; collagen; PLGA-PEG; or poly(glycerol sebacate) (PGS). In some embodiments of any of the aspects, the one or more structural polymers comprises poly(lactic-co-glycolic) acid (PLGA); a combination of PLGA and poly (D,L-lactide-co-glycolide); a combination of PLGA and poly (D,L-lactide-co-glycolide) with acid ends; a combination of PLGA and poly (D,L-lactide-co-glycolide) at a 50:50 molar ratio; or PLGA-PEG. In some embodiments of any of the aspects, the one or more structural polymers comprises, consists of, or consists essentially of PLGA. In some embodiments of any of the aspects, the one or more structural polymers comprises, consists of, or consists essentially of PLGA-PEG. In some embodiments of any of the aspects, the one or more structural polymers comprises, consists of, or consists essentially of a combination of PLGA and PLGA-PEG. In some embodiments of any of the aspects, the one or more structural polymers comprises polyvinyl alcohol (PVA).

In some embodiments of any of the aspects, a hydrogel backpack comprises a single polymer region or layer, the region or layer comprising one or more biotinylated structural polymers. In some embodiments of any of the aspects, a hydrogel backpack comprises a single polymer region or layer, the region or layer comprising one or more biotinylated structural polymers bound to one or more binding reagents comprising streptavidin molecules.

In some embodiments of any of the aspects, a hydrogel backpack comprises a single polymer region or layer, the region or layer comprising one or more structural polymers comprising malemide molecules. In some embodiments of any of the aspects, a hydrogel backpack comprises a single polymer region or layer, the region or layer comprising one or more structural polymers malemide molecules covalently conjugated to one or more binding reagents.

In some embodiments of any of the aspects, a hydrogel backpack comprises a single polymer region or layer, the region or layer comprising a hydrogel comprising PLGA; a combination of PLGA and poly (D,L-lactide-co-glycolide); a combination of PLGA and poly (D,L-lactide-co-glycolide) with acid ends; or a combination of PLGA and poly (D,L-lactide-co-glycolide) at a 50:50 molar ratio.

In some embodiments of any of the aspects, a hydrogel backpack comprises, consists of, or consists essentially of a single polymer region or layer, the region or layer comprising a hydrogel of one or more structural polymers. In some embodiments of any of the aspects, a hydrogel backpack comprises, consists of, or consists essentially of a single polymer region or layer, the region or layer comprising a) a hydrogel of one or more structural polymers, and b) one or more binding reagents. In some embodiments of any of the aspects, a hydrogel backpack comprises, consists of, or consists essentially of a single polymer region or layer, the region or layer comprising a) a hydrogel of one or more structural polymers, b) one or more binding reagents, c) one or more polarizing agents. In some embodiments of any of the aspects, a hydrogel backpack comprises, consists of, or consists essentially of a single polymer region or layer, the region or layer comprising a) a hydrogel of one or more structural polymers, and b) one or more polarizing agents.

In some embodiments of any of the aspects, the one or more structural polymers comprise, consist essentially of, or consist of one or more of poly(lactic-co-glycolic) acid (PLGA), PLGA-PEG, PLGA-PEG-maleimide, PLGA-PEG-biotin; a combination of PLGA and poly (D,L-lactide-co-glycolide); a combination of PLGA and poly (D,L-lactide-co-glycolide) with acid ends; or a combination of PLGA and poly (D,L-lactide-co-glycolide) at a 50:50 molar ratio. In some embodiments of any of the aspects, the one or more structural polymers comprise, consist essentially of, or consist of PLGA-PEG. In some embodiments of any of the aspects, the one or more structural polymers comprise, consist essentially of, or consist of PLGA. In some embodiments of any of the aspects, the one or more structural polymers comprise, consist essentially of, or consist of a combination of PLGA and PLGA-PEG.

In some embodiments of any of the aspects, a polymeric particle described herein further comprises one or more cell adhesive molecules. In some embodiments of any of the aspects, a polymeric particle described herein further comprises a single polymer region, the region comprising one or more one or more cell adhesive molecules. In some embodiments of any of the aspects, a polymeric particle described herein further comprises a single polymer region or layer, the region or layer comprising a hydrogel comprising one or more one or more cell adhesive molecules.

Cell adhesive molecules can be any molecule which will adhere to the surface of a cell, e.g., monocyte, macrophage, natural killer cell, T cell, or neutrophil. Non-limiting examples of suitable cell adhesive molecules include polyelectrolytes, immunoglobulins, ligands for receptors on a cell surface, and/or monocyte-targeting and/or macrophage-targeting ligands. Characteristics that can enhance cell adhesion include, e.g., high surface free energy, hydrophilic protein content, low surface hydration, and low surface charge density. Exemplary, non-limiting cell adhesive molecules can include poly (glycidyl methacrylate) (PGMA); polycaprolactone (PCL); polydimethylsiloxane (PDMS); poly(hexamethyldisiloxane) (PHMDSO); superhydrophobic perfluoro-substituted PEDOT (PEDOT-F); superhydrophobic polystyrene (PS); plasma-treated poly (methyl methacrylate) (PMMA); plasma-treated poly-3-hydroxybutyrate (P3HB); phosphatidylethanolamine (PE); and carboxymethyl chitin (CMCH). Cell adhesive molecules can also include, or comprise, e.g., RGD peptides, collagen, fibronectin, gelatin, and collagen. Further discussion of cell adhesive molecules can be found, e.g., at Lih et al. Progress in Polymer Science 44:28-61 (2015) and Chen et al. Materials Today (2017); which are incorporated by reference herein in their entireties.

In some embodiments of any of the aspects, cell adhesive polyelectrolytes comprise hyaluronic acid, poly(allylamine) hydrochloride, and/or hyaluronic acid modified to comprise aldehyde groups.

Ligands for the receptors on a given cell surface and/or which target a monocyte or macrophage are known in the art and can include natural or synthetic ligands. Exemplary ligands for macrophages and/or monocytes can include, by way of non-limiting example, IL-4; CX3CL1; IL-17A; IL-17F; M-CSF; GM-CSF; LDL; ApoE; IL-2; IFN-γ; Hsp60; Hsp70; complement C5A; leukotriene B4; CCL2; CCL4; CCL3; CCL5; CCL7; CCL8; CXCL8; CXCL9; CXCL10; and/or CXCL11. In some embodiments of any of the aspects, monocyte-targeting and/or macrophage-targeting ligand is IgG, an antibody (e.g., an antibody specific for a molecule (e.g., a receptor) on the monocyte or macrophage cell surface), a polypeptide, or an aptamer.

In some embodiments of any of the aspects, the cell adhesive molecules can be specific for one or more cell types, e.g., macrophages and/or monocytes. However, the particles can be adhered to isolated cell populations in vitro, and thus such specificity is not required in all embodiments. In some embodiments of any of the aspects, the cell adhesive molecules are not specific for a specific cell type(s).

In some embodiments of any of the aspects, a region comprising at least one cell adhesive molecule comprises a single type of cell adhesive molecule. In some embodiments of any of the aspects a region comprising at least one cell adhesive molecule comprises two or more types of cell adhesive molecules, e.g., two cell adhesive polyelectrolytes and/or a cell adhesive polyelectrolyte and an immunoglobulin.

In some embodiments of any of the aspects, the cell adhesive molecules comprise one or more of cell adhesive polyelectrolytes, immunoglobulins, or ligands for receptors on cell surfaces.

In some embodiments of any of the aspects, the cell adhesive polyelectrolytes comprise one or more of: hyaluronic acid (HA), methylacrylated HA, hyaluronic acid-aldehyde, bovine serum albumin (BSA), PEG, PEG dimethylacrylate, and/or poly(allylamine) hydrochloride (PAH). In some embodiments of any of the aspects, the hyaluronic acid is modified to comprise aldehyde groups. In some embodiments of any of the aspects, the cell adhesive polyelectrolytes comprise one or more of hyaluronic acid (HA), methylacrylated HA, and hyaluronic acid-aldehyde; and one or more of PEG and PEG dimethylacrylate. In some embodiments of any of the aspects, the cell adhesive polyelectrolytes comprise one or more of hyaluronic acid (HA) and methylacrylated HA; and one or more of PEG and PEG dimethylacrylate. In some embodiments of any of the aspects, the cell adhesive polyelectrolytes comprise HA and PEG. In some embodiments of any of the aspects, the cell adhesive polyelectrolytes comprise methylacrylated HA and PEG dimethylacrylate.

In some embodiments of any of the aspects, a hydrogel backpack comprises, consists of, or consists essentially of a single polymer region or layer, the region or layer comprising a) a hydrogel of one or more one or more cell adhesive molecules, b) one or more binding reagents, c) and one or more MRI contrast reagents. In some embodiments of any of the aspects, a hydrogel backpack comprises, consists of, or consists essentially of a single polymer region or layer, the region or layer comprising a) a hydrogel of one or more one or more cell adhesive molecules, and b) one or more MRI contrast reagents. In some embodiments of any of the aspects, the one or more cell adhesive molecules comprise, consist essentially of, or consist of: hyaluronic acid (HA), methylacrylated HA, PEG, PEG dimethylacrylate, and combinations thereof. In some embodiments of any of the aspects, the one or more cell adhesive molecules comprise, consist essentially of, or consist of: methylacrylated HA and PEG dimethylacrylate. In some embodiments of any of the aspects, a hydrogel backpack comprises, consists of, or consists essentially of a single polymer region or layer, the region or layer comprising a) a hydrogel of methylacrylated HA and PEG dimethylacrylate, and b) one or more methacrylated MRI contrast reagents.

A two-layer backpack comprises at least two distinct regions or layers. In an uncoated two-layer backpack, the layers are stacked and substantially parallel to each other. Uncoated two-layer backpacks can be created by, e.g., stamping each layer consecutively. In an coated two-layer backback, the second layer (applied last), wraps around the sides of the backpack, such that only one face of the first layer is exposed to the exterior environment. In a coated two-layer backpack, the sides of the first or bottom layer may be partially coated or entirely coated but the face of the first or bottom layer most distal from the other layer is not coated. Coated two-layer backpacks can be created by e.g, stamping the first layer and then spin-coating the second layer.

In some embodiments of any of the aspects, the polymeric particle comprises: a) a first region comprising the one or more binding reagents and one or more cell adhesive molecules (e.g., polyelectrolytes) and b) a second region comprising one or more structural polymers and optionally one or more binding reagents. In some embodiments of any of the aspects, the polymeric particle comprises: a) a first region comprising one or more cell adhesive molecules (e.g., polyelectrolytes) and optionally the one or more binding reagents and b) a second region comprising one or more cell adhesive molecules (e.g., polyelectrolytes) and optionally one or more binding reagents. In some embodiments of any of the aspects, the polymeric particle comprises: a) a first region comprising a hydrogel of one or more structural polymers; and b) a second region comprising one or more cell adhesive molecules (e.g., polyelectrolytes) and optionally one or more binding reagents. In some embodiments of any of the aspects, the polymeric particle comprises: a) a first region comprising a hydrogel of one or more structural polymers; and b) a second region comprising one or more cell adhesive molecules (e.g., polyelectrolytes) and one or more binding reagents.

In some embodiments of any of the aspects, the first cell adhesive molecule is hyaluronic acid (HA), methylacrylated HA, hyaluronic acid-aldehyde, or bovine serum albumin (BSA); the second cell adhesive molecule is poly(allylamine) hydrochloride (PAH); and the structural polymer is one or more of: poly(lactic-co-glycolic) acid (PLGA), PLGA-PEG, PLGA-PEG-maleimide, or PLGA-PEG-biotin. In some embodiments of any of the aspects, the cell adhesive molecule is hyaluronic acid (HA), methylacrylated HA, hyaluronic acid-aldehyde, or bovine serum albumin (BSA); the second cell adhesive molecule is poly(allylamine) hydrochloride (PAH); and the structural polymer is one or more of: poly(lactic-co-glycolic) acid (PLGA), PLGA-PEG, PLGA-PEG-maleimide, or PLGA-PEG-biotin. In some embodiments of any of the aspects, the PLGA structural polymer is a combination of PLGA and poly (D,L-lactide-co-glycolide); a combination of PLGA and poly (D,L-lactide-co-glycolide) with acid ends; or a combination of PLGA and poly (D,L-lactide-co-glycolide) at a 50:50 molar ratio.

In some embodiments of any of the aspects, a two-layer backpack comprises a first layer of HA, and a second layer of PAH. In some embodiments of any of the aspects, a two-layer backpack comprises a first layer of HA and a second layer of PLGA hydrogel. In some embodiments of any of the aspects, a two-layer backpack comprises a first layer of BSA, and a second layer of PAH. In some embodiments of any of the aspects, a two-layer backpack comprises a first layer of BSA and a second layer of PLGA hydrogel.

A sandwich backpack comprises at least three distinct regions or layers. In an uncoated sandwich backpack, the layers are stacked and substantially parallel to each other, such that the first and third layer are not in contact with each other. Uncoated sandwich backpacks can be created by, e.g., stamping each layer consecutively. In an coated sandwich backpack, the third layer (applied last), wraps around the sides of the backpack, such that in cross-section of a backpack, its furthest edges are at least partially in contact with the edges of the first layer. In a coated sandwich backpack, the second or middle layer may be partially or entirely coated. In a coated sandwich backpack, the sides of the first or bottom layer may be partially coated or entirely coated but the face of the first or bottom layer most distal from the other layers is not coated. Coated sandwich backpacks can be created by e.g, stamping the first and second layers consecutively and then spin-coating the third layer.

In some embodiments of any of the aspects, the second region is located between the first region and third region and/or the second region separates the first region and third region from each other.

In some embodiments of any of the aspects, the polymeric particle comprises: a) a first region comprising the one or more binding reagents and one or more cell adhesive molecules (e.g., polyelectrolytes) and b) a second region comprising one or more structural polymers.

In some embodiments of any of the aspects, the polymeric particle comprises: a) a first region comprising one or more cell adhesive molecules (e.g., polyelectrolytes) and optionally the one or more binding reagents; b) a second region comprising one or more structural polymers; and c) a third region comprising one or more cell adhesive molecules (e.g., polyelectrolytes) and optionally the one or more binding reagents.

In some embodiments of any of the aspects, the polymeric particle comprises: a) a first region comprising one or more cell adhesive molecules (e.g., polyelectrolytes) and optionally the one or more binding reagents; b) a second region comprising one or more cell adhesive molecules (e.g., polyelectrolytes); and b) a third region comprising one or more structural polymers and optionally the one or more binding reagents. In some embodiments of any of the aspects, the polymeric particle comprises: a) a first region comprising one or more cell adhesive molecules (e.g., polyelectrolytes) and one or more binding reagents; b) a second region comprising one or more cell adhesive molecules (e.g., polyelectrolytes); and b) a third region comprising one or more structural polymers and one or more binding reagents. In some embodiments of any of the aspects, the polymeric particle comprises: a) a first region comprising one or more cell adhesive molecules (e.g., polyelectrolytes); b) a second region comprising one or more cell adhesive molecules (e.g., polyelectrolytes); and b) a third region comprising one or more structural polymers and one or more binding reagents. In some embodiments of any of the aspects, the polymeric particle comprises: a) a first region comprising one or more cell adhesive molecules (e.g., polyelectrolytes) and one or more binding reagents; b) a second region comprising one or more cell adhesive molecules (e.g., polyelectrolytes); and b) a third region comprising one or more structural polymers.

In some embodiments of any of the aspects, the polymeric particle comprises: a) a first region comprising a first cell adhesive molecule (e.g., polyelectrolyte); b) a second region comprising a second cell adhesive molecule (e.g., polyelectrolyte); and c) a third region comprising one or more structural polymers and optionally the one or more binding reagents. In some embodiments of any of the aspects, the polymeric particle comprises: a) a first region comprising a first cell adhesive molecule (e.g., polyelectrolyte); b) a second region comprising a second cell adhesive molecule (e.g., polyelectrolyte); and c) a third region comprising one or more structural polymers and one or more binding reagents.

In some embodiments of any of the aspects, the third region comprises one or more binding reagents and the first region does not comprise one or more binding reagents.

Where a polymeric particle comprises two or more regions/layers which each comprise a cell adhesive molecule, the regions/layers can comprise the same or different cell adhesive molecules.

In some embodiments of any of the aspects, a sandwich backpack comprises a first layer of HA, a second layer of PAH, and third layer of PLGA (e.g, the LbL HA-PAH design of Example 2). In some embodiments of any of the aspects, a sandwich backpack comprises a first layer of HA, a second layer of PAH, and third layer of PLGA hydrogel. In some embodiments of any of the aspects, a sandwich backpack comprises a first layer of BSA, a second layer of PAH, and third layer of PLGA PLGA (e.g, the LbL BSA-PAH design of Example 2). In some embodiments of any of the aspects, a sandwich backpack comprises a first layer of BSA, a second layer of PAH, and third layer of PLGA hydrogel.

In some embodiments of any of the aspects, the polymeric particle comprises a a) a first region comprising one or more cell adhesive molecules (e.g., polyelectrolytes); b) a second region comprising one or more cell adhesive molecules (e.g., polyelectrolytes); and b) a third region comprising one or more structural polymers, and the first cell adhesive molecule is hyaluronic acid (HA), methylacrylated HA, hyaluronic acid-aldehyde, or bovine serum albumin (BSA); the second cell adhesive molecule is poly(allylamine) hydrochloride (PAH); and the structural polymer is one or more of: poly(lactic-co-glycolic) acid (PLGA), PLGA-PEG, PLGA-PEG-maleimide, or PLGA-PEG-biotin.

In some embodiments of any of the aspects, the polymeric particle comprises a a) a first region comprising one or more cell adhesive molecules (e.g., polyelectrolytes) comprising streptavidin molecules; b) a second region comprising one or more cell adhesive molecules (e.g., polyelectrolytes); and b) a third region comprising one or more structural polymers, and the first cell adhesive molecule is hyaluronic acid (HA), methylacrylated HA, hyaluronic acid-aldehyde, or bovine serum albumin (BSA); the second cell adhesive molecule is poly(allylamine) hydrochloride (PAH); and the structural polymer is one or more of: poly(lactic-co-glycolic) acid (PLGA), PLGA-PEG, PLGA-PEG-maleimide, or PLGA-PEG-biotin. In some embodiments of any of the aspects, the polymeric particle comprises a a) a first region comprising one or more cell adhesive molecules (e.g., polyelectrolytes) comprising streptavidin molecules bound to biotinylated binding reagents; b) a second region comprising one or more cell adhesive molecules (e.g., polyelectrolytes); and b) a third region comprising one or more structural polymers, and the first cell adhesive molecule is hyaluronic acid (HA), methylacrylated HA, hyaluronic acid-aldehyde, or bovine serum albumin (BSA); the second cell adhesive molecule is poly(allylamine) hydrochloride (PAH); and the structural polymer is one or more of: poly(lactic-co-glycolic) acid (PLGA), PLGA-PEG, PLGA-PEG-maleimide, or PLGA-PEG-biotin. In some embodiments of any of the aspects, the PLGA structural polymer is a combination of PLGA and poly (D,L-lactide-co-glycolide); a combination of PLGA and poly (D,L-lactide-co-glycolide) with acid ends; or a combination of PLGA and poly (D,L-lactide-co-glycolide) at a 50:50 molar ratio.

In some embodiments of any of the aspects, the polymeric particle comprises a a) a first region comprising one or more cell adhesive molecules (e.g., polyelectrolytes) covalently conjugated to one or more binding reagents; b) a second region comprising one or more cell adhesive molecules (e.g., polyelectrolytes); and b) a third region comprising one or more structural polymers, and the first cell adhesive molecule is hyaluronic acid (HA), methylacrylated HA, hyaluronic acid-aldehyde, or bovine serum albumin (BSA); the second cell adhesive molecule is poly(allylamine) hydrochloride (PAH); and the structural polymer is one or more of: poly(lactic-co-glycolic) acid (PLGA), PLGA-PEG, PLGA-PEG-maleimide, or PLGA-PEG-biotin. In some embodiments of any of the aspects, the PLGA structural polymer is a combination of PLGA and poly (D,L-lactide-co-glycolide); a combination of PLGA and poly (D,L-lactide-co-glycolide) with acid ends; or a combination of PLGA and poly (D,L-lactide-co-glycolide) at a 50:50 molar ratio.

In some embodiments of any of the aspects, the polymeric particle comprises: a) a first region comprising one or more structural polymers and optionally the one or more binding reagents; b) a second region comprising one or more structural polymers; and c) a third region comprising one or more structural polymers and optionally the one or more binding reagents. In some embodiments of any of the aspects, the polymeric particle comprises: a) a first region comprising PLGA and optionally the one or more binding reagents; b) a second region comprising PVA; and c) a third region comprising PLGA and optionally the one or more binding reagents.

In some embodiments of any of the aspects, the second region comprises PVA. In some embodiments of any of the aspects, the second region consists essentially of PVA and a payload reagent(s). In some embodiments of any of the aspects, the PVA is present at a concentration of less than 1% by weight. In some embodiments of any of the aspects, the PVA is present at a concentration of 0.5% or less by weight.

In some embodiments of any of the aspects, the first selection of one or more structural polymers comprises PLGA, the second selection of one or more structural polymers comprises PVA; and the third selection of one or more structural polymers comprises PLGA. In some embodiments of any of the aspects, the first selection of one or more structural polymers consists of PLGA, the second selection of one or more structural polymers consists of PVA; and the third selection of one or more structural polymers consists of PLGA.

In some embodiments of any of the aspects, a region comprising a structural polymer is, or is formed using, an about 5-20 wt. % solution of the structural polymer. In some embodiments of any of the aspects, a region comprising a structural polymer is, or is formed using, a 5-20 wt. % solution of the structural polymer. In some embodiments of any of the aspects, a region comprising a structural polymer is, or is formed using, an about 1-20 wt. % solution of the structural polymer. In some embodiments of any of the aspects, a region comprising a structural polymer is, or is formed using, a 1-20 wt. % solution of the structural polymer. In some embodiments of any of the aspects, a region comprising a structural polymer is, or is formed using, an about 8-12 wt. % solution of the structural polymer. In some embodiments of any of the aspects, a region comprising a structural polymer is, or is formed using, an 8-12 wt. % solution of the structural polymer.

In some embodiments of any of the aspects, a region comprising a structural polymer is, or is formed using, an about 10 wt. % solution of the structural polymer. In some embodiments of any of the aspects, a region comprising a structural polymer is, or is formed using, a 10 wt. % solution of the structural polymer.

Discoidal particles displayed favorable characteristics, e.g., for being retained on the cell surface without altering the cell's behavior. In some embodiments of any of the aspects, the polymeric particle is substantially discoidal in shape. In some embodiments of any of the aspects, the polymeric particle is discoidal in shape. As used herein, “discoidal” refers to a particle having a disk-like shape, with substantially flat, concave or convex faces.

In some embodiments of any of the aspects, a polymeric particle described herein has a disk-like shape, wherein the diameter of the circular face(s) is from about 4× to about 35× the size of the height (e.g., or depth) of the particle. In some embodiments of any of the aspects, a polymeric particle as described herein has a disk-like shape, wherein the diameter of the circular face(s) is from 4× to 35× the size of the height of the particle. In some embodiments of any of the aspects, a polymeric particle as described herein has a disk-like shape, wherein the diameter of the circular face(s) is from about 10× to about 35× the size of the height (e.g., or depth) of the particle. In some embodiments of any of the aspects, a polymeric particle as described herein has a disk-like shape, wherein the diameter of the circular face(s) is from 10× to 35× the size of the height of the particle. In some embodiments of any of the aspects, a polymeric particle as described herein has a disk-like shape, wherein the diameter of the circular face(s) is from about 18× to about 26× the size of the height of the particle. In some embodiments of any of the aspects, a polymeric particle as described herein has a disk-like shape, wherein the diameter of the circular face(s) is from 18× to 26× the size of the height of the particle.

In some embodiments of any of the aspects, a substantially discoidal particle has two substantially opposing and circular faces and the diameter of each face is at least 10× the height (e.g., depth) of the particle. In some embodiments of any of the aspects, a substantially circular face's widest diameter is no more than 150% of the shortest diameter of that face.

In some embodiments of any of the aspects, the polymeric particle has a shape which is a rod, a cylinder, a cube, cuboid, hexahedron, or pyramid.

In some embodiments of any of the aspects, the diameter of the polymeric particle is from about 50 nm to about 20 μm. In some embodiments of any of the aspects, the diameter of the polymeric particle is from 50 nm to 20 μm. In some embodiments of any of the aspects, the diameter of the polymeric particle is from about 100 nm to about 10 μm. In some embodiments of any of the aspects, the diameter of the polymeric particle is from 100 nm to 10 μm. In some embodiments of any of the aspects, the diameter of the polymeric particle is from about 1 μm to about 10 μm. In some embodiments of any of the aspects, the diameter of the polymeric particle is from 1 μm to 10 μm.

In some embodiments of any of the aspects, the polymeric particle is about 3 μm×150 nm in size to about 12 μm×500 nm in size. In some embodiments of any of the aspects, the polymeric particle is 3 μm×150 nm in size to 12 μm×500 nm in size. In some embodiments of any of the aspects, the polymeric particle is about 6 μm×500 nm in size. In some embodiments of any of the aspects, the polymeric particle is 6 μm×500 nm in size. In some embodiments of any of the aspects, the polymeric particle is about 6 μm×250 nm in size. In some embodiments of any of the aspects, the polymeric particle is 6 μm×250 nm in size.

In some embodiments of any of the aspects, the polymeric particle is about 0.5-5 μm×5-15 μm in size. In some embodiments of any of the aspects, the polymeric particle is 0.5-5 μm×5-15 μm in size. In some embodiments of any of the aspects, the polymeric particle is about 1-2 μm×7-9 μm in size. In some embodiments of any of the aspects, the polymeric particle is 1-2 μm×7-9 μm in size. In some embodiments of any of the aspects, the polymeric particle is about 1.5 μm×8 μm in size.

In some embodiments of any of the aspects, a region can be a layer. In some embodiments of any of the aspects, a region can be a face of the discoidal shape of the particle. In some embodiments of any of the aspects, a region can be the interior space (or a portion thereof) of the discoidal shape of the particle.

The binding reagents described herein can be attached or conjugated to one or more components of a polymeric particle by any method known in the art, e.g, by direct chemical bonding or via a linker molecule. The term “conjugation,” refers to two or more molecular structures that are linked by a direct or indirect covalent or non-covalent bond. Non-covalent interactions include, but are not limited to, electrostatic interactions, hydrogen bonding interactions, van der Waals interactions, dipole-dipole interactions, π-πstacking, magnetic interactions, and metal coordination. Preferably, the conjugation is via covalent bonds.

The linkers may be homo-bifunctional or hetero-bifunctional. In some instances, combinations of homo-bifunctional linkers and hetero-bifunctional linkers are used. Examples of homo-bifunctional linkers include, but are not limited to adipic acid dihydrazide, amino acids such as glycine, aldehydes such as ethanedial, pyruvaldehyde, 2-formyl-malonaldehyde, glutaraldehyde, adipaldehyde, heptanedial, octanedial; di-glycidyl ether, diols such as 1,2-ethanediol, 1,3-propanediol, 1,4-butanediol, 2,3-butanediol, 1,5-pentanediol, benzene-1,4-diol, 1,6-hexanediol, tetra(ethylene glycol)diol), PEG, di-thiols such as 1,2-ethanedithiol, 1,3-propanedithiol, 1,4-butanedithiol, 2,3-butanedithiol, 1,5-pentanedithiol, benzene-1,4-dithiol, 1,6-hexanedithiol, tetra(ethylene glycol)dithiol), di-amine such as ethylene diamine, propane-1,2-diamine, propane-1,3-diamine, N-methylethylenediamine, N,N′-dimethylethylenediamine, pentane-1,5-diamine, hexane-1,6-diamine, spermine and spermidine, divinyladipate, divinylsebacate, diamine-terminated PEG, double-ester PEG-N-hydroxysuccinimide, and di-isocyanate-terminated PEG. In a preferred embodiment, the homo-bifunctional linker is adipic acid dihydrazide. Examples of hetero-bifunctional linkers include, but are not limited to, epichlorohydrin, S-acetylthioglycolic acid N-hydroxysuccinimide ester, 5-azido-2-nitrobenzoic acid N-hydroxysuccinimide ester, 4-azidophenacyl bromide, bromoacetic acid N-hydroxysuccinimide ester, N-(3-dimethylaminopropyl)-N′-ethylcarbodiimide, Iodoacetic acid N-hydroxysuccinimide ester, 4-(N-mMaleimido)benzophenone 3-(2-pyridyldithio)propionic acid N-hydroxysuccinimide ester 3-maleimidobenzoic acid N-hydroxysuccinimide ester, N,N′-cystamine-bis-acrylamide, N,N′-methylene-bis-acrylamide and N,N′-ethylene-bis-acrylamide.

One method of attaching or conjugating a binding reagent is via streptavidin-biotin linkage. In some embodiments of any of the aspects, the binding reagent further comprises a streptavidin molecule. In some embodiments of any of the aspects, the binding reagent further comprises a streptavidin molecule and one or more other components of the polymeric particle comprise a biotin molecule. In some embodiments of any of the aspects, the binding reagent further comprises a biotin molecule. In some embodiments of any of the aspects, the binding reagent further comprises a biotin molecule and one or more other components of the polymeric particle comprise a streptavidin molecule.

In some embodiments of any of the aspects, the component of the polymeric particle comprising biotin and/or streptavidin can be one or more structural polymers. In some embodiments of any of the aspects, the component of the polymeric particle comprising biotin can be one or more structural polymers. In some embodiments of any of the aspects, the structural polymer of the polymeric particle comprising biotin and/or streptavidin can be PLGA-PEG. In some embodiments of any of the aspects, the structural polymer of the polymeric particle comprising biotin can be PLGA-PEG-biotin. In some embodiments of any of the aspects, the polymeric particle comprises a binding reagent bound to a structural polymer via a streptavidin-biotin linkage. In some embodiments of any of the aspects, the polymeric particle comprises a binding reagent bound to PLGA-PEG via a streptavidin-biotin linkage.

One method of attaching or conjugating a binding reagent is via a linkage to maleimide via a DTT catalyzed-reaction. In some embodiments of any of the aspects, the binding reagent further comprises a maleimide molecule. In some embodiments of any of the aspects, one or more other components of the polymeric particle other than a binding reagent comprise a maleimide molecule. In some embodiments of any of the aspects, the component of the polymeric particle comprising a maleimide molecule can be one or more structural polymers. In some embodiments of any of the aspects, the structural polymer of the polymeric particle comprising malemide can be PLGA-PEG. In some embodiments of any of the aspects, the polymeric particle comprises a binding reagent bound to a structural polymer comprising a malemide molecule via DTT catalyzed-reaction. In some embodiments of any of the aspects, the polymeric particle comprises a binding reagent bound to PLGA-PEG-maleimide via DTT catalyzed-reaction.

In some embodiments of any of the aspects, a polymeric particle described herein can further comprise one or more imaging agents. As used herein “imaging agent” refers to refers to an element, functional group, or molecule that allows for its detection and/or imaging. The imaging agent can be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber. In some embodiments of any of the aspects, the imaging agent can be a contrast agent. As used herein the term “contrast agent” refers to any molecule that changes the optical properties of tissue or organ containing the molecule. Optical properties that can be changed include, but are not limited to, absorbance, reflectance, fluorescence, birefringence, optical scattering and the like. In some embodiments, the detectable labels also encompass any imaging agent (e.g., but not limited to, a bubble, a liposome, a sphere, a contrast agent, or any detectable label described herein) that can facilitate imaging or visualization of a tissue or an organ in a subject.

Suitable optical reporters include, but are not limited to, fluorescent reporters and chemiluminescent groups. A wide variety of fluorescent reporter dyes are known in the art. Typically, the fluorophore is an aromatic or heteroaromatic compound and can be a pyrene, anthracene, naphthalene, acridine, stilbene, indole, benzoindole, oxazole, thiazole, benzothiazole, cyanine, carbocyanine, salicylate, anthranilate, coumarin, fluorescein, rhodamine or other like compound.

Exemplary fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP -Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine 0; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodaminelsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Many suitable forms of these fluorescent compounds are available and can be used.

Other exemplary detectable labels include luminescent and bioluminescent markers (e.g., biotin, luciferase (e.g., bacterial, firefly, click beetle and the like), luciferin, and aequorin), radiolabels (e.g., 3H, 125I, 35S, 14C, or 32P), enzymes (e.g., galactosidases, glucorinidases, phosphatases (e.g., alkaline phosphatase), peroxidases (e.g., horseradish peroxidase), and cholinesterases), and calorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference.

Suitable echogenic gases include, but are not limited to, a sulfur hexafluoride or perfluorocarbon gas, such as perfluoromethane, perfluoroethane, perfluoropropane, perfluorobutane, perfluorocyclobutane, perfluropentane, or perfluorohexane. Suitable non-metallic isotopes include, but are not limited to, ¹C, 1⁴C, 1³N, 1⁸F, ¹²³I, ¹²⁴I, and ¹²⁵I. Suitable radioisotopes include, but are not limited to, ⁹⁹mTc, ⁹⁵Tc, ¹¹¹In, ⁶²Cu, ⁶⁴Cu, Ga, ⁶⁸Ga, and ¹⁵³Gd. Suitable paramagnetic metal ions include, but are not limited to, Gd(III), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir.

In some embodiments, the radionuclide is bound to a chelating agent or chelating agent-linker attached to the heme-binding molecule and/or composition. Suitable radionuclides for direct conjugation include, without limitation, ¹⁸F, ¹²⁴I, ¹²⁵I, ¹³¹I, and mixtures thereof Suitable radionuclides for use with a chelating agent include, without limitation, ⁴⁷Sc, ⁶⁴Cu, ⁶⁷Cu, ⁸⁹Sr, ⁸⁶Y ⁸⁷Y ⁹⁰Y 105hR ¹¹¹Ag, ¹¹¹In, ¹¹⁷mSn, ¹⁴⁹Pm, ¹⁵³Sm, ¹⁶⁶Ho, ⁷⁷Lu, ¹⁸⁶Re, ¹⁸⁸Re, ²¹¹At, ²¹²Bi, and mixtures thereof. Suitable chelating agents include, but are not limited to, DOTA, BAD, TETA, DTPA, EDTA, NTA, HDTA, their phosphonate analogs, and mixtures thereof. One of skill in the art will be familiar with methods for attaching radionuclides, chelating agents, and chelating agent-linkers to molecules such as the heme-binding molecule and/or composition and carrier scaffolds disclosed herein.

Means of detecting such labels are well known to those of skill in the art. Thus, for example, radiolabels can be detected using photographic film or scintillation counters, fluorescent markers can be detected using a photo-detector to detect emitted light. Enzymatic labels are typically detected by providing the enzyme with an enzyme substrate and detecting the reaction product produced by the action of the enzyme on the enzyme substrate, and calorimetric labels can be detected by visualizing the colored label. Exemplary methods for in vivo detection or imaging of detectable labels include, but are not limied to, radiography, magnetic resonance imaging (MRI), Positron emission tomography (PET), Single-photon emission computed tomography (SPECT, or less commonly, SPET), Scintigraphy, ultrasound, CAT scan, photoacoustic imaging, thermography, linear tomography, poly tomography, zonography, orthopantomography (OPT or OPG), and computed Tomography (CT) or Computed Axial Tomography (CAT scan).

Imaging agents can include one or more contrast agents, one or more MRI contrast agents, one or more microbubbles, one or more metal ions, one or more radioactive isotopes, one or more optical imaging agents, one or more SPECT imaging agents, and one or more PET imaging agents.

Targeted delivery of MRI contrast reagents has proven difficult to address, as conjugation, encapsulation, or other means for attaching or associating contrast agents to carriers or targeting moieties often removes or reduces their ability to be detected by MRI. In particular, MRI contrast reagents, particularly gadolinium-based reagents must remain in contact with water to function as MRI contrast reagents, but simultaneously must be attached with the carrier or targeting moietiy. It is demonstrated herein that attachment of MRI contrast reagents to the instant polymeric particles can avoid such deleterious effects. Accordingly, in one aspect of the embodiments, provided herein is a polymeric particle comprising at least one MRI contrast reagent.

In some embodiments of any of the aspects, described herein is a polymeric particle that comprises, consists of, or consists essentially of a single polymer region or layer, the region or layer comprising a) a hydrogel of one or more one or more cell adhesive molecules, b) one or more binding reagents, c) and one or more MRI contrast reagents. In some embodiments of any of the aspects, described herein is a polymeric particle that comprises, consists of, or consists essentially of a single polymer region or layer, the region or layer comprising a) a hydrogel of one or more one or more cell adhesive molecules, and b) one or more MRI contrast reagents. In some embodiments of any of the aspects, the one or more cell adhesive molecules comprise, consist essentially of, or consist of: hyaluronic acid (HA), methylacrylated HA, PEG, PEG dimethylacrylate, and combinations thereof. In some embodiments of any of the aspects, the one or more cell adhesive molecules comprise, consist essentially of, or consist of: methylacrylated HA and PEG dimethylacrylate. In some embodiments of any of the aspects, described herein is a polymeric particle that comprises, consists of, or consists essentially of a single polymer region or layer, the region or layer comprising a) a hydrogel of methylacrylated HA and PEG dimethylacrylate, and b) one or more methacrylated MRI contrast reagents.

MRI contrast agents can comprise one or more of a gadoliunium-based contrast agent, superparamagnetic iron oxide, ultrasmall superparamagnetic iron oxide, superparamagnetic iron-platinum, manganese chelates, iron salts, and perflubron. In some embodiments of any of the aspects, a gadoliunium-based contrast agent comprises one or more of gadolinium, gadoxetate, gadobutrol, gadoterate, gadoteridol, gadopentetate, gadobenate, gadopentetic acid dimegulmine, gadoxentate, gadoversetamide, gadodiamide, gadofosveset, gadocoletic acid, gadomelitol, gadomer 17, and gadoxetic acid.

Imaging agents can be conjugated or attached to a polymeric particle described herein by any of the methods described herein, e.g., in relation to conjugation or attachment of binding agent. In some embodiments of any of the aspects, the one or more imaging agents further comprise methylacrylate. In some embodiments of any of the aspects, one or more imaging agents are methylacrylate cross-linked to or more of methylacrylated HA and PEG dimethylacrylate.

In some embodiments of any of the aspects, a polymeric particle described herein can further comprise at least one polarizing agent, e.g., wherein the particle is located on the cell surface of the monocyte or macrophage. In some embodiments of any of the aspects, the cell is a monocyte cell. In some embodiments of any of the aspects the cell is a monocyte cell at the time the polymeric particle is adhered to the cell (e.g., the cell, either under the influence of the particle, or independently thereof, may differentiate to a macrophage after adherence). In some embodiments of any of the aspects, the cell is a macrophage cell, e.g., an M0, M1, M2, M1-polarized, or M2-polarized macrophage.

The presence of the polymeric particle on the surface of the cell can, by contacting the cell with the polarizing agent, direct or regulate the phenotype of the cell, e.g., increase the likelihood, duration, magnitude, or rate of development of N1/M1 or N2/M2 phenotypic characteristics. In some embodiments of any of the aspects, the macrophage is substantially driven to an N1/M1 or N2/M2 phenotype by adherence of the polymeric particle. In some embodiments of any of the aspects, the phenotype of the macrophage is regulated by the release of the one or more polarizing agent from the polymeric particle, e.g., induced or non-induced release of the cytokine and/or induced or non-induced degradation of the polymeric particle.

An M1 or M1-polarized macrophage, also referred to as “killer” macrophages, promote inflammation and have anti-tumor activity. They secrete high levels of IL-12 and low levels of IL-10. M1 macrophages can be characterized by the expression of, e.g., CCL3, CCL5, CD80, CCR7, iNOS and INF-γ. An M2 or M2-polarized macrophage, also referred to as a “repair” macrophage, contributes to wound healing and tissue repair. M2 macrophages can suppress the immune system and/or inflammation, e.g., by producing high levels of IL-10. An M2-polarized macrophage can be characterized by the expression of, e.g., CCL22, CD206, CD163, YM1, Fizz1, and arginase 1. Similar phenotypes (N1 and N2) are known for neutrophils.

As described herein, a “polarizing agent” is an agent, that when contacted with a macrophage and/or monocyte, alters the likelihood, persistence, magnitude, or rate of development of a particular macrophage phenotype (e.g., either N1/M1 or N2/M2 phenotype) as compared to the absence of the polarizing agent. A polarizing agent can be an N1/M1-polarizing agent, e.g., it increases the likelihood, persistence, or rate of development of an N1/M1 phenotype, or an N2/M2-polarizing agent, e.g., it increases the likelihood, persistence, or rate of development of an N2/M2 phenotype. Exemplary N1/M1 and N2/M2 phenotypes are described herein and are well known in the art. Further details can be found, e.g., in Mills et al. “M1/M2 macrophages” Frontiers Media SA (2015) and Kloc “Macrophages: Origin, Function, and Biointervention” Spring (2017); each of which are incorporated by reference herein in their entireties.

Polarizing agents for the N1/M1 and N2/M2 macrophage phenotypes are known in the art, and can include, by way of non-limiting example, the N1/M1-polarizing Toll-like receptor (TLR) agonists (e.g., LPS, muramyl dipeptide, or lipoteichoic acid); the N1/M1-polarizing cytokines IFN-γ (e.g., NCBI Gene ID: 3458); TNF (e.g., NCBI Gene ID: 7124); IL-12 (e.g., NCBI Gene ID: 3592 and 3593); GM-CSF (eg., NCBI Gene ID: 1438); IL-1β (e.g., NCBI Gene ID: 3553); IL-6 (e.g., NCBI Gene ID: 3569); CDiib (e.g., NCBI Gene ID: 3684) and IL-23 (e.g., NCBI Gene ID: 51561) and the N2/M2-polarizing cytokines IL-4 (e.g., NCBI Gene ID: 3565); IL-10 (e.g., NCBI Gene ID: 3586); glucocortoids (e.g., cortisol, cortisone, prednisone, prednisolone, methylprednisonolone, dexamethasone, betamethasone, triamcinolone, fludrocortisone acetate, and deoxycorticosterone acetate); M-CSF (e.g., NCBI Gene ID: 1435), TGF-beta (e.g. NCBI Gene ID: 7040); IL-6 (e.g., NCBI Gene ID: 3569); and IL-13 (e.g., NCBI Gene ID: 3596). TLR agonists are known in the art and can include, by way of non-limiting example LPS, dsRNA; flagella; bacterial lipoprotein; ssRNA; cpG DNA; bacterial peptidoglycans; profillin; rRNA; imiquimod; resiquimod; IMO-2055; picibanil; monophsophoryl lipid A (MPL); polyribocytidylic acid (polyL:C); CpG-28; MGN1703; glucopyranosyl lipid A; entolimod; and ODN2006. Further details on TLR agonists can be found, e.g., in Kaczanowska et al. 2013 J Leukoc. Biol. 93:847-863; which is incorporated by reference herein in its entirety. TLR agonists are also available commercially, e.g., TLRi-9 Agonist Kit (Cat. No. tlrl-kitlhw; Invitrogen; San Diego, CA).

The polarizing agent(s) can be present in the first region, the second region, a third region forming layer in between the first and second regions, in the interior space of the polymeric particle, or any combination thereof. In some embodiments of any of the aspects, the third region can comprise a different structural polymer or mixture of structural polymers than the first and second regions.

Placement of the polarizing agent can be influenced by whether the polarizing effect should be exerted immediately following adherence of the particle, or if it is desired to induce the polarizing effect by controlled degradation of the polymeric particle as described below herein. In some embodiments of any of the aspects, the polarizing agent can be present in the first region. In some embodiments of any of the aspects, the first region comprises the polarizing agent. In some embodiments of any of the aspects, only the first region comprises the polarizing agent.

In one aspect of any of the embodiments, described herein is a method of providing or preparing an N1 neutrophil and/or M1 macrophage, the method comprising contacting a neutrophil and/or macrophage with a polymeric particle as described herein. In some embodiments of any of the aspects, described herein is a method of inducing a N1/M1 immune response in a subject, comprising administering to the subject an engineered cellular composition comprising a neutrophil or macrophage and a polymeric particle. In some embodiments of any of the aspects, the polymeric particle does not comprise a polarizing agent. In some embodiments of any of the aspects, the polymeric particle comprises a N1/M1 polarizing agent.

In one aspect of any of the embodiments, described herein is a method of providing or preparing an N2 neutrophil and/or M2 macrophage, the method comprising contacting a neutrophil and/or macrophage with a polymeric particle comprising a N2/M2 polarizing agent. In some embodiments of any of the aspects, described herein is a method of inducing a N2/M2 immune response in a subject, comprising administering to the subject an engineered cellular composition comprising a neutrophil or macrophage and a polymeric particle comprising a N2/M2 polarizing agent.

Embodiments of the particles described herein can be controllably-degraded, e.g., either to control delivery of a payload (e.g., temporally or spatially) or to regulate the effect of the particle on the carrier cell (e.g., the monocyte or macrophage). One approach to such controllable-degradation is to utilize particles in which a region comprises degradable polymers or polymer linkers. In some embodiments of any of the aspects, the region comprising degradable polymers or polymer linkers is a second or third region.

In some embodiments of any of the aspects, the degradable polymers or polymer linkers is present in admixture with the structural polymer. In some embodiments of any of the aspects, the degradable polymers or polymer linkers is present in a second region of the polymeric particle which is located between the first and third regions, e.g., as a layer between the first and third regions, or in the interior space of the particle.

In some embodiments of any of the aspects, the degradable polymers or polymer linkers comprise or further comprise a near-infrared degradable polymer or near-infrared degradable polymer linker. Non-limiting examples of such near-infrared degradable materials can include those comprising quinone-methide light-sensitive groups, which are described in more detail in Fomina et al. J. Am. Chem. Soc. 132:9540-9542; which is incorporated by reference herein in its entirety.

In some embodiments of any of the aspects, the polymeric particle further comprises one or more cell-targeting ligands. The ligands can be located in the first region, the second region, the third region, or in more than one regions. In some embodiments of any of the aspects, the polymeric particle further comprises one or more further cell-targeting ligands. In some embodiments of any of the aspects, the polymeric particle further comprises one or more cell-targeting ligands in the first region. Such targeting ligands can also act as polarizing agents, or may have no effect on the phenotype of the cell, functioning only to increase binding affinity and/or specificity of the particle.

The particles described herein can comprise payload reagents, e.g., therapeutic molecules (e.g., a chemotherapeutic molecule or anti-inflammatory molecule), imaging molecules, or the like. The payload reagent can act on the monocyte or macrophage, or on a second cell/cell type. Payload reagents can be any type of agent. In some embodiments of any of the aspects, the payload reagent is a small molecule or polypeptide.

In some embodiments of any of the aspects, the composition comprises a further active agent or ingredient, e.g., a drug, e.g., a drug for a condition or disease. As used herein, an “active compound” or “active agent” is any agent which will exert an effect on a target cell or organism. The terms “compound” and “agent” refer to any entity which is normally not present or not present at the levels being administered and/or provided to a cell, tissue or subject. An agent can be selected from a group comprising: chemicals; small organic or inorganic molecules; signaling molecules; nucleic acid sequences; nucleic acid analogues; proteins; peptides; enzymes; aptamers; peptidomimetic, peptide derivative, peptide analogs, antibodies; intrabodies; biological macromolecules, extracts made from biological materials such as bacteria, plants, fungi, or animal cells or tissues; naturally occurring or synthetic compositions or functional fragments thereof. In some embodiments, the agent is any chemical, entity or moiety, including without limitation synthetic and naturally-occurring non-proteinaceous entities. Agents can be known to have a desired activity and/or property, or can be selected from a library of diverse compounds.

In some embodiments of any of the aspects, the first region comprises one or more payload agents. In some embodiments of any of the aspects, the second region comprises one or more payload agents. In some embodiments of any of the aspects, the third region comprises one or more payload agents. In some embodiments of any of the aspects, the second region and/or the third region comprises one or more payload agents. In some embodiments of any of the aspects, the second region and/or third region each comprises one or more payload agents.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

In some embodiments of any of the aspects, the payload reagent is a therapeutic compound or drug. In some embodiments of any of the aspects, the active compound can be a therapeutic compound or drug, e.g., an agent or compound which is therapeutically effective for the treatment of at least one condition in a subject. Therapeutic compounds are known in the art for a variety of conditions, see, e.g., the database available on the world wide web at drugs.com or the catalog of FDA-approved compounds available on the world wide web at catalog.data.gov/dataset/drugsfda-database; each of which is incorporated by reference herein in its entirety.

In some embodiments of any of the aspects, the payload reagent is present in admixture with the structural polymer. In some embodiments of any of the aspects, the payload reagent is present in a second region of the polymeric particle which is located between the first and third regions, e.g., as a layer between the first and third regions, or in the interior space of the particle.

In some embodiments of any of the aspects, the one or more paylod agents are N2/M2-polarizing agents. In some embodiments of any of the aspects, the one or more payload agents are IL-4 and/or dexamethasone. In some embodiments of any of the aspects, the one or more payload agents are N2/M2-polarizing agents. In some embodiments of any of the aspects, the second region comprises IL-4 and the third region comprises dexamethasone. In some embodiments of any of the aspects, the second region comprises IL-4 and heparin and the third region comprises dexamethasone.

In some embodiments of any of the aspects, the one or more payload agents are N1/M1-polarizing agents.

As described herein, certain embodiments of the particles described herein can be disrupted or degraded in a controllable and/or inducible manner. The particles can also be localizable. One approach to providing such functionality is to incorporate into the particle a liposome or nanoparticle that can be disrupted or removed by a controllable external stimulus. For example, echogenic liposomes are known in the art and can be disrupted by certain frequency of sound, e.g., ultrasound waves. For further details, see, e.g., Paul et al. 2014 Comput. Mech. 53(3) 413-435; Immordino et al. 2006 Int. J. Nanomedicine 1(3):294-315; Nahire et al. 2014 Mol. Pharmaceutics 11(11):4059-4068; U.S. Pat. No. 6,261,537; and US Patent Publication 2001/0051131; each of which is incorporated by reference herein in its entirety. Magnetic and gold nanoparticles are responsive to magnetic and electromagnetic fields respectively, and this functionality can be used to localize the particles, localize the cells they are adhered to, and/or to disrupt the particles. Further details of such nanoparticles and their use in such methods can be found, e.g., in Thanh “Magnetic Nanoparticles” 2012 CRC Press; Khan et al. 2015 Curr. Drug Metab. 16:685-704; Yeh et al. 2012 Nanoscale 6; Sengani et al. 2017 OpenNano 2:37-46; and Menon et al. 2017 Resource-Efficient Technologies 3:516-527; each of which is incorporated by reference herein its entirety. Further discussion of the foregoing exemplary embodiments and other means of controlled release are described in detail in Mishra “Handbook of Encapsulation and Controlled Release” CRC Press (2015); which is incorporated by reference herein in its entirety. In some embodiments of any of the aspects, foregoing liposomes and/or nanoparticles can be in the second region of the polymeric particle.

In some embodiments of any of the aspects, the release of one or more of the polarizing agents is triggered by contacting the particle with a small molecule or nucleic acid. Exemplary, non-limiting examples of such methods and reagents include particles comprising phenylboronic acid (PBA), which will release cargo in response to insulin. Further details of this approach are described in Shiino et al. Biomaterials 15:121-128 (1994); which is incorporated by reference herein in its entirety.

In one aspect of any of the embodiments, described herein is an engineered cellular composition comprising: a cell; and a polymeric particle as described herein, wherein the particle is located on the cell surface of the cell. In some embodiments of any of the aspects, the cell can be a monocyte, macrophage, natural killer cell, T cell, or neutrophil. In some embodiments ofany of the aspects, the composition further comprises media or serum.

In some embodiments of any of the aspects, the polymeric particle comprises a binding reagent selected from Table 3 and the cell is of a corresponding type indicated in Table 3. In some embodiments of any of the aspects, the polymeric particles comprises a binding reagent selected from Table 4 and the cell is of a corresponding type indicated in Table 4. In some embodiments of any of the aspects, the polymeric particles comprises a binding reagent selected from Table 4, attached to the backpack by the linkage indicated in Table 4, and the cell is of a corresponding type indicated in Table. 4.

TABLE 3

| Binding Reagent Target | Cell Type |
|---|---|
| CD11b | Neutrophils |
| CD11b | Neutrophils, Monocyte, |
| | macrophage, NK cells |
| CD11a | T cells, NK cells |
| CD3 | T cell |
| CD19 | B Cells |
| CD27 | T cells |
| CD44 | T cell |
| CD45 | Neutrophil, T cell, |
| | monocyte, NK cell, |
| CD49b | T cell, NK cells |
| CD56 | natural killer cell, |
| | T cell |
| NKG2D | NK cells, |
| NKp30 | NK cells |
| NKp46 | NK cells |
| ICAM1 | NK cells |

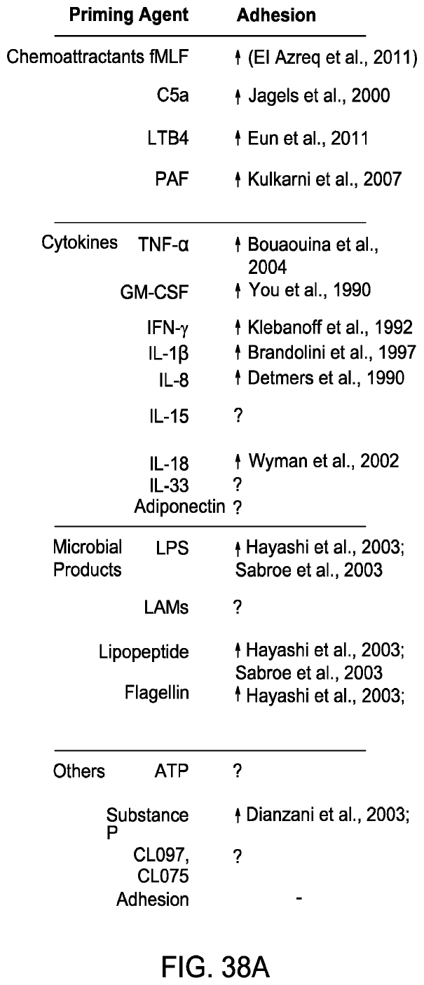

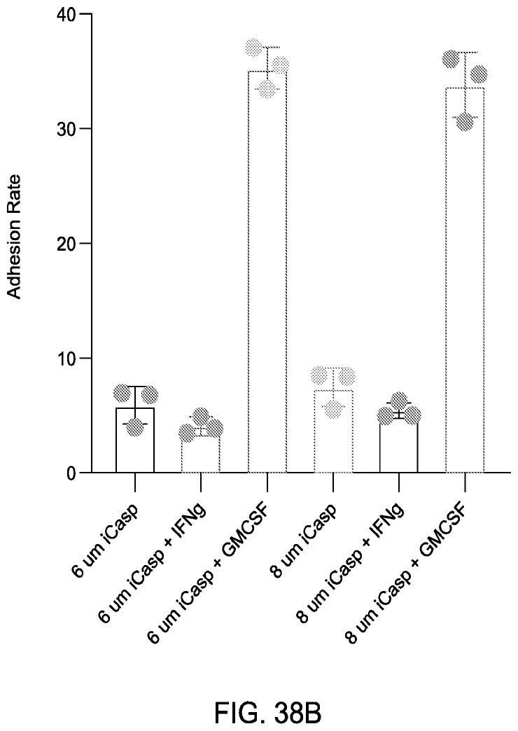

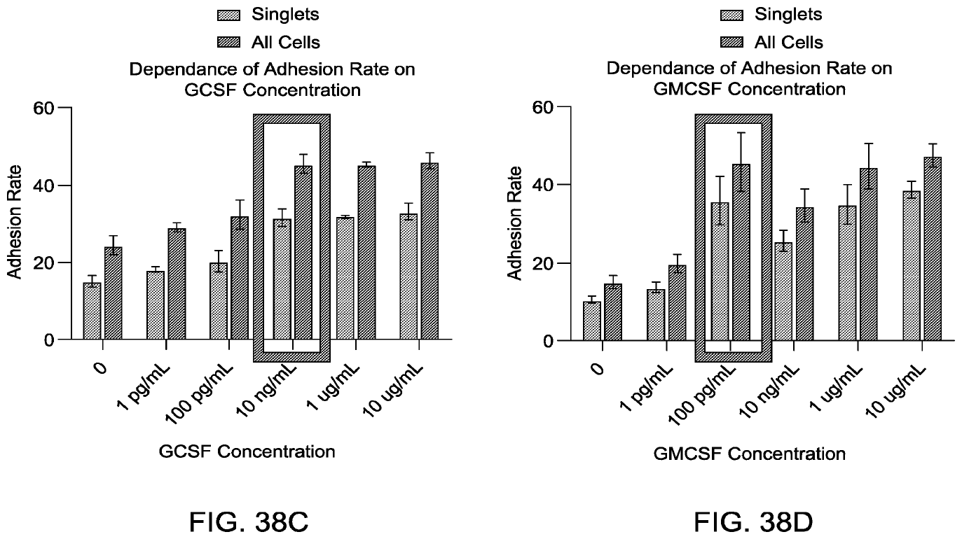

In some embodiments of any of the aspects, the polymeric particle described herein further comprises at least one priming agent. In some embodiments of any of the aspects, the methods described herein further comprise contacting a cell with at least one primary agent before, during, or after contacting the cell with a polymeric particle as described herein. As used herein, “priming agent” refers to an agent that induces attachment and/or increased viability, e.g., of neutrophils. Such agents are known in the art, e.g., as demonstrated by the references listed in FIG. 38A, each of which is incorporated by reference herein in its entirety.

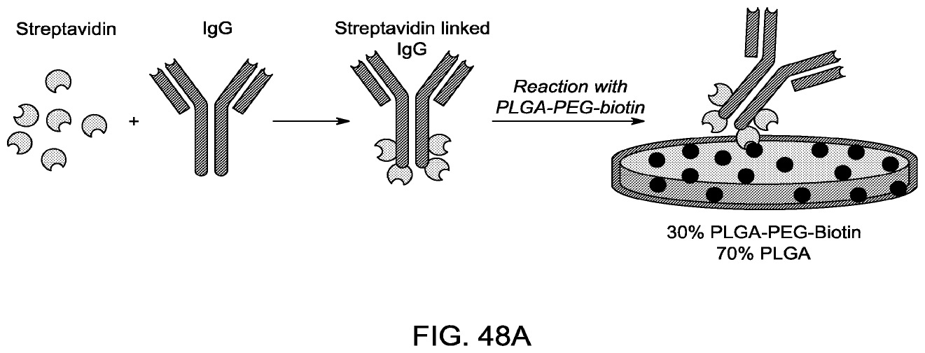

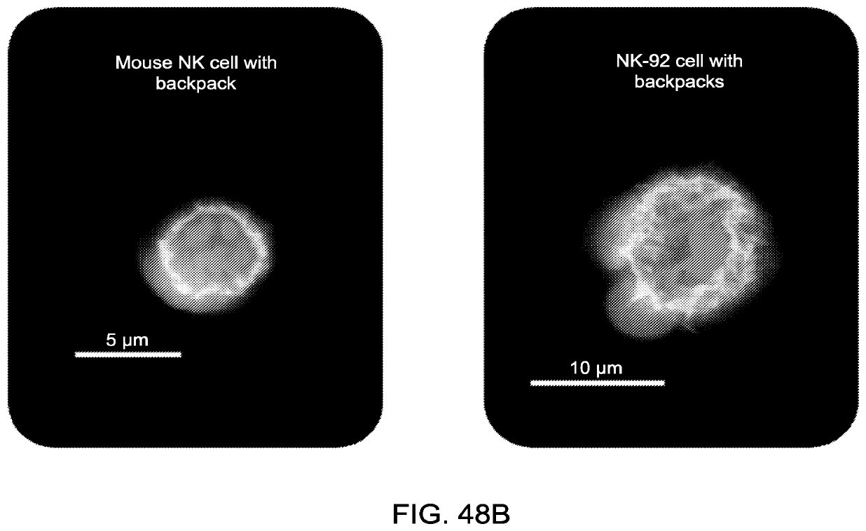

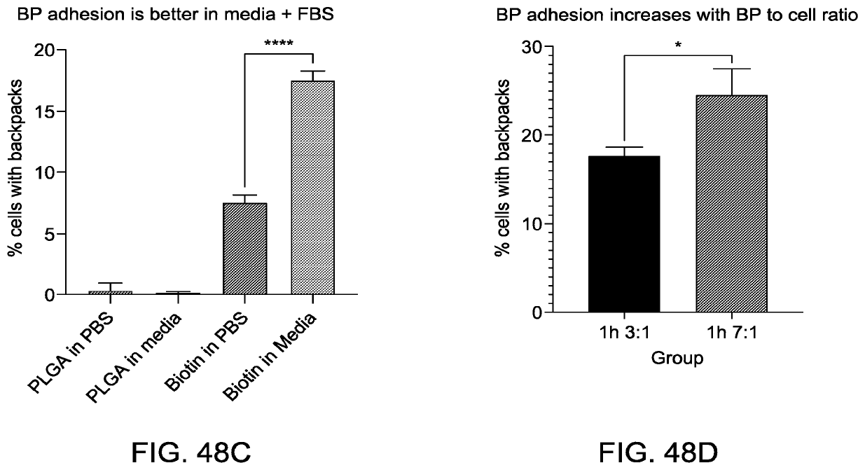

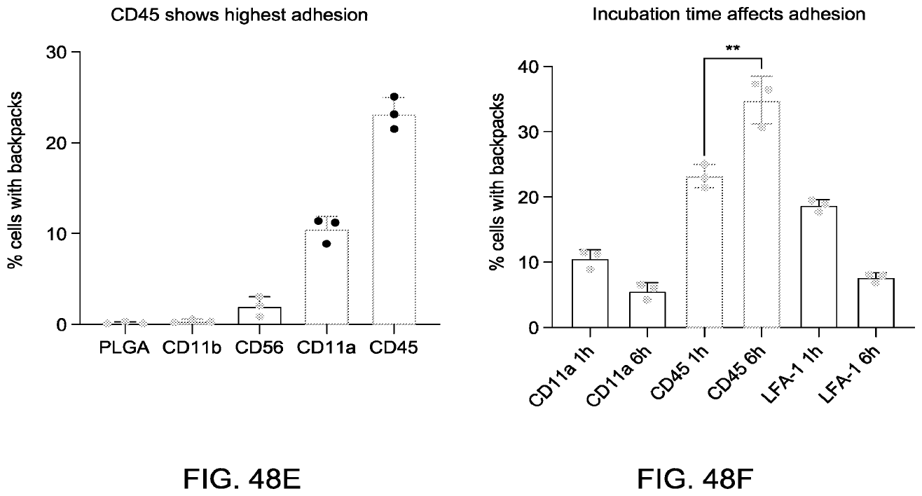

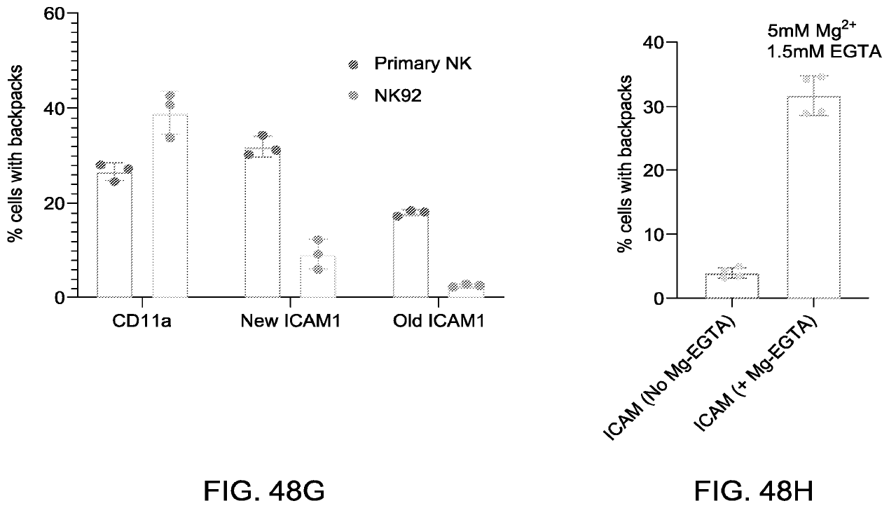

Non-limiting examples of priming agents can include a caspase inhibitor; fMLF; C5A; LTB4; PAF; TNF-α; GCSF; GM-CSF; IFN-γ; IL-1β; IL8; IL-15; IL-18; IL-33; adiponectin; LPS; LAMs; lipopeptide; flagellin; ATP; substance P; CL097; and CL075. FIG. 48A sets forth such priming agents and their effects as known in the art. In some embodiments of any of the aspects, the at least one priming agent is selected from the group consisting of: a caspase inhibitor; GCSF; and GM-CSF. In some embodiments of any of the aspects, the caspase inhibitor is a pan-caspase inhibitor (e.g., iCASP, Z-VAD-FMK,Q-VD(OMe)-OPh, Z-VAD(OMe)-FMK, Boc-D-FMK, and the like). In some embodiments of any of the aspects, the at least one priming agent is selected from the group consisting of: a caspase inhibitor; GCSF; and GM-CSF. In some embodiments of any of the aspects, the at least one priming agent is selected from the group consisting of: IL-2 and IL-15.

The polymeric particles described herein can be made by one or more of layer-by-layer technology, stamping, soft lithography, spin coating, and printing. Suitable methods of production are exemplied in the Examples herein and described in detail in International Patent Publications WO 2019/139892 and WO 2020/247576; which are incorporated by reference herein in their entireties. Layer-by-layer techniques, which, along with general procedures for suitable methods of fabrication, are also described in detail in US Patent Publication 2004/01152791; Park et al. AdvancedMaterials 2005 17:2575-2579; and Decher et al. “Multilayer Thin Films: Sequential Assembly of Nanocomposite Materials” 2012 John Wiley & Sons; each of which is incorporated by reference herein in its entirety.

In some embodiments of any of the aspects, described herein is a composition as described herein comprising two or more different polymeric particles as described herein, e.g., two structurally different polymeric particles. In some embodiments of any of the aspects, described herein is an engineered cellular composition as described herein comprising individual cells with two or more adhered polymeric particles as described herein, e.g., two structurally different polymeric particles. In some embodiments of any of the aspects, described herein is an engineered cellular composition as described herein comprising two different cells that are different either in their cell type or the structure of the polymeric particles adhered to each cell.

In one aspect of any of the embodiments, described herein is a method of obtaining images (e.g., MRI images) of a subject in need thereof, the method comprising administering to the subject: a polymeric particle as described herein wherein the particle comprises an imaging agent (e.g., an MRI contract agent) and/or an engineered cellular composition comprising polymeric particle as described herein wherein the particle comprises an imaging agent (e.g., an MRI contract agent) and then subjecting the subject to an imagining exposure or scan (e.g, a MRI scan.). In some embodiments of any of the aspects, the cell is a macrophage, monocyte, or T cell.

In one aspect of any of the embodiments, described herein is a method of treating cancer and/or a tumor in a subject in need thereof, the method comprising administering to the subject a polymeric particle or an engineered cellular composition as described herein. In some embodiments of any of the aspects, when the method relates to treating a cancer and/or tumor, the polymeric particle comprises a polarizing agent which is an M1-polarizing agent. In some embodiments of any of the aspects, when the method relates to treating a cancer and/or tumor, the polymeric particle comprises one or more payload agents comprising a chemotherapeutic agent.

In one aspect of any of the embodiments, described herein is a method of treating a fracture, wound, injury (e.g., TBI) or infection in a subject in need thereof, the method comprising administering to the subject a polymeric particle or an engineered cellular composition as described herein. In some embodiments of any of the aspects, when the method relates to treating a fracture, wound, injury, or infection, the polymeric particle comprises a polarizing agent which is an M2-polarizing agent. In some embodiments of any of the aspects, when the method relates to treating a fracture, wound, injury, or infection, the polymeric particle comprises one or more payload agents comprising an antibiotic, antiviral, antimicrobial, a hemostatic agent, an analgesic, and/or an anti-inflammatory agent.

As used herein, “antiviral” refers to any chemical or biological agent with therapeutic usefulness in the inhibition of viral transmission, activity, or replication. Categories of antivirals can include, but are not limited to entry inhibitors, uncoating inhibitors, viral synthesis inhbitiors, assembly inhibitors, and release inhibitors. Exemplary, non-limiting antivirals include enfuvirtide, amantadine, rimantadine, pleconaril, acyclovir, zidovudine, lamivudine, fomivirsen, rifampicin, zanamivir, oseltamivir, peramivir, abacavir, acyclovir, adefovir, amprenavir, baloxavir marboxil, boceprevir, cobicistat, combivir, daclatasvir, doravirine, etravirine, ganciclovir, ibalizumab, letermovir, rilpivirine, simeprevir, telbivudine, and valciclovir. One of skill in the art can readily identify an antiviral agent of use e.g. see Antiviral Drugs, Wieslaw M. Kazmierski (ed.) Wiley and Sons (2011); Antiviral Drugs, John S. Driscoll. Wiley and Sons (2005); each of which is incorporated by reference herein in its entirety.

As used herein, “antibiotic” refers to any chemical or biological agent with therapeutic usefulness in the inhibition of bacterial cell growth or in killing bacteria, e.g, those that are bactericidal or bacteriostatic. Categories of antibiotics can include, but are not limited to those that target the bacterial cell wall (e.g., penicillins, cephalosporins), those that target the bacterial cell membrane (e.g., polymyxins), those that target bacterial enzymes (e.g., rifamycins, lipiarmycins, quinolones, sulfonamides), protein synthesis inhibitors (e.g., macrolides, lincosamides, and tetracyclines), aminoglycosides, cyclic lipopeptides, glycyclines, oxazolidinones, beta-lactams, and lipiarmycins. Exemplary, non-limiting antibiotics include penicillin, methicilling, nafcillin, oxacillin, cloxacillin, dicloxacillin, flucloxacillin, ampicillin, amoxicillin, pivampicillin, hetacillin, bacampicillin, metampicillin, talamipicillin, epicillin, cabenicillin, ticaricillin, temocillin, mezlocillin, piperacillin, azolocillin, clavulanic acid, sulbactam, tazobactam, cafadroxil, cephalexin, cefalotin, cefapirin, cefazolin, cefradine, cefaclor, cefonicid, cefprozil, cefuroxime, loracarbef, cefmetazole, cefotetan, cefoxitin, cefotiam, cefdinir, cefixime, cefotaxime, cefovecin, cefpodoxime, ceftibuten, ceftiofur, ceftizoxime, ceftriaxone, cefoperazone, ceftazimdime, latamoxef, cefepime, cefiderocol, cefpriome, rifampicin, rifabutin, rifapentine, rifamixin, fidaxomicin, ciproflaxicin, moxifloxacin, levofloxacin, sulfafurzole, azithromycin, clarithromycin, erythromycin, fidaxomicin, spiramycin, telihtromycin, lincomycin, clindamycin, pirlimycin, tetracycline, eravacycline, sarecycline, omadacycline, doxycycline, kanamycin, tobramycin, gentamicin, neomycin, streptomycin, vancomycin, tigecycline, linezolid, posizolid, tedizolid, radezolid, cycloserine, contezolid, and daptomycin. One of skill in the art can readily identify an antibiotic agent of use e.g. see Antibiotics in Laboratory Medicine, Victor Lorian (ed.) Wolters Kluwer; and Antibotics Manual, David Schlossberg and Rafik Samuel, John Wiley and Sons (2017); each of which is incorporated by reference herein in its entirety.

Hemostatic agents are known in the art. As used herein, “hemostatic agent” refers to an agent that promotes clotting or coagulation and/or stops bleeding. Exemplary hemostatic agents can include microfibrillar collagen, gelatin, factor concentrators (e.g., QuikClot™ (Z-Medica LLC., Newington, CT, USA), QuikClot ACS™ (advanced clotting sponge) (Z-Medica LLC., Newington, CT, USA), TraumaDex™ (Medafor Inc, Minneapolis, MN, USA), and self-expanding hemostatic polymer (Payload Systems Inc., Cambridge, MA, USA)), muscoadhesive agents (e.g., HemCon™ (HemCon Medical Technologies Inc. Portland, OR, USA) and Celox™ (Medtrade Products Ltd. Crewe, UK)), procoagulatn supplementors (e.g., dry fribrin sealant dressing), fibrin, thrombin, collagent, adrenaline, VBP, CBP, Factor VIII, Factor IX, and agents as described in US Patent Publication 2018/0311378, which is incorporated by reference herein in its entirety.

Anti-inflammatory agents are known in the art. Exemplary anti-inflammatories include, but are not limited to, non-steroidal anti-inflammatory drugs (NSAIDs -such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; opiates (e.g. endorphins, enkephalins, and dynorphin), steroids, analgesics, barbiturates, oxycodone, morphine, lidocaine, and the like. In some embodiments, the anti-inflammatory agent can be a steroid (e.g., a corticosteroid or glucocorticoid); a calcineurin inhibitor (e.g. cyclosporine, tacrolimus, pimecrolimus, or FK506); an mTOR inhibitor (e.g., everolimus, temsirolimus, rapamycin, deforolimus, TOP216, OSI-027, TAFA93, nab-rapamycin, tacrolimus, biolimus, CI-779, ABT-578, AP-23675, BEZ-235, QLT-0447, ABI-009, BC-210, salirasib, AP-23841, AP-23573, KU-0059475, 32-deoxorapamycin, 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-O-(2-hydroxyethyl)-rapamycin, 40-O-(2-hydroxyethyl)-rapamycin, 32-deoxorapamycin; 16-pent-2-ynyloxy-32(S)-dihydrorapamycin; socalledrapalogs; AP23464; PI-103, PP242, PP30, Torin1; and derivatives or pharmaceutically acceptable salts thereof as well as and compounds described in, e.g. U.S. Patent Publications 2011/0178070; 2011/0021515; 2007/0112005; 2011/0054013; International Patent Publications WO98/02441; WO01/14387; WO99/15530; WO07/135411; WO03/64383; WO96/41807; WO95/16691; WO94/09010; European Patent No. EP1880723; and U.S. Pat. Nos. 8,163,775; 6,329,386; 6,200,985; 6,117,863; 6,015,815; 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; and 5,023,262; which are incorporated by reference herein in their entireties.); rapamycin (sirolimus) or an analogue therof (e.g. everolimus, temsirolimus, ridaforolimus, deforolimus); or an anti-prolferative agent (e.g. mycophenoloate moefitil, azathioprine). In some embodiments, the mTOR inhibitor can be rapamycin or an analogue thereof, e.g. everolimus, temsirolimus, ridaforolimus, or deforolimus. Anti-proliferative agents can include, by way of non-limiting example, alkylating agents (e.g. cyclophosphamide, platinum compounds, and nitrosoureass), antimetabolites (e.g. methotrexate, azathioprine, mercaptopurine, fluorouracil, etc), and cytotoxic antibiotics (e.g., dactinomycin, anthracyclines, mitomycin C, bleomycin, and mithramycin).

Pain relief agents or analgesics are known in the art and can include, by way of non-limiting example, acetaminophen, NSAIDs (e.g., aspirin, ibuprofen, naproxen), COX-2 inhibitors (e.g., rofecoxib, celecoxib, etoricoxib), opioids (e.g., codeine, oxycodone, hydrocodone, dihydromorphine, pethidine, tramadol, venlafaxine, tapentadol, cannabanoids, opioid potentiators (e.g, hydroxyzine, promethazine, carisoprodol, or tripelennamine), adjuvant analgesics (e.g., orphenadrine, mexiletine, pregabalin, gabapentin, cyclobenzaprine, hyoscine (scopolamine)), carbamazepine, and gabapentionids.

In one aspect of any of the embodiments, described herein is a method of treating inflammation in a subject in need thereof, the method comprising administering to the subject a polymeric particle or an engineered cellular composition as described herein. In some embodiments of any of the aspects, when the method relates to treating inflammation, the polymeric particle comprises a polarizing agent which is an M2-polarizing agent. In some embodiments of any of the aspects of treating inflammation, the M2-polarizing agent is a cytokine, e.g., an IL-4 polypeptide. In some embodiments of any of the aspects, when the method relates to treating inflammation, the polymeric particle comprises one or more payload agents comprising an anti-inflammatory agent.

In some embodiments of any of the aspects, the inflammation is in the lungs and is caused by or arises from infection or injury. In some embodiments of any of the aspects, the inflammation is in the joints and is caused by or arises from arthritis. In some embodiments of any of the aspects, the inflammation is in the skin and is caused by or arises from infection or autoimmune disorder. In some embodiments of any of the aspects, the inflammation is caused by, arises from, or is a symptom of acute respiratory distress (ARDS), arthritis, infection, or an autoimmune disorder.

In one aspect of any of the embodiments, described herein is a method of treating an autoimmune condition in a subject in need thereof, the method comprising administering to the subject a polymeric particle or an engineered cellular composition as described herein. In some embodiments of any of the aspects, when the method relates to treating an autoimmune condition, the polymeric particle comprises a polarizing agent which is an M2-polarizing agent. In some embodiments of any of the aspects of treating inflammation, the M2-polarizing agent is a cytokine, e.g., an IL-4 polypeptide. In some embodiments of any of the aspects, when the method relates to treating an autoimmune condition, the polymeric particle comprises one or more payload agents comprising an immunosuppressive agent.

As used herein, the term “autoimmune disease” or “autoimmune disease or disorder” herein is a disease or disorder arising from and directed against an individual's own tissues or cells or manifestation thereof or resulting condition therefrom. Auto-immune related diseases and disorders arise from an overactive and/or abnormal immune response of the body against substances (autoantigens) and tissues normally present in the body, otherwise known as self or autologous substance. This dysregulated inflammatory reaction causes an exaggerated response by macrophages, granulocytes, and/or T-lymphocytes leading to abnormal tissue damage and cell death. Subsequent loss of function is associated with inflammatory tissue damage.

In some embodiments of any the aspects, the autoimmune condition is rheumatoid arthritis, lupus, or celiac disease. In one embodiment of any one of the method described, the autoimmune disorder is selected from the group consisting of thyroiditis, type 1 diabetes mellitus, Hashimoto's thyroidits, Graves' disease, celiac disease, multiple sclerolsis, Guillain-Barre syndrome, Addison's disease, and Raynaud's phenomenon, Goodpasture's disease, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, diabetic large-artery disorder, immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and giant-cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, an immune complex disorder such as immune complex nephritis, antibody-mediated nephritis, polyneuropathies, chronic neuropathy such as IgM polyneuropathies or IgM-mediated neuropathy, and autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, lupoid hepatitis, giant-cell hepatitis, autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies including channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, Sampter's syndrome, Caplan's syndrome, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, cyclitis such as chronic cyclitis, heterochronic cyclitis, iridocyclitis (acute or chronic), or Fuch's cyclitis, Henoch-Schonlein purpura, SCID, sepsis, endotoxemia, post-vaccination syndromes, Evan's syndrome, autoimmune gonadal failure, Sydenham's chorea, post-streptococcal nephritis, thromboangitis ubiterans, thyrotoxicosis, tabes dorsalis, chorioiditis, giant-cell polymyalgia, chronic hypersensitivity pneumonitis, keratoconjunctivitis sicca, idiopathic nephritic syndrome, minimal change nephropathy, benign familial and ischemia-reperfusion injury, transplant organ reperfusion, retinal autoimmunity, aphthae, aphthous stomatitis, arteriosclerotic disorders, aspermiogenesis, autoimmune hemolysis, Boeck's disease, enteritis allergica, erythema nodosum leprosum, idiopathic facial paralysis, chronic fatigue syndrome, febris rheumatica, Hamman-Rich's disease, sensoneural hearing loss, ileitis regionalis, leucopenia, transverse myelitis, primary idiopathic myxedema, ophthalmia symphatica, polyradiculitis acuta, pyoderma gangrenosum, acquired spenic atrophy, vitiligo, toxic-shock syndrome, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, rheumatic diseases, mixed connective tissue disease, nephrotic syndrome, insulitis, polyendocrine failure, autoimmune polyglandular syndrome type I, adult-onset idiopathic hypoparathyroidism (AOIH), myocarditis, nephrotic syndrome, primary sclerosing cholangitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, granulomas containing eosinophils, seronegative spondyloarthritides, polyendocrine autoimmune disease, sclerosing cholangitis, sclera, episclera, Bruton's syndrome, transient hypogammaglobulinemia of infancy, Wiskott-Aldrich syndrome, ataxia telangiectasia syndrome, angiectasis, autoimmune disorders associated with collagen disease, rheumatism, allergic hypersensitivity disorders, glomerulonephritides, reperfusion injury, ischemic re-perfusion disorder, lymphomatous tracheobronchitis, inflammatory dermatoses, dermatoses with acute inflammatory components, and autoimmune uveoretinitis (AUR).

Treatments for autoimmune disorders are known in the art and can include, but are not limited to immunosuppressive drugs, e.g., Cyclosporine (Neoral, Sandimmune, Gengraf, and Restasis), Tacrolimus (Prograf, Protopic, Astagraf XL, and Envarsus XR), Methotrexate (Trexall, Rasuvo, Rheumatrex, and Otrexup (PF)), Sirolimus (Rapamune), Mycophenolic acid (Myfortic and CellCept), Rituximab (Rituxan), etanercept (Enbrel), pentostatin (Nipent), ruxolitinib (Jakafi); Chemotherapies, e.g., Methotrexate (Trexall, Rasuvo, Rheumatrex, and Otrexup (PF)), antithymocyte globulin (Atgam, Thymoglobulin); Steroids, e.g,. Prednisone (Deltasone, Rayos, and Prednisone Intensol), Methylprednisolone (Medrol, Solu-Medrol, and Depo-Medrol), budesonide (Entocort EC, Uceris); Antifungal, e.g., Posaconazole (Noxafil); Antiviral drugs, e.g., Acyclovir (Zovirax and Sitavig), Valacyclovir (Valtrex); and Antibiotics, e.g., Sulfamethoxazole/Trimethoprim (Bactrim, Sulfatrim, and Bactrim DS); Protease inhibitors, e.g. alphal-proteinase inhibitor (Zemaira); extracorporeal photopheresis; monoclonal antibodies (daclizumab (Zinbryta), basiliximab (Simulect)), Brentuximab vedotin (Adcetris), Alemtuzumab (Campath, Lemtrada), Tocilizumab (Actemra); infusion of mesenchymal stromal cells.

In one aspect of any of the embodiments, described herein is a method of providing hemostatic treatment to a subject in need thereof, the method comprising administering to the subject a polymeric particle or an engineered cellular composition as described herein. In some embodiments of any of the aspects, when the method relates to providing hemostatic treatment, the polymeric particle comprises a polarizing agent which is an M2-polarizing agent. In some embodiments of any of the aspects, when the method relates to providing hemostatic treatment, the polymeric particle comprises one or more payload agents comprising a hemostatic agent.

A subject in need of hemostatic treatment can be a subject who is bleeding, has a wound, has trauma, has a bleeding disorder (e.g., hemophilia, Von Willebrand disease, disseminated intravascular coagulation, thrombocytopenia, end-stage liver failure, congential afibrogenemia, Glanzmann's thrombasthenia, or Bernard-Soulier syndrome), has Acute Traumatic Coagulopathy, or is a subject undergoing surgery.

In one aspect of any of the embodiments, described herein is a method of treating a neurodegenerative disorder, a central nervous system disorder, or a peripheral nervous system disorder in a subject in need thereof, the method comprising administering to the subject a polymeric particle or an engineered cellular composition as described herein. In some embodiments of any of the aspects, when the method relates to treating neurodegenerative disorder, a central nervous system disorder, or a peripheral nervous system disorder, the polymeric particle comprises one or more payload agents comprising a therapeutic agent for the neurodegenerative disorder, a central nervous system disorder, or a peripheral nervous system disorder.

In one aspect of any of the embodiments, described herein is a method of providing a payload reagent to the central nervous system of a subject, the method comprising administering to the subject a polymeric particle or an engineered cellular composition as described herein. In some embodiments of any of the aspects, when the method relates to delivering a payload to the central nervous system, the cell is a monocyte and/or the binding reagent is CD11b. In some embodiments of any of the aspects, the payload to be delivered to the central nervous system can be a therapeutic agent for a neurodegenerative disorder, a central nervous system disorder, or a peripheral nervous system disorder or a therapeutic for a disease or disorder found in the CNS (e.g., a chemotherapeutic for a glioblastoma).

Non-limiting examples of a neurodegenerative disorder, a central nervous system disorder, or a peripheral nervous system disorder can include cancer; a brain tumor; encephalitis; hydrocephalus; Parksinson's disease; neuropathic pain; a condition treated by the administration of psychiatric drugs; a neurodegenerative disease; multiple sclerosis; Huntington's disease; Pick's disease; ALS; dementia; stroke; or Alzheimer's disease. In some embodiments of any of the aspects, the method is a method of treating a subject. In some embodiments of any of the aspects, the method is a method of treating brain cancer; a brain tumor; encephalitis; hydrocephalus; Parksinson's disease; neuropathic pain; a condition treated by the administration of psychiatric drugs; a neurodegenerative disease; multiple sclerosis; Huntington's disease; Pick's disease; ALS; dementia; stroke; and Alzheimer's disease in a subject.

Agents that are therapeutic for neurodegenerative disorders, a central nervous system disorder, or a peripheral nervous system disorder are known in the art and can include, by way of nonlimiting example, antibiotics, antibodies, gabapentin, chemotherapeutics, anti-inflammatories, neurotransmitters, morphines, peptides, polypeptides, nucleic acids (e.g. RNAi-based therapies), psychiatric dugs, and/or therapeutic agents for the treatment of brain cancer; encephalitis; hydrocephalus; Parksinson's disease; neuropathic pain; and a condition treated by the administration of psychiatric drugs. The identity of such CNS therapeutic agents are known in the art and described, e.g. in Ghose et al. J Comb Chem 1999 1:55-68 and Pardridge. NeuroRx 2005 2:3-14; each of which is incorporated by reference herein in its entirety.

In one aspect, described herein is a method of vaccinating a subject, immunizing a subject, or inducing an immune response in a subject in need thereof, the method comprising administering to the subject the polymeric particle or the engineered cellular composition as described herein, wherein the polymeric particle comprises a payload reagent that is an antigen. In some embodiments of any of the aspects, the cell is a B cell.

The terms “immunize” and “vaccinate” tend to be used interchangeably in the field. However, in reference to the administration of the vaccine compositions as described herein to provide protection against disease, e.g., infectious disease caused by a pathogen, it should be understood that “vaccinate” refers to the administration of a vaccine composition and the term “immunize” refers to the process of conferring, increasing, or inducing the passive protection conferred by the administered vaccine composition.

As described herein, an “antigen” is a molecule that is specifically bound by a B cell receptor (BCR), T cell receptor (TCR), and/or antibody, thereby activating an immune response. An antigen can be pathogen-derived, or originate from a pathogen. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule. In some embodiments of any of the aspects, a vaccine or composition described herein comprises a nucleic acid encoding an antigen.

In some embodiments of any of the aspects, the antigen can be a molecule or motif obtained or derived froma pathogen, e.g., a coronavirus; a SARS-CoV-2 virus; a pneumococcus; an influenza virus; a hepatitis B virus (HBV); Bordetella pertussis; Corynebacterium diphtheria; Clostridium tetani; a hepatitis A virus (HAV); and a meningococcus. In some embodiments of any of the aspects, the antigen can be a molecule found in a coronavirus; a SARS-CoV-2 virus; a pneumococcus; an influenza virus; a hepatitis B virus (HBV); Bordetella pertussis; Corynebacterium diphtheria; Clostridium tetani; a hepatitis A virus (HAV); and a meningococcus. In some embodiments of any of the aspects, the antigen can be a molecule (or antigenic portion thereof) with at least 60%, at least 65%, at least 70%, at least 75%, at least 80%, at least 85%, at least 90%, at least 95%, at least 98% or more sequence identity (nucleotide or amino acid) with a molecule found in a pathogen, e.g., a coronavirus; a SARS-CoV-2 virus; a pneumococcus; an influenza virus; a hepatitis B virus (HBV); Bordetella pertussis; Corynebacterium diphtheria; Clostridium tetani; a hepatitis A virus (HAV); and a meningococcus. In some embodiments of any of the aspects, the antigen can be a nucleic acid encoding a protein (or antigenic portion thereof) with at least 60%, at least 65%, at least 70%, at least 75%, at least 80%, at least 85%, at least 90%, at least 95%, at least 98% or more sequence identity with a protein found in a pathogen, e.g., a coronavirus; a SARS-CoV-2 virus; a pneumococcus; an influenza virus; a hepatitis B virus (HBV); Bordetella pertussis; Corynebacterium diphtheria; Clostridium tetani; a hepatitis A virus (HAV); and a meningococcus. In some embodiments of any of the aspects, a protein with a specified sequence identity to a protein found in a pathogen retains the wild-type activity of the reference protein found in the pathogen.

In some embodiments of any of the aspects, the antigen can be a viral spike protein or antigenic portion thereof, e.g., a coronavirus or a SARS-CoV-2 virus spike protein or antigenic portion thereof. In some embodiments of any of the aspects, the antigen can be a protein with at least 60%, at least 65%, at least 70%, at least 75%, at least 80%, at least 85%, at least 90%, at least 95%, at least 98% or more sequence identity with a viral spike protein, e.g., a coronavirus or a SARS-CoV-2 virus spike protein or antigenic portion thereof.

The scientific name for coronavirus is Orthocoronavirinae or Coronavirinae. Coronaviruses belong to the family of Coronaviridae, order Nidovirales, and realm Riboviria. They are divided into alphacoronaviruses and betacoronaviruses which infect mammals - and gammacoronaviruses and deltacoronaviruses which primarily infect birds. Non limiting examples of alphacoronaviruses include: Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, and Feline Infectious Peritonitis Virus (FIPV, also referred to as Feline Infectious Hepatitis Virus). Non limiting examples of betacoronaviruses include: Betacoronavirus 1 (e.g., Bovine Coronavirus, Human coronavirus OC43), Human coronavirus HKU1, Murine coronavirus (also known as Mouse hepatitis virus (MHV)), Pipistrellus bat coronavirus HKU5, Rousettus bat coronavirus HKU9, Severe acute respiratory syndrome-related coronavirus (e.g., SARS-CoV, SARS-CoV-2), Tylonycteris bat coronavirus HKU4, Middle East respiratory syndrome (MERS)-related coronavirus, and Hedgehog coronavirus 1 (EriCoV). Non limiting examples of gammacoronaviruses include: Beluga whale coronavirus SW1, and Infectious bronchitis virus. Non limiting examples of deltacoronaviruses include: Bulbul coronavirus HKU11, and Porcine coronavirus HKU15.

In some embodiments of any of the aspects, the coronavirus is selected from the group consisting of: severe acute respiratory syndrome-associated coronavirus (SARS-CoV); severe acute respiratory syndrome-associated coronavirus 2 (SARS-CoV-2); Middle East respiratory syndrome-related coronavirus (MERS-CoV); HCoV-NL63; and HCoV-HKul. In some embodiments of any of the aspects, the coronavirus is severe acute respiratory syndrome coronavirus 2 (SARS-CoV-2), which causes coronavirus disease of 2019 (COVID19 or simply COVID). In some embodiments of any of the aspects, the coronavirus is severe acute respiratory syndrome coronavirus (SARS-CoV or SARS-CoV-1), which causes SARS. In some embodiments of any of the aspects, the coronavirus is Middle East respiratory syndrome-related coronavirus (MERS-CoV), which causes MERS.

Nucleic acids and proteins for the foregoing pathogens are known in the art, e.g., the complete genome of SARS-CoV-2 Jan. 2020/NC_045512.2 Assembly (wuhCorl) is available on the world wide web.

In some embodiments of any of the aspects, the vaccine can include one or more adjuvants. In some embodiments of any of the aspects, the payload molecules can include one or more adjuvants.

In some embodiments of any of the aspects, the at least one antigen is comprised by a vaccine. In some embodiments of any of the aspects, the vaccine is an attenuated vaccine. Attenuated vaccines comprise weakened or compromised versions or variants of a disease-causing microbe. Attenuated vaccines can include mutated or engineered strains of a microbe and/or strains which have been passaged in culture, thereby resulting in a loss of pathogenicity.

In some embodiments of any of the aspects, the vaccine can be a subunit vaccine, including a recombinant subunit vaccine. A subunit vaccine does not comprise entire disease-causing microbes, but only a subset of antigens obtained from or derived from the disease-causing microbe. A subunit vaccine can comprise multiple different antigens. Subunit vaccines in which the antigens are produced via recombinant technologies are termed recombinant subunit vaccines.

In some embodiments of any of the aspects, the at least one antigen is comprised by a conjugate vaccine. In conjugate vaccines, polysaccharides from a disease-causing microbe (e.g., polysaccahrides found on the surface of the microbe) are administered in combination with (e.g., conjugated to) an antigen which the patient's immune system already recognizes or which the patient's immune system will readily respond to. This increases the patient's response to the polysaccharides and provides increased protection against live versions of the disease-causing microbe. In some embodiments of any of the aspects, the antigen is a polysaccharide.

Exemplary, non-limiting vaccines suitable for use in the methods and compositions described herein can include a coronavirus vaccine; a SARS-CoV-2 vaccine; a pneumococcal vaccine; an influenza vaccine; a hepatitis B (HBV) vaccine; an acellular pertussis (aP) vaccine; a diphtheria tetanus acellular pertussis (DTaP) vaccine; a hepatitis A (HAV) vaccine; a meningococcal (MV) vaccine; and/or pneumococcal conjugate vaccine (PCV)13.

In some embodiments of any of the aspects, multiple antigens are administered. In some embodiments of any of the aspects, multiple vaccines are administered.

As used herein, an “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus (e.g., to an vaccine). In some embodiments of the aspects described herein, the response is specific for a particular antigen (an “antigen-specific response”), and refers to a response by a CD4 T cell, CD8 T cell, or B cell via their antigen-specific receptor. In some embodiments of the aspects described herein, an immune response is a T cell response, such as a CD4+response or a CD8+response. Such responses by these cells can include, for example, cytotoxicity, proliferation, cytokine or chemokine production, trafficking, or phagocytosis, and can be dependent on the nature of the immune cell undergoing the response. Stimulation of an immune response refers to an induction or increase of the immune response.

CD4+ T cells can display a Th1 or a Th2 phenotype. Pro-inflammatory CD4+ T cells are responsible for the release of inflammatory, Th1 type cytokines. Cytokines characterized as Th1 type include interleukin 2 (IL-2), y-interferon, TNFα and IL-12. In some embodiments, cytokines characterized as Th1 type include interleukin 2 (IL-2), interferon γ, and TNFα. Such pro-inflammatory cytokines act to stimulate the immune response, in many cases resulting in the destruction of autologous tissue. Cytokines associated with suppression of T cell response are the Th2 type, and include IL-10, IL-4 and TGF-β. It has been found that Th1 and Th2 type T cells may use the identical antigen receptor in response to an immunogen; in the former producing a stimulatory response and, in the latter, a suppressive response.

In some embodiments of any of the aspects, an immune response can be an increase in or induction of a Th1 or Th2 immune response, cytokine production/release, or levels of T cells displaying a Th1 or Th2 phenotype. In some embodiments of any of the aspects, the increase is relative to the level or number in the absence of the vaccine.

In some embodiments of any of the aspects, an immune response can be a Th1 response. In some embodiments of any of the aspects, an immune response can be cytokine production by Th1 cells. In some embodiments of any of the aspects, an immune response can be an increase in the level of Th1 antigen-specific CD4+cells. In some embodiments of any of the aspects, an immune response can be an increase in the level of Th1 CD4+cells. In some embodiments of any of the aspects, an immune response can be an increase in the level of Th1 cells. In some embodiments of any of the aspects, an immune response can be an increase in the level of CD4+cells. In some embodiments of any of the aspects, the increase is relative to the level or number in the absence of the vaccine.

In some embodiments of any of the aspects, the immune response is an increase in the IgG2a/c subclass.

In some embodiments of any of the aspects, an immune response can be an increase in activation and/or infiltration of dendritic cells. In some embodiments of any of the aspects, an immune response can be an increase in the number of and/or infiltration of CD4+cells. In some embodiments of any of the aspects, an immune response can be an increase in the number of CD4+cells. In some embodiments of any of the aspects, an immune response can be an increase in the infiltration of CD4+cells. In some embodiments of any of the aspects, an immune response can be an increase in the number of and/or infiltration of Th1 CD4+cells. In some embodiments of any of the aspects, an immune response can be an increase in the number of NK and/or CD8+cells. In some embodiments of any of the aspects, an immune response can be an increase in the number of NK cells. In some embodiments of any of the aspects, an immune response can be an increase in the number of CD8+cells. In some embodiments of any of the aspects, the increase is relative to the level or number in the absence of the vaccine.

An immune response to an antigen can be the development in a subject of a humoral and/or a cell-mediated immune response to molecules present in the antigen or vaccine composition of interest. For purposes of the present invention, a “humoral immune response” is an antibody-mediated immune response and involves the induction and generation of antibodies that recognize and bind with some affinity for the antigen in the immunogenic composition of the invention, while a “cell-mediated immune response” is one mediated by T-cells and/or other white blood cells. A “cell-mediated immune response” is elicited by the presentation of antigenic epitopes in association with Class I or Class II molecules of the major histocompatibility complex (MHC), CD1 or other non-classical MHC-like molecules. This activates antigen-specific CD4+T helper cells or CD8+cytotoxic lymphocyte cells (“CTLs”). CTLs have specificity for peptide antigens that are presented in association with proteins encoded by classical or non-classical MHCs and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide or other antigens in association with classical or non-classical MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376.

In one aspect of any of the embodiments, described herein is a method of providing a gene therapy vector to a subject in need thereof, the method comprising administering to the subject a polymeric particle or an engineered cellular composition as described herein, wherein the polymeric particle comprises a payload reagent that is a gene therapy vector. Gene therapy vectors can include plasmids, CRISPR-Cas components or vectors encoding the same, plasmids, expression vectors, viral vectors, or viral particles.

In one aspect of any of the embodiments, described herein is a method of providing a vector to a subject or cell, the method comprising administering to the subject (or contacting the cell with) a polymeric particle or an engineered cellular composition as described herein, wherein the polymeric particle comprises a payload reagent that is a vector.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optomized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

Viral vector systems which can be utilized in the present invention include, but are not limited to, (a) adenovirus vectors; (b) retrovirus vectors, e.g., lentivirus vectors, murine moloney leukemia virus, etc.; (c) adeno-associated virus vectors; (d) herpes simplex virus vectors; (e) SV40 vectors; (f) polyoma virus vectors; (g) papilloma virus vectors; (h) picornavirus vectors; (i) pox virus vectors such as an orthopox, e.g., vaccinia virus vectors or avipox, e.g., canary pox or fowl pox; and (j) a helper-dependent or gutless adenovirus. Replication-defective viruses can also be advantageous. In some embodiments, the vector is an adeno-associated virus vector.

In some embodiments, a viral vector such as an adeno-associated virus (AAV) vector is used. AAVs, which normally infect mammals, including humans, but are non-pathogenic, have been developed and employed as gene therapy vectors in clinical trials in the United States and Europe (Daya and Berns, Clinical Microbiology Reviews 2008, 21, 583-593). AAV vectors may be prepared using any one of a number of methods available to those of ordinary skill in the art. Exemplary AAV vectors are disclosed in Walsh et al., Proc. Soc. Exp. Biol. Med. 204:289-300 (1993); U.S. Pat. No. 5,436,146 which is incorporated herein by reference; Gao et al., Gene Therapy 2005, 5, 285-297; Vandenberghe et al., Gene Therapy 2009, 16, 311-319; Gao et al., PNAS 2002, 99, 11854-11859; Gao et al., PNAS 2003, 100, 6081-6086; Gao et al., J. of Virology 2004, 78, 6381-6388.

In some embodiments, the vector is an adeno-associated virus (AAV) vector. In some embodiments, the AAV vector is an AAV1, AAV2, AAV4, AAV5, AAV6, AAV7, AAV8, AAV9, AAV9.HR, AAVrh.10, AAVMYO, or AAV2.5. In some embodiments, the AAV is AAV9.

In some embodiments, the vector is episomal. The use of a suitable episomal vector provides a means of maintaining the nucleotide of interest in the subject in high copy number extra chromosomal DNA thereby eliminating potential effects of chromosomal integration.

As non-limiting examples, in some embodiments, a plasmid expression vector can be used. Plasmid expression vectors include, but are not limited to, pcDNA3.1, pET vectors (Novagen®), pGEX vectors (GE Life Sciences), and pMAL vectors (New England labs. Inc.) for protein expression in E. coli host cell such as BL21, BL21(DE3) and AD494(DE3)pLysS, Rosetta (DE3), and Origami(DE3) (Novagen®); the strong CMV promoter-based pcDNA3.1 (Invitrogen™ Inc.) and pCIneo vectors (Promega) for expression in mammalian cell lines such as CHO, COS, HEK-293, Jurkat, and MCF-7; replication incompetent adenoviral vector vectors pAdeno X, pAd5F35, pLP-Adeno-X-CMV (Clontech®), pAd/CMV/V5-DEST, pAd-DEST vector (Invitrogen™ Inc.) for adenovirus-mediated gene transfer and expression in mammalian cells; pLNCX2, pLXSN, and pLAPSN retrovirus vectors for use with the Retro-X™ system from Clontech for retroviral-mediated gene transfer and expression in mammalian cells; pLenti4/V5-DEST™, pLenti6/V5-DEST™, and pLenti6.2/V5-GW/lacZ (INVITROGEN™ Inc.) for lentivirus-mediated gene transfer and expression in mammalian cells; adenovirus-associated virus expression vectors such as pAAV-MCS, pAAV-IRES-hrGFP, and pAAV-RC vector (Stratagene®) for adeno-associated virus-mediated gene transfer and expression in mammalian cells.

A retroviral vector can also be used (see Miller et al., Meth. Enzymol. 217:581-599 (1993)). These retroviral vectors contain the components necessary for the correct packaging of the viral genome and integration into the host cell DNA. In another embodiment, the vector is a pox virus such as a vaccinia virus, for example an attenuated vaccinia such as Modified Virus Ankara (MVA) or NYVAC, an avipox such as fowl pox or canary pox. In another embodiment, lentiviral vectors are used, such as the HIV based vectors described in U.S. Pat. Nos. 6,143,520; 5,665,557; and 5,981,276, which are herein incorporated by reference. The vector may or may not be incorporated into the genome of a cell. The constructs may include viral sequences for transfection, if desired. Alternatively, the vector can be capable of episomal replication, e.g., EPV and EBV vectors.

As used herein, “viral particle” refers to a particle comprising at least one viral capsid polypeptide and a nucleic acid molecule, e.g., a viral genome and/or viral vector. Viral vectors are discussed elsewhere herein.

The engineered cellular compositions can comprise cells, which are autologous to or heterologous to the subject to be treated. In some embodiments of any of the aspects, the method of treatment can comprise a first step of obtaining the cell from a donor and/or the subject and contacting the cell with the polymeric particle ex vivo. The cell can be isolated, e.g., isolated from a blood sample obtained from the donor/subject prior to performing the contacting/adhering step, or the contacting/adhering can take place in a sample comprising multiple cell types, e.g., in a blood sample.

Alternatively, the method of treatment can comprise administering only polymeric particles, which will bind/contact/adhere to the subject's own cell in vivo.

The methods described herein can further comprise steps of localizing the engineered cellular composition to a desired location or disrupting/degrading/releasing the polymeric particle at a desired time or location. Described above herein are polymeric particles that are responsive to such controlled and/or inducible stimuli. In some embodiments of any of the aspects, the second region of the polymeric particle comprises poly(lactic-co-caprolactone) (PLCL) and the method further comprises increasing the temperature of at least one area of the subject in order to permit the cell to phagocytose the polymeric particles. In some embodiments of any of the aspects, the second region of the polymeric particle comprises a near-infrared degradable polymer or polymer linker and the method further comprises subject at least one area of the subject to near-infrared light in order to permit the cell to phagocytose the polymeric particles. In some embodiments of any of the aspects, the polymeric particle comprises an echogenic liposome and the method further comprises subject at least one area of the subject to ultrasound in order to permit the cell to phagocytose the polymeric particles or to release a payload reagent. In some embodiments of any of the aspects, the polymeric particle comprises a magnetic nanoparticle and the method further comprises subject at least one area of the subject to a magnetic field in order to permit the cell to phagocytose the polymeric particles or to release a payload reagent. In some embodiments of any of the aspects, the polymeric particle comprises a gold nanoparticle and the method further comprises subject at least one area of the subject to an electromagnetic wave in order to permit the cell to phagocytose the polymeric particles or to release a payload reagent.

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastatses. Cancers that migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm.; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulvar cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs syndrome.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

As used herein, “inflammation” refers to the complex biological response to harmful stimuli, such as pathogens, damaged cells, or irritants. Inflammation is a protective attempt by the organism to remove the injurious stimuli as well as initiate the healing process for the tissue. Accordingly, the term “inflammation” includes any cellular process that leads to the production of pro-inflammatory cytokines, inflammation mediators and/or the related downstream cellular events resulting from the actions of the cytokines thus produced, for example, fever, fluid accumulation, swelling, abscess formation, and cell death. Inflammation can include both acute responses (i.e., responses in which the inflammatory processes are active) and chronic responses (i.e., responses marked by slow progression and formation of new connective tissue). Acute and chronic inflammation may be distinguished by the cell types involved. Acute inflammation often involves polymorphonuclear neutrophils; whereas chronic inflammation is normally characterized by a lymphohistiocytic and/or granulomatous response.

An inflammatory condition is any disease state characterized by inflammatory tissues (for example, infiltrates of leukocytes such as lymphocytes, neutrophils, macrophages, eosinophils, mast cells, basophils and dendritic cells) or inflammatory processes which provoke or contribute to the abnormal clinical and histological characteristics of the disease state. Inflammatory conditions include, but are not limited to, inflammatory conditions of the skin, inflammatory conditions of the lung, inflammatory conditions of the joints, inflammatory conditions of the gut, inflammatory conditions of the eye, inflammatory conditions of the endocrine system, inflammatory conditions of the cardiovascular system, inflammatory conditions of the kidneys, inflammatory conditions of the liver, inflammatory conditions of the central nervous system, or sepsis-associated conditions. In some embodiments, the inflammatory condition is associated with wound healing. In some embodiments, the inflammation to be treated according to the methods described herein can be skin inflammation; inflammation caused by substance abuse or drug addiction; inflammation associated with infection; inflammation of the cornea; inflammation of the retina; inflammation of the spinal cord; inflammation associated with organ regeneration; and pulmonary inflammation.

In some embodiments, an inflammatory condition can be an autoimmune disease. Non-limiting examples of autoimmune diseases can include: Type 1 diabetes; systemic lupus erythematosus; rheumatoid arthritis; psoriasis; inflammatory bowel disease; Crohn's disease; and autoimmune thyroiditis.

In some embodiments, a subject in need of treatment for inflammation can be a subject having, or diagnosed as having temporomandibular joint disorders; COPD; smoke-induced lung injury; renal dialysis associated disorders; spinal cord injury; graft vs. host disease; bone marrow transplant or complications thereof, infection; trauma; pain; incisions; surgical incisions; a chronic pain disorder; a chronic bone disorder; mastitis; and joint disease. In some embodiments, trauma can include battle-related injuries or tissue damage occurring during a surgery. Smoke-induced lung injury can result from exposure to tobacco smoke, environmental pollutants (e.g. smog or forest fires), or industrial exposure. By way of non-limiting example, inflammatory conditions can be inflammatory conditions of the skin, such as Sweet's syndrome, pyoderma gangrenosum, subcorneal pustular dermatosis, erythema elevatum diutinum, Behcet's disease or acute generalized exanthematous pustulosis, a bullous disorder, psoriasis, a condition resulting in pustular lesions, acne, acne vulgaris, dermatitis (e.g. contact dermatitis, atopic dermatitis, seborrheic dermatitis, eczematous dermatitides, eczema craquelee, photoallergic dermatitis, phototoxicdermatitis, phytophotodermatitis, radiation dermatitis, stasis dermatitis or allergic contact dermatitis), eczema, ulcers and erosions resulting from trauma, burns, ischemia of the skin or mucous membranes, several forms of ichthyoses, epidermolysis bullosae, hypertrophic scars, keloids, cutaneous changes of intrinsic aging, photoaging, frictional blistering caused by mechanical shearing of the skin, cutaneous atrophy resulting from the topical use of corticosteroids, and inflammation of mucous membranes (e.g., cheilitis, chapped lips, nasal irritation, mucositis and vulvovaginitis).

By way of non-limiting example, inflammatory conditions can be inflammatory conditions of the lung, such as asthma, bronchitis, chronic bronchitis, bronchiolitis, pneumonia, sinusitis, emphysema, adult respiratory distress syndrome, pulmonary inflammation, pulmonary fibrosis, and cystic fibrosis (which may additionally or alternatively involve the gastro-intestinal tract or other tissue(s)). By way of non-limiting example, inflammatory conditions can be inflammatory conditions of the joints, such as rheumatoid arthritis, rheumatoid spondylitis, juvenile rheumatoid arthritis, osteoarthritis, gouty arthritis, infectious arthritis, psoriatic arthritis, and other arthritic conditions. By way of non-limiting example, inflammatory conditions can be inflammatory conditions of the gut or bowel, such as inflammatory bowel disease, Crohn's disease, ulcerative colitis and distal proctitis. By way of non-limiting example, inflammatory conditions can be inflammatory conditions of the eye, such as dry eye syndrome, uveitis (including iritis), conjunctivitis, scleritis, and keratoconjunctivitis sicca. By way of non-limiting example, inflammatory conditions can be inflammatory conditions of the endocrine system, such as autoimmune thyroiditis (Hashimoto's disease), Graves' disease, Type I diabetes, and acute and chronic inflammation of the adrenal cortex. By way of non-limiting example, inflammatory conditions can be inflammatory conditions of the cardiovascular system, such as coronary infarct damage, peripheral vascular disease, myocarditis, vasculitis, revascularization of stenosis, artherosclerosis, and vascular disease associated with Type II diabetes. By way of non-limiting example, inflammatory conditions can be inflammatory conditions of the kidneys, such as glomerulonephritis, interstitial nephritis, lupus nephritis, and nephritis secondary to Wegener's disease, acute renal failure secondary to acute nephritis, post-obstructive syndrome and tubular ischemia. By way of non-limiting example, inflammatory conditions can be inflammatory conditions of the liver, such as hepatitis (arising from viral infection, autoimmune responses, drug treatments, toxins, environmental agents, or as a secondary consequence of a primary disorder), biliary atresia, primary biliary cirrhosis and primary sclerosing cholangitis. By way of non-limiting example, inflammatory conditions can be inflammatory conditions of the central nervous system, such as multiple sclerosis and neurodegenerative diseases such as Alzheimer's disease or dementia associated with HIV infection. By way of non-limiting example, inflammatory conditions can be inflammatory conditions of the central nervous system, such as MS; all types of encephalitis and meningitis; acute disseminated encephalomyelitis; acute transverse myelitis; neuromyelitis optica; focal demyelinating syndromes (e.g., Balo's concentric sclerosis and Marburg variant of MS); progressive multifocal leukoencephalopathy; subacute sclerosing panencephalitis; acute haemorrhagic leucoencephalitis (Hurst's disease); human T-lymphotropic virus type-lassociated myelopathy/tropical spactic paraparesis; Devic's disease; human immunodeficiency virus encephalopathy; human immunodeficiency virus vacuolar myelopathy; peripheral neuropathies; Guillain-Barre Syndrome and other immune mediated neuropathies; and myasthenia gravis. By way of non-limiting example, inflammatory conditions can be sepsis-associated conditions, such as systemic inflammatory response syndrome (SIRS), septic shock or multiple organ dysfunction syndrome (MODS). Further non-limiting examples of inflammatory conditions include, endotoxin shock, periodontal disease, polychondritis; periarticular disorders; pancreatitis; system lupus erythematosus; Sjogren's syndrome; vasculitis sarcoidosis amyloidosis; allergies; anaphylaxis; systemic mastocytosis; pelvic inflammatory disease; multiple sclerosis; multiple sclerosis (MS); celiac disease, Guillain-Barre syndrome, sclerosing cholangitis, autoimmune hepatitis, Raynaud's phenomenon, Goodpasture's syndrome, Wegener's granulomatosis, polymyalgia rheumatica, temporal arteritis/giant cell arteritis, chronic fatigue syndrome CFS), autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, fibromyalgia (FM), autoinflammatory PAPA syndrome, Familial Mediaterranean Fever, polymyalgia rheumatica, polyarteritis nodosa, churg strauss syndrome; fibrosing alveolitis, hypersensitivity pneumonitis, allergic aspergillosis, cryptogenic pulmonary eosinophilia, bronchiolitis obliterans organising pneumonia; urticaria; lupoid hepatitis; familial cold autoinflammatory syndrome, Muckle-Wells syndrome, the neonatal onset multisystem inflammatory disease, graft rejection (including allograft rejection and graft-v-host disease), otitis, chronic obstructive pulmonary disease, sinusitis, chronic prostatitis, reperfusion injury, silicosis, inflammatory myopathies, hypersensitivities and migraines. In some embodiments, an inflammatory condition is associated with an infection, e.g., viral, bacterial, fungal, parasite or prion infections. In some embodiments, an inflammatory condition is associated with an allergic response. In some embodiments, an inflammatory condition is associated with a pollutant (e.g., asbestosis, silicosis, or berylliosis).

In some embodiments, the inflammatory condition can be a local condition, e.g., a rash or allergic reaction. In some embodiments, the inflammation is associated with a wound.

In some embodiments, the technology described herein relates to methods of promoting wound healing. As used herein, “wound” refers broadly to injuries to an organ or tissue of an organism that typically involves division of tissue or rupture of a membrane (e.g., skin), due to external violence, a mechanical agency, or infectious disease. A wound can be an epithelial, endothelial, connective tissue, ocular, or any other kind of wound in which the strength and/or integrity of a tissue has been reduced, e.g. trauma has caused damage to the tissue. The term “wound” encompasses injuries including, but not limited to, lacerations, abrasions, avulsions, cuts, burns, velocity wounds (e.g., gunshot wounds), penetration wounds, puncture wounds, contusions, diabetic wounds, hematomas, tearing wounds, and/or crushing injuries. In one aspect, the term “wound” refers to an injury to the skin and subcutaneous tissue initiated in any one of a variety of ways (e.g., pressure sores from extended bed rest, wounds induced by trauma, cuts, ulcers, burns and the like) and with varying characteristics. As used herein, the term “wound healing” refers to a process by which the body of a wounded organism initiates repair of a tissue at the wound site (e.g., skin). The wounds healing process requires, in part, angiogenesis and revascularization of the wounded tissue. Wound healing can be measured by assessing such parameters as contraction, area of the wound, percent closure, percent closure rate, and/or infiltration of blood vessels as known to those of skill in the art. In some embodiments, the particles and compositions described herein can be applied topically to promote wound healing.

The compositions and methods described herein can be administered to a subject having or diagnosed as having one of the conditions described herein. In some embodiments, the methods described herein comprise administering an effective amount of compositions described herein, e.g. a polymeric particle and/or an engineered cellular composition to a subject in order to alleviate a symptom of a condition described herein. In some embodiments of any of the aspects, a therapeutically effective dose of the composition is administered. As used herein, “alleviating a symptom” is ameliorating any condition or symptom associated with the disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to parenteral, intravenous, intramuscular, subcutaneous, transdermal, airway (aerosol), pulmonary, cutaneous, injection, or intratumoral administration. Administration can be local or systemic.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular therapeutic effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g. for tumor size and/or inflammatory markers, among others. The dosage can be determined by a physician and adjusted, as necessary, to suit observed effects of the treatment.

In some embodiments of any of the aspects, a composition described herein can be a pharmaceutical composition. In some embodiments, the technology described herein relates to a pharmaceutical composition comprising a polymeric particle and/or an engineered cellular composition as described herein, and optionally a pharmaceutically acceptable carrier. In some embodiments, the active ingredients of the pharmaceutical composition comprise a polymeric particle and/or an engineered cellular composition as described herein. In some embodiments, the active ingredients of the pharmaceutical composition consist essentially of a polymeric particle and/or an engineered cellular composition as described herein. In some embodiments, the active ingredients of the pharmaceutical composition consist of a polymeric particle and/or an engineered cellular composition as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, as described herein.

In some embodiments, the pharmaceutical composition comprising a polymeric particle and/or an engineered cellular composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry or lyophilized products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of an engineered cellular composition as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

In some embodiments of any of the aspects, the polymeric particle and/or engineered cellular composition described herein is administered as a monotherapy, e.g., another treatment for the condition is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include radiation therapy, surgery, gemcitabine, cisplastin, paclitaxel, carboplatin, bortezomib, AMG479, FK506, vorinostat, acriflavine, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaIl (see, e.g., Agnew. Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE.RTM. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb.RTM.); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

In addition, the methods of treatment can further include the use of radiation or radiation therapy. Further, the methods of treatment can further include the use of surgical treatments.

By way of non-limiting example, if a subject is to be treated for inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs -such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g., endorphins, enkephalins and dynorphin), steroids, analgesics, barbiturates, oxycodone, morphine, lidocaine and the like.

In certain embodiments, an effective dose of a composition comprising a polymeric particle and/or an engineered cellular composition as described herein can be administered to a patient once. In certain embodiments, an effective dose of a composition can be administered to a patient repeatedly. In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the composition. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising a polymeric particle and/or an engineered cellular composition as described herein can be administered over a period of time, such as over a 5 minute, 10 minute, 15 minute, 20 minute, or 25 minute period.

The dosage ranges for the administration of the compositions described herein, according to the methods described herein depend upon, for example, the potency of the particles or cells, and the extent to which symptoms, markers, or indicators of a condition described herein are desired to be reduced, for example the percentage reduction desired for tumor growth or the extent to which, for example, wound healing are desired to be induced. The dosage should not be so large as to cause adverse side effects, such as excessive inflammation or immunosuppression. Generally, the dosage will vary with the age, condition, and sex of the patient and can be determined by one of skill in the art. The dosage can also be adjusted by the individual physician in the event of any complication.

The efficacy of a polymeric particle and/or an engineered cellular composition in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of cancer. When using an experimental animal model, efficacy of treatment is evidenced when a statistically significant change in a marker is observed, e.g., tumor growth, tumor size, inflammation, wound size, etc.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies. Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

Antibodies and/or antibody reagents can include an immunoglobulin molecule, a monoclonal antibody, a chimeric antibody, a CDR-grafted antibody, a humanized antibody, a fully human antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof.

As used herein, the term “nanobody” or single domain antibody (sdAb) refers to an antibody comprising the small single variable domain (VHH) of antibodies obtained from camelids and dromedaries. Antibody proteins obtained from members of the camel and dromedary (Camelus baclrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994; which is incorporated by reference herein in its entirety).

A region of the camelid antibody which is the small single variable domain identified as VHH can be obtained by genetic engineering to yield a small protein having high afiinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J. 17: 3512-3520; each of which is incorporated by reference herein in its entirety. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in camelid nanobodies being extremely thermostable, stable to extreme pH and to proteolytic digestion, and poorly antigenic. See U.S. patent application 20040161738 published Aug. 19, 2004; which is incorporated by reference herein in its entirety. These features combined with the low antigenicity to humans indicate great therapeutic potential.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

As used herein, the term “polymer” refers to oligomers, co-oligomers, polymers and copolymers, e.g., random block, multiblock, star, grafted, gradient copolymers and combination thereof. The average molecular weight of the polymer, as determined by gel permeation chromatography, can range from 500 to about 500,000, e.g., from 20,000 to about 500,000. Without limitation, any polymeric material known in the art can be used in the invention. Accordingly, in some embodiments, the polymer is selected from the group consisting of polysaccharides, polypeptides, polynucleotides, copolymers of fumaric/sebacic acid, poloxamers, polylactides, polyglycolides, polycaprolactones, copolymers of polylactic acid and polyglycolic acid, polyanhydrides, polyepsilon caprolactone, polyamides, polyurethanes, polyesteramides, polyorthoesters, polydioxanones, polyacetals, polyketals, polycarbonates, polyorthocarbonates, polydihydropyrans, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, polymethyl methacrylate, chitin, chitosan, copolymers of polylactic acid and polyglycolic acid, poly(glycerol sebacate) (PGS), gelatin, collagen, silk, alginate, cellulose, poly-nucleic acids, cellulose acetates (including cellulose diacetate), polyethylene, polypropylene, polybutylene, polyethylene terphthalate (PET), polyvinyl chloride, polystyrene, polyamides, nylon, polycarbonates, polysulfides, polysulfones, hydrogels (e.g., acrylics), polyacrylonitrile, polyvinylacetate, cellulose acetate butyrate, nitrocellulose, copolymers of urethane/carbonate, copolymers of styrene/maleic acid, poly(ethylenimine), hyaluron, heparin, agarose, pullulan, and copolymers, terpolymers, and copolymers comprising any combinations thereof.

In some embodiments, the polymer is a biocompatible polymer. As used herein, the term “biocompatible” means exhibition of essentially no cytotoxicity or immunogenicity while in contact with body fluids or tissues. The term “biocompatible polymer” refers to polymers which are non-toxic, chemically inert, and substantially non-immunogenic when used internally in a subject and which are substantially insoluble in blood. The biocompatible polymer can be either non-biodegradable or preferably biodegradable. Preferably, the biocompatible polymer is also non-inflammatory when employed in situ.

Biodegradable polymers are disclosed in the art. Examples of suitable biodegradable polymers include, but are not limited to, linear-chain polymers such as polypeptides, polynucleotides, polysaccharides, polylactides, polyglycolides, polycaprolactones, copolymers of polylactic acid and polyglycolic acid, polyanhydrides, polyepsilon caprolactone, polyamides, polyurethanes, polyesteramides, polyorthoesters, polydioxanones, polyacetals, polyketals, polycarbonates, polyorthocarbonates, polydihydropyrans, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, polymethyl methacrylate, chitin, chitosan, copolymers of polylactic acid and polyglycolic acid, poly(glycerol sebacate) (PGS), fumaric acid, sebacic acid, and copolymers, terpolymers including one or more of the foregoing. Other biodegradable polymers include, for example, gelatin, collagen, silk, chitosan, alginate, cellulose, poly-nucleic acids, etc.

Suitable non-biodegradable biocompatible polymers include, by way of example, cellulose acetates (including cellulose diacetate), polyethylene, polypropylene, polybutylene, polyethylene terphthalate (PET), polyvinyl chloride, polystyrene, polyamides, nylon, polycarbonates, polysulfides, polysulfones, hydrogels (e.g., acrylics), polyacrylonitrile, polyvinylacetate, cellulose acetate butyrate, nitrocellulose, copolymers of urethane/carbonate, copolymers of styrene/maleic acid, poly(ethylenimine), Poloxamers (e.g., Pluronic such as Poloxamers 407 and 188), hyaluronic acid, heparin, agarose, Pullulan, and copolymers including one or more of the foregoing, such as ethylene/vinyl alcohol copolymers (EVOH).

In some embodiments, the biocompatible polymer is a copolymer of polylactic acid and polyglycolic acid, poly(glycerol sebacate) (PGS), poly(ethylenimine), Pluronic (Poloxamers 407, 188), hyaluronic acid, heparin, agarose, or Pullulan.

In some embodiments, the polymer is a homopolymer, a copolymer or a block polymer.

In some embodiments, the polymer comprises side chains selected from the group consisting of amide or ester groups. In some embodiments, the polymer is biodegradable, biocompatible, and non-toxic.

The polymer can be derivatized with a second polymer and the first polymer and the second polymer can be the same or different. For example, the polymer can be derivatized with a polyethylene glycol (PEG).

In some embodiments, polymers or portions of polymers can be connected by linkers. In some embodiments, components of a polymeric particle, e.g., a payload reagent or monocyte-targeting and/or macrophage-targeting ligand can be connected via a linker. As used herein, the term “linker” refers to a moiety that connects two parts of a compound. Linkers typically comprise a direct bond or an atom such as oxygen or sulfur, a unit such as NR₁, C(O), C(O)O, C(O)NR₁, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R₁)₂, C(O), cleavable linking group, substituted or unsubstituted aryl, substituted or unsubstituted heteroaryl, substituted or unsubstituted heterocyclic; where R₁ is hydrogen, acyl, aliphatic or substituted aliphatic.

The linker can be a branched linker. The branch-point of the branched linker can be at least divalent, but can be a trivalent, tetravalent, pentavalent or hexavalent atom, or a group presenting such multiple valencies. In certain embodiments, the branch-point can be, —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In some embodiments, the branch-point can be an acrylate, cyanoacrylate, or methylacrylate.

In various embodiments, the linker is a cleavable linker. A cleavable linker means that the linker can be cleaved to release the two parts the linker is holding together. A cleavable linker can be susceptible to cleavage agents, such as, but not limited to, enzymes, pH, redox potential or the presence of degradative molecules. Examples of such agents: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

In some embodiments, the linker is polyethylene glycol. In some embodiments, the linker is a peptide comprising the sequence DEVD (SEQ ID NO: 1). In a further embodiment, the linker is a peptide comprising the sequence KDEVDAP (SEQ ID NO: 2). In still a further embodiment, the linker is a peptide comprising the sequence GKDEVDAP (SEQ ID NO: 3). In some embodiments, the cleavable linker is cleavable by an enzyme.

In some embodiments, the cleavable linker is selected from a group consisting of small molecules. In some preferred embodiments, the cleavable linker is selected from a group consisting of peptides or polypeptides.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a disease. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

The terms “compound” and “agent” refer to any entity which is normally not present or not present at the levels being administered and/or provided to a cell, tissue or subject. An agent can be selected from a group comprising: chemicals; small organic or inorganic molecules; signaling molecules; nucleic acid sequences; nucleic acid analogues; proteins; peptides; enzymes; aptamers; peptidomimetic, peptide derivative, peptide analogs, antibodies; intrabodies; biological macromolecules, extracts made from biological materials such as bacteria, plants, fungi, or animal cells or tissues; naturally occurring or synthetic compositions or functional fragments thereof. In some embodiments, the agent is any chemical, entity or moiety, including without limitation synthetic and naturally occurring non-proteinaceous entities. In certain embodiments the agent is a small molecule having a chemical moiety. For example, chemical moieties include unsubstituted or substituted alkyl, aromatic, or heterocyclyl moieties including macrolides, leptomycins and related natural products or analogues thereof. Agents can be known to have a desired activity and/or property or can be selected from a library of diverse compounds.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. the M1-polarizing activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide, which retains at least 50% of the wild type reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum. Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route, which results in at least partial delivery of the agent at a desired site. Pharmaceutical compositions comprising the compounds disclosed herein can be administered by any appropriate route, which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

In some events, such as with Hylauronic acid with adehyde modifications, the specific binding can be accompanied by covalent binding to stengthen the cell/particle interaction.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example”.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness ofthe dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:

1. A polymeric particle comprising at least one imaging reagent.

2. The polymeric particle of paragraph 1, wherein the at least one imaging reagent is at least one MRI contrast reagent.

3. The polymeric particle of paragraph 1 or 2, further comprising at least one binding reagent selected from the group consisting of: a CD11b binding reagent; a CD3 binding reagent; a CD19 binding reagent; a CD49b binding reagent; and a CD56 binding reagent

4. A polymeric particle comprising at least one binding reagent selected from the group consisting of: a CD11b binding reagent; a CD3 binding reagent; a CD19 binding reagent; a CD49b binding reagent; and a CD56 binding reagent.

5. The polymeric particle of any of the preceding paragraphs, wherein the binding reagent is a CD11b binding reagent; a CD3 binding reagent; or a CD19 binding reagent.

6. The polymeric particle of any of the preceding paragraphs, wherein the binding reagent is an antibody or an antibody reagent.

7. The polymeric particle of any of the preceding paragraphs, wherein the antibody or antibody reagent is an anti-CD11b antibody or anti-CD11b antibody reagent.

8. The polymeric particle of any of paragraphs 6-7, wherein the antibody or the antibody reagent comprises one or more CDRs of an antibody or antibody reagent selected from Table 2.

9. The polymeric particle of any of paragraphs 6-8, wherein the antibody or the antibody reagent comprises the six CDRs of an antibody or antibody reagent selected from Table 2.

10. The polymeric particle of any of the preceding paragraphs, wherein the binding reagent further comprises a streptavidin molecule or biotin molecule.

11. The polymeric particle of any of the preceding paragraphs, wherein the polymeric particle further comprises one or more cell adhesive molecules (e.g., polyelectrolytes).

12. The polymeric particle of any of the preceding paragraphs, wherein the polymeric particle comprises a single region comprising a hydrogel of one or more cell adhesive molecules (e.g., polyelectrolytes).

13. The polymeric particle of any of paragraphs 1-10, wherein the polymeric particle further comprises one or more structural polymers.

14. The polymeric particle of paragraph 13, wherein the polymeric particle comprises a single region comprising a hydrogel of one or more structural polymers.

15. The polymeric particle of any of paragraphs 1-10, wherein the polymeric particle comprises:
a) a first region comprising a first selection of one or more cell adhesive molecules (e.g., polyelectrolytes) and optionally the one or more binding reagents;
b) a second region comprising a second selection of one or more cell adhesive molecules (e.g., polyelectrolytes); and
c) a third region comprising one or more structural polymers and optionally the one or more binding reagents.

16. The polymeric particle of paragraph 15, wherein the first selection of one or more cell adhesive molecules comprises one or more of: hyaluronic acid (HA) and bovine serum albumin (BSA); the second selection of one or more cell adhesive molecules is poly(allylamine) hydrochloride (PAH); and the one or more structural polymers is one or more of: poly(lactic-co-glycolic) acid (PLGA), PLGA-PEG, PLGA-PEG-maleimide, and PLGA-PEG-biotin.

17. The polymeric particle of any of paragraphs 1-10, wherein the polymeric particle comprises:
a) a first region comprising a first selection of one or more structural polymers and optionally the one or more binding reagents; and
b) a second region comprising a second selection of one or more structural polymers.

18. The polymeric particle of any of paragraphs 1-10, wherein the polymeric particle comprises:
a) a first region comprising a first selection of one or more structural polymers and optionally the one or more binding reagents;
b) a second region comprising a second selection of one or more structural polymers; and
c) a third region comprising a third selection of one or more structural polymers and optionally the one or more binding reagents.

19. The polymeric particle of any of paragraphs 15-18, wherein the second region is located between the first region and third region and/or the second region separates the first region and third region from each other.

20. The polymeric particle of any of paragraphs 10-19, wherein the cell adhesive molecules comprise one or more of cell adhesive polyelectrolytes, immunoglobulins, or ligands for receptors on cell surfaces.

21. The polymeric particle of paragraph 20, wherein the cell adhesive polyelectrolytes comprise one or more of: hyaluronic acid (HA), methylacrylated HA, hyaluronic acid-aldehyde, bovine serum albumin (BSA), PEG, PEG dimethylacrylate, and/or poly(allylamine) hydrochloride (PAH).

22. The polymeric particle of paragraph 21, wherein the cell adhesive polyelectrolytes comprise one or more of: hyaluronic acid (HA), methylacrylated HA, and hyaluronic acid-aldehyde; and one or more of PEG and PEG dimethylacrylate.

23. The polymeric particle of any of paragraphs 10-22, wherein the structural polymer comprises one or more of: poly(lactic-co-glycolic) acid (PLGA); a combination of PLGA and poly (D,L-lactide-co-glycolide); a combination of PLGA and poly (D,L-lactide-co-glycolide) with acid ends; a combination of PLGA and poly (D,L-lactide-co-glycolide) at a 50:50 molar ratio; -polyvinyl alcohol (PVA); hyaluronic acid (HA); gelatin; collagen;PLGA-PEG; or poly(glycerol sebacate) (PGS).

24. The polymeric particle of any of paragraphs 15-23, wherein the second region further comprises poly(lactic-co-caprolactone) (PLCL).

25. The polymeric particle of any of paragraphs 15-24, wherein the second region comprises or further comprises a near-infrared degradable polymer or polymer linker.

26. The polymeric particle of any of paragraphs 1-25, wherein the polymeric particle comprises a binding reagent comprising a streptavidin molecule bound to a structural polymer further comprising biotin.

27. The polymeric particle of paragraph 26, wherein the polymeric particle comprises a binding reagent comprising a streptavidin molecule bound to PLGA-PEG-biotin.

28. The polymeric particle of any of paragraphs 1-26, wherein the polymeric particle comprises a binding reagent bound to PLGA-PEG-maleimide via DTT catalyzed-reaction.

29. The polymeric particle of any of paragraphs 1-25, wherein the polymeric particle comprises a binding reagent comprising a biotin molecule bound to a structural polymer further comprising streptavidin.

30. The polymeric particle of any of the preceding paragraphs, further comprising one or more imaging agents.

31. The polymeric particle of paragraph 30, wherein the one or more imaging agents are selected from the group consisting of: one or more contrast agents, one or more MRI contrast agents, one or more microbubbles, one or more metal ions, one or more radioactive isotopes, one or more optical imaging agents, one or more SPECT imaging agents, and one or more PET imaging agents.

32. The polymeric particle of paragraph 31, wherein the one or more MRI contrast agents comprise one or more of: a gadoliunium-based contrast agent, superparamagnetic iron oxide, ultrasmall superparamagnetic iron oxide, superparamagnetic iron-platinum, manganese chelates, iron salts, and perflubron.

33. The polymeric particle of paragraph 32, wherein the gadoliunium-based contrast agent comprises one or more of: gadolinium, gadoxetate, gadobutrol, gadoterate, gadoteridol, gadopentetate, gadobenate, gadopentetic acid dimegulmine, gadoxentate, gadoversetamide, gadodiamide, gadofosveset, gadocoletic acid, gadomelitol, gadomer 17, and gadoxetic acid.

34. The polymeric particle of any of paragraphs 30-33, wherein the one or more imaging agents further comprise methylacrylate.

35. The polymeric particle of paragraph 30-34, wherein the one or more imaging agents are methylacrylate cross-linked to or more of methylacrylated HA and PEG dimethylacrylate.

36. The polymeric particle of any of the preceding paragraphs, wherein the polymeric particle is substantially discoidal in shape.

37. The polymeric particle of paragraph 36, wherein the polymeric particle is discoidal in shape.

38. The polymeric particle of any of the preceding paragraphs, wherein the diameter of the polymeric particle is from about 100 nm to about 10 μm.

39. The polymeric particle of any of the preceding paragraphs, wherein the diameter of the polymeric particle is from about 100 nm to about 1 μm.

40. The polymeric particle of any of the preceding paragraphs, wherein the polymeric particle is about 6 μm×500 nm in size.

41. The polymeric particle of any of the preceding paragraphs, wherein the polymeric particle is about 6 μm×250 nm in size.

42. The polymeric particle of any of the preceding paragraphs, wherein the polymeric particle is 1-2 μm×7-9 μm in size.

43. The polymeric particle of any of the preceding paragraphs, wherein the polymeric particle has a volume of 0.5×10⁻¹¹ cm³ to 10×10⁻¹¹ cm³.

44. The polymeric particle of any of the preceding paragraphs, wherein the polymeric particle has a volume of 1.25×10⁻¹¹ cm³ to 5×10⁻¹¹ cm³.

45. The polymeric particle of any of the preceding paragraphs, wherein the polymeric particle has a shape which is a rod, a cylinder, a cube, a cuboid, a hexahedron, or a pyramid.

46. The polymeric particle of any of the preceding paragraphs, wherein the polymeric particle further comprises one or more cell-targeting ligands.

47. The polymeric particle of paragraph 46, wherein the cell-targeting ligand is located in a region comprising cell adhesive molecules (e.g., polyelectrolytes).

48. The polymeric particle of any of paragraphs 46-47, wherein the cell-targeting ligand is IgG, an antibody, a polypeptide, or an aptamer.

49. The polymeric particle of any of the preceding paragraphs, wherein the polymeric particle further comprises one or more payload reagents.

50. The polymeric particle of paragraph 49, wherein the payload reagent is a therapeutic molecule.

51. The polymeric particle of paragraph 49, wherein the payload reagent is a small molecule or polypeptide.

52. The polymeric particle of any of paragraphs 50-51, wherein the payload reagent is present in admixture with the structural polymer.

53. The polymeric particle of any of paragraphs 50-52, wherein the payload reagent is present in the second region.

54. The polymeric particle of any of the preceding paragraphs, wherein the polymeric particle further comprises an echogenic liposome.

55. The polymeric particle of any of the preceding paragraphs, wherein the polymeric particle further comprises a magnetic nanoparticle.

56. The polymeric particle of any of the preceding paragraphs, wherein the polymeric particle further comprises a gold nanoparticle.

57. The polymeric particle of any of the preceding paragraphs, wherein a region is a layer.

58. The polymeric particle of any of the preceding paragraphs, further comprising least one polarizing agent.

59. The polymeric particle of paragraph 58, wherein the polarizing agent is an M1-polarizing agent.

60. The polymeric particle of paragraph 58, wherein the polarizing agent is an M2-polarizing agent.

61. The polymeric particle of paragraph 59, wherein the M1-polarizing agent is selected from the group consisting of:
IFN-γ; TNF; TNF-alpha; a Toll-like receptor agonist (e.g., LPS, muramyl dipeptide, or lipoteichoic acid); GM-CSF; IL-1β; IL-6; IL-12; IL-23, and CD11b.

62. The polymeric particle of paragraph 60, wherein the M2-polarizing agent is selected from the group consisting of:
IL-4; IL-10; glucocortoids (e.g., cortisol, cortisone, prednisone, prednisolone, methylprednisonolone, dexamethasone, betamethasone, triamcinolone, fludrocortisone acetate, and deoxycorticosterone acetate); M-CSF, TGF-beta, IL-6; and IL-13.

63. The polymeric particle of any of paragraphs 58-62, wherein the release of one or more of the polarizing agents is triggered by contacting the particle with a small molecule or nucleic acid.

64. The polymeric particle of any of paragraphs 58-63, whereby the phenotype of a macrophage is regulated by the release of the one or more polarizing agents.

65. An engineered cellular composition comprising:
a) a cell; and
b) a polymeric particle of any of the preceding paragraphs, wherein the particle is located on the cell surface of the cell.

66. The composition of paragraph 65, wherein the cell is a monocyte, macrophage, natural killer cell, or neutrophil.

67. The composition of paragraph 66, wherein the macrophage is an M0 macrophage.

68. The composition of paragraph 66, wherein the macrophage is an M1-polarized macrophage.

69. The composition of paragraph 66, wherein the macrophage is an M2-polarized macrophage.

70. The composition of paragraph 66, whereby the macrophage is substantially driven to an M1 or M2 phenotype.

71. A method of obtaining images of a subject, the method comprising administering to the subject the polymeric particle of any of paragraphs 1-64 or the engineered cellular composition of any of paragraphs 65-70, wherein the polymeric particle comprises an imaging reagent, and then subjecting the subject to an imaging scan that can detect the imaging reagent.

72. The method of paragraph 71, wherein the cell is a macrophage, monocyte, or T cell.

73. A method of treating cancer and/or a tumor in a subject in need thereof, the method comprising administering to the subject the polymeric particle of any of paragraphs 1-64 or the engineered cellular composition of any of paragraphs 65-70.

74. The method of paragraph 73, wherein the polymeric particle comprises a payload reagent that is a chemotherapeutic.

75. The method of paragraph 73 or 74, further comprising administering radiation or at least one chemotherapy to the subject.

76. The method of any one of paragraphs 73-75, wherein the cell is a macrophage, NK cell, or T cell.

77. A method of treating a fracture, wound, injury, or infection in a subject in need thereof, the method comprising administering to the subject the polymeric particle of any of paragraphs 1-64 or the engineered cellular composition of any of paragraphs 65-70.

78. The method of paragraph 77, wherein the polymeric particle comprises a payload reagent that is an antibiotic, antiviral, antimicrobial, a hemostatic agent, an anti-inflammatory agent, or a an analgesic.

79. A method of treating inflammation in a subject in need thereof, the method comprising administering to the subject the polymeric particle of any of paragraphs 1-64 or the engineered cellular composition of any of paragraphs 65-70.

80. The method of paragraph 79, wherein the polymeric particle comprises a payload reagent that is an anti-inflammatory agent.

81. The method of paragraph 79 or 80, wherein the inflammation is in the lungs, joints, or skin.

82. The method of any of paragraphs 77-80, wherein the polymeric particle comprises IL-4.

83. The method of any one of paragraphs 77-82, wherein the cell is a neutrophil.

84. A method of treating an autoimmune condition in a subject in need thereof, the method comprising administering to the subject the polymeric particle of any of paragraphs 1-64 or the engineered cellular composition of any of paragraphs 65-70.

85. The method of paragraph 84, wherein the polymeric particle comprises a payload reagent that is an immunosuppressive agent.

86. The method of paragraph 84 or 85, wherein the autoimmune condition is multiple scelarosis, diabetes, or arthritis.

87. The method of any one of paragraphs 84-86, wherein the cell is a macrophage or T cell.

88. A method of providing hemostatic treatment to a subject in need thereof, the method comprising administering to the subject the polymeric particle of any of paragraphs 1-64 or the engineered cellular composition of any of paragraphs 65-70.

89. The method of paragraph 88, wherein the polymeric particle comprises a payload reagent that is a hemostatic agent.

90. A method of treating a neurodegenerative disorder, a central nervous system disorder, or a peripheral nervous system disorder in a subject in need thereof, the method comprising administering to the subject the polymeric particle of any of paragraphs 1-64 or the engineered cellular composition of any of paragraphs 65-70.

91. The method of paragraph 90, wherein the polymeric particle comprises a payload reagent that is therapeutic for the neurodegenerative disorder, the central nervous system disorder, or the peripheral nervous system disorder.

92. A method of providing delivering a payload reagent to the central nervous system of a subject in need thereof, the method comprising administering to the subject the polymeric particle of any of paragraphs 1-64 or the engineered cellular composition of any of paragraphs 65-70, wherein the cell is a monocyte and/or the binding reagent is an anti-CD11b binding reagent.

93. A method of providing a gene therapy vector to a subject in need thereof, the method comprising administering to the subject the polymeric particle of any of paragraphs 1-64 or the engineered cellular composition of any of paragraphs 65-70, wherein the polymeric particle comprises a payload reagent that is a gene therapy vector.

94. The method of paragraph 93, wherein the gene therapy vector is an AAV.

95. A method of vaccinating a subject or inducing an immune response in a subject in need thereof, the method comprising administering to the subject the polymeric particle of any of paragraphs 1-64 or the engineered cellular composition of any of paragraphs 65-70, wherein the polymeric particle comprises a payload reagent that is an antigen.

96. The method of paragraph 95, wherein the cell is a B cell.

97. The method of any of paragraphs 71-96, wherein the cell is autologous to the subject.

98. The method of any of paragraphs 71-96, wherein the cell is heterologous to the subject.

99. The method of any of paragraphs 71-98, further comprising a first step of obtaining the cell from a donor and/or the subject and contacting the cell with the polymeric particle ex vivo.

100. The method of any of paragraphs 71-99, wherein a therapeutically effective dose of the polymeric particle or engineered cellular composition is administered.

In some embodiments, the present technology may be defined in any of the following numbered paragraphs:

1. A polymeric particle comprising at least one imaging reagent.

2. The polymeric particle of paragraph 1, wherein the at least one imaging reagent is at least one MRI contrast reagent.

3. The polymeric particle of paragraph 1 or 2, further comprising at least one binding reagent selected from the group consisting of: a CD11b binding reagent; a CD3 binding reagent; a CD19 binding reagent; a CD49b binding reagent; a CD56 binding reagent; a CD11a binding reagent; a CD27 binding reagent; a CD44 binding reagent; a CD45 binding reagent; a NKG2D binding reagent; a NKp30 binding reagent; a NKp46 binding reagent; and an ICAM1 binding reagent.

4. A polymeric particle comprising at least one binding reagent selected from the group consisting of: a CD11b binding reagent; a CD3 binding reagent; a CD19 binding reagent; a CD49b binding reagent; a CD56 binding reagent; a CD11a binding reagent; a CD27 binding reagent; a CD44 binding reagent; a CD45 binding reagent; a NKG2D binding reagent; a NKp30 binding reagent; a NKp46 binding reagent; and an ICAM1 binding reagent.

5. The polymeric particle of any of the preceding paragraphs, wherein the binding reagent is a CD11b binding reagent; a CD3 binding reagent; or a CD19 binding reagent.

6. The polymeric particle of any of the preceding paragraphs, wherein the binding reagent is a CD11 b binding reagent.

7. The polymeric particle of any of the preceding paragraphs, wherein the binding reagent is a CD45 binding reagent.

8. The polymeric particle of any of the preceding paragraphs, wherein the binding reagent is an antibody or an antibody reagent.

9. The polymeric particle of any of the preceding paragraphs, wherein the antibody or antibody reagent is an anti-CD11b antibody or anti-CD11b antibody reagent.

10. The polymeric particle of any of paragraphs 8-9, wherein the antibody or the antibody reagent comprises one or more CDRs of an antibody or antibody reagent selected from Table 2.

11. The polymeric particle of any of paragraphs 8-10, wherein the antibody or the antibody reagent comprises the six CDRs of an antibody or antibody reagent selected from Table 2.

12. The polymeric particle of any of the preceding paragraphs, wherein the binding reagent further comprises a streptavidin molecule or biotin molecule.

13. The polymeric particle of any one of the preceding paragraphs, further comprising ICAM1, NKp30, and/or NKp46.

14. A polymeric particle comprising ICAM1, NKp30, and/or NKp46.

15. The polymeric particle of any of the preceding paragraphs, comprising ICAM1 and NKp30.

16. The polymeric particle of any of the preceding paragraphs, wherein the polymeric particle further comprises one or more cell adhesive molecules (e.g., polyelectrolytes).

17. The polymeric particle of any of the preceding paragraphs, wherein the polymeric particle comprises a single region comprising a hydrogel of one or more cell adhesive molecules (e.g., polyelectrolytes).

18. The polymeric particle of any of paragraphs 1-17, wherein the polymeric particle further comprises one or more structural polymers.

19. The polymeric particle of paragraph 18, wherein the polymeric particle comprises a single region comprising a hydrogel of one or more structural polymers.

20. The polymeric particle of any of paragraphs 1-18, wherein the polymeric particle comprises:
a) a first region comprising a hydrogel of one or more structural polymers; and
b) a second region comprising one or more cell adhesive molecules (e.g., polyelectrolytes) and the one or more binding reagents.

21. The polymeric particle of any of paragraphs 1-18, wherein the polymeric particle comprises:
a) a first region comprising a first selection of one or more cell adhesive molecules (e.g., polyelectrolytes) and optionally the one or more binding reagents;
b) a second region comprising a second selection of one or more cell adhesive molecules (e.g., polyelectrolytes); and
c) a third region comprising one or more structural polymers and optionally the one or more binding reagents.

22. The polymeric particle of paragraph 21, wherein the first selection of one or more cell adhesive molecules comprises one or more of: hyaluronic acid (HA) and bovine serum albumin (BSA); the second selection of one or more cell adhesive molecules is poly(allylamine) hydrochloride (PAH); and the one or more structural polymers is one or more of: poly(lactic-co-glycolic) acid (PLGA), PLGA-PEG, PLGA-PEG-maleimide, and PLGA-PEG-biotin.

23. The polymeric particle of any of paragraphs 1-18, wherein the polymeric particle comprises:
a) a first region comprising a first selection of one or more structural polymers and optionally the one or more binding reagents; and
b) a second region comprising a second selection of one or more structural polymers.

24. The polymeric particle of any of paragraphs 1-18, wherein the polymeric particle comprises:
a) a first region comprising a first selection of one or more structural polymers and optionally the one or more binding reagents;
b) a second region comprising a second selection of one or more structural polymers; and
c) a third region comprising a third selection of one or more structural polymers and optionally the one or more binding reagents.

25. The polymeric particle of any of paragraphs 21, 22, and 24, wherein the second region is located between the first region and third region and/or the second region separates the first region and third region from each other.

26. The polymeric particle of any of paragraphs 21, 22, 24, and 25, wherein the third region comprises one or more binding reagents and the first region does not comprise one or more binding reagents.

27. The polymeric particle of any of paragraphs 21, 22, and 24-26, wherein the first selection of one or more structural polymers comprises or consists of PLGA, the second selection of one or more structural polymers comprises or consists of PVA; and the third selection of one or more structural polymers comprises or consists of PLGA.

28. The polymeric particle of any one of paragraphs 21, 22, and 24-27, wherein the second region and/or third region comprises one or more active agents.

29. The polymeric particle of any one of paragraphs 21, 22, and 24-28, wherein the second region and third region each comprise one or more payload agents.

30. The polymeric particle of any one of paragraphs 28 and 29, wherein the one or more payload agents are N2/M2-polarizing agents.

31. The polymeric particle of any one of paragraphs 28-30, wherein the second region comprises IL-4 and the third region comprises dexamethasone.

32. The polymeric particle of any one of paragraphs 28-31, wherein the second region comprises IL-4 and heparin and the third region comprises dexamethasone.

33. The polymeric particle of any of paragraphs 16-32, wherein the cell adhesive molecules comprise one or more of cell adhesive polyelectrolytes, immunoglobulins, or ligands for receptors on cell surfaces.

34. The polymeric particle of paragraph 33, wherein the cell adhesive polyelectrolytes comprise one or more of: hyaluronic acid (HA), methylacrylated HA, hyaluronic acid-aldehyde, bovine serum albumin (BSA), PEG, PEG dimethylacrylate, and/or poly(allylamine) hydrochloride (PAH).

35. The polymeric particle of paragraph 33, wherein the cell adhesive polyelectrolytes comprise:
a) one or more of: hyaluronic acid (HA), methylacrylated HA, and hyaluronic acid-aldehyde; and one or more of PEG and PEG dimethylacrylate;
b) poly(allylamine) hydrochloride (PAH) and one or more of: hyaluronic acid (HA), methylacrylated HA, and hyaluronic acid-aldehyde;
c) poly(allylamine) hydrochloride (PAH) and hyaluronic acid-aldehyde;
d) pol(allylamine) hydrochloride (PAH) and hyaluronic acid (HA); or
e) poly(allylamine) hydrochloride (PAH) and bovine serum albumin (BSA).

36. The polymeric particle of any of paragraphs 18-35, wherein the structural polymer comprises one or more of: poly(lactic-co-glycolic) acid (PLGA); a combination of PLGA and poly (D,L-lactide-co-glycolide); a combination of PLGA and poly (D,L-lactide-co-glycolide) with acid ends; a combination of PLGA and poly (D,L-lactide-co-glycolide) at a 50:50 molar ratio; -polyvinyl alcohol (PVA); hyaluronic acid (HA); gelatin; collagen;PLGA-PEG; or poly(glycerol sebacate) (PGS).

37. The polymeric particle of any of paragraphs 18-36, wherein the structural polymer comprises or consists of poly(lactic-co-glycolic) acid (PLGA)-PEG.

38. The polymeric particle of any of paragraphs 20-37, wherein the second region further comprises poly(lactic-co-caprolactone) (PLCL).

39. The polymeric particle of any of paragraphs 20-38, wherein the second region comprises or further comprises a near-infrared degradable polymer or polymer linker.

40. The polymeric particle of any of paragraphs 1-39, wherein the polymeric particle comprises a binding reagent comprising a streptavidin molecule bound to a structural polymer further comprising biotin.

41. The polymeric particle of any of paragraphs 1-40, wherein the polymeric particle comprises a binding reagent comprising a streptavidin molecule bound to PLGA-PEG-biotin.

42. The polymeric particle of any of paragraphs 1-41, wherein the polymeric particle comprises a binding reagent bound to PLGA-PEG-maleimide via DTT catalyzed-reaction.

43. The polymeric particle of any of paragraphs 1-42, wherein the polymeric particle comprises a binding reagent comprising a biotin molecule bound to a structural polymer further comprising streptavidin.

44. The polymeric particle of any of the preceding paragraphs, further comprising one or more imaging agents.

45. The polymeric particle of paragraph 44, wherein the one or more imaging agents are selected from the group consisting of: one or more contrast agents, one or more MRI contrast agents, one or more microbubbles, one or more metal ions, one or more radioactive isotopes, one or more optical imaging agents, one or more SPECT imaging agents, and one or more PET imaging agents.

46. The polymeric particle of paragraph 45, wherein the one or more MRI contrast agents comprise one or more of: a gadoliunium-based contrast agent, superparamagnetic iron oxide, ultrasmall superparamagnetic iron oxide, superparamagnetic iron-platinum, manganese chelates, iron salts, and perflubron.

47. The polymeric particle of paragraph 46, wherein the gadoliunium-based contrast agent comprises one or more of: gadolinium, gadoxetate, gadobutrol, gadoterate, gadoteridol, gadopentetate, gadobenate, gadopentetic acid dimegulmine, gadoxentate, gadoversetamide, gadodiamide, gadofosveset, gadocoletic acid, gadomelitol, gadomer 17, and gadoxetic acid.

48. The polymeric particle of any one of paragraphs 44-47, wherein the one or more imaging agents further comprise methylacrylate.

49. The polymeric particle of any one of paragraph 44-48, wherein the one or more imaging agents are methylacrylate cross-linked to or more of methylacrylated HA and PEG dimethylacrylate.

50. The polymeric particle of any of the preceding paragraphs, wherein the polymeric particle is substantially discoidal in shape.

51. The polymeric particle of paragraph 50, wherein the polymeric particle is discoidal in shape.

52. The polymeric particle of any one of the preceding paragraphs, wherein the diameter of the polymeric particle is from about 100 nm to about 10 μm.

53. The polymeric particle of any one of the preceding paragraphs, wherein the diameter of the polymeric particle is from about 100 nm to about 1 μm.

54. The polymeric particle of any one of the preceding paragraphs, wherein the polymeric particle is about 6 μm×500 nm in size.

55. The polymeric particle of any one of the preceding paragraphs, wherein the polymeric particle is about 6 μm×250 nm in size.

56. The polymeric particle of any one of the preceding paragraphs, wherein the polymeric particle is 1-2 μm×7-9 μm in size.

57. The polymeric particle of any one of the preceding paragraphs, wherein the polymeric particle has a volume of 0.5×10⁻¹¹ cm³ to 10×10⁻¹¹ cm³.

58. The polymeric particle of any one of the preceding paragraphs, wherein the polymeric particle has a volume of 1.25×10⁻¹¹ cm³ to 5×10⁻¹¹ cm³.

59. The polymeric particle of any one of the preceding paragraphs, wherein the polymeric particle has a shape which is a rod, a cylinder, a cube, a cuboid, a hexahedron, or a pyramid.

60. The polymeric particle of any one of the preceding paragraphs, wherein the polymeric particle further comprises one or more cell-targeting ligands.

61. The polymeric particle of paragraph 60, wherein the cell-targeting ligand is located in a region comprising cell adhesive molecules (e.g., polyelectrolytes).

62. The polymeric particle of any one of paragraphs 60-61, wherein the cell-targeting ligand is IgG, an antibody, a polypeptide, or an aptamer.

63. The polymeric particle of any one of the preceding paragraphs, wherein the polymeric particle further comprises one or more payload reagents.

64. The polymeric particle of paragraph 63, wherein the payload reagent is a therapeutic molecule.

65. The polymeric particle of paragraph 63, wherein the payload reagent is a small molecule or polypeptide.

66. The polymeric particle of any one of paragraphs 63-65, wherein the payload reagent is present in admixture with the structural polymer.

67. The polymeric particle of any one of paragraphs 63-66, wherein the payload reagent is present in the second region.

68. The polymeric particle of any one of the preceding paragraphs, wherein the polymeric particle further comprises an echogenic liposome.

69. The polymeric particle of any one of the preceding paragraphs, wherein the polymeric particle further comprises a magnetic nanoparticle.

70. The polymeric particle of any one of the preceding paragraphs, wherein the polymeric particle further comprises a gold nanoparticle.

71. The polymeric particle of any one of the preceding paragraphs, wherein a region is a layer.

72. The polymeric particle of any one of the preceding paragraphs, further comprising at least one priming agent.

73. The polymeric particle of paragraph 72, wherein the at least one priming agent is selected from the group consisting of: a caspase inhibitor; fMLF; C5A; LTB4; PAF; TNF-α; GCSF; GM-CSF; IFN-γ; IL-1β; IL8; IL-15; IL-18; IL-33; adiponectin; LPS; LAMs; lipopeptide; flagellin; ATP; substance P; CL097; and CL075.

74. The polymeric particle of any one of paragraphs 72-73, wherein the at least one priming agent is selected from the group consisting of: a caspase inhibitor; GCSF; and GM-CSF.

75. The polymeric particle of any one of paragraphs 73-74, wherein the caspase inhibitor is a pan-caspase inhibitor.

76. The polymeric particle of any one of paragraphs 72-73, wherein the at least one priming agent is selected from the group consisting of IL-2 and IL-15.

77. The polymeric particle of any of the preceding paragraphs, further comprising least one polarizing agent.

78. The polymeric particle of paragraph 77, wherein the polarizing agent is an N1/M1-polarizing agent.

79. The polymeric particle of paragraph 77, wherein the polarizing agent is an N2/M2-polarizing agent.

80. The polymeric particle of paragraph 78, wherein the N1/M1-polarizing agent is selected from the group consisting of:
IFN-γ; TNF; TNF-alpha; a Toll-like receptor agonist (e.g., LPS, muramyl dipeptide, or lipoteichoic acid); GM-CSF; IL-1β; IL-6; IL-12; IL-23, and CD11b.

81. The polymeric particle of paragraph 79, wherein the N2/M2-polarizing agent is selected from the group consisting of:
IL-4; IL-10; glucocortoids (e.g., cortisol, cortisone, prednisone, prednisolone, methylprednisonolone, dexamethasone, betamethasone, triamcinolone, fludrocortisone acetate, and deoxycorticosterone acetate); M-CSF, TGF-beta, IL-6; and IL-13.

82. The polymeric particle of any of paragraphs 77-81, wherein the release of one or more of the polarizing agents is triggered by contacting the particle with a small molecule or nucleic acid.

83. The polymeric particle of any of paragraphs 77-82, whereby the phenotype of a macrophage is regulated by the release of the one or more polarizing agents.

84. An engineered cellular composition comprising:
a) a cell; and
b) a polymeric particle of any of the preceding paragraphs, wherein the particle is located on the cell surface of the cell.

85. The composition of paragraph 84, wherein the cell is a monocyte, macrophage, natural killer cell, or neutrophil.

86. The composition of any one of paragraphs 84-85, wherein the cell is a neutrophil and the polymeric particle comprises a) at least one region comprising or consisting of PLGA and b) a CD11 b binding reagent.

87. The composition of any one of paragraphs 84-85, wherein the cell is a NK cell and the polymeric particle comprises a) at least one region comprising or consisting of PLGA and/or PLGA-PEG; b) a CD45 binding reagent; and c) a NKp30 binding reagent.

88. The composition of any one of paragraphs 84-85, wherein the cell is a monocyte and the polymeric particle comprises a) a first region comprising a first selection of one or more structural polymers; b) a second region comprising a second selection of one or more structural polymers; and c) a third region comprising a CD11b binding reagent and a third selection of one or more structural polymer.

89. The composition of paragraph 85, wherein the macrophage is an M0 macrophage.

90. The composition of paragraph 85, wherein the macrophage is an M1-polarized macrophage.

91. The composition of paragraph 85, wherein the macrophage is an M2-polarized macrophage.

92. The composition of paragraph 85, whereby the macrophage, monocyte, or neutrophil is substantially driven to an N1/M1 or N2/M2 phenotype.

93. A method of preparing an engineered cellular composition of any of paragraphs 84-91, comprising contacting the cell with the polymeric particle.

94. The method of paragraph 93, further comprising contacting the cell with at least one priming agent before or during the step of contacting the cell with the polymeric particle.

95. The method of paragraph 94, wherein the at least one priming agent is selected from the group consisting of: a caspase inhibitor; fMLF; C5A; LTB4; PAF; TNF-α; GCSF; GM-CSF; IFN-γ; IL-1β; IL8; IL-15; IL-18; IL-33; adiponectin; LPS; LAMs; lipopeptide; flagellin; ATP; substance P; CL097; and CL075; and optionally the cell is a neutrophil.

96. The method of any one of paragraphs 94-95, wherein the at least one priming agent is selected from the group consisting of: a caspase inhibitor; GCSF; and GM-CSF.

97. The method of any one of paragraphs 95-96 wherein the caspase inhibitor is a pan-caspase inhibitor.

98. The method of paragraph 94, wherein the at least one priming agent is selected from the group consisting of IL-2; and IL-15; and optionally the cell is a NK cell.

99. The method of any one of paragraphs 93-98, wherein the step of contacting the cell with the polymeric particle occurs in the well of a multi-well plate.

100. The method of any one of paragraphs 93-99, wherein the step of contacting the cell with the polymeric particle occurs in the well of a 96 well multi-well plate.

101. The method of any one of paragraphs 93-100, wherein the step of contacting the cell with the polymeric particle occurs in medium comprising serum.

102. The method of any one of paragraphs 100-101, wherein the step of contacting the cell with the polymeric particle occurs in medium comprising fetal bovine serum (FBS).

103. The method of any one of paragraphs 100-102, wherein the step of contacting the cell with the polymeric particle occurs medium comprising serum; and the method further comprises contacting the cell with a capsase inhibitor and GMCSF before or during the step of contacting the cell with the polymeric particle.

104. The method of any one of paragraphs 100-103, wherein the step of contacting the cell with the polymeric particle occurs medium comprising serum; and the method further comprises contacting the cell with GMCSF before or during the step of contacting the cell with the polymeric particle.

105. The method of any one of paragraphs 99-104, wherein the cell is a neutrophil.

106. A method of obtaining images of a subject, the method comprising administering to the subject the polymeric particle of any of paragraphs 1-83 or the engineered cellular composition of any of paragraphs 84-92, wherein the polymeric particle comprises an imaging reagent, and then subjecting the subject to an imaging scan that can detect the imaging reagent.

107. The method of paragraph 106, wherein the cell is a macrophage, monocyte, or T cell.

108. A method of treating cancer and/or a tumor in a subject in need thereof, the method comprising administering to the subject the polymeric particle of any of paragraphs 1-83 or the engineered cellular composition of any of paragraphs 84-92.

109. The method of paragraph 108, wherein the polymeric particle comprises a payload reagent that is a chemotherapeutic.

110. The method of any one of paragraphs 108-109, further comprising administering radiation or at least one chemotherapy to the subject.

111. The method of any one of paragraphs 108-110, wherein the cell is a macrophage, NK cell, or T cell.

112. A method of treating a fracture, wound, injury, or infection in a subject in need thereof, the method comprising administering to the subject the polymeric particle of any of paragraphs 1-83 or the engineered cellular composition of any of paragraphs 84-92.

113. The method of paragraph 112, wherein the polymeric particle comprises a payload reagent that is an antibiotic, antiviral, antimicrobial, a hemostatic agent, an anti-inflammatory agent, or an analgesic.

114. A method of treating inflammation in a subject in need thereof, the method comprising administering to the subject the polymeric particle of any of paragraphs 1-83 or the engineered cellular composition of any of paragraphs 84-92.

115. The method of paragraph 114, wherein the polymeric particle comprises a payload reagent that is an anti-inflammatory agent.

116. The method of any one of paragraphs 114-115, wherein the inflammation is in the lungs, joints, or skin.

117. The method of any one of paragraphs 114-116, wherein the polymeric particle comprises IL-4.

118. The method of any one of paragraphs 114-117, wherein the cell is a neutrophil.

119. A method of treating an autoimmune condition in a subject in need thereof, the method comprising administering to the subject the polymeric particle of any of paragraphs 1-83 or the engineered cellular composition of any of paragraphs 84-92.

120. The method of paragraph 119, wherein the polymeric particle comprises a payload reagent that is an immunosuppressive agent.

121. The method of paragraph 119 or 120, wherein the autoimmune condition is multiple scelarosis, diabetes, or arthritis.

122. The method of any one of paragraphs 119-121, wherein the cell is a macrophage or T cell.

123. A method of providing hemostatic treatment to a subject in need thereof, the method comprising administering to the subject the polymeric particle of any of paragraphs 1-83 or the engineered cellular composition of any of paragraphs 84-92.

124. The method of paragraph 123, wherein the polymeric particle comprises a payload reagent that is a hemostatic agent.

125. A method of treating a neurodegenerative disorder, a central nervous system disorder, or a peripheral nervous system disorder in a subject in need thereof, the method comprising administering to the subject the polymeric particle of any of paragraphs 1-83 or the engineered cellular composition of any of paragraphs 84-92.

126. The method of paragraph 125, wherein the polymeric particle comprises a payload reagent that is therapeutic for the neurodegenerative disorder, the central nervous system disorder, or the peripheral nervous system disorder.

127. A method of providing delivering a payload reagent to the central nervous system of a subject in need thereof, the method comprising administering to the subject the polymeric particle of any of paragraphs 1-83 or the engineered cellular composition of any of paragraphs 84-92, wherein the cell is a monocyte and/or the binding reagent is an anti-CD11b binding reagent.

128. A method of providing a gene therapy vector to a subject in need thereof, the method comprising administering to the subject the polymeric particle of any of paragraphs 1-83 or the engineered cellular composition of any of paragraphs 84-92, wherein the polymeric particle comprises a payload reagent that is a gene therapy vector.

129. The method of paragraph 128, wherein the gene therapy vector is an AAV.

130. A method of vaccinating a subject or inducing an immune response in a subject in need thereof, the method comprising administering to the subject the polymeric particle of any of paragraphs 1-83 or the engineered cellular composition of any of paragraphs 84-92, wherein the polymeric particle comprises a payload reagent that is an antigen.

131. The method of paragraph 130, wherein the cell is a B cell.

132. The method of any of paragraphs 106-131, wherein the cell is autologous to the subject.

133. The method of any of paragraphs 106-132, wherein the cell is heterologous to the subject.

134. The method of any of paragraphs 106-133, further comprising a first step of obtaining the cell from a donor and/or the subject and contacting the cell with the polymeric particle ex vivo.

135. The method of any of paragraphs 106-134, wherein a therapeutically effective dose of the polymeric particle or engineered cellular composition is administered.

The technology described herein is further illustrated by the following examples which in no way should be construed as being further limiting.

### EXAMPLES

### Example 1

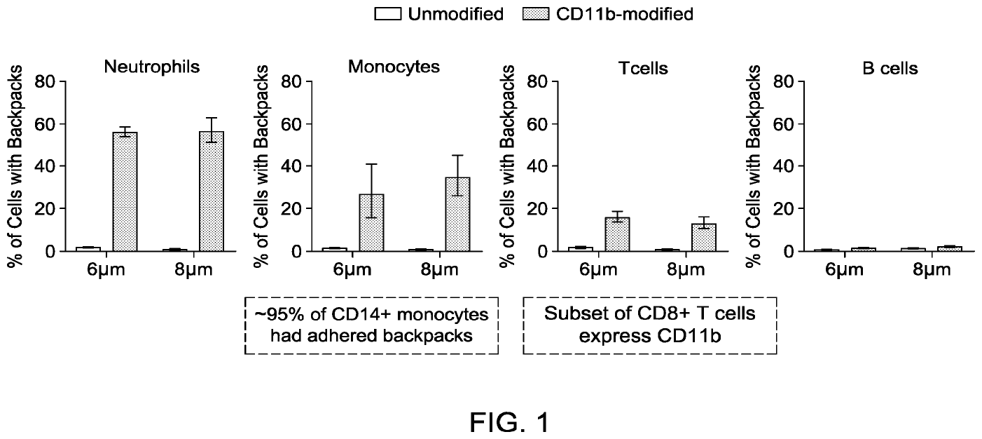

Polymeric particles (backpacks) were prepared which included a CD11b antibody (M1/70 clone, available from Biolegend as Purified anti-mouse/human CD 11b Antibody Cat #101201/101202). The inclusion of the CDllb-binding reagent significantly increased backpack binding in whole blood to neutrophils, monocytes, and T cells (FIG. 1). In particular, 95% of CD14+monocytes had adhered backpacks.

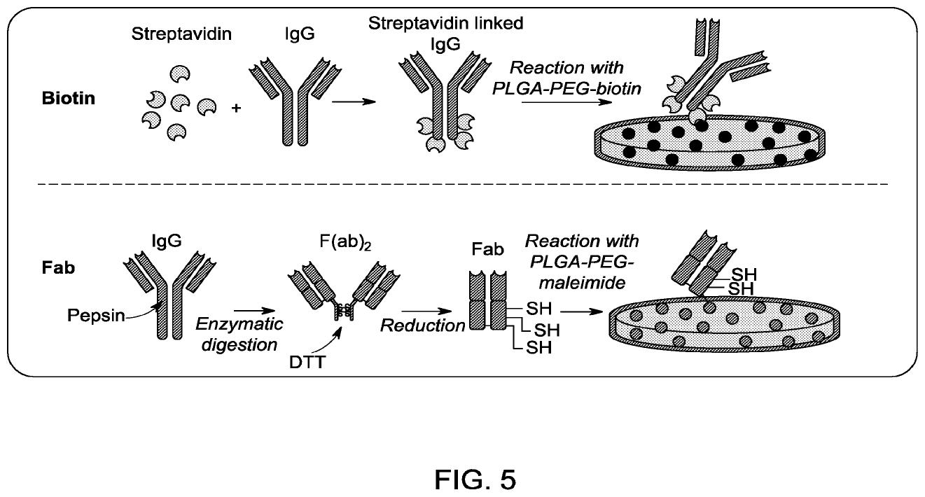

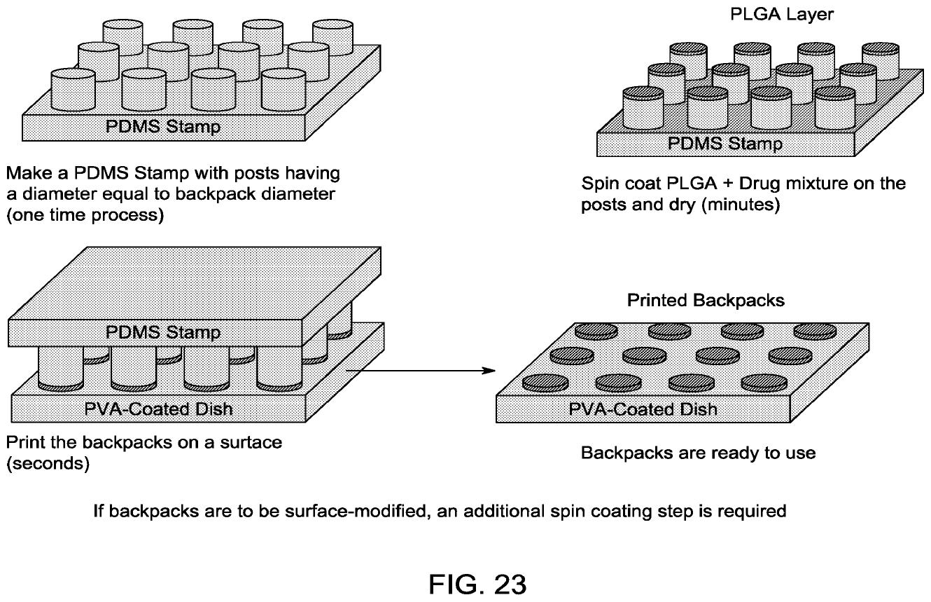

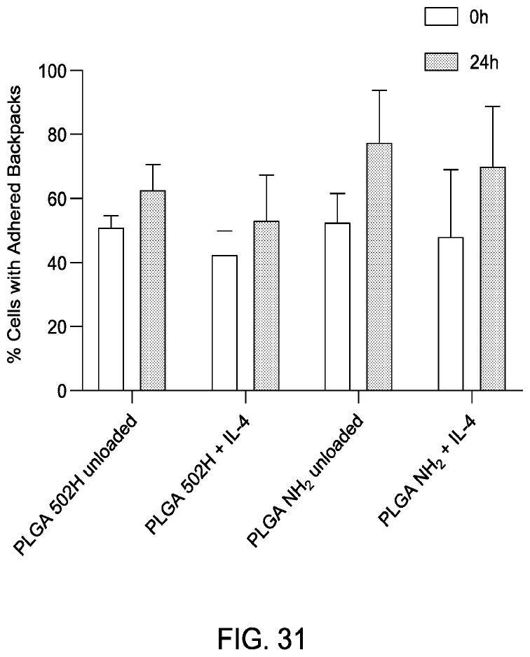

PLGA+anti-CD11b backpacks were prepared using PLGA502H (Sigma-Aldrich). These backpacks adhered to a significant fraction of monocytes and remained attached for at least 24 hours. Loading of the backpacks with drugs did not impact the adhesion (FIG. 31). The process for preparing the PLGA backbacks is depicted in FIG. 23. Anti-CD11b was conjugated to the surface of the backpacks. FIG. 5 depicts one means of attaching anti-CD11b to backpacks.

### Example 2

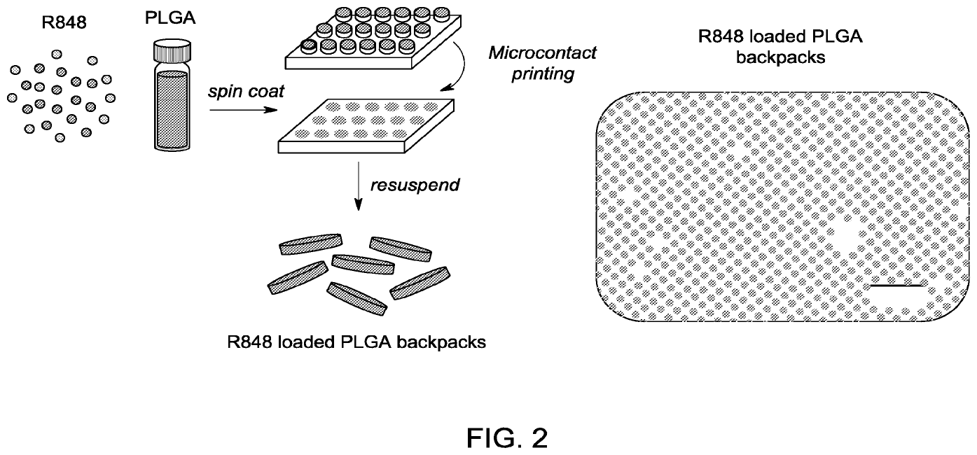

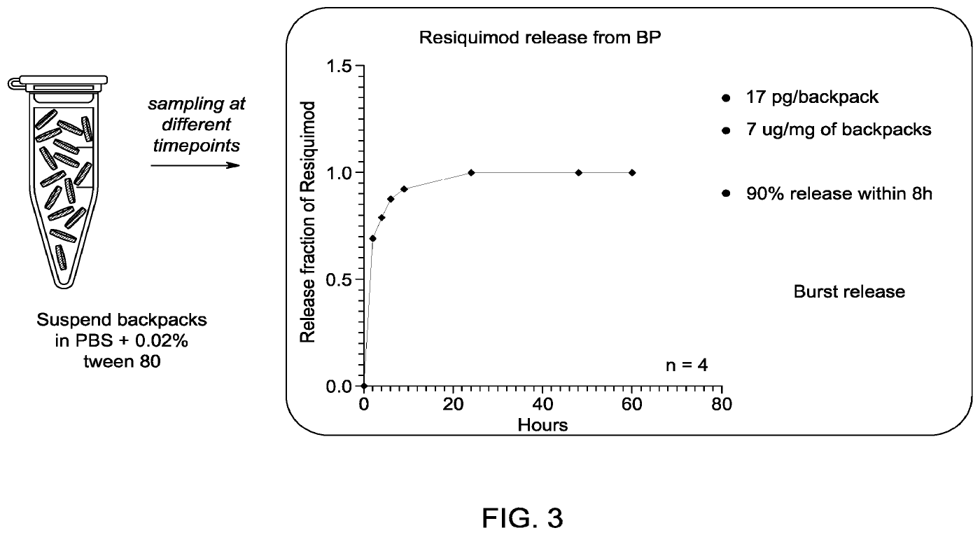

PLGA backpacks loaded with resiquimod were prepared as shown in FIG. 2. The backpacks were found to provide burst release of resiquimod (FIG. 3).

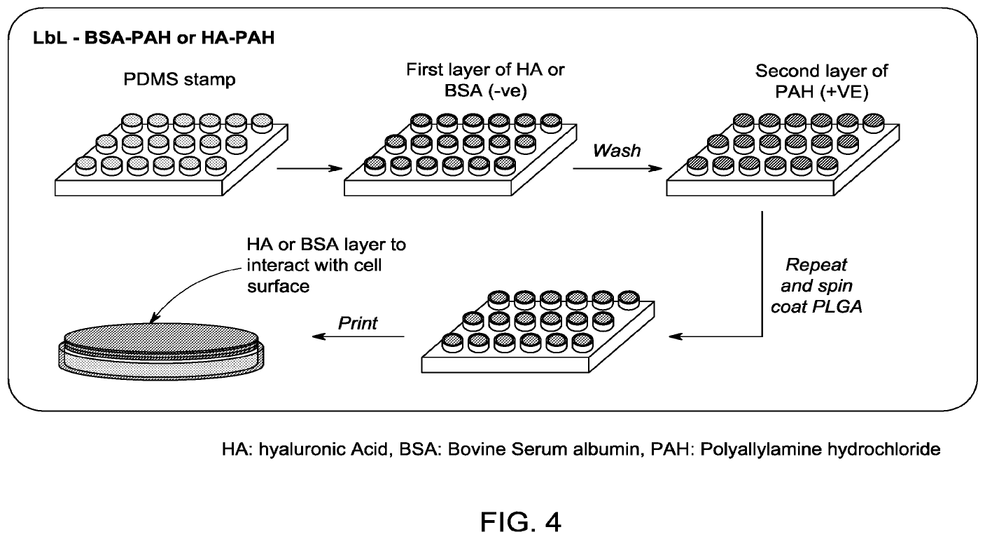

Layered backpacks comprising a first layer of HA or BSA, a second layer of PAH, and coating of PLGA were prepared as shown in FIG. 4. Backpacks comprising anti-CD11b, anti-CD49b, or anti-CD56 reagents were also prepared, either by 1) linking the antibody reagent to streptavidin and binding it to PLGA-PEG-biotin provided in the PLGA coating of the backpacks, or by 2) pepsin-digesting the antibody reagent, reacting the resulting F(ab)₂ fragment with DTT, and then binding it to PLGA-PEG-maleimide provided in the PLGA coating of the backpacks (FIG. 5). Adhesion of these backpacks was then tested using NK-92 cells or mouse NK cells.

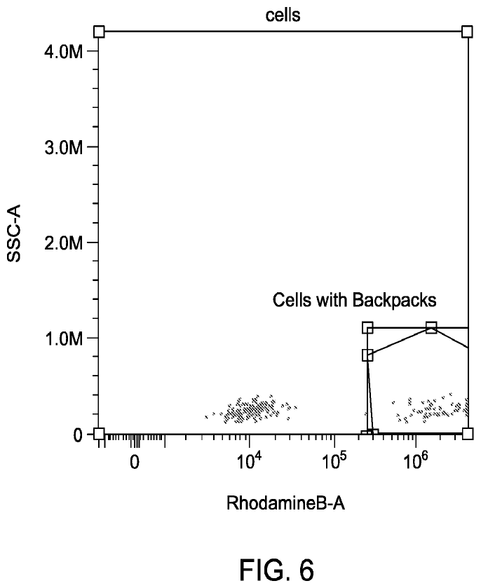

For experiments with NK-92 cells, 10⁶ cells/ml were used with 1:1 to 3:1 Backpack to cell ratios. Incubation proceeded for 1-3 hours in PBS. The designs tested with NK-92 cells were BSA-PAH, HA-PAH, Biotin-CD11b, Biotin-CD56. For experiments with mouse NK cells, 10⁶ cells/ml were used with 1:1 to 7:1 Backpack to cell ratios. Incubation proceeded for 1-3 hours in PBS and media. The designs tested with NK-92 cells were BSA-PAH, Biotin-CD11b, and Biotin-CD49b. Adhesion can be quantified by flow cytometry gated via an FSC-SSC plot, as backpacks on NK cells undergo a significant shift along the Rhodamine-B axis (FIG. 6).

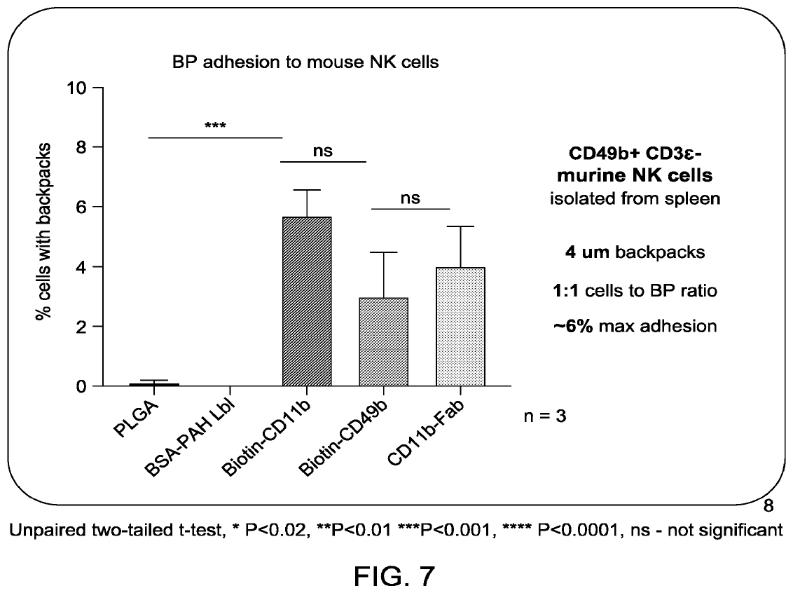

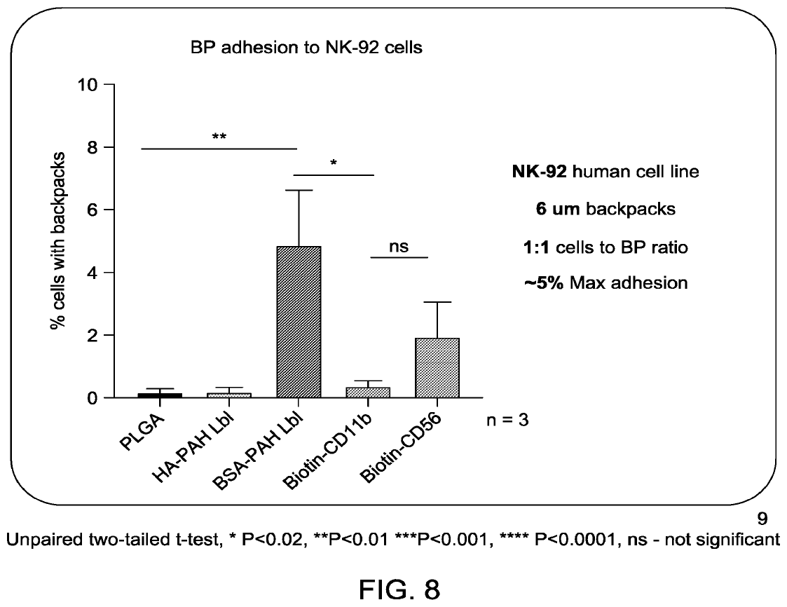

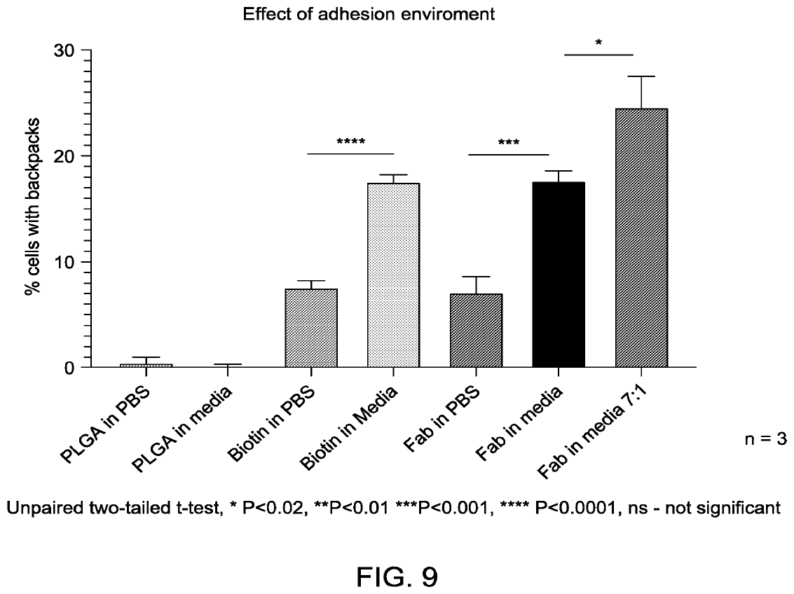

Backpacks incorporating an anti-CD11b reagent were demonstrated to adhere to primary mouse NK cells (FIG. 7). BSA-PAH backpacks were demonstrated to adhere to NK-92 cells (FIG. 8). The results demonstrate that adhesion can be improved under different conditions (FIG. 9). In particular, a more than 2-fold increase was observed in presence of media vs PBS. Additionally, a 7:1 backpack to cell ratio leads to higher adhesion as compared to a 3:1 ratio. When binding to NK-92 cells, the BSA-PAH design adhered better than PLGA designs. When binding to mouse NK cells, Biotin-CD11b, Biotin-CD49b, and Fab CD11b designs adhered better than PLGA designs. When binding to mouse NK cells, better adhesion was obtained in RPMI-1640 supplemented with 10% FBS as compared to PBS.

### Example 3

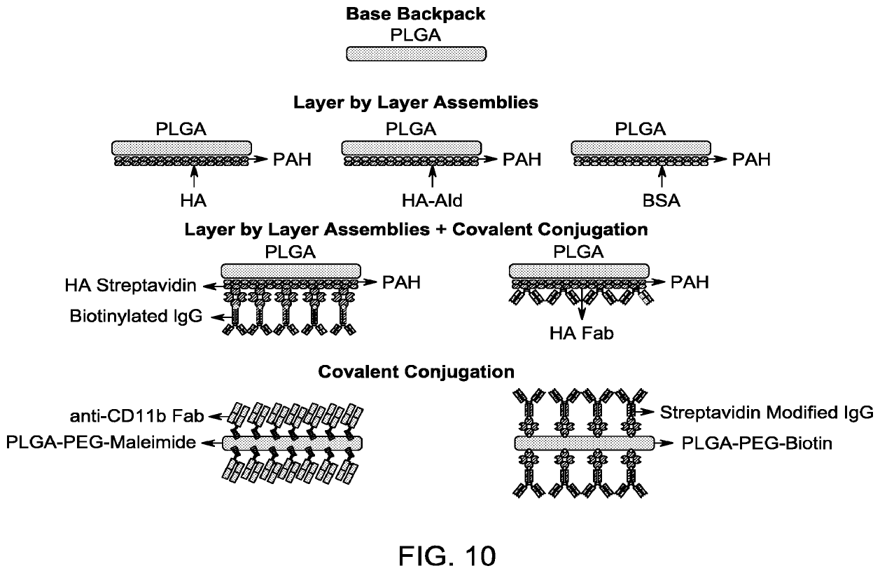

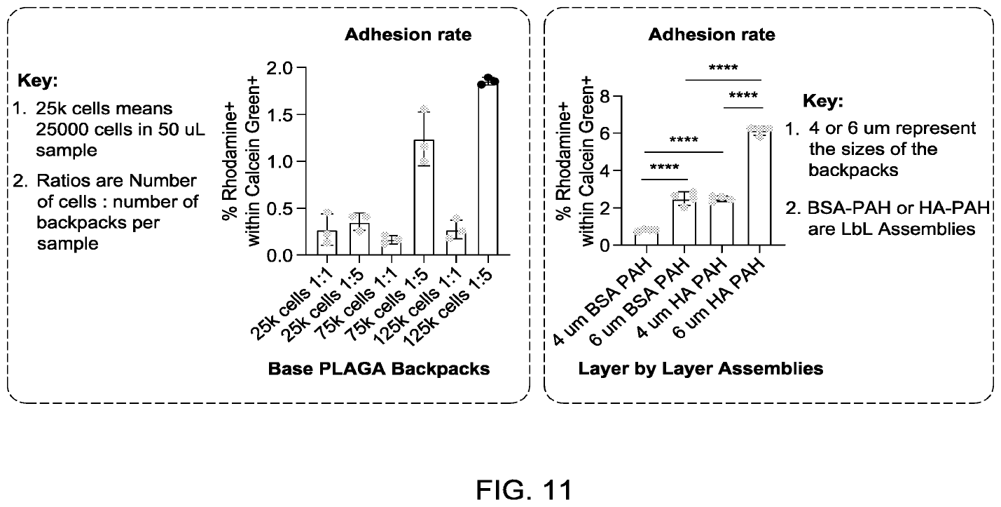

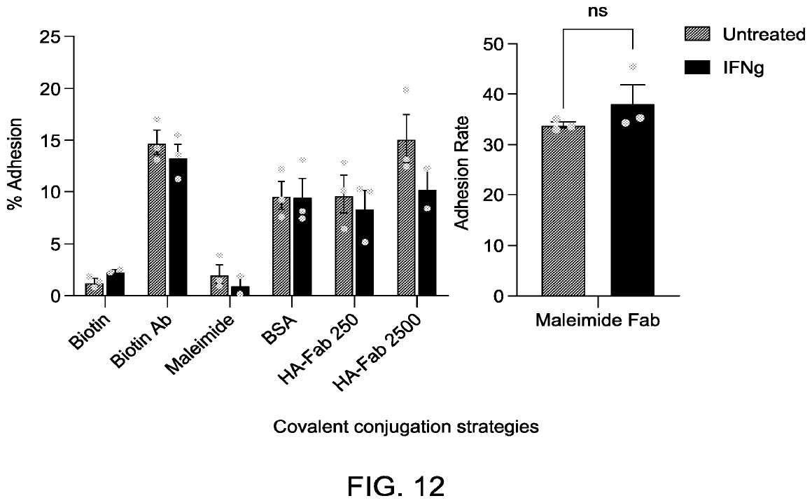

Several different backpacks (polymeric particles) were designed and assembled as depicted in FIG. 10, and their rate of adhesion to neutrophils was assessed (FIG. 11). Different covalent conjugation strategies were explored, namely:
Biotin: BP fabricated from PLGA PEG Biotin
Biotin Ab: BP fabricated from PLGA PEG Biotin modified with antibody
Maleimide: BP fabricated from PLGA PEG Maleimide
Maleimide Fab: BP fabricated from PLGA PEG Maleimide modified with antibody
BSA: LbL of HA modified with BSA with PAH
HA-Fab 250: LbL of 250 kDa HA modified with Fab with PAH
HA-Fab 2500: LbL of 2500 kDa HA modified with Fab with PAH
Untreated: Control group (incubated in cell culture medium)
IFNg: Cells Treated with interferon for 20 minutes
   The adhesion rate, defined as percentage of cells with at least one backpack as quantified by flow cytometry, for each of these different conjugation strategies is depicted in FIG. 12.

Single layer PLGA backpacks were used to determine the bio-distribution of discoidal micro-particles particle when injected intravenously. Backpacks were fabricated using spin coating of 8 w % PLGA (50:50 L:G) in dichloromethane followed by micro-contact printing on PVA coated dishes. Backpacks were labelled with a far red fluorophore FPI-749 for visualisation using fluorescence imaging.

6-8 week old Balb/c mice were injected with approximately 1 million backpacks. Animals were euthanised and vascular organs were harvested after 1, 6 and 24 hours (n=3 for each timepoint). Fluorescence in the organs was visualized after normalisation with a control organs (from un injected mice) using IVIS Spectrum.

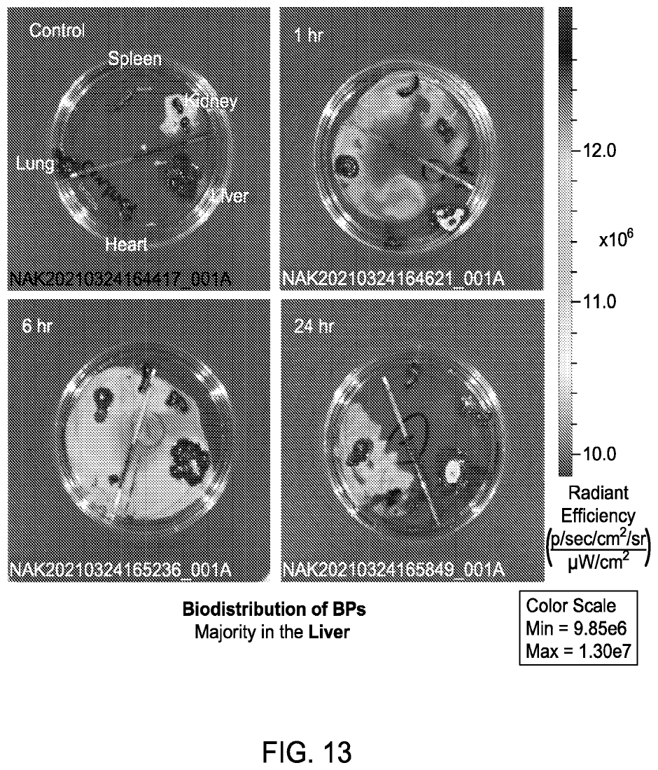

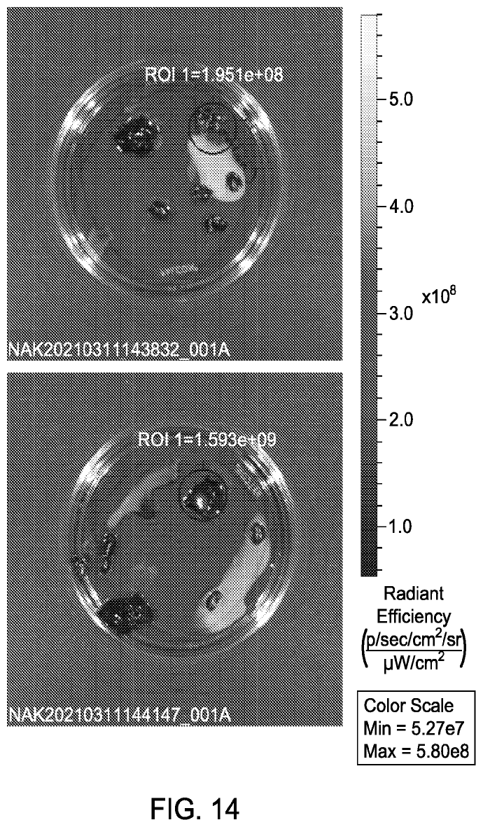

Biodistribution of the backpacks (FIG. 13) and neutrophils (FIG. 14) in animals was then assessed. The majority of the backpacks were found in the liver, while the majority of the neutrophils were found in the lung.

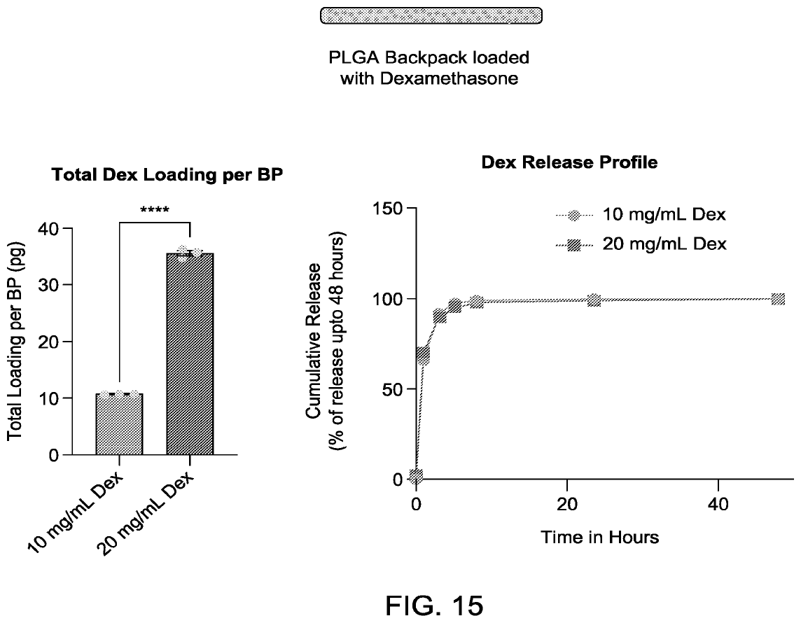

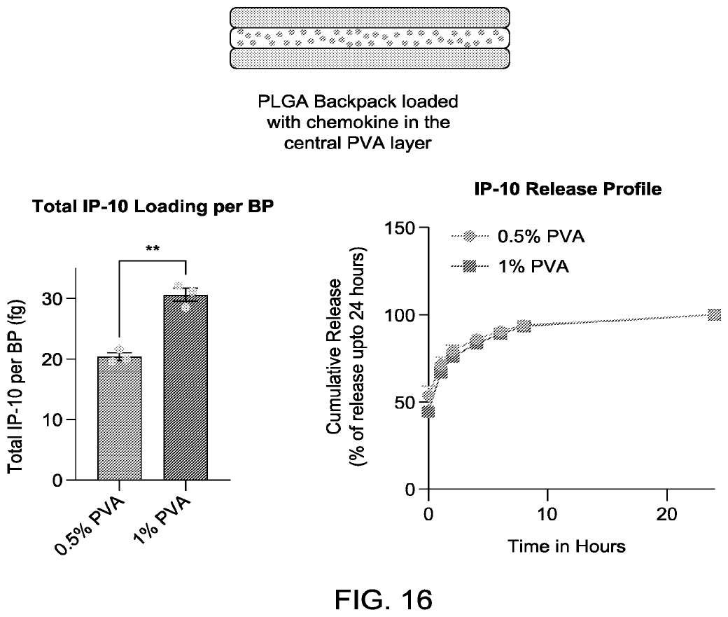

Delivery of small molecule therapeutics by backpacks was assessed by loading a PLGA backpack with dexamethasone. 10 or 20 mg/mL of dexamethasone was dissolved along with PLGA in the spin coating solution. Loading and release profiles of the two formulations are shown in FIG. 15. Delivery of chemokine therapeutics by backpacks was assessed by loading a PLGA backpack with IP-10 (CXCL10) in a central PVA layer. 0.5% or 1% PVA represents the concentration of PVA used in the central layer in order to entrain the chemokine in the backpack. Loading and release profiles of the two formulations are shown in FIG. 16.

### Example 4

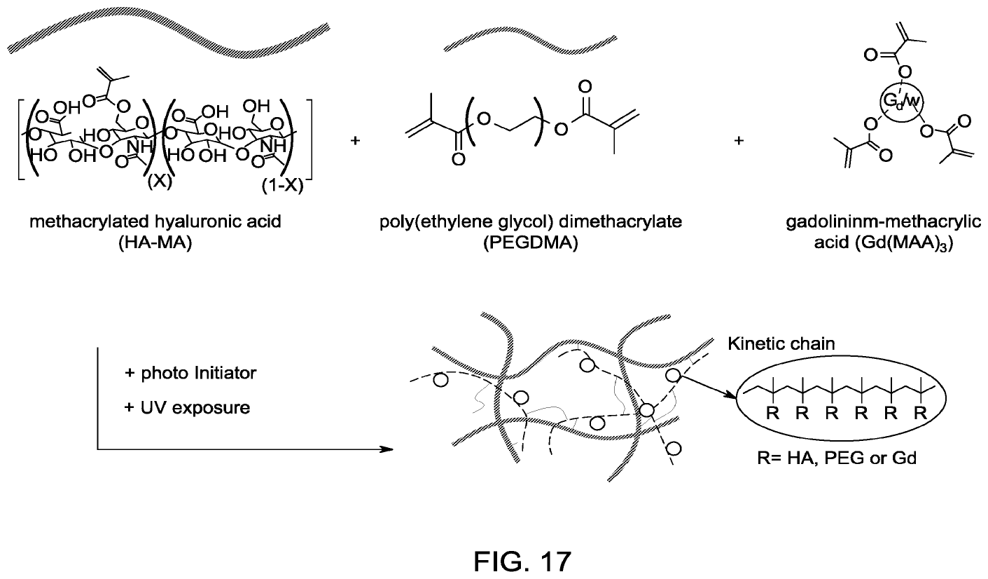

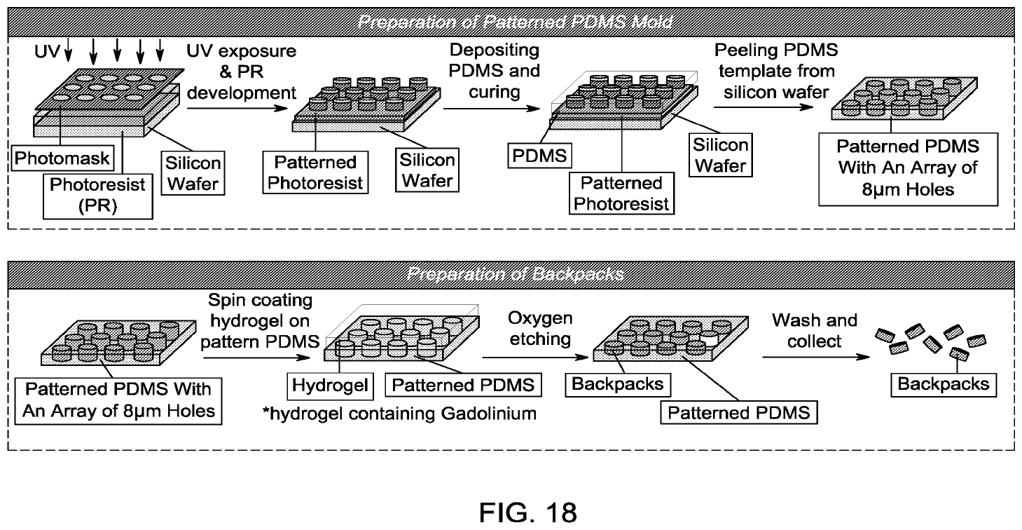

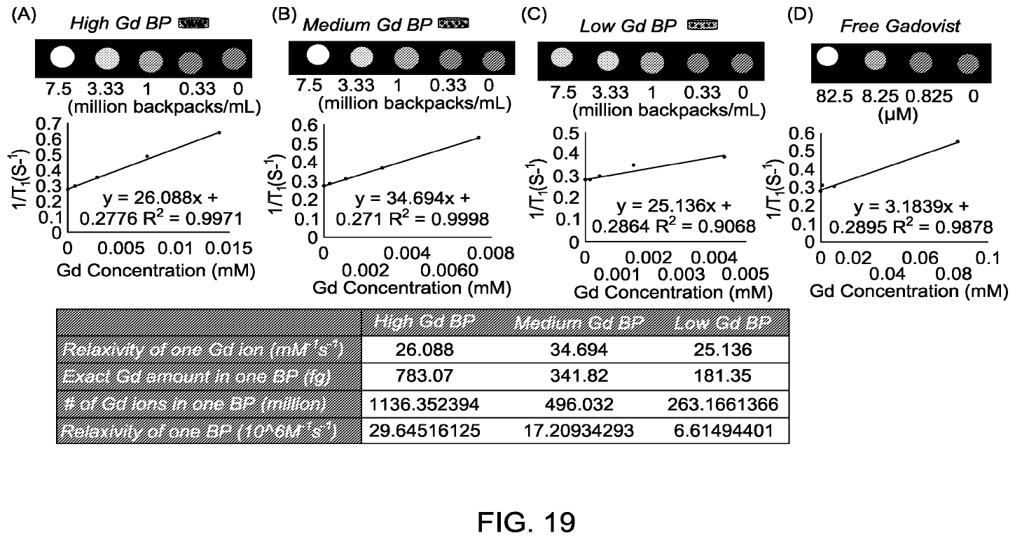

Gadolinium-loaded HA/PEG backpacks with methylacrylate cross-linking were prepared as shown in FIGS. 17 and 18. Backpacks with different levels of gadolinium were prepared and their relaxivities were measured and compared to that of Gadovist (free gadolinium). The different amounts of Gd displayed various relaxivities (FIG. 19, 30A-30C). The relaxivity of gadolinium within backpacks are much higher than that of gadovist (D; 3.18 mM-is-1).

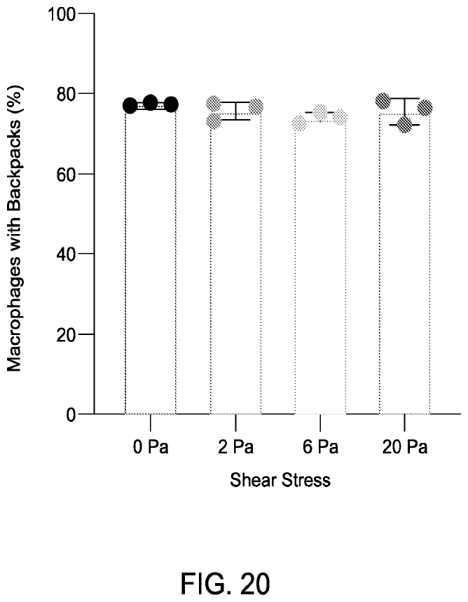

The gadolinium-loaded backpacks were able to attach to cell surfaces without being internalized (data not shown). The attachment to murine macrophages was stable under various shear forces of 2 to 20 Pa (FIG. 20).

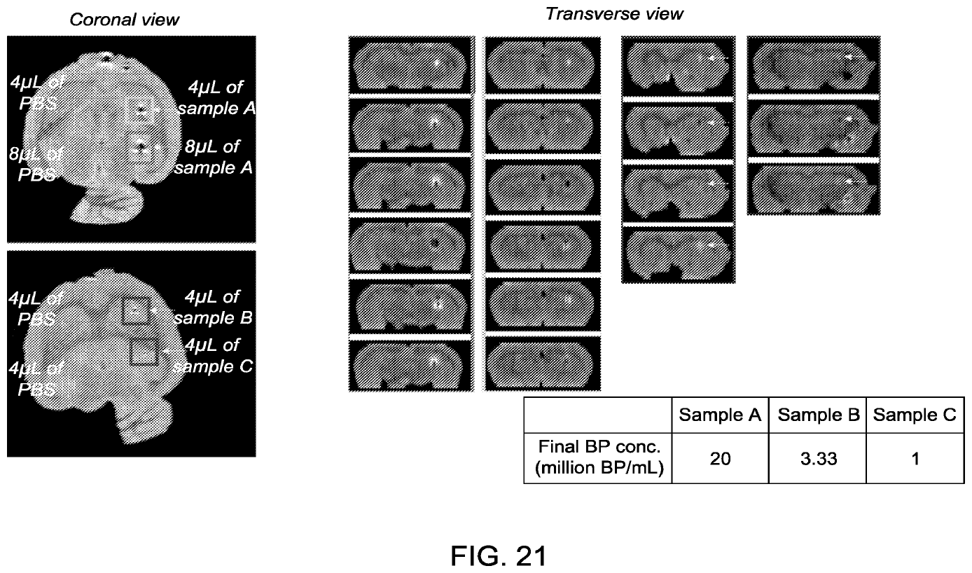

When the gadolinium-loaded backpacks were injected into ex vivo rat brain tissue without being adhered to cells, the backpacks were observable by MRI (FIG. 21).

### Example 5

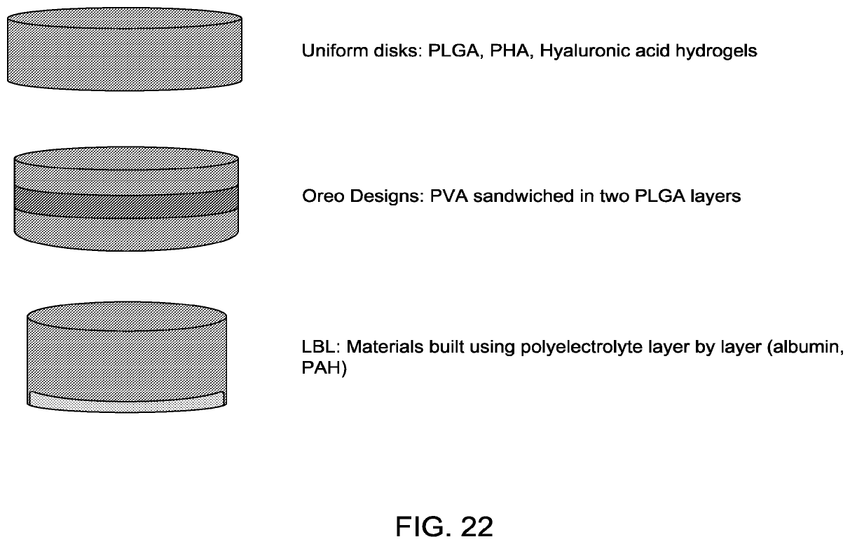

Exemplary backpack structures are depicted in FIG. 22. The single-region PLGA/PHA designs are hydrophobic, non-porous, and exhibit sustained duration. They are particularly suited for encapsulation of small molecules such as immunostimulants, immunosuppressors, and peptides. The “Oreo” design with three regions provides a hydrophilic layer for protein encapsulation and is particularly suited for delivery of cytokines, proteins, mAbs, and/or blends thereof. Hydrogels are hydrophilic and highly porous. They are particularly suited for delivery of MRI contrast agents and nanoparticles.

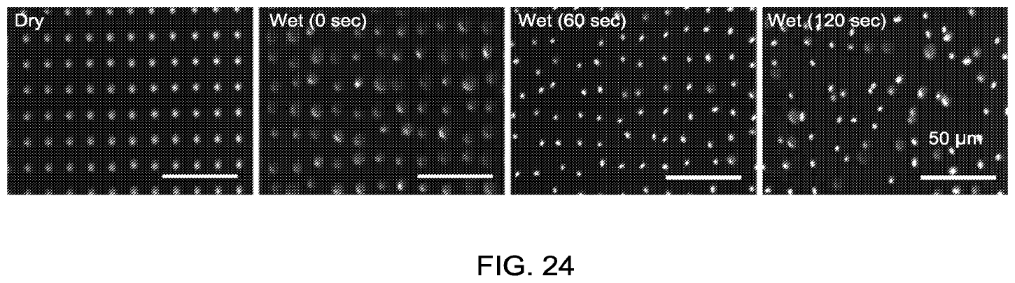

An exemplary procedure for printing backpacks is shown in FIG. 23. The printed backpacks lift off from the surface (FIG. 24). A single printing step can print -40 million backpacks on a 100 sq cm surface. The process can repeated/parallelized to produce billions of backpacks in hours. A typical human dose of monocytes/macrophages is 0.5-1 billion per patient and each cell is expected to have an average of 1 backpack.

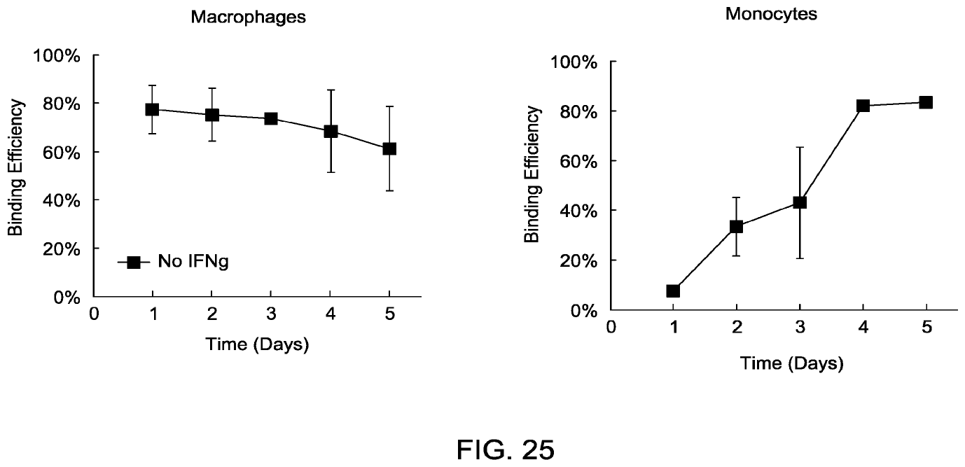

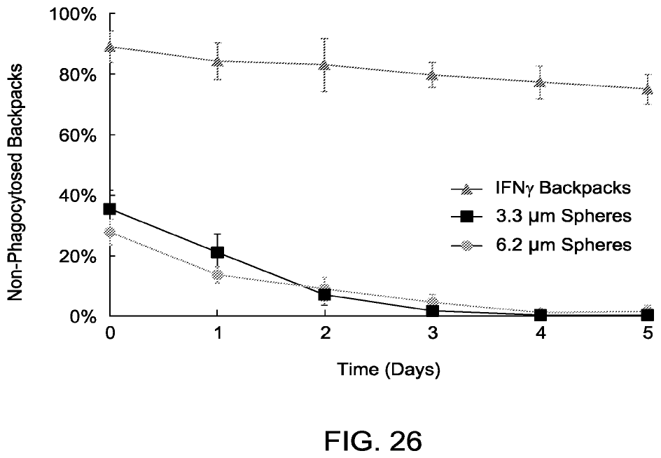

Hyaluronic acid (HA) backpacks can be prepared using the process depicted in FIG. 23. HA is spin-coated on the posts and cross-linked to create stable backpacks. HA backpacks bind well to macrophages (FIG. 25) and remain attached to macrophages without being internalized (FIG. 26).

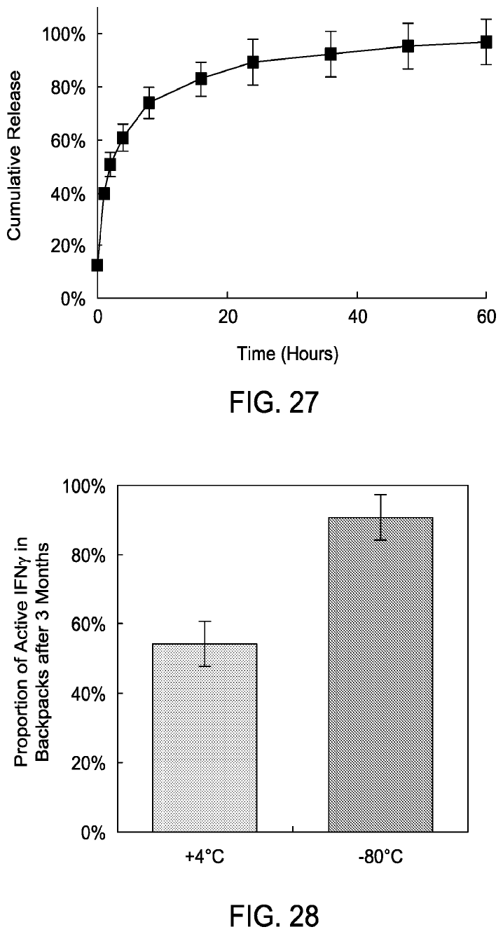

Backpacks utilizing the “Oreo” design, with top and bottom layers of PLGA and a middle lawyer of PVA loaded with IFN-γ were prepared. 80 fg of IFN-γ per backpack was utilized. This deisng releases IFN-γ over a period of time (FIG. 27). Under storage conditions, the IFN-γ is stable within the backpacks (FIG. 28).

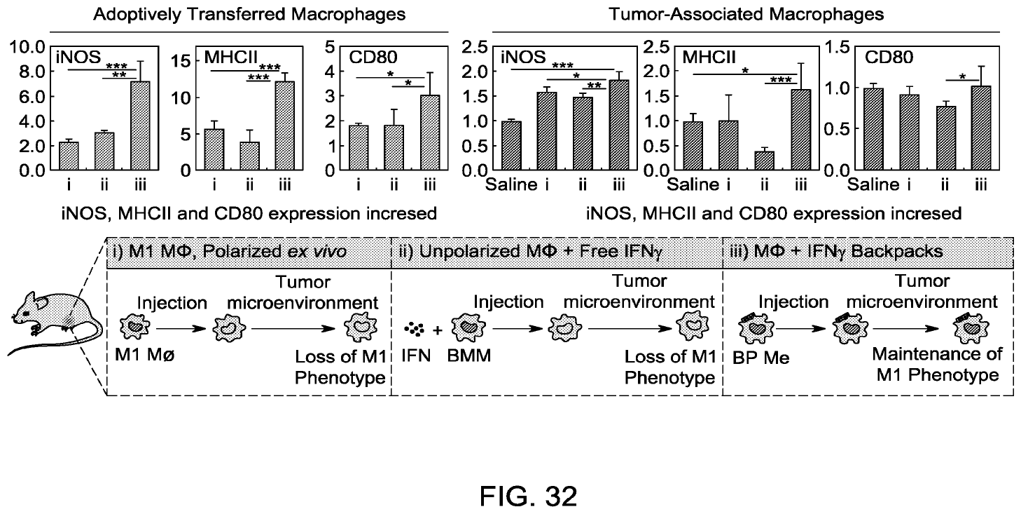

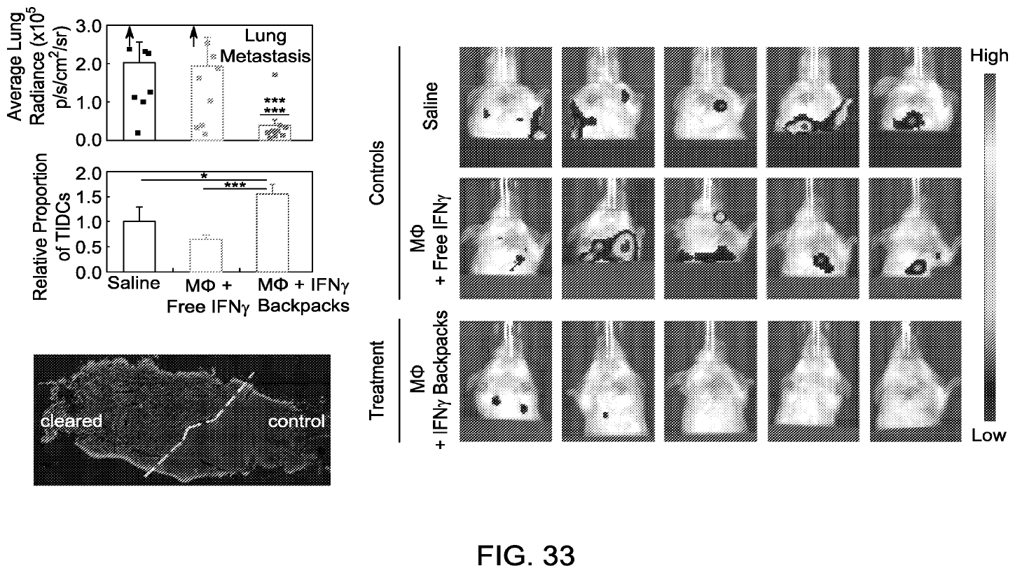

Backpacks loaded with IFN-γ were further demonstrated to influence macrophage phenotypes (FIG. 32). Backpacks permitted maintenance of macrophage phenotype to M1 in the in vivo tumor environment (FIG. 32 —adoptively transferred macrophages, Case iii). Macrophages polarized with free IFN-g do not achieve the same effect (FIG. 32 —adoptively transferred macrophages, Cases ii vs iii). Backpacks also impacted pre-existing macrophages in the tumor microenvironment (FIG. 32 —tumor-associated macrophages). Therapeutically, the IFN-γ backpacks, when administered to mice with cancer, reduced lung metastasis and improved survival (FIG. 33). Similar therapeutic efficacy against solid tumors (breast, ovarian, pancreatic), ARDS, and autoimmune disease is contemplated herein.

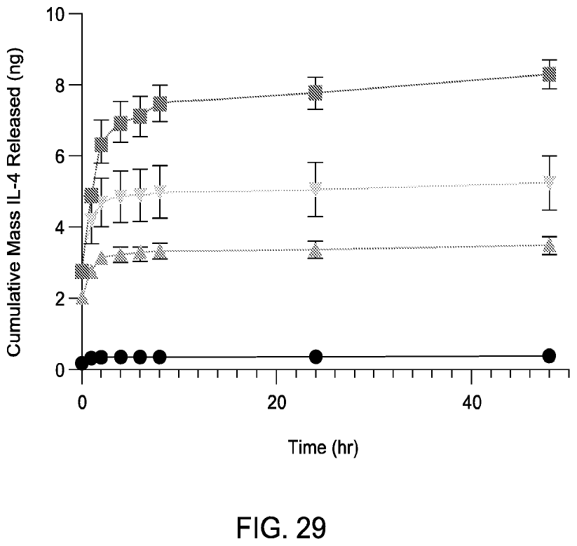

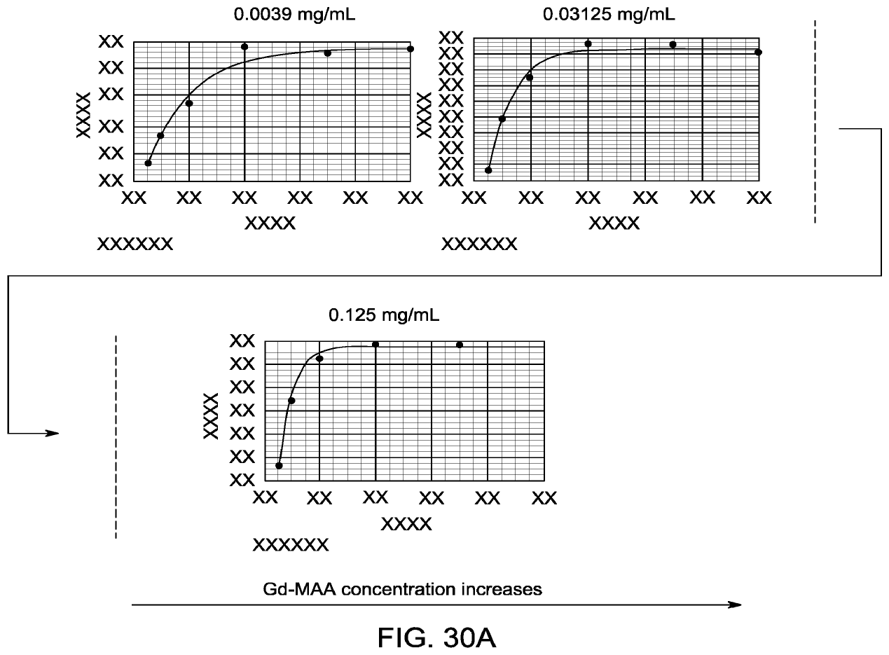

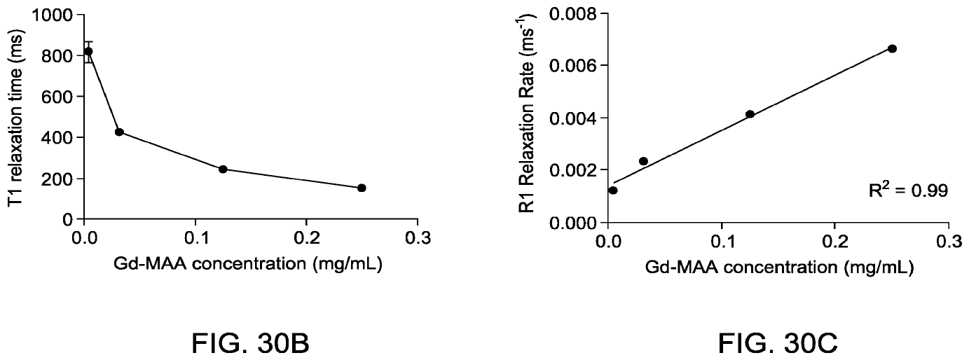

These experiments utilized sandwich backpacks with a few different design iterations (PVA, laponite) (Table 1). Release of IL-4 from the backpacks was dose-dependent (FIG. 29). The top curve (blue) is 4% PLGA sandwich with 2.5 mg/mL laponite. The next curve (orange) is 8 PLGA with 0.5 wt % PVA and 2.5 mg/mL laponite. The 3rd curve (teal) is 4 PLGA with 0.5w^{t} PVA and 2.5 mg/mL laponite. The bottom curve (purple) is 2% PHA-E with 0.5 wt % PVA and 2.5 mg/mL PG-3T laponite.

TABLE 1
Backpack design and IL-4 release

| Design | IL-4 per BP (fg) |
|---|---|
| 2% PHA-E + LAP + PVA | 2.15 |
| 4% PLGA + LAP + Heat | 46.14 |
| 4% PLGA + LAP + PVA | 19.93 |
| 8% PLGA + LAP + PVA | 29.14 |

### EXAMPLE 6

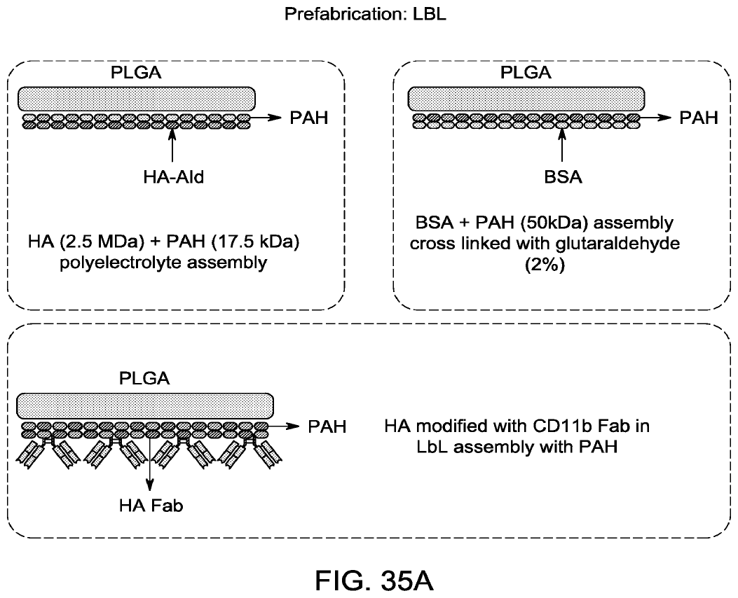

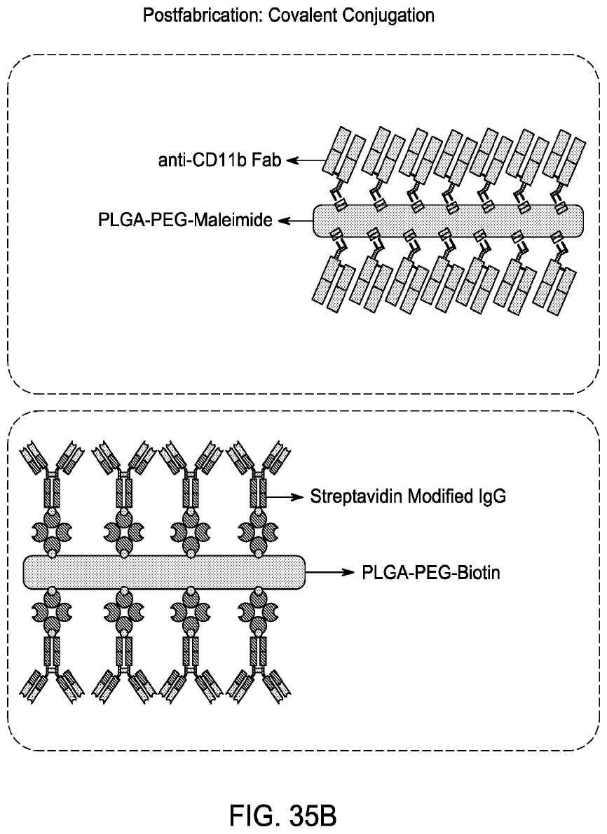

Different embodiments of backpacks were prepared and adhered to cells as shown in Table 4. The modifications were performed as shown in FIGS. 35A-35B.

TABLE 4

| | | | | | Method of | |
|---|---|---|---|---|---|---|
| | | | | | linkage | Approximate |
| | | | | | (Maleimide | % |
| Cell Type | Species | Source | Activation status | Ligand | or Biotin) | Attachment |
| Neutrophils | Mouse | Bone Marrow | — | CD11b | Maleimide | 40-60% |
| Neutrophils | Human | Peripheral Blood | — | CD11b | Maleimide | 40-50% |
| Neutrophil | Mouse | Bone Marrow | — | CD45 | Maleimide | 7-15% |
| T cells | Mouse | Spleen | Activated CD8+ | CD45 | Biotin | 35-45% |
| T cells | Mouse | Spleen | Activated CD8+ | CD44 | Biotin | 25-35% |
| T cells | Mouse | Spleen | Activated CD8+ | CD11a | Biotin | 30-35% |
| T cells | Mouse | Spleen | Activated CD8+ | CD27 | Biotin | 14-15% |
| Monocytes | Mouse | Bone Marrow | — | CD11b | Maleimide | 40-60% |
| Monocytes | Mouse | Bone Marrow | — | CD45 | Maleimide | 40-60% |
| Monocytes | Human | Peripheral Blood | — | CD11b | Maleimide | 60-80% |
| Monocytes | Human | Peripheral Blood | — | CD45 | Maleimide | 60-70% |
| NK cells | Mouse | Spleen | — | CD11b | Biotin | 25-30% |
| NK cells | Mouse | Spleen | — | CD49b | Biotin | 15-20% |
| NK cells | Mouse | Spleen | — | Cd45 | Biotin | 50-60% |
| NK cells | Human | NK-92 | IL-2 activated | CD11a | Biotin | 30-40% |
| NK cells | Human | NK-92 | IL-2 activated | CD45 | Biotin | 30-40% |
| NK cells | Human | NK-92 | IL-2 activated | NKG2D | Biotin | 10-15% |
| NK cells | Human | NK-92 | IL-2 activated | NKp46 | Biotin | 1-2% |
| NK cells | Human | NK-92 | IL-2 activated, | ICAM-1 | Biotin | 25-30% |
| | | | Mg-EDTA | | | |
| NK cells | Human | NK-92 | IL-2 activated | NKp30 | Biotin | 5-6% |
| NK cells | Human | Peripheral Blood | IL-2 activated | CD11a | Biotin | 30-40% |
| NK cells | Human | Peripheral Blood | IL-2 activated | CD45 | Biotin | 30-40% |
| NK cells | Human | Peripheral Blood | IL-2 activated | NKG2D | Biotin | 10-15% |
| NK cells | Human | Peripheral Blood | IL-2 activated | NKp46 | Biotin | 1-2% |
| NK cells | Human | Peripheral Blood | IL-2 activated | ICAM-1 | Biotin | 25-30% |

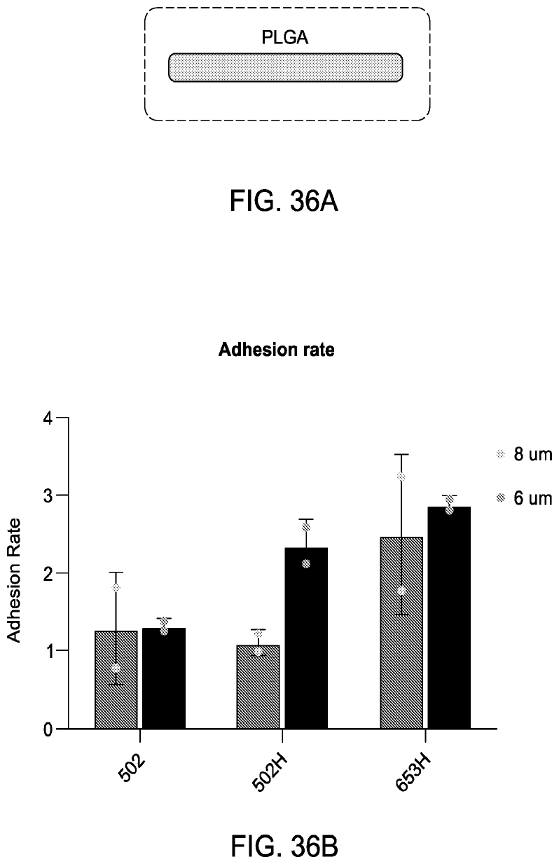

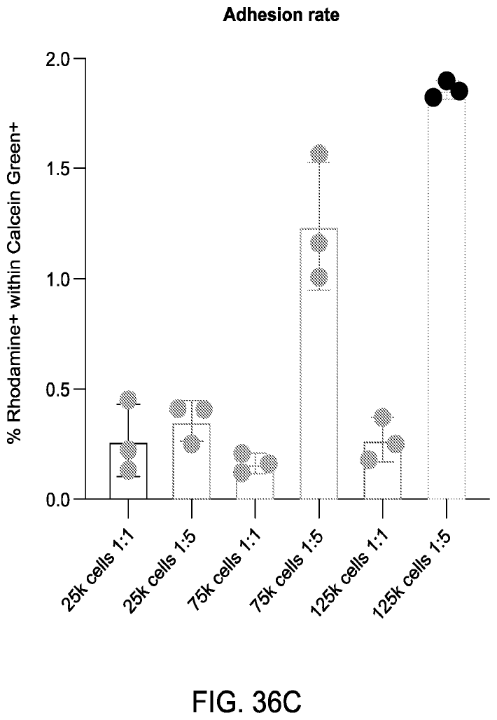

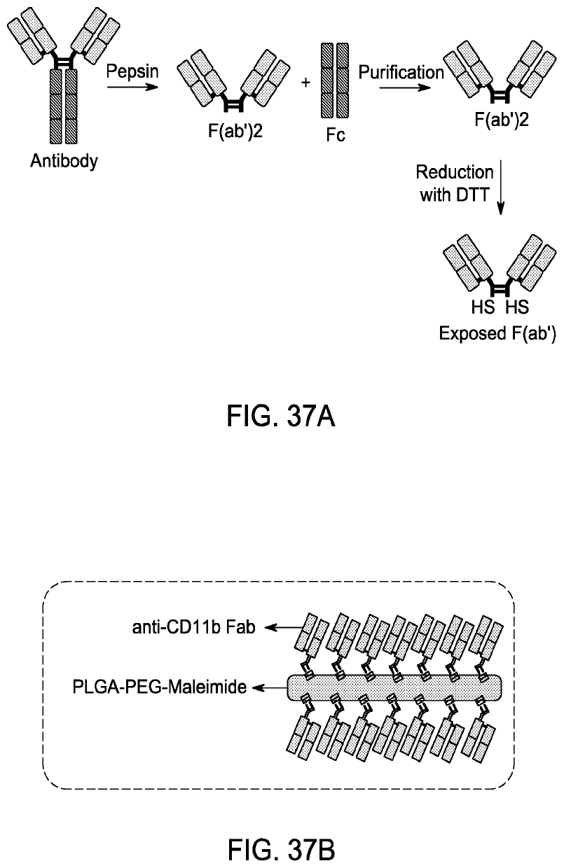

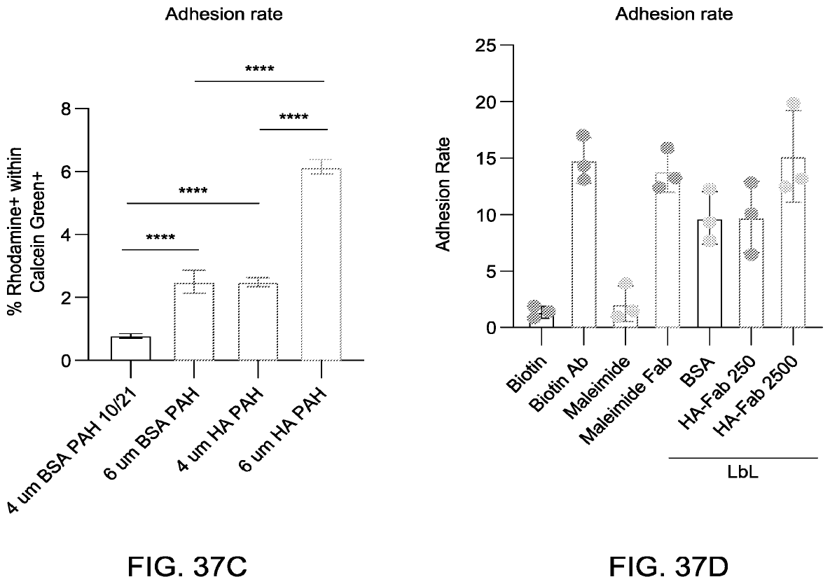

Unmodified PLGA backpacks did not attach well to neutrophils (FIGS. 36A-36C). Inclusion of anti-CD11b in the backpacks increased adhesion (FIGS. 37A-37D).

Attachment to neutrophils is also increased by modifications of the neutrophils themselves, specifically with priming agents such as IFNg, GCSF, GMCSF. Such agents lead to better attachment of neutrophils to blood vessels during extravasion and this phenomena can be utilized to improve backpack adhesion to neutrophils (FIG. 381B-38D)). Treatment with a caspse inhibitor (pan-caspase inhibitor iCasp) also prolonged neutrophil viability.

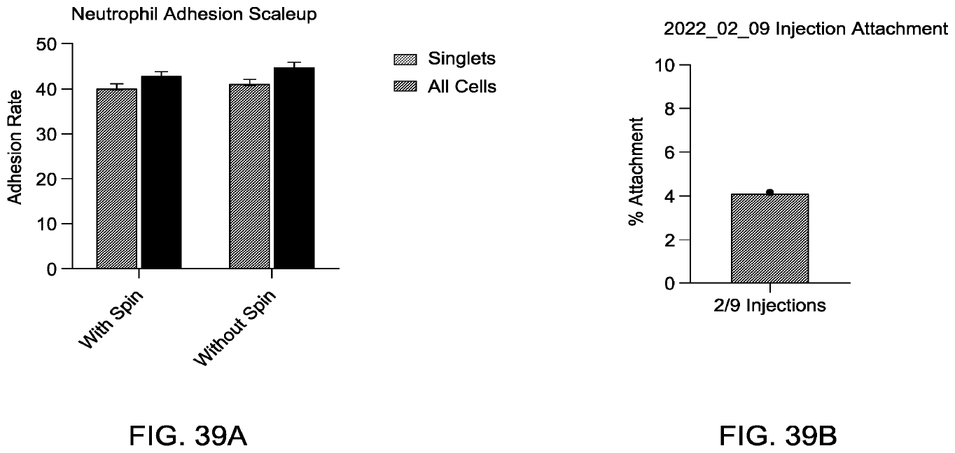

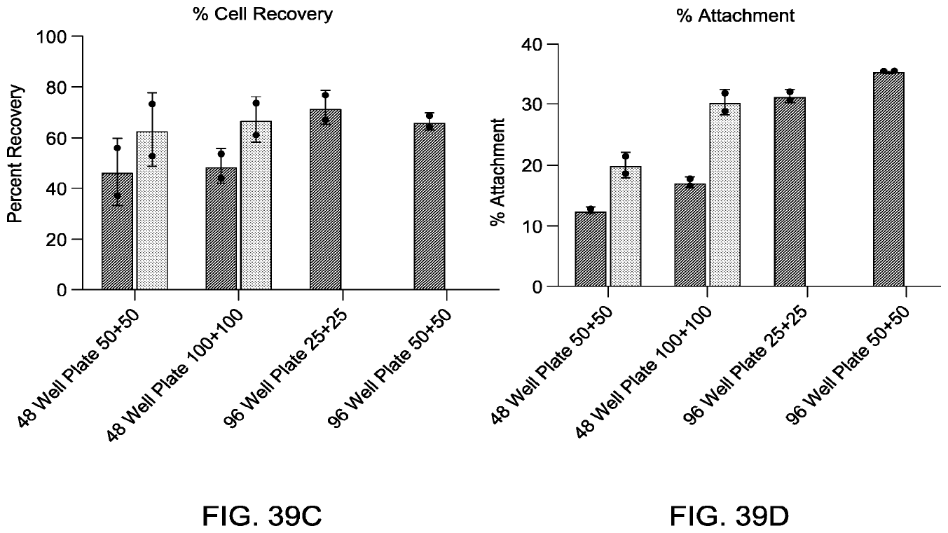

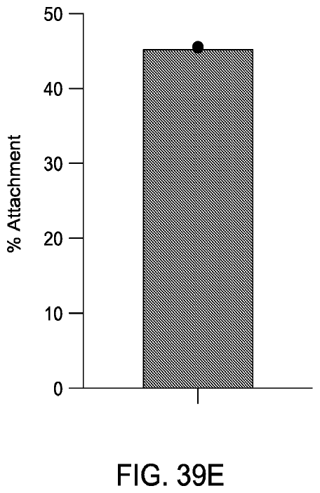

The ability to scale up backpack attachment was tested, e.g., in 48 well plates, 96 well plates, and 15 mL centrifuge tubes (FIGS. 39A-39E). Changing the reactor vessel significantly impacts the attachment, possibly because of lower area for settling in a 15 mL tube. To compare 48 well and 96 well plates, the 20M/mL Cells and 30 million(M/)mL BPs, in the volumes indicated in FIGS. 39C-39D, were mixed together. Cells from 48 well plates were collected into 1.5 mL tubes and spun down. 96 well plates were spun down directly. 48 well plates were TC treated, cells stuck to the bottom. Scraping was required to take them out. Viability was reduced. Approximately 60% of the cells that were stuck to bottom were BP positive. High levels of attachment were seen when injections were made into multiple wells of a 96 well plate, incubated for 2 hours, collected in a 15 mL tube, and then spun down (FIG. 39E).

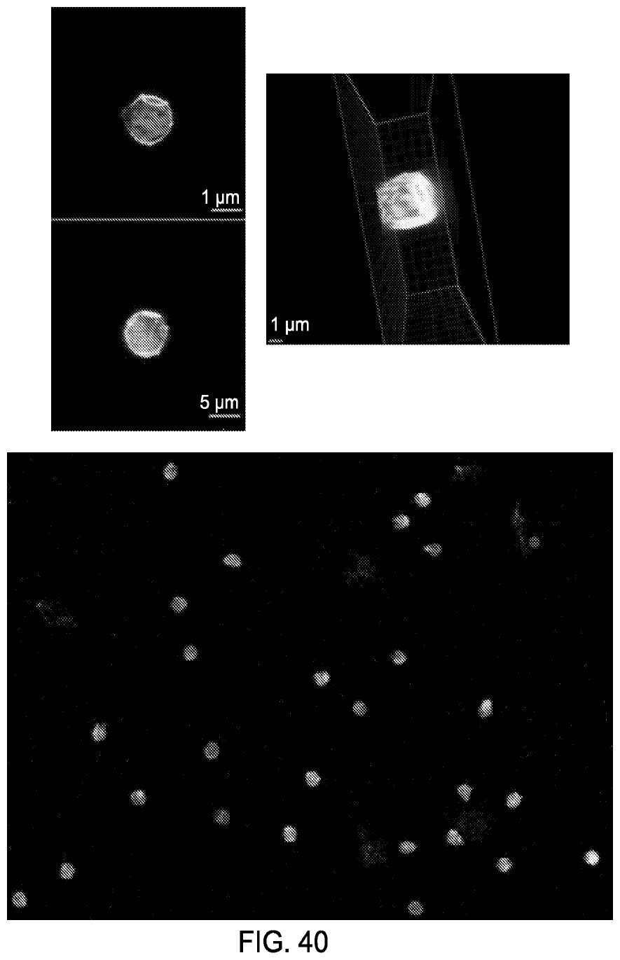

Backpacks, when attached, are retained on the surface of the neutrophils (FIG. 40).

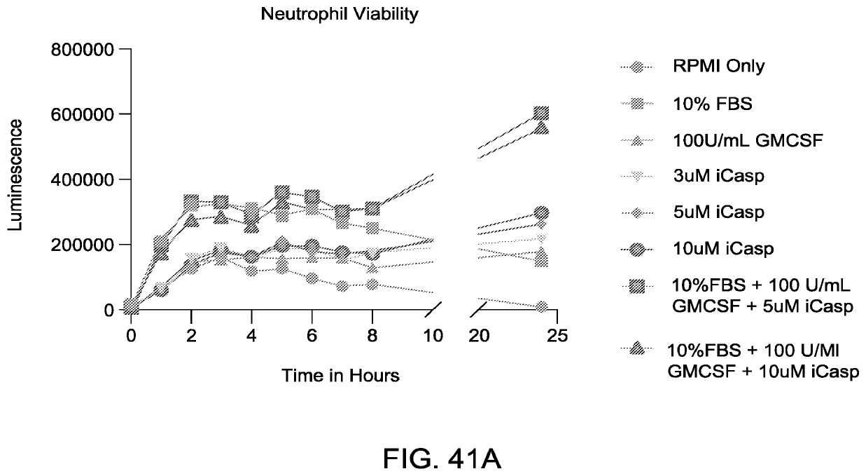

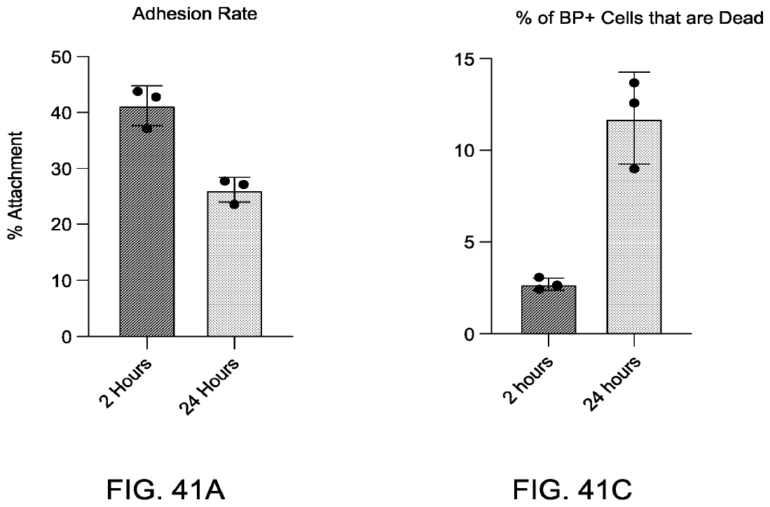

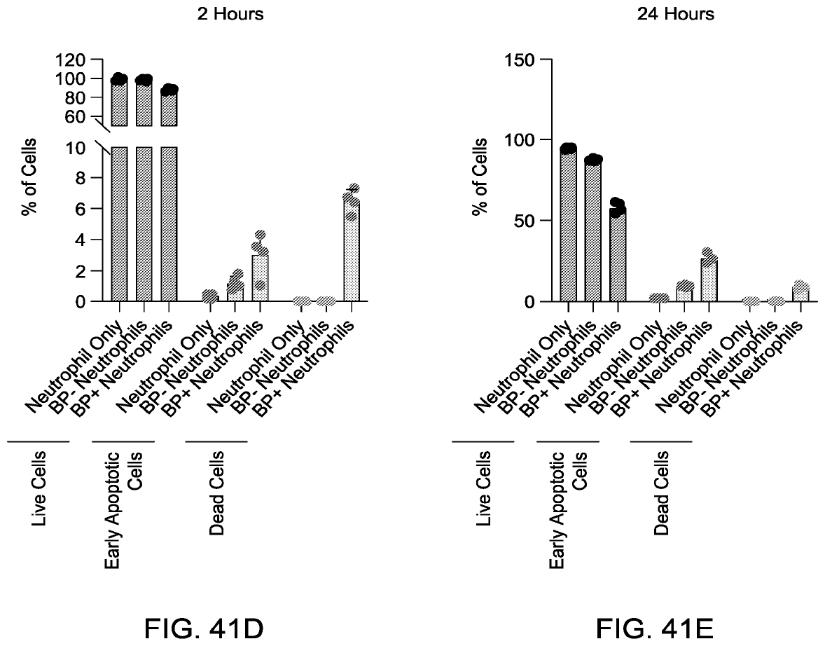

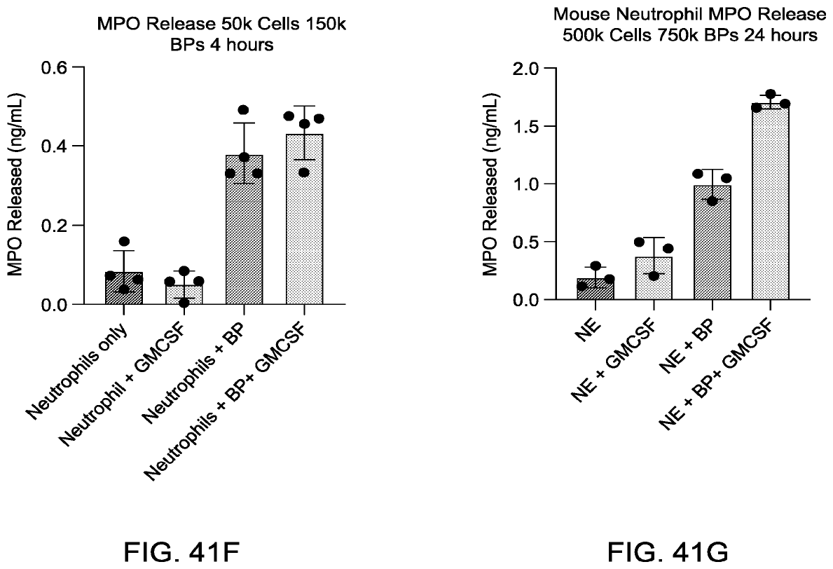

Initial neutrophil viability is optimal with FBS (FIG. 41A). Addition of GMCSF or other cytokines provides maximal viability overtime, up to 24 hours (FIG. 41A). Attachment in a medium comprising serum is contemplated for maintenance of maximum viability for a short term.

The effect of backpacks on viability was tested by mixing 20M Cells/mL+30M BPs/mL (25 uL+25 uL). Cell viability was tested at 2 and 24 hours by staining with DAPI. At 2 hours ˜97% of BP+cells are alive which means that at short term the BPs are not significantly affecting the neutrophil viability (FIGS. 41B-41C). Adhesion rate reduces after long incubation. It is contemplated that this reduction is due to cell death. The majority of BP- Cells are alive at both timepoints.

Neutrophils were incubated with backpacks at a ration of 1:1.5 in complete medium for 2 or 24 hours. The cells were then stained with AnnexinV/PI. At 2 hours, any difference in viability is not pronounced which confirms the results from DAPI staining assay (FIG. 41D). The dead Cell stain (PI) overlaps to some extent with BP stain (FPI749).

Neutrophil degranulation upon backpack attachment was measured by MPO release (FIGS. 41F-41G). The priming of the neutrophils by backpacks is independent of GMCSF, at least in the short term.

MPO release is important for neutrophil mediated tumor killing (see, e.g., Nguyen et al. 2017; Chen et al. 2017). N1-type neutrophils, associated with IFN-β, are antitumor. Polarization to N1 phenotypes can be induced by TGF-β inhibitors such as SM16. N2-type neutrophils, associated with TGF-β, are protumor. See, e.g., Ohms et al. 2020. N1 neutrophils are highly activated, shedding CD62L and having granules containing CD66b and CD11b. In the event of activation, granules fuse with the membrane leading to increase in expression of these adhesion molecules. Activated neutrophils also release TNF and IP10.

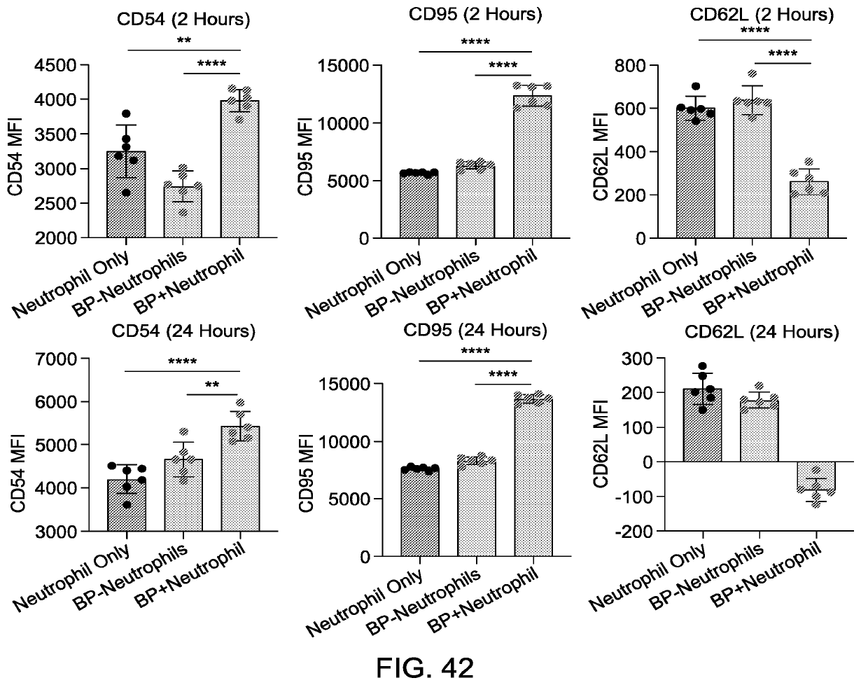

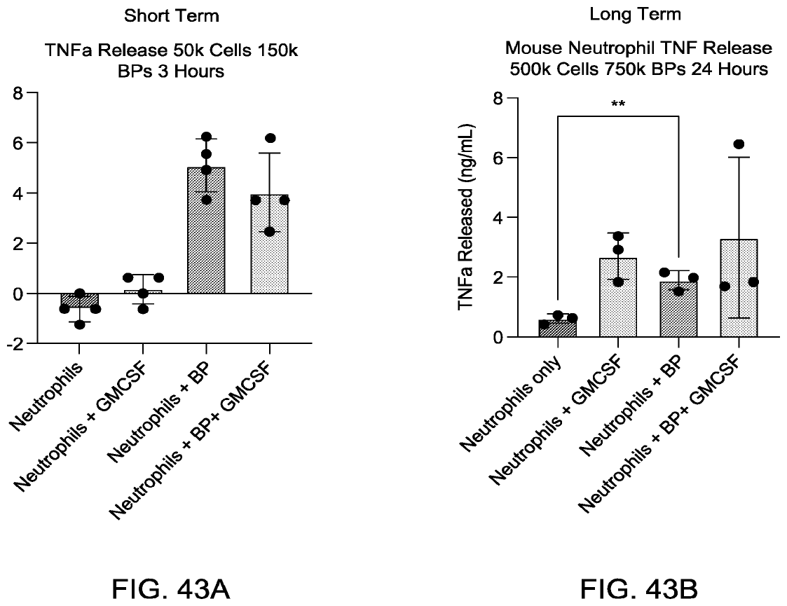

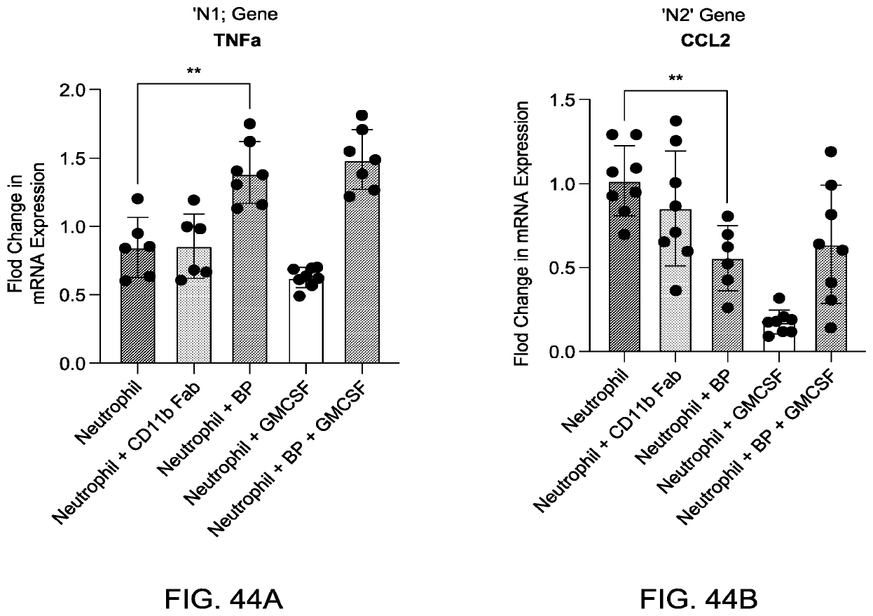

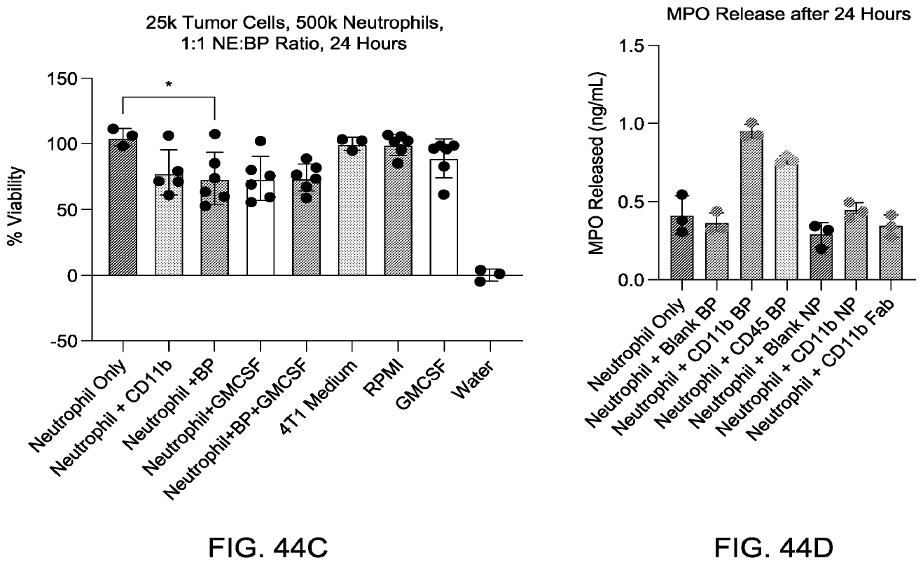

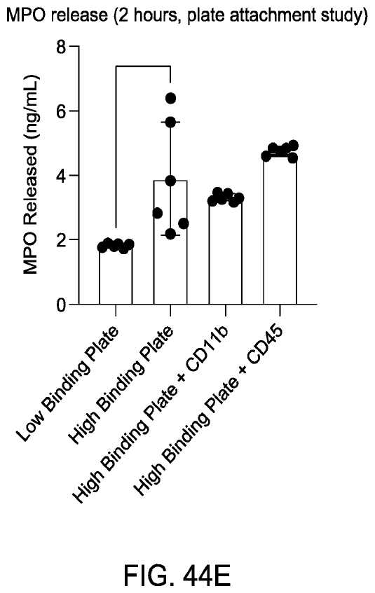

200,000 human neutrophils were incubated with 300,000 backpacks. The cells were then stained for CD54/ICAM1 and CD95/FasR (both N1 markers), and CD62L, and activated marker. Backpacks themselves were able to polarize neutrophils to the N1 phenotype (FIG. 42). The neutrophils were also found to release TNFa after backpack attachment, another indication of neutrophil activation (FIGS. 43A-43B). Expression of N1 and N2 marker genes were also altered after backpack attachment (FIGS. 44A-44B).

The phenotype was reflected in the activity of the neutrophils. After backpack attachment, neutrophils displayed in vitro toxicity against 4T1 cells (FIG. 44C). 4T1-Luc Cells were used at a 1:20 Target to Effector Ratio. Viability was determined by reading luminescence after treatment with Luciferin. Cells treated 4T1 Medium were considered 100% viable and those with water were considered 0% viable.

This effect on neutrophile type/phenotype was specific to backpacks, as nanoparticles did not elicit similar responses (FIG. 44D). Nanoparticles prepared using nanoprecipitation. An equal amount of PLGA was treated with 1M BPs=0.2 mg PLGA.

To investigate the mechanism of activation by backpacks, Neutrophils were incubated in: 1) Low binding plate where the cells are not expected to attach, 2) High Binding plate where the cells are expected to attach, 3) a CD11b coated plate and 4) CD45 coated plate. Attachment to the surface without any ligand leads to activation as assessed by MPO release (FIG. 44E).

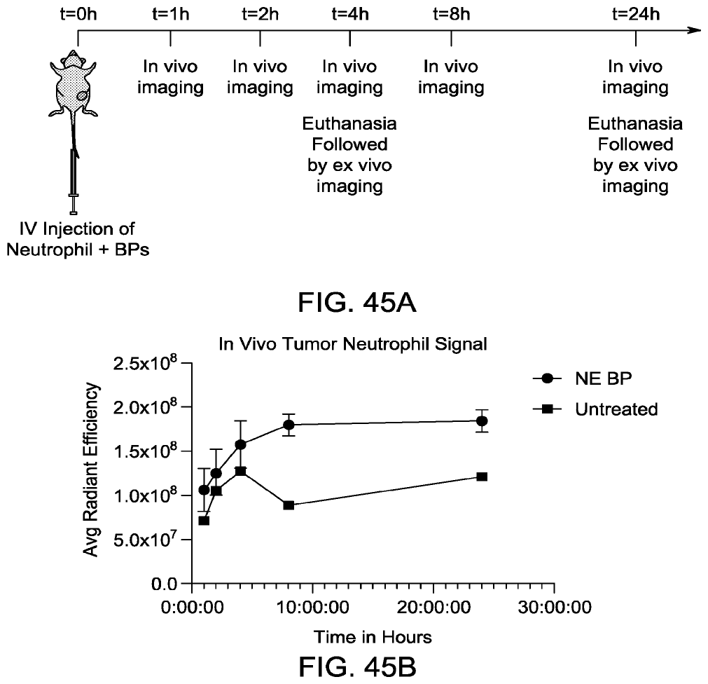

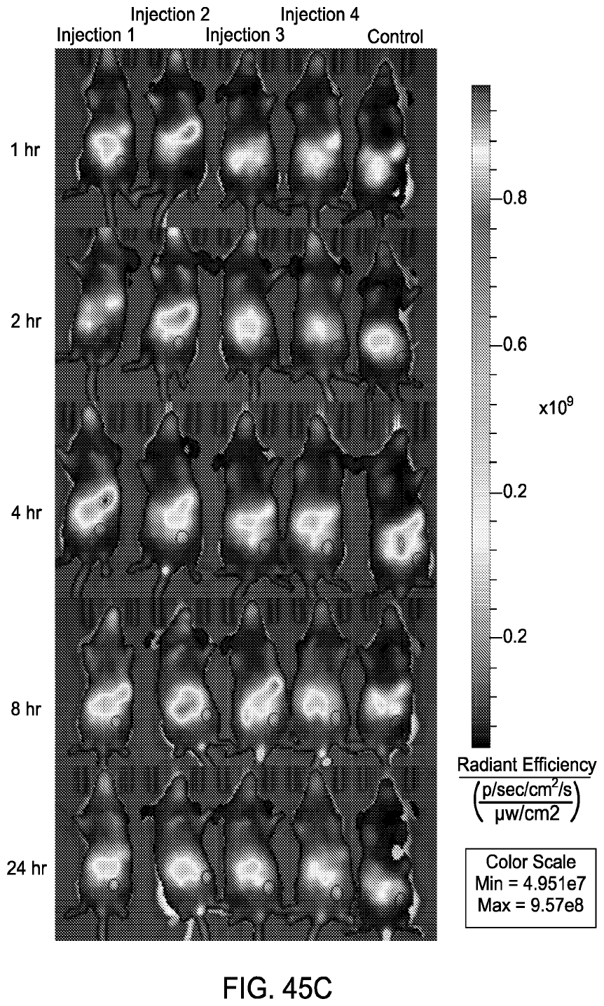

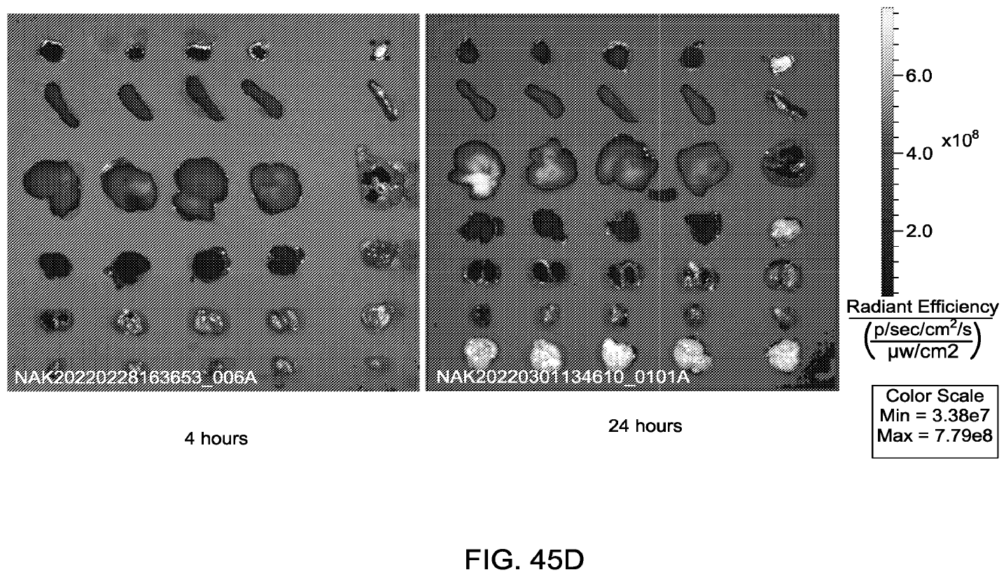

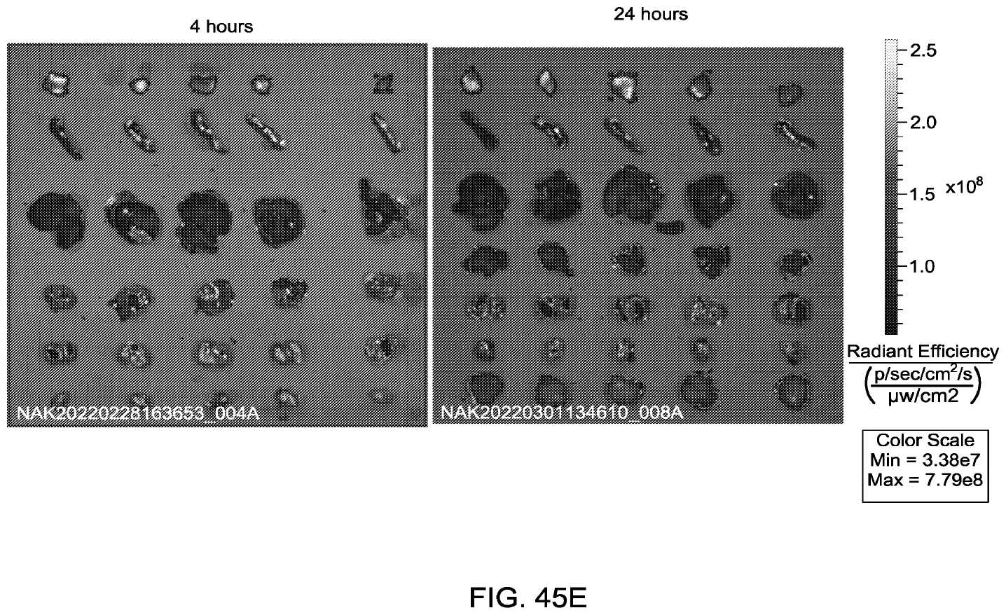

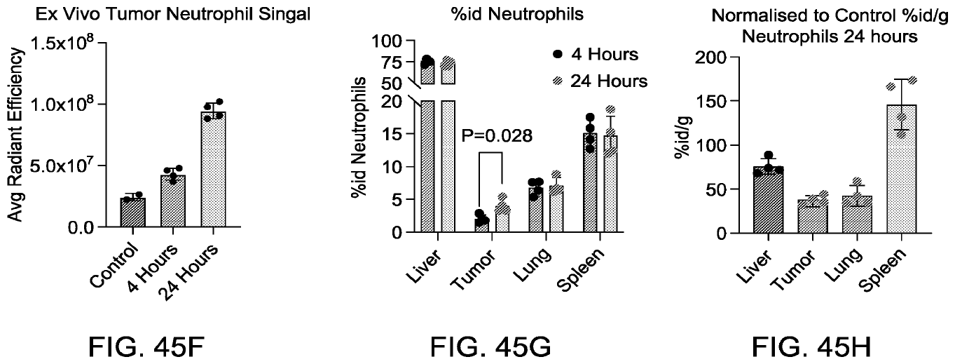

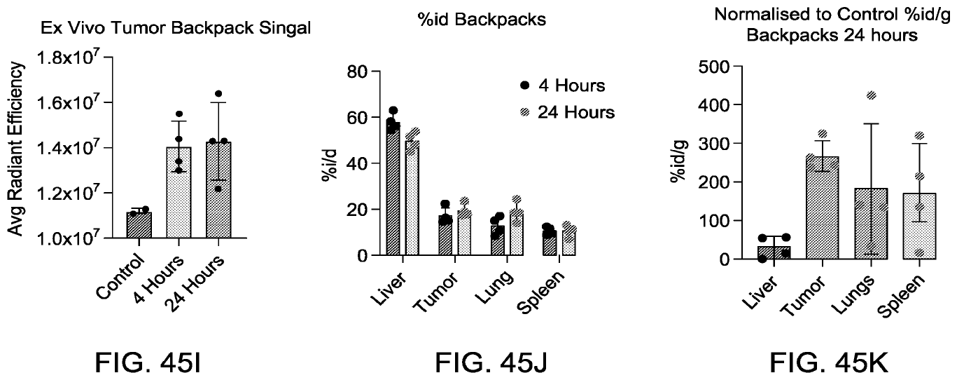

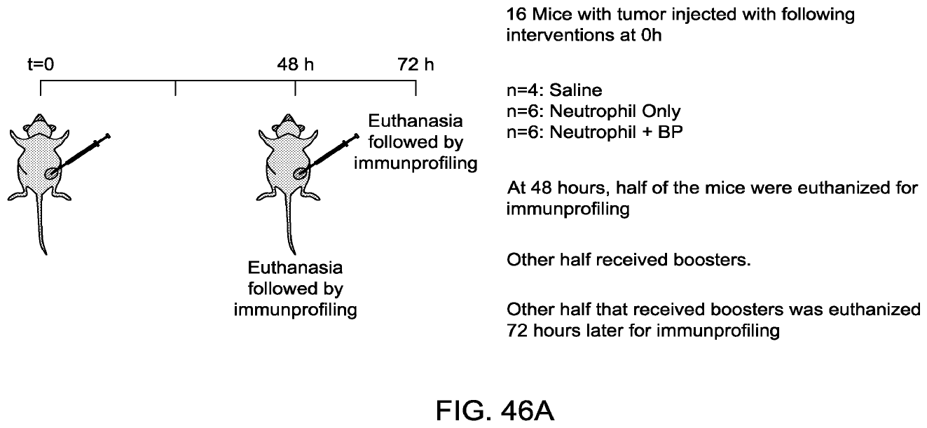

Neutrophils activated by backpacks more quickly accumulate in tumors, reaching the tumors within 4 hours (FIGS. 45A-45K). The accumulation of cells and backpacks in various organs was also examined.

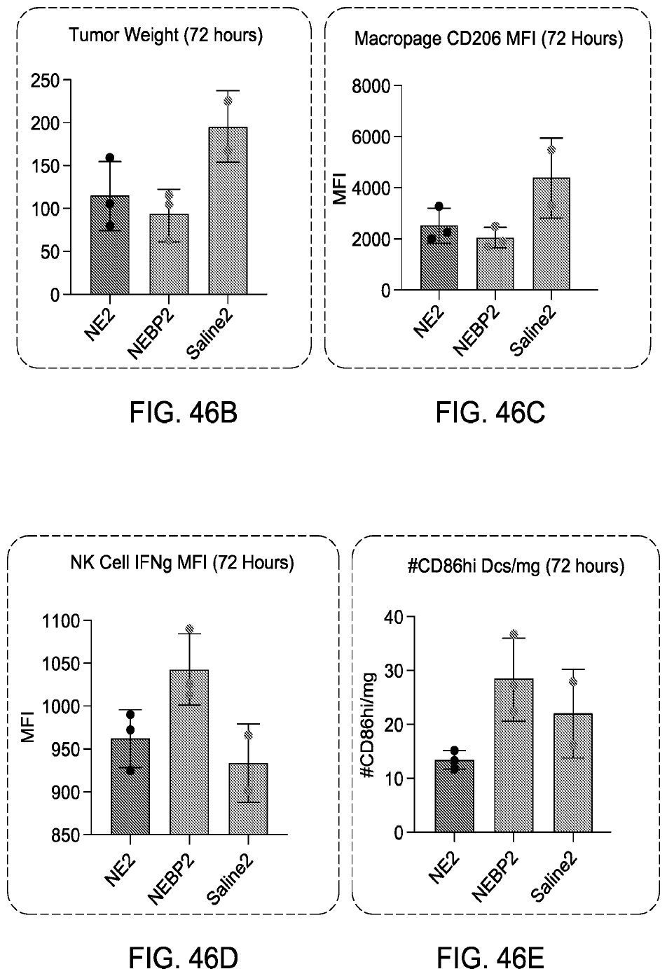

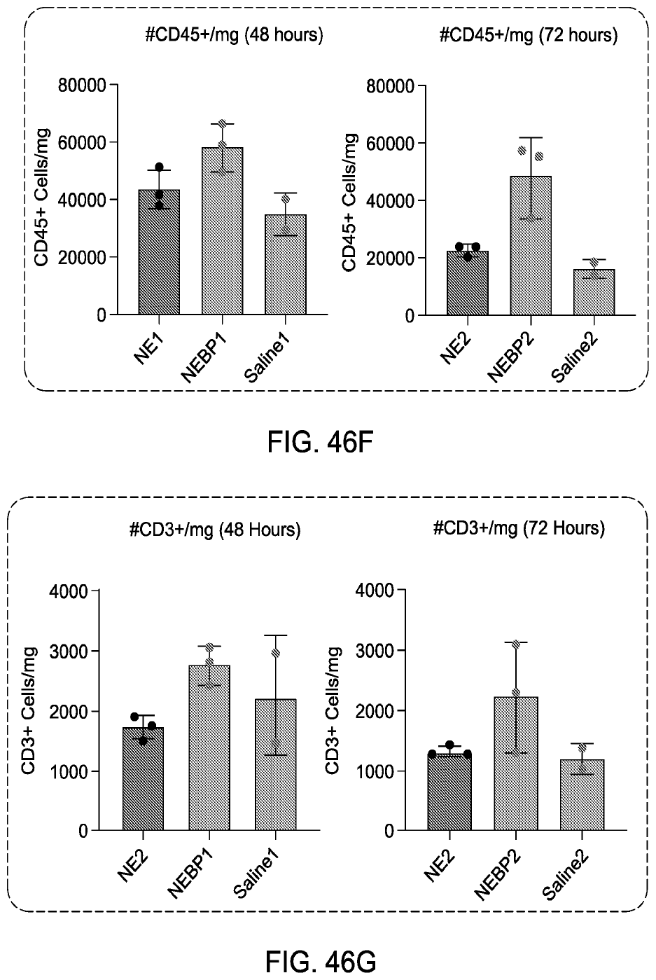

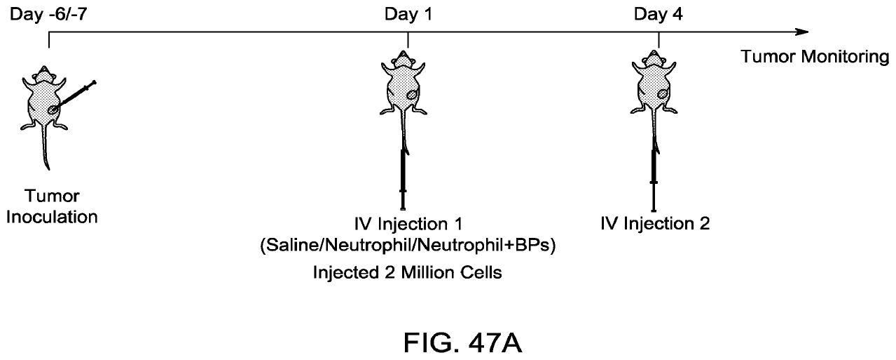

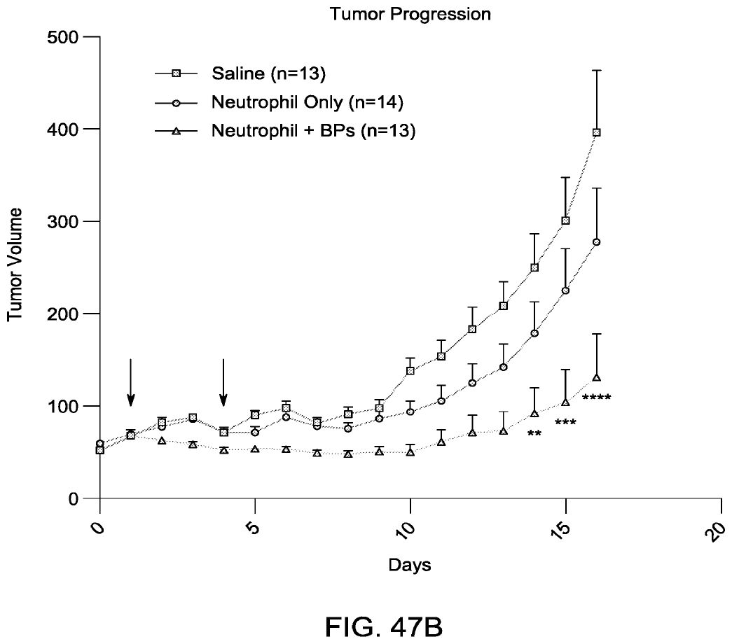

The immune response of mice to neutrophils with backpacks was examined (FIGS. 46A-46G) and it was found that the neutrophil backpacks alter the TME. It was next asked whether the neutrophil backpacks were efficacious in treating the tumors (FIG. 47A). Administration of neutrophil backpacks significantly decreased tumor volume (FIG. 47B).

### Example 7

The effect of backpacks on natural killer (NK) cells was next examined.

Backpacks attach to the surface of NK cells (FIG. 48B) and this adhesion is improved by media+FBS and by a higher backpack: cell ratio (FIGS. 48C-48D). A number of ligands were tested for backpack adherence to human NK-92 cells (Table 5). CD45 showed the highest rate of adhesion (FIG. 48E) and incubation time also affected adhesion (FIG. 48F). Binding to ICAM-1 on NK-92 cells was improved by Mg2+EGTA, whereas binding on primary NK cells was constitutive (FIGS. 48G-48H).

TABLE 5

| Antibody/Ligand | |
|---|---|
| against: | Receptor/Marker |
| CD11b | CD11b/CD18 |
| CD11a | LFA-1 (Lymphocyte) |
| CD11a/CD18 | LFA-1 (Lymphocyte) |
| CD56 | CD56 (NK cell) |
| CD45 | CD45 (Lymphocyte) |
| ICAM-1 (Fc conjugated) | LFA-1 |

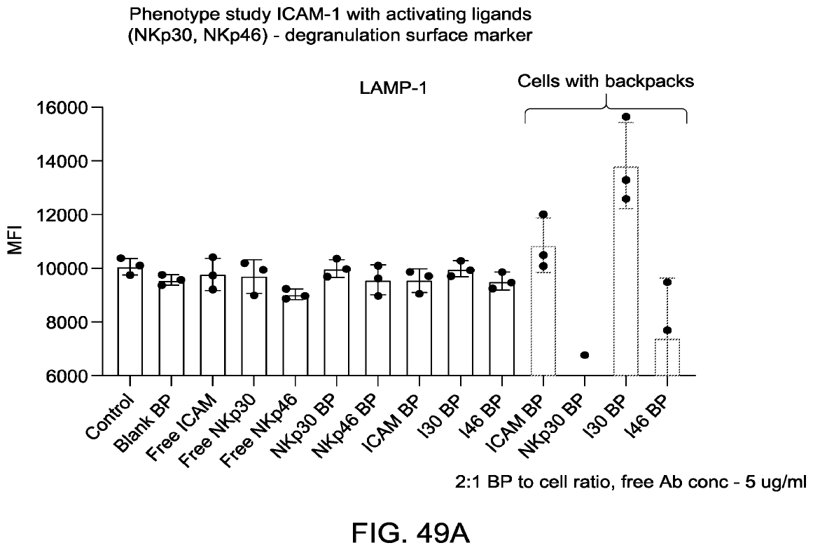

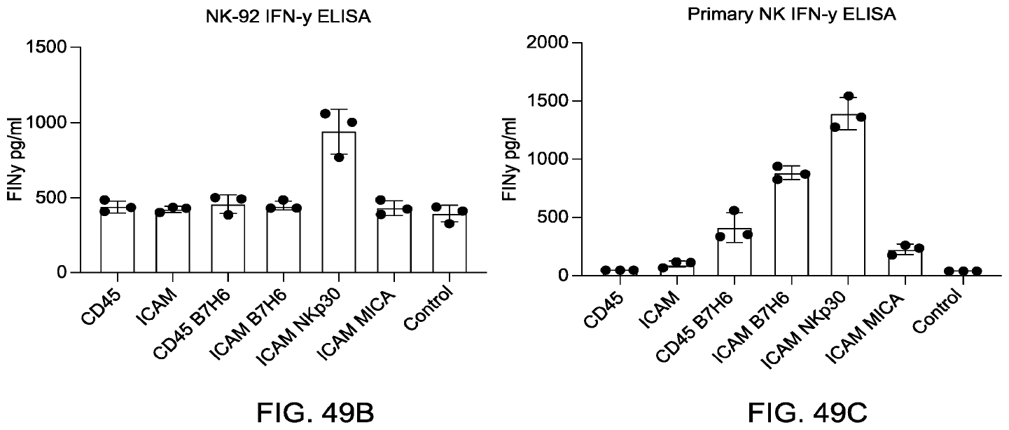

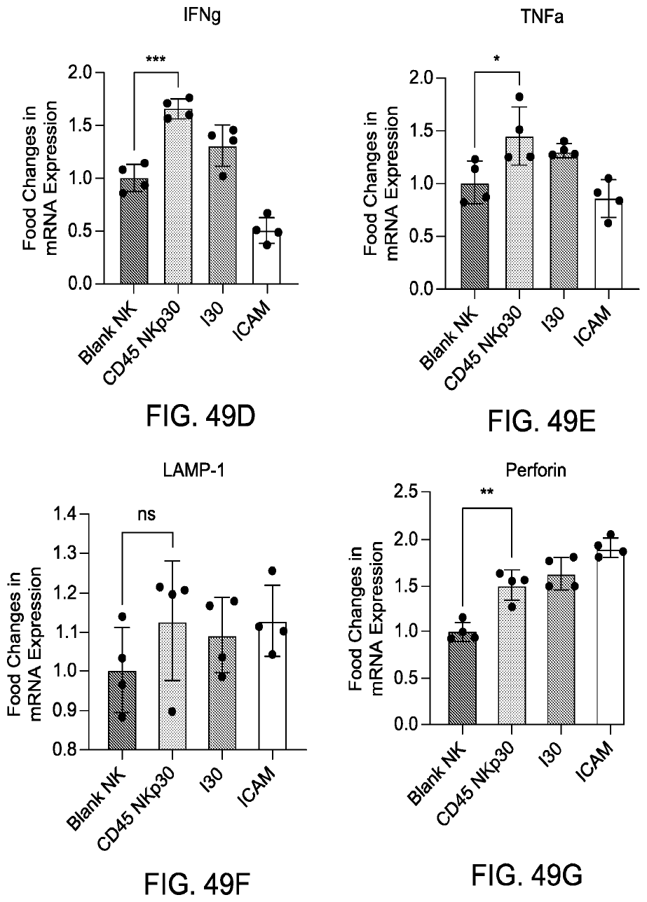

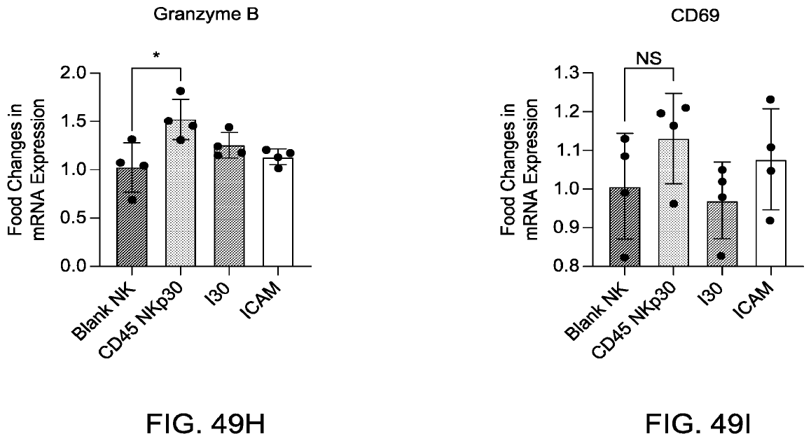

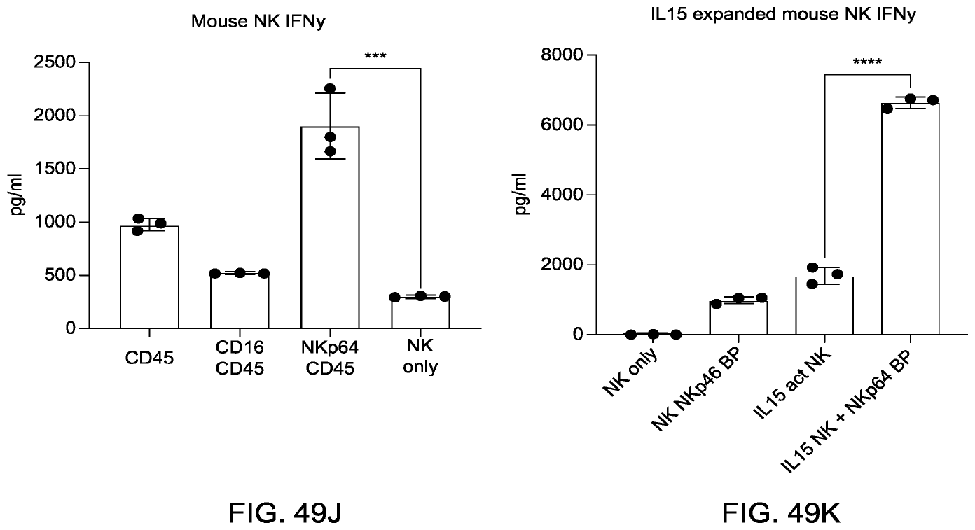

Different backpack formulations (Table 6) were screened for the ability to activate NK cells. The antibodies and proteins of Table 6 were attached to the backpack as described in the above Examples for binding agents. ICAM NKp30 backpacks cause highest degranulation of NK-92 cells as observed by LAMP-1 upregulation (FIG. 49A). ICAM NKp30 backpacks also cause highest IFN-y secretion of NK-92 and primary NK cells (FIGS. 49B-49C). Backpack attachment causes upregulation of intracellular mRNA transcripts of cytokines IFN-y, TNF-a and cytolytic effector proteins Perforin and Granzyme B in human primary NK activation (FIGS. 49D-49I). CD45 NKp46 backpacks cause highest IFN-y secretion from mouse NK cells (FIG. 49J). This effect is enhanced by pre-activation of mouse NK by IL-15 (FIG. 49K)

TABLE 6

| | Antigen of the |
|---|---|
| Group Name | antibody on the BP |
| Blank BP | N/A |
| ICAM BP | ICAM 100% |
| NKp30 BP | NKp30 100% |
| I30 BP | ICAM 50%, NKp30 50% |
| I46 BP | ICAM 50%, NKp46 50% |

Backpacks can be coupled to human and mouse NK cells through various ligands. Backpack adhesion causes upregulation of activation markers and cytokine secretion by mouse and human NK cells depending on the type of antibody used for adhesion with attached surface ligands. Pre-activation with cytokines such as IL-2 or IL-15 further sensitizes NK cells to backpack activation. Free antibody/ligand does not elicit same level of activation compared to when presented through backpacks, potentially due to receptor crosslinking induced by backpacks. Primary NK cells exhibit -10-fold higher activation as compared to NK-92. Under these conditions, the CD45-NKp30 combination exhibited activity that was superior for human NK, CD45-NKp46 best for mouse NK in terms of highest cell activation and adhesion.

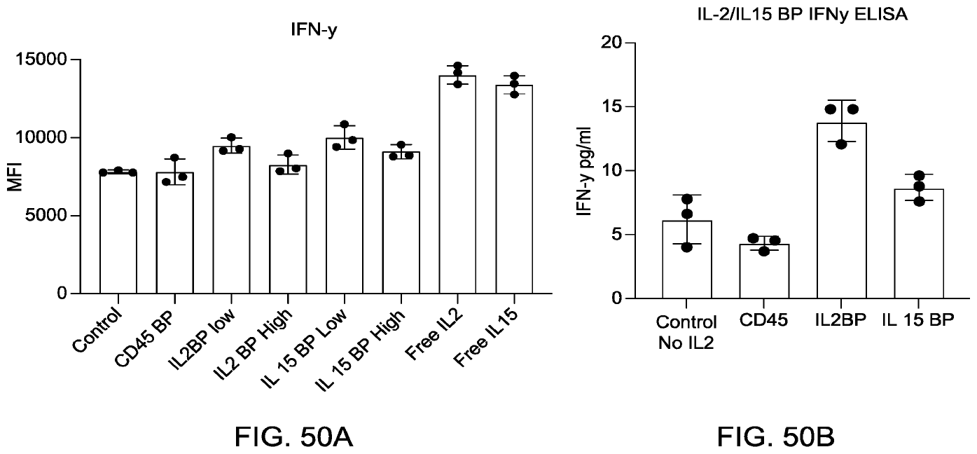

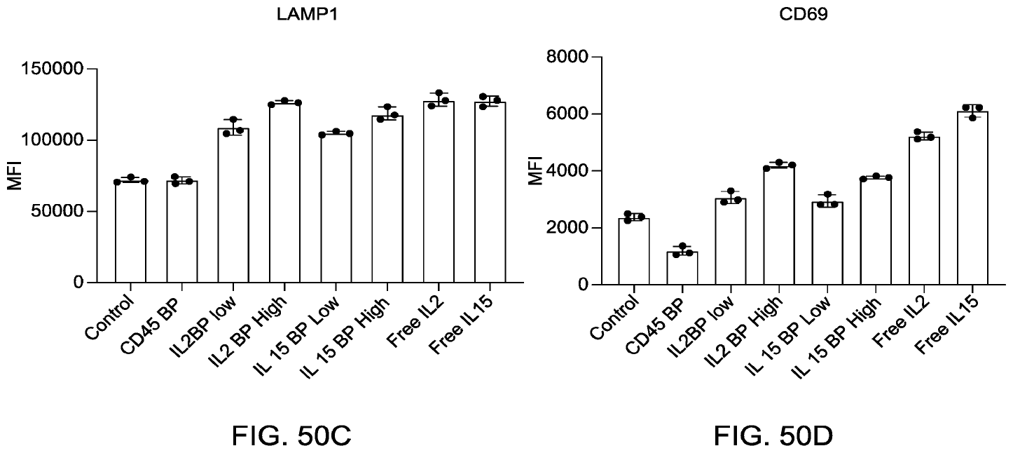

A further means of improving NK persistence and activation is to include cytokines in the backpacks. In this approach, the cytokine on the backpack is presented to the cognate receptor on the NK cells, causing the NK cell to internalize the cytokine. IL-2 and IL-15 were tested in this approach and shown to improve IFN-gamma secretion of NK-92 cells (FIGS. 50A-50B). Cytokine backpacks also increase degranulation and surface activation marker expression of NK-92 cells (FIGS. 50C-50D).

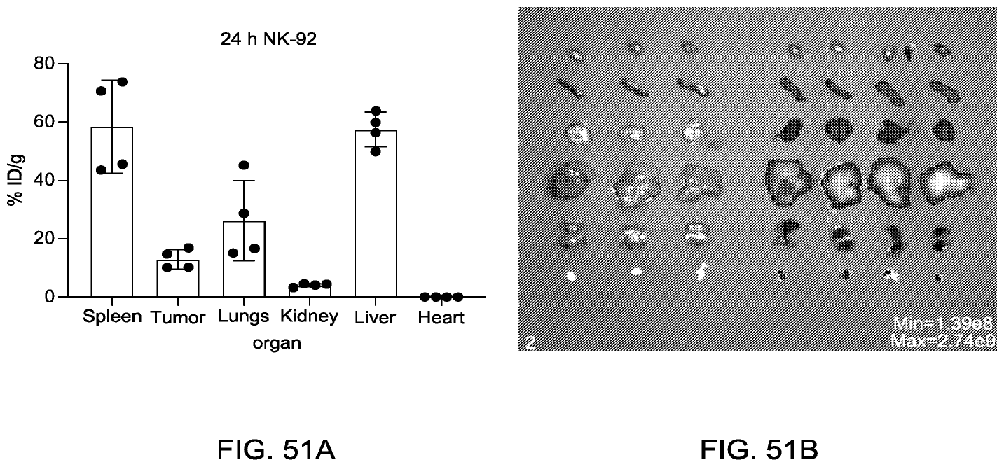

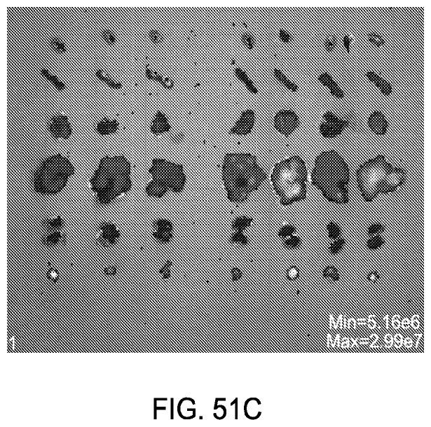

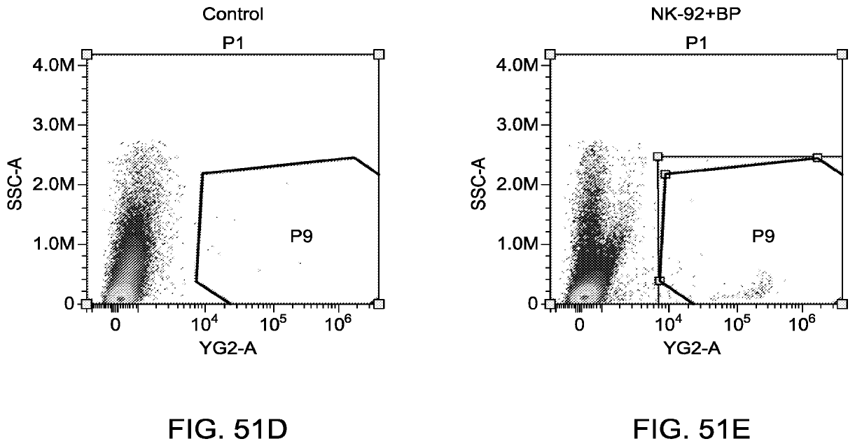

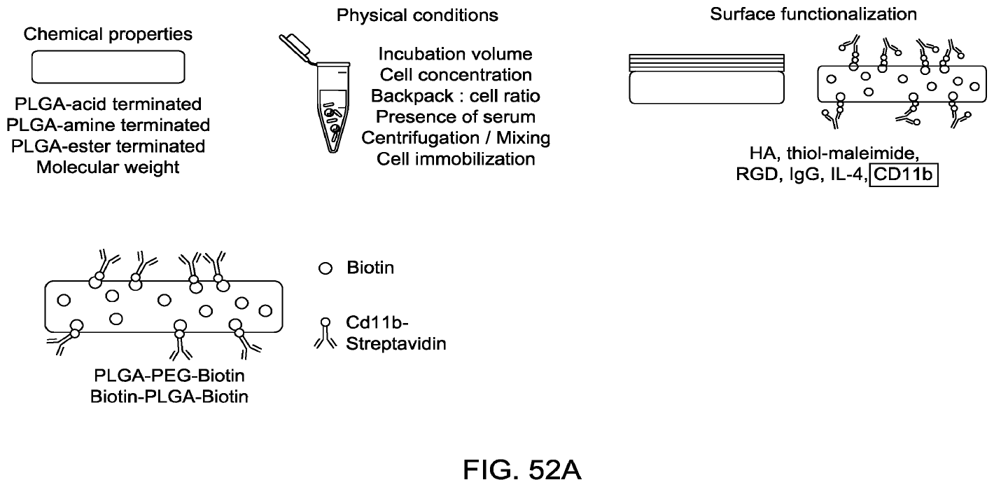

The biodistribution of NK-92 cells, and NK-92 cells with adhered backpacks was examined in mice (FIGS. 51A-51E).

### Example 8

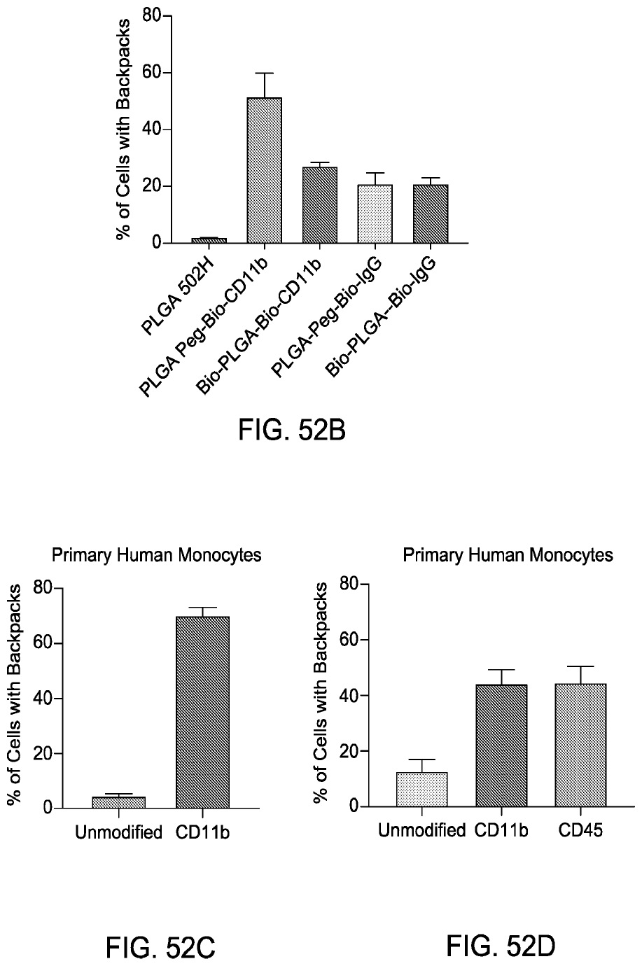

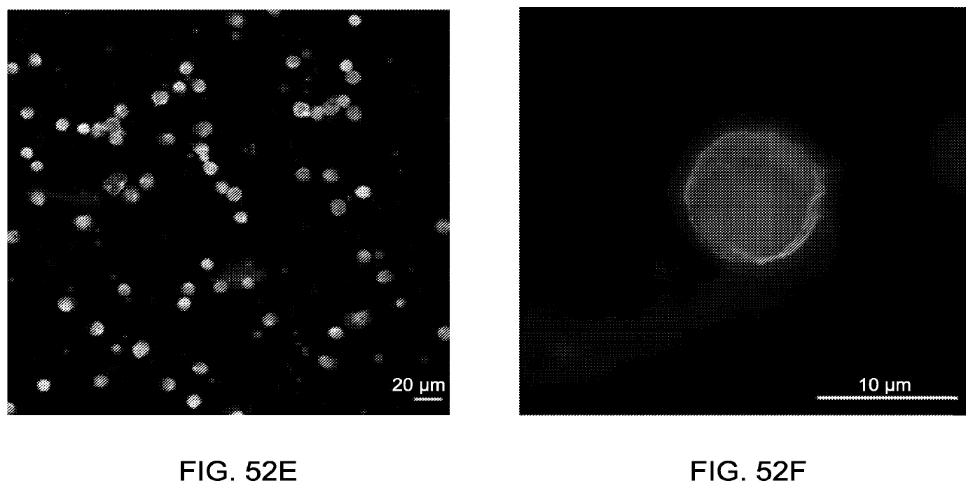

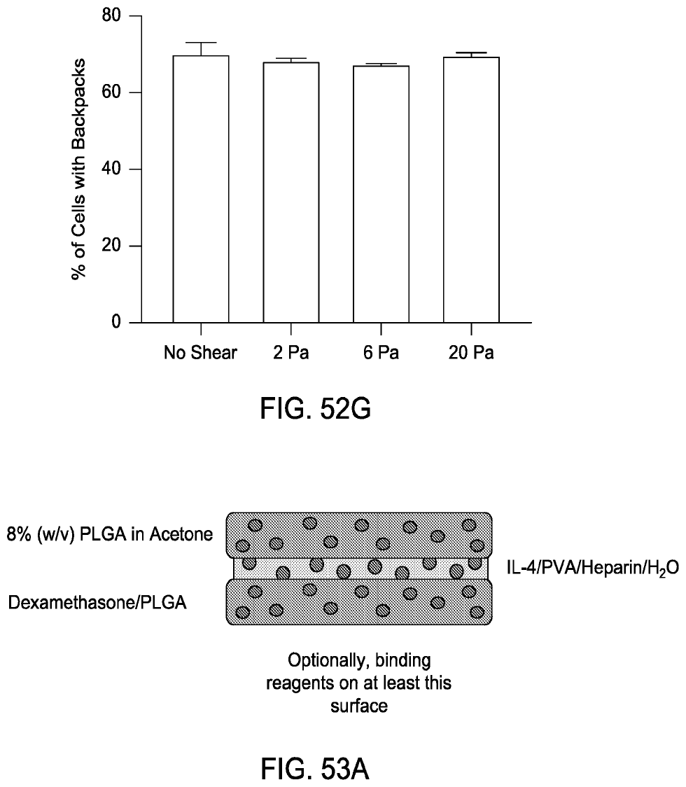

The effect of adhesion of backpacks to monocytes was also examined. A summary of adhesion methods is provided in FIG. 52A, and the rate of adhesion of a number of backpack formulations is shown in FIG. 52B. Backpacks adhere to primary murine and human monocytes (FIGS. 52C-52F). Antibody mediated backpack adhesion to monocytes is maintained under varying shear conditions (FIG. 52G).

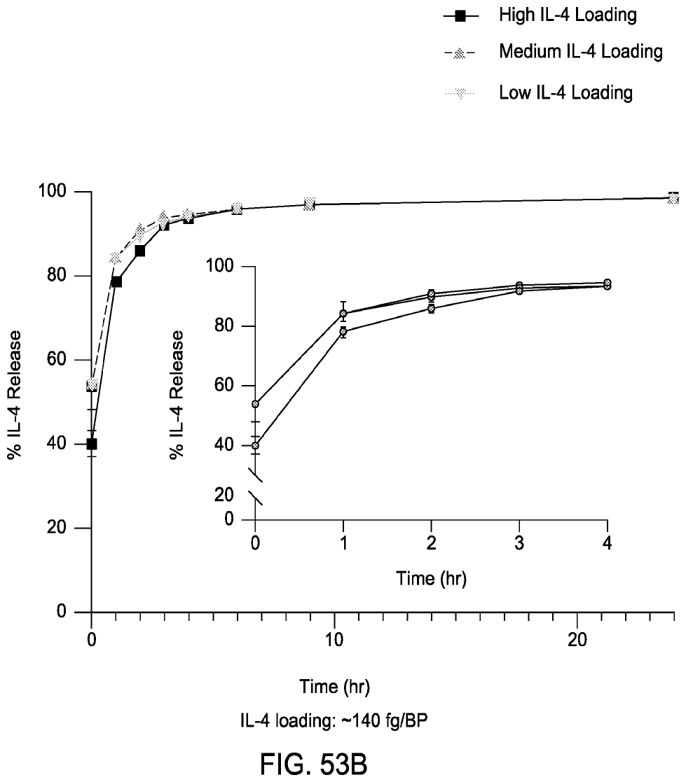

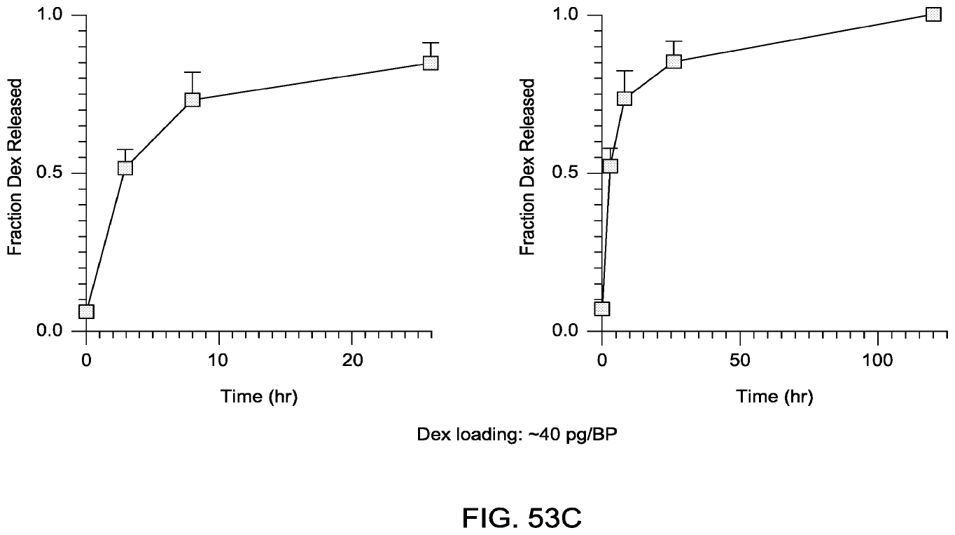

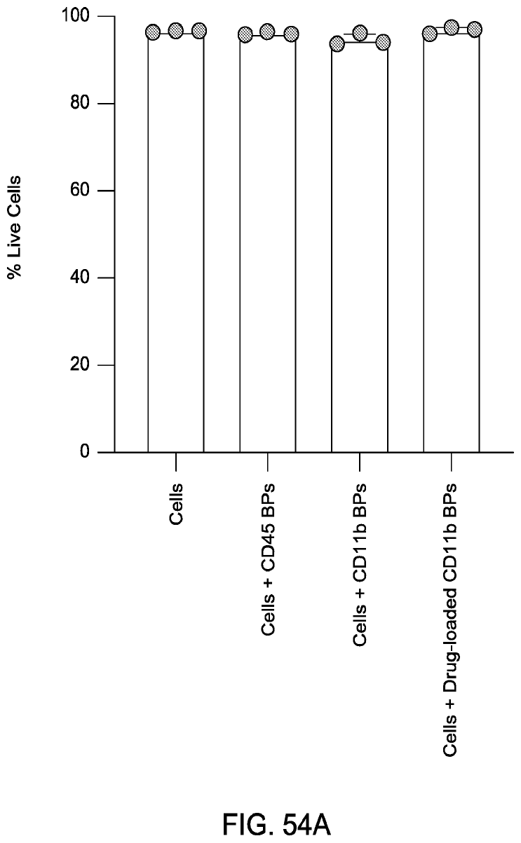

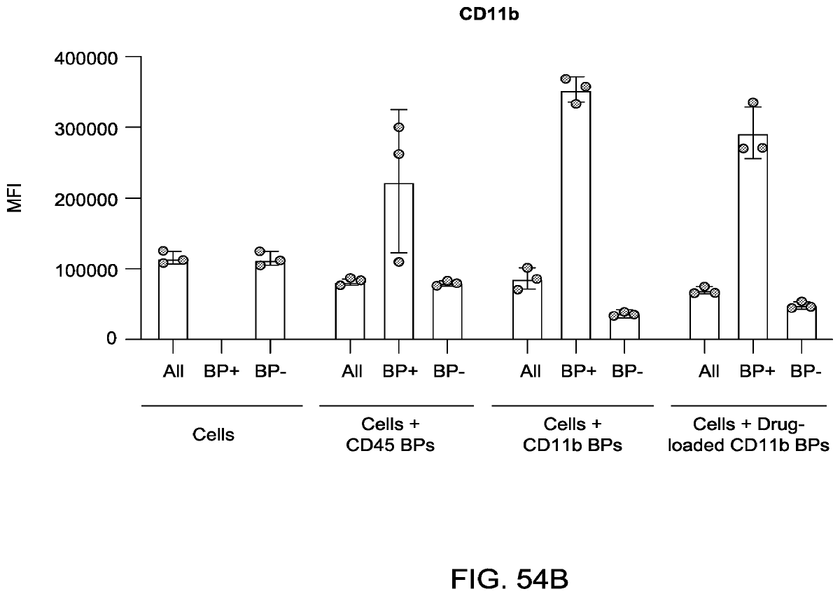

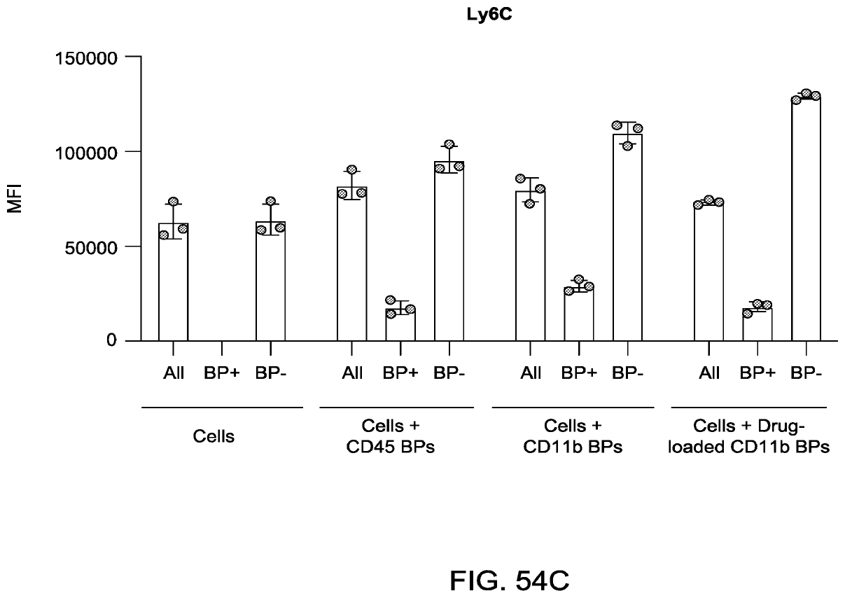

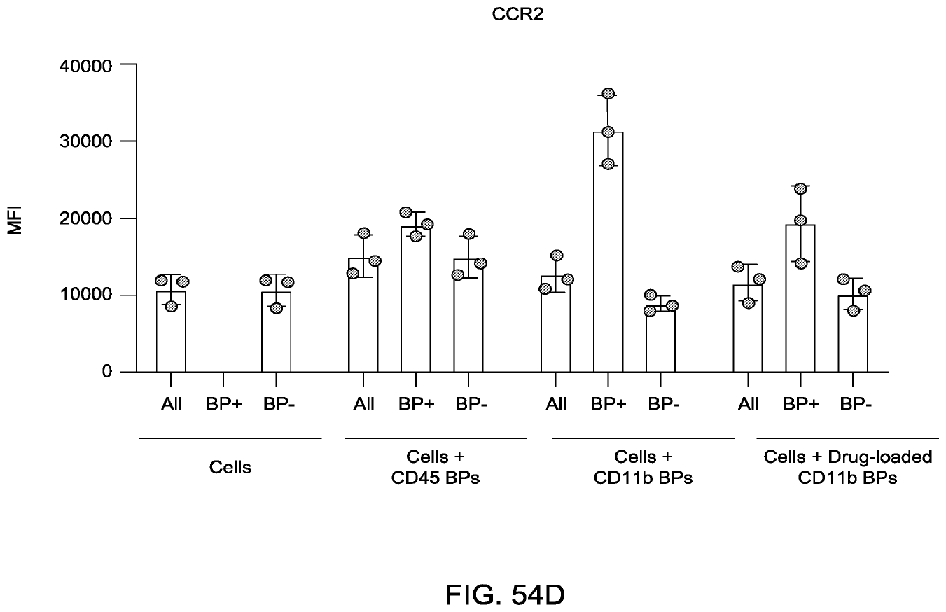

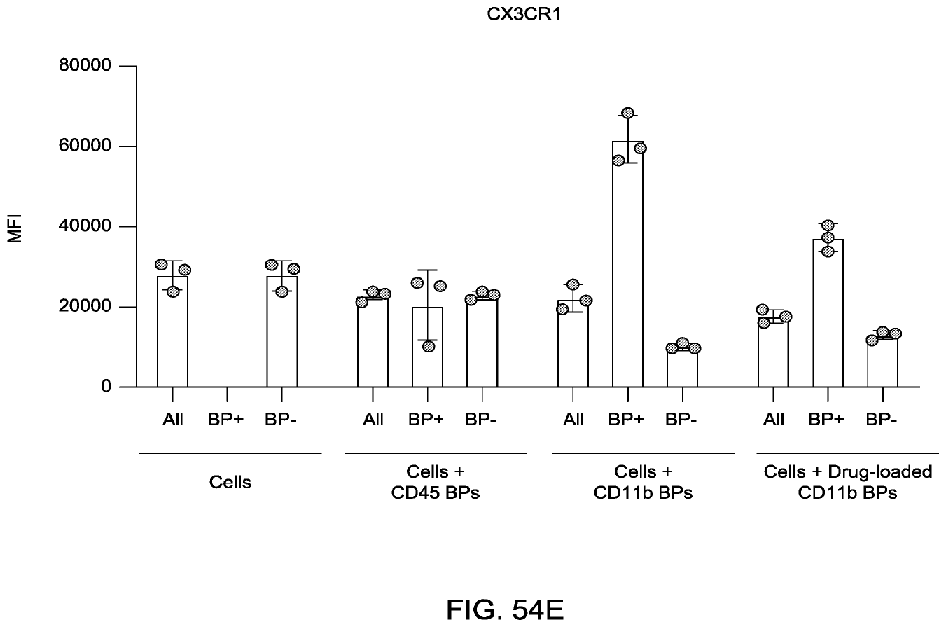

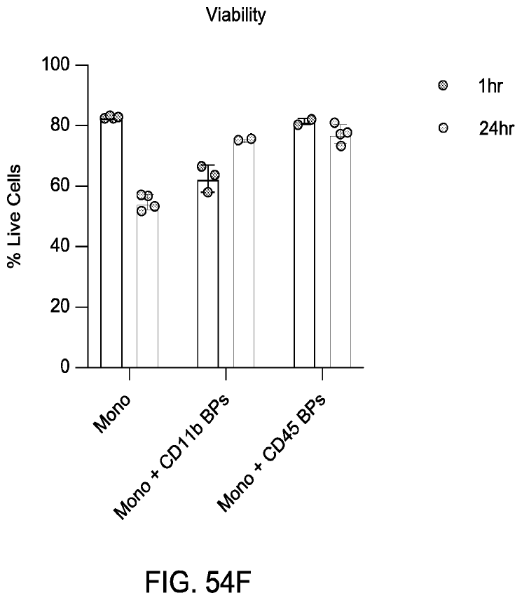

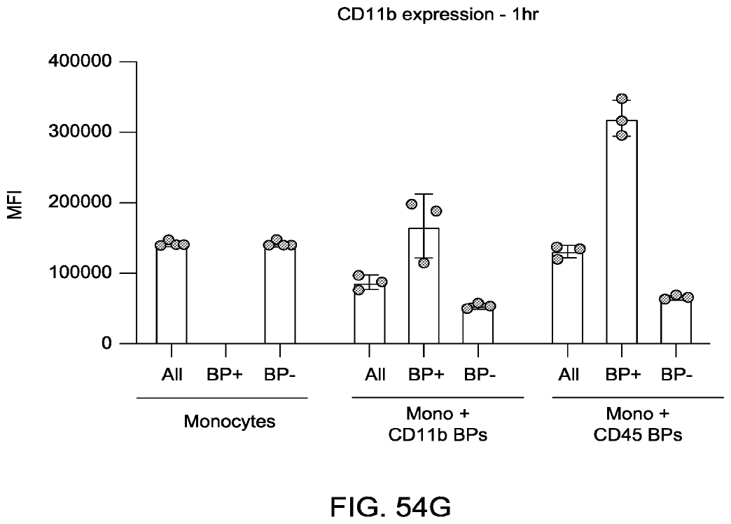

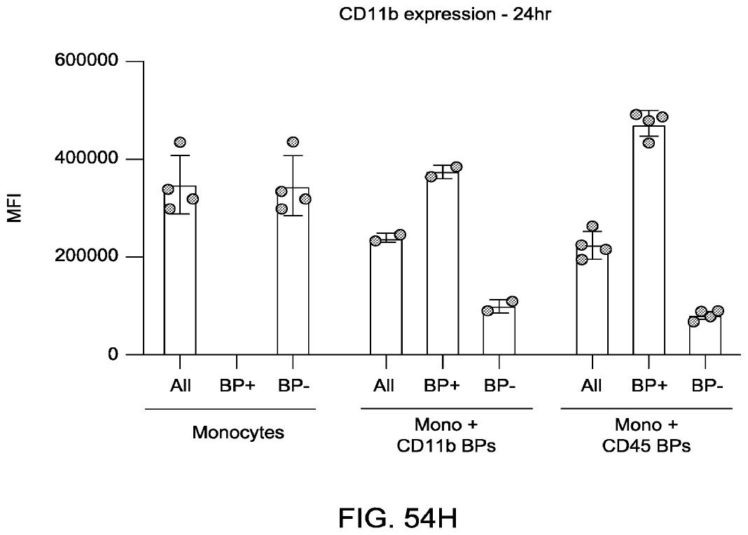

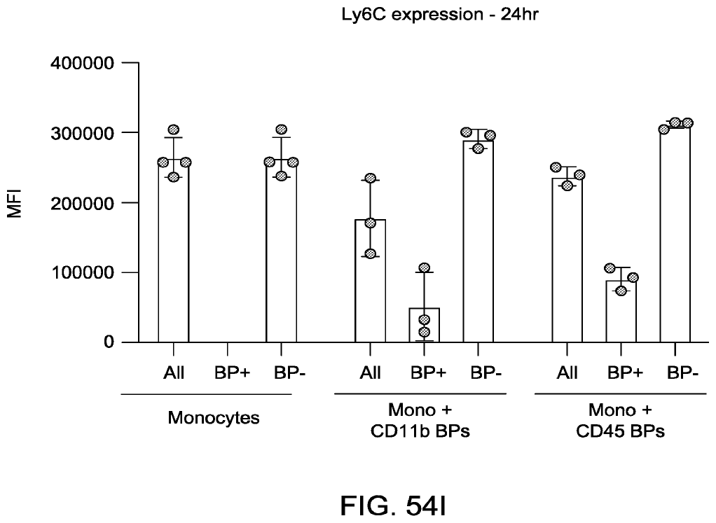

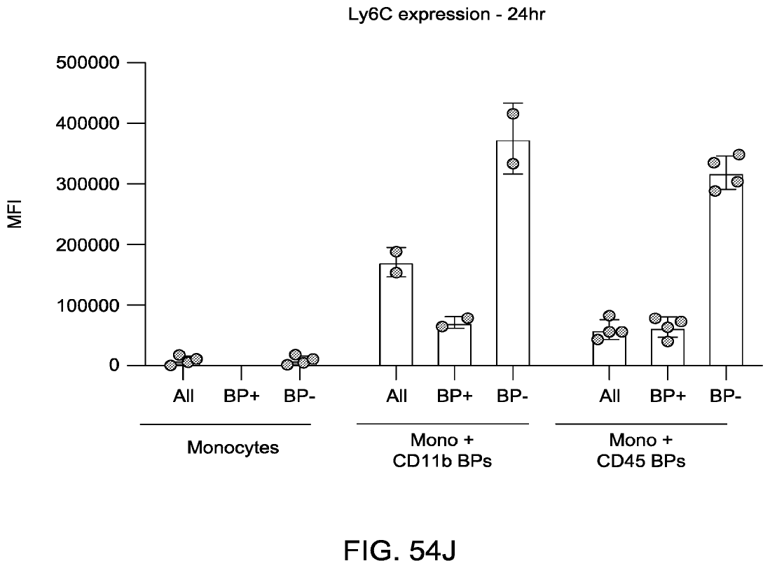

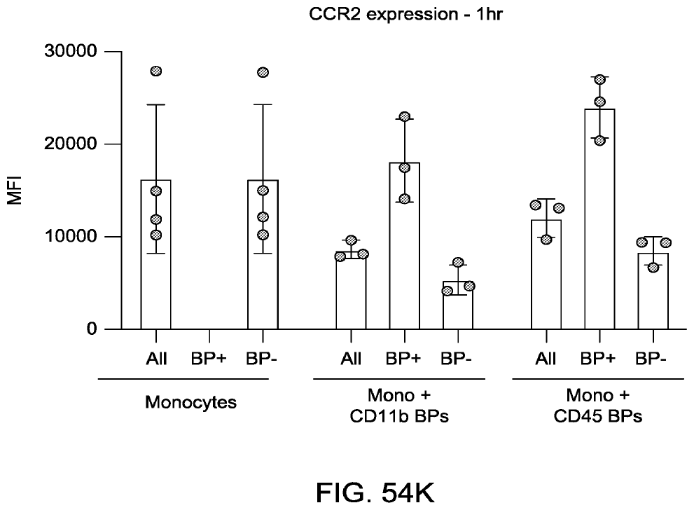

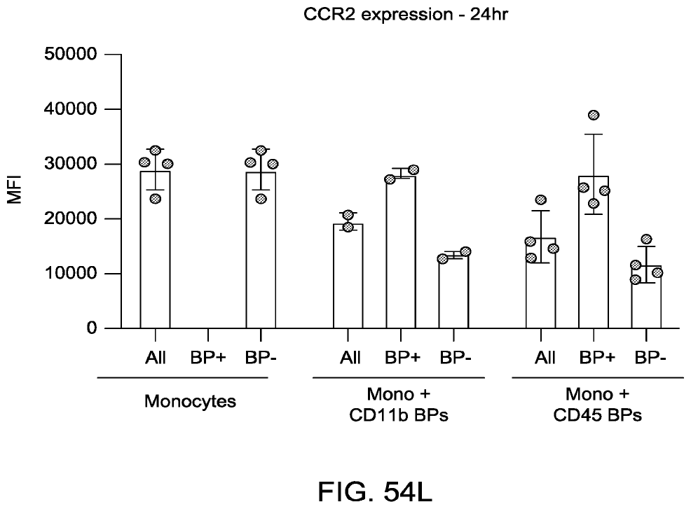

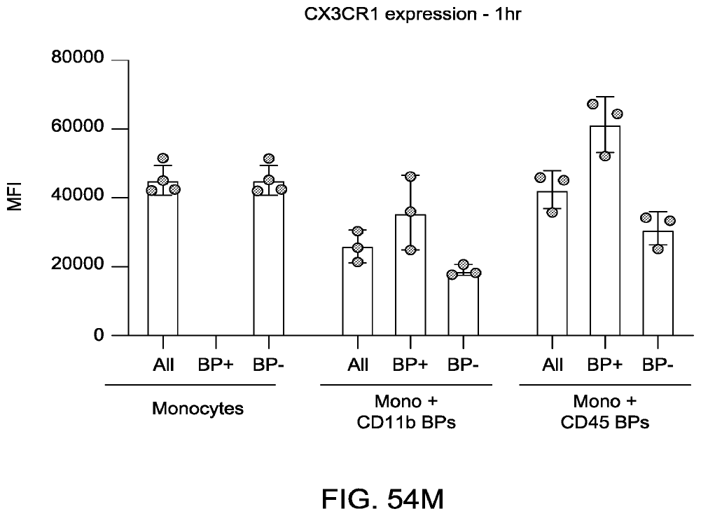

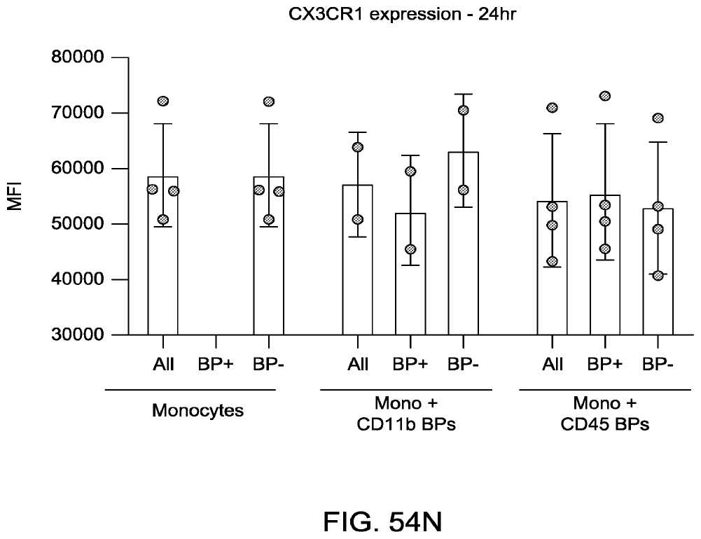

IL-4 (cytokine) and dexamethasone (small molecule) can be loaded into backpacks (FIGS. 53A-53C). Cells retain viability after backpack adherence (FIG. 54A). Monocyte expression of CD11b and Ly6C after backpack adherence was examined (FIGS. 54B-54C). Monocyte chemokine receptor expression is retained after backpacks are adhered (FIGS. 54D-54E). Carrier monocyte retains viability after 24h with antibody backpacks adhered (FIG. 54F). The longitudinal effects of antibody mediated backpack adhesion on carrier monocyte were determined (FIGS. 54G-54N).

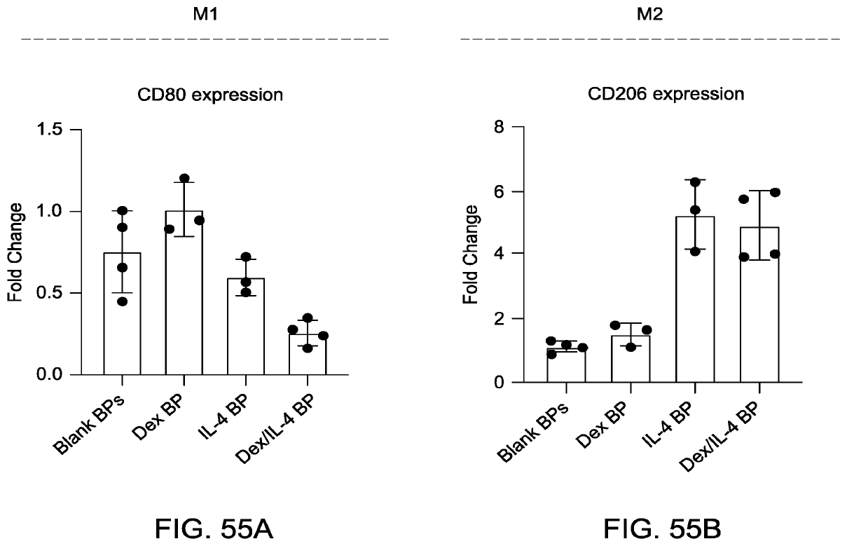

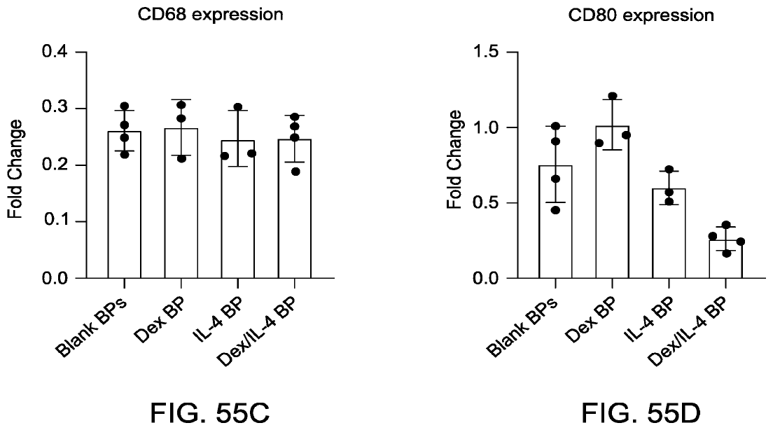

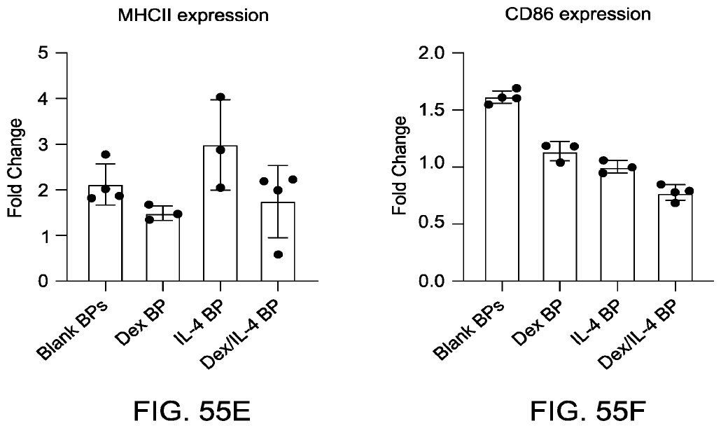

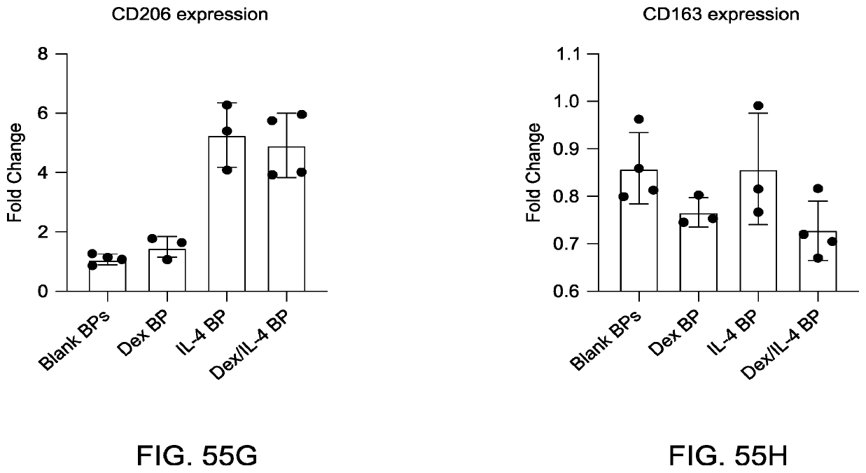

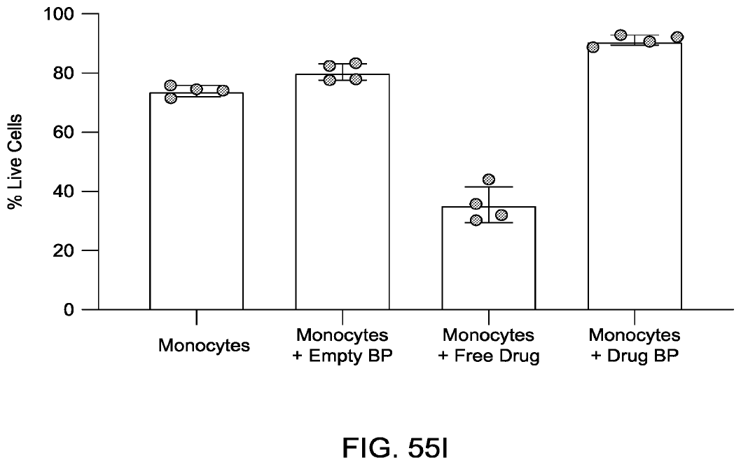

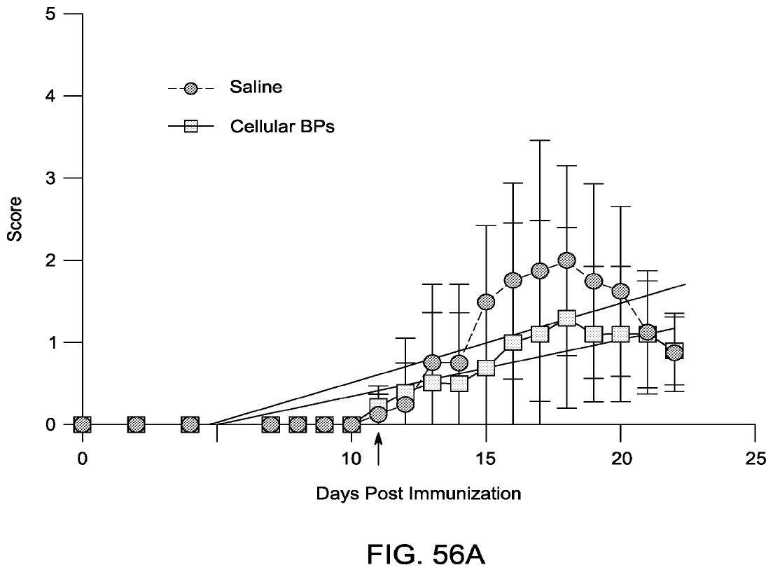

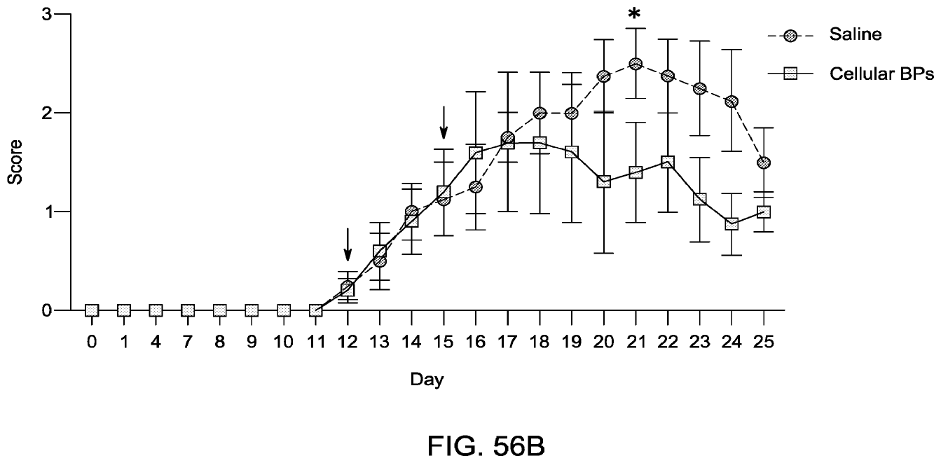

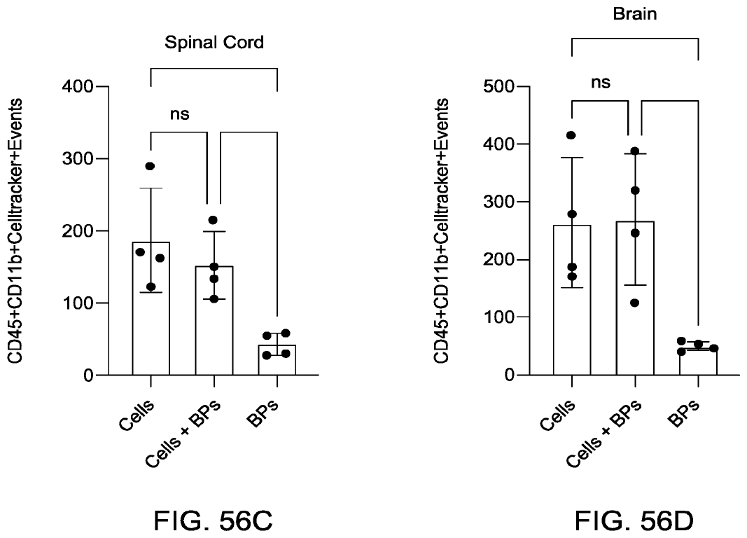

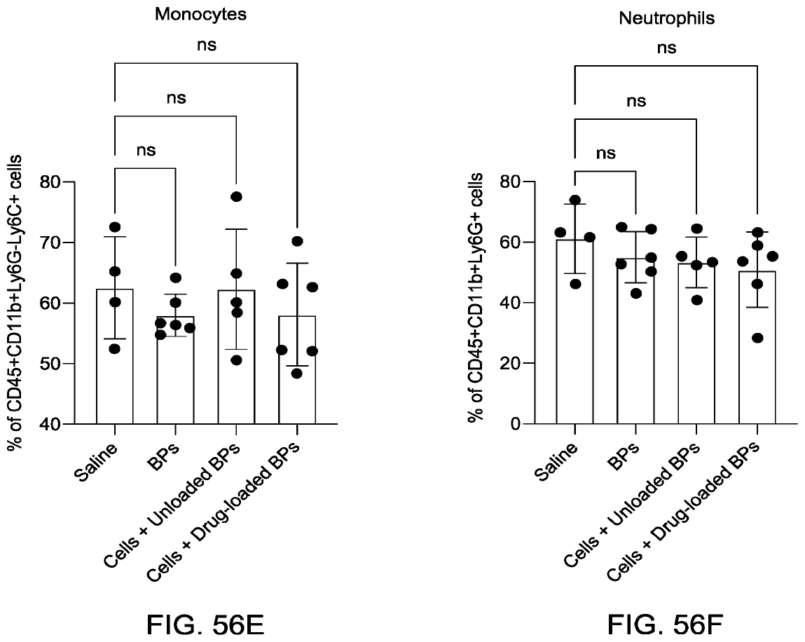

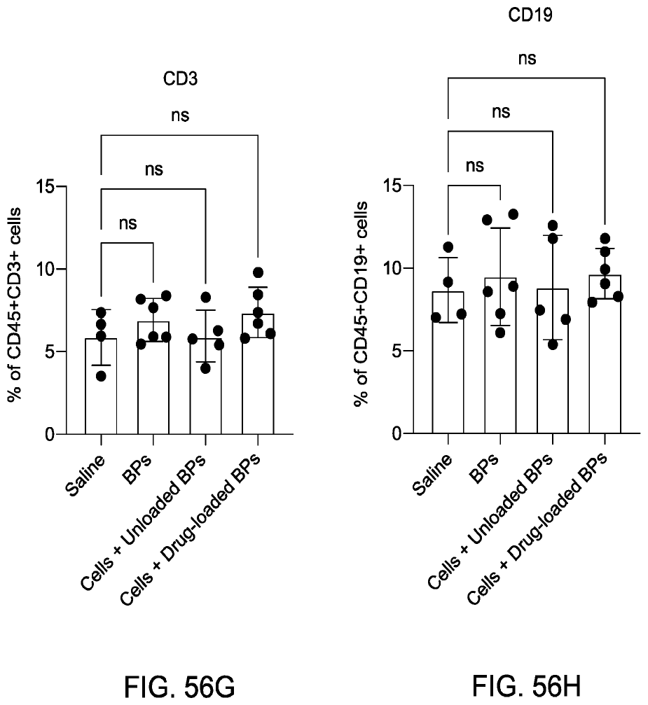

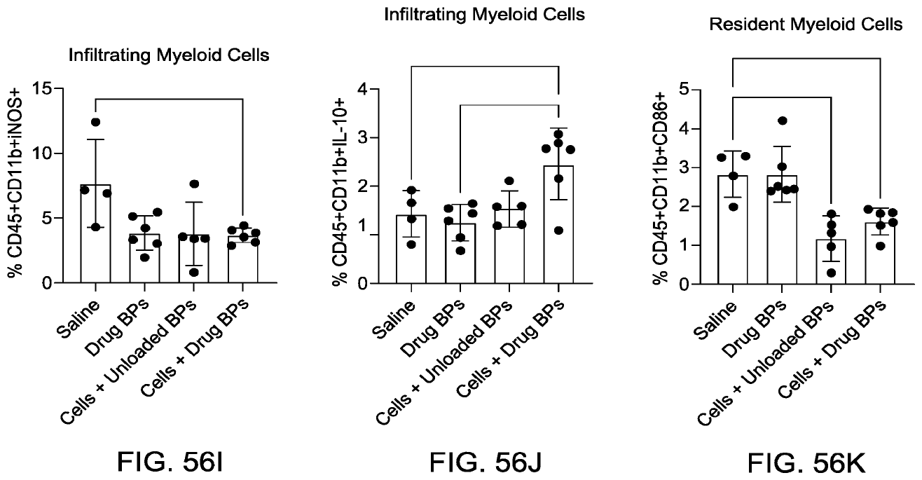

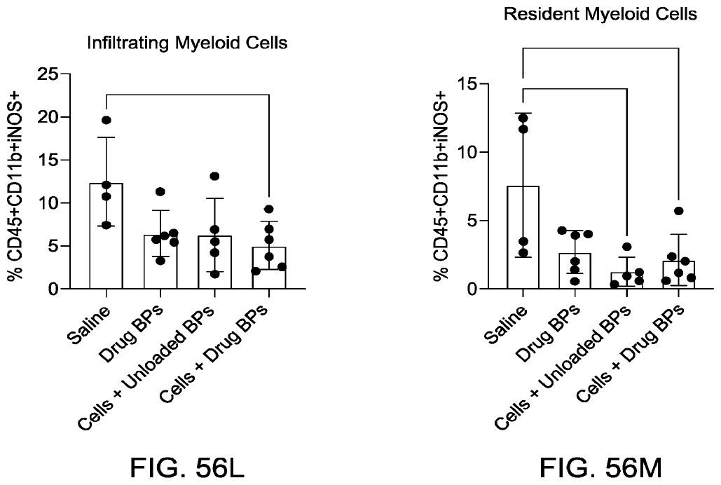

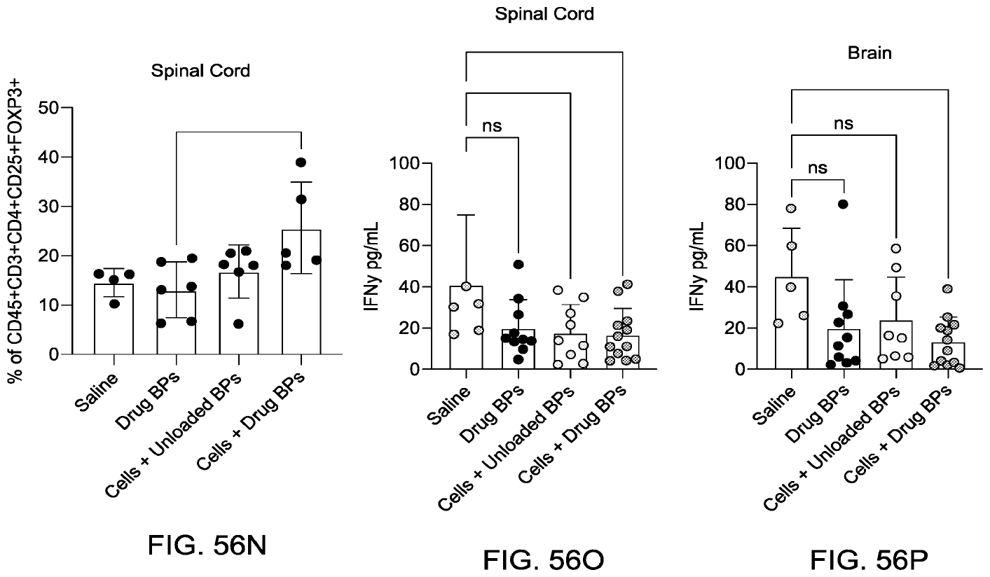

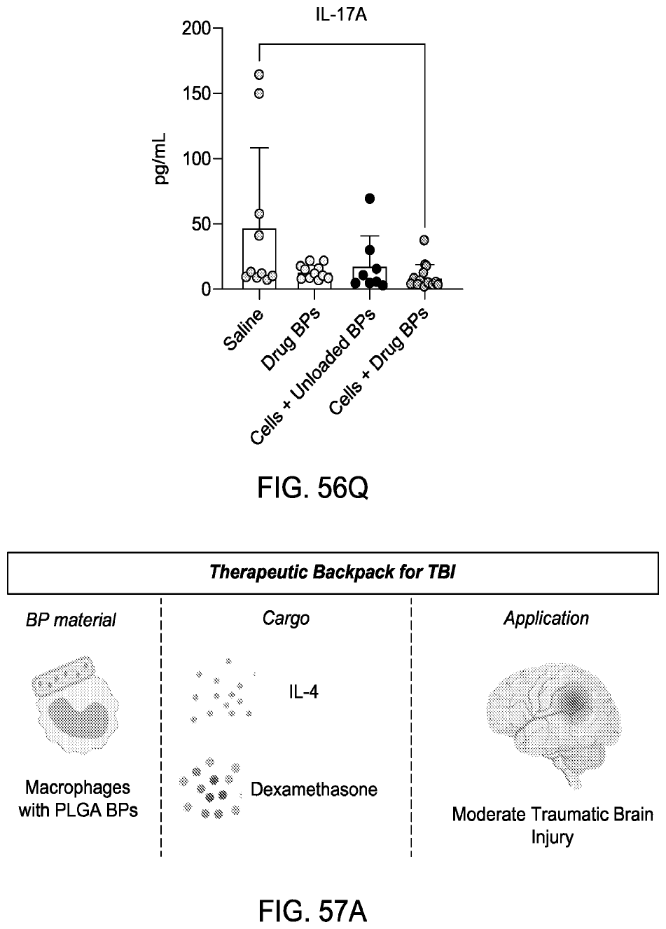

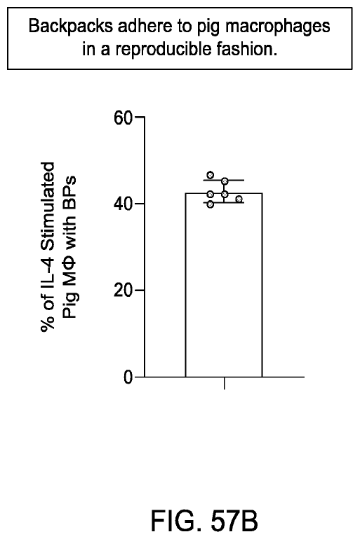

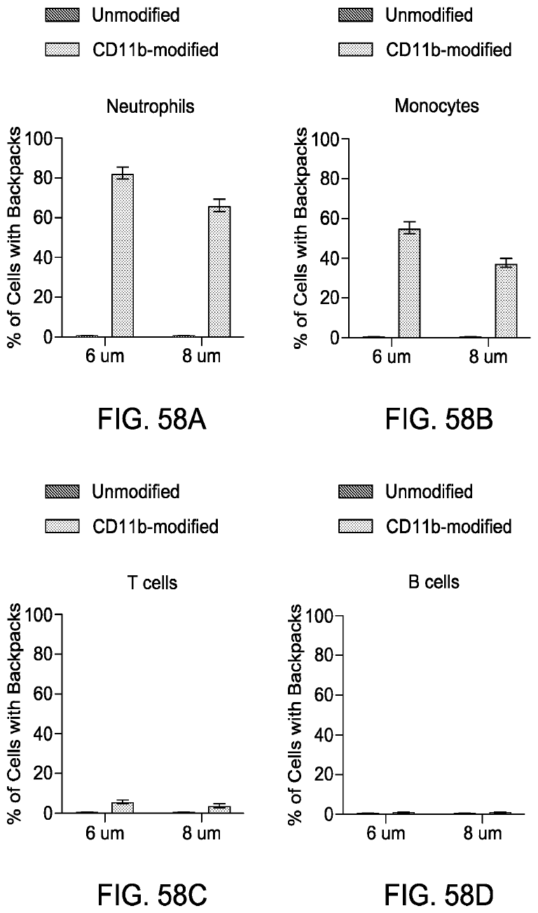

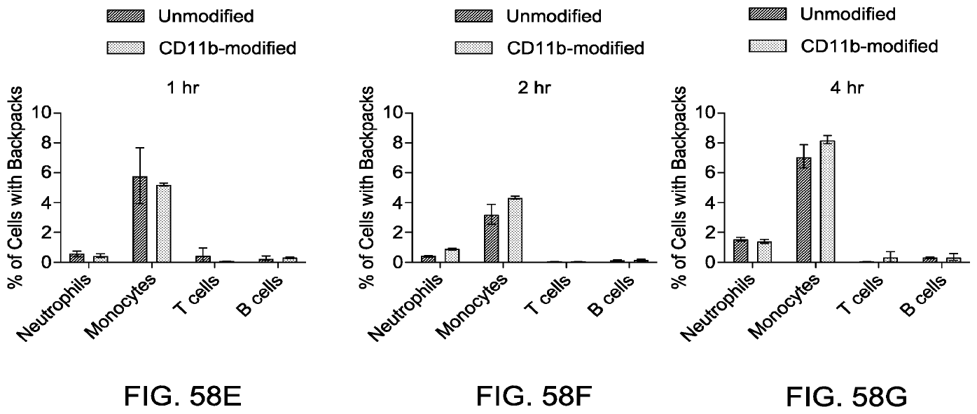

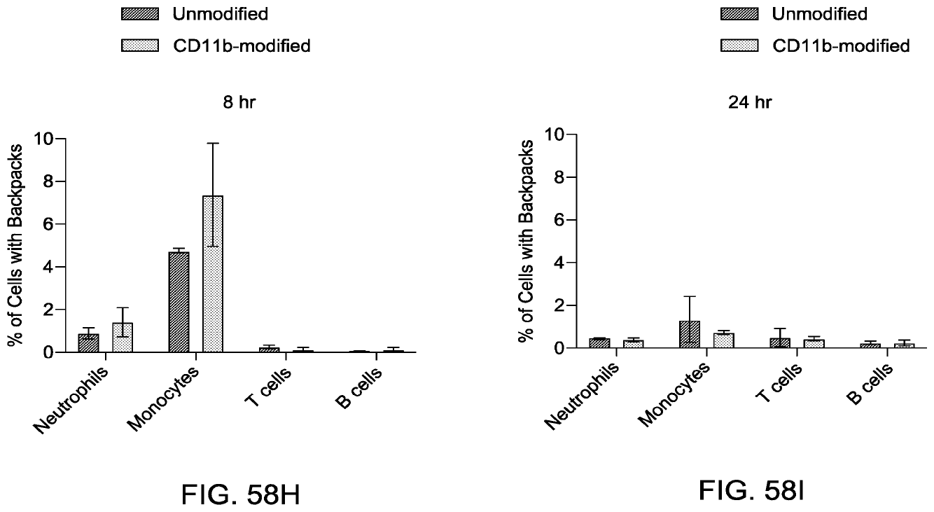

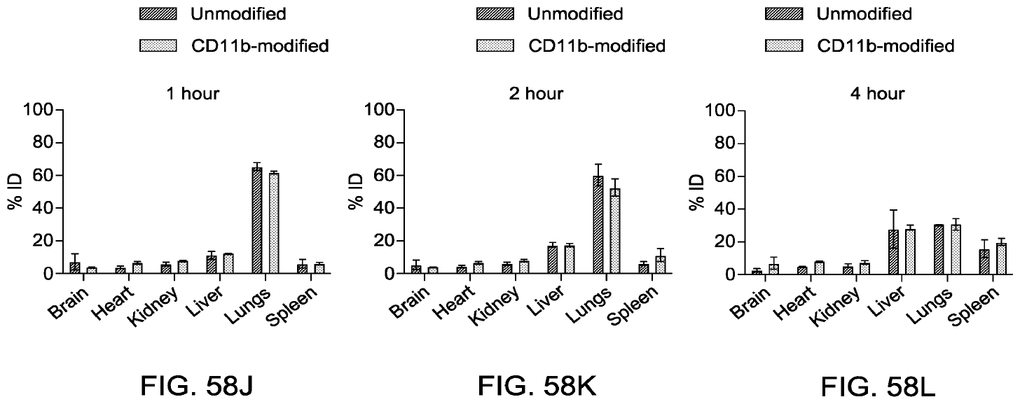

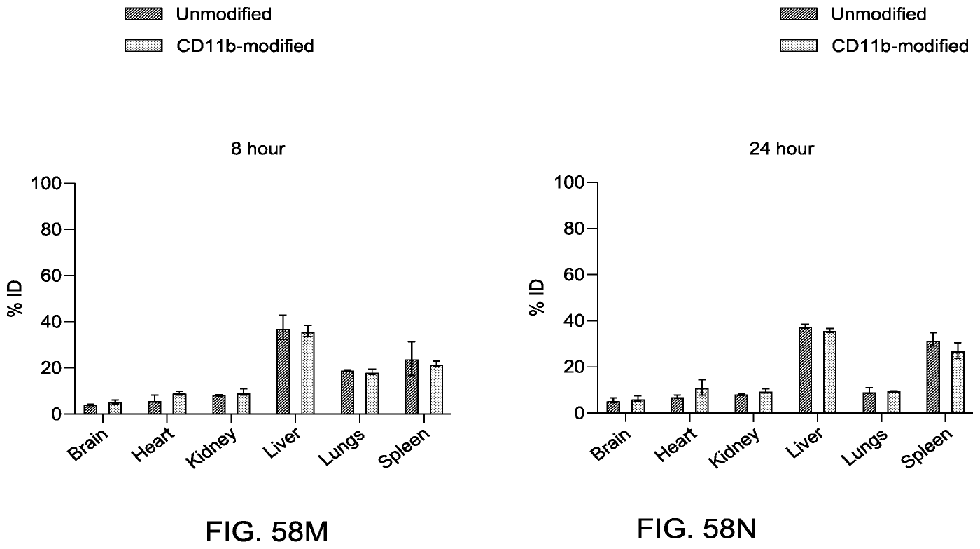

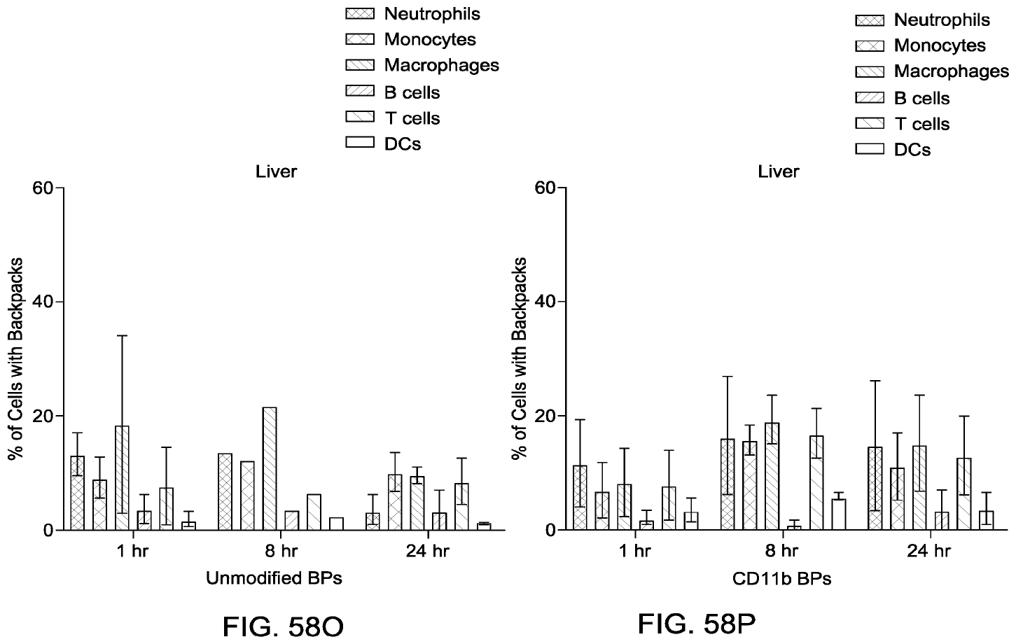

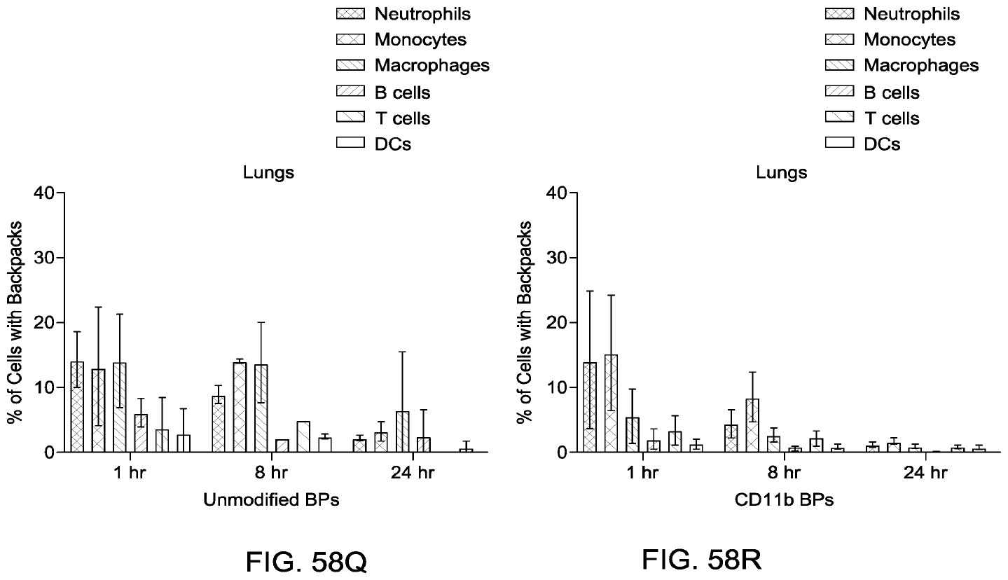

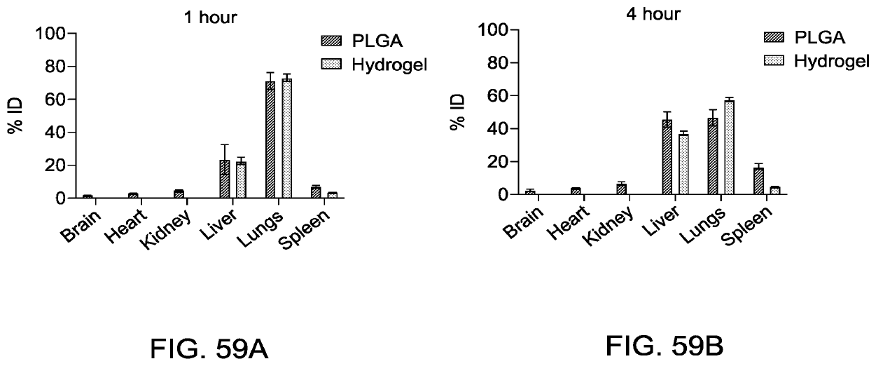

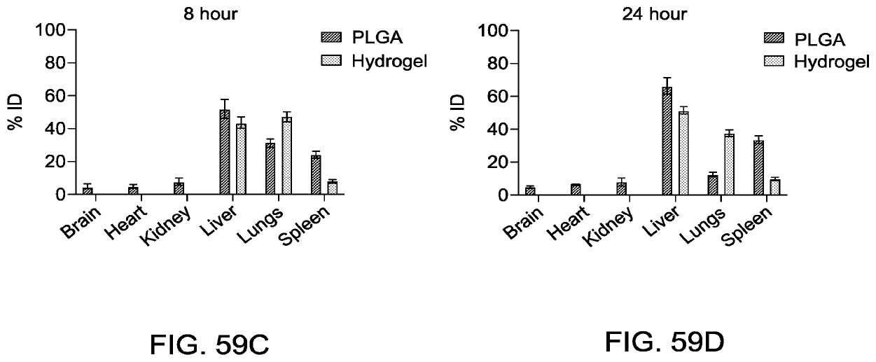

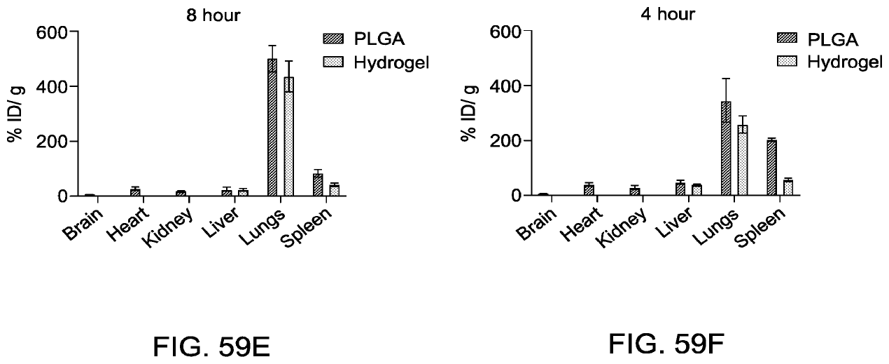

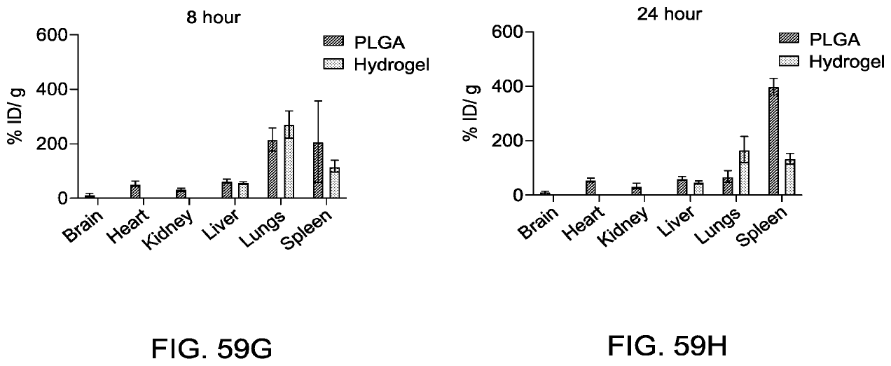

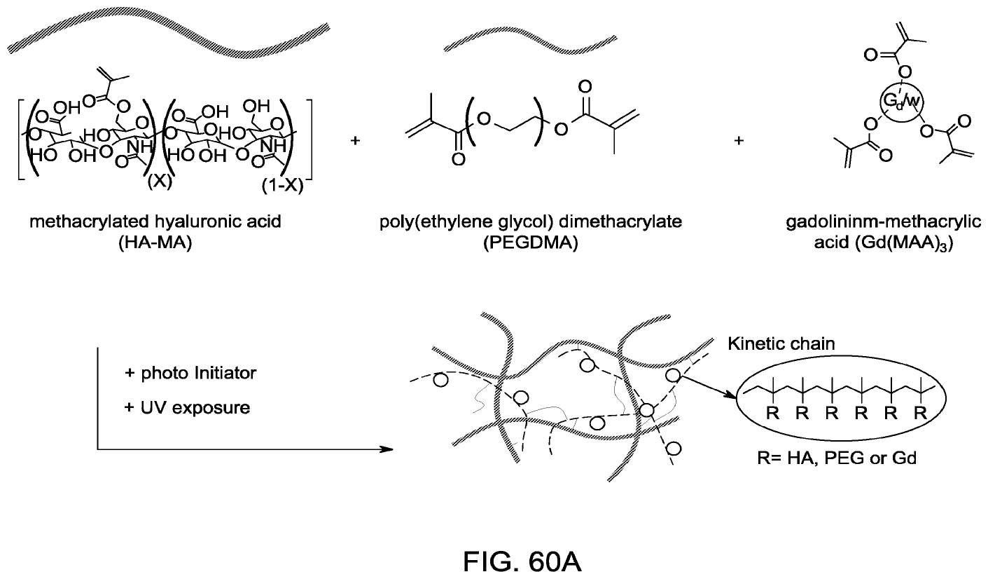

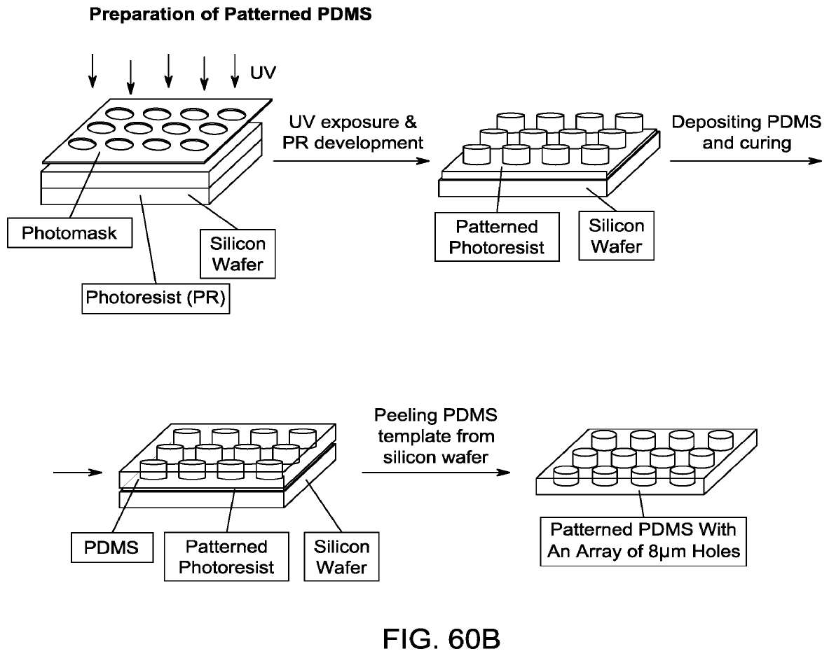

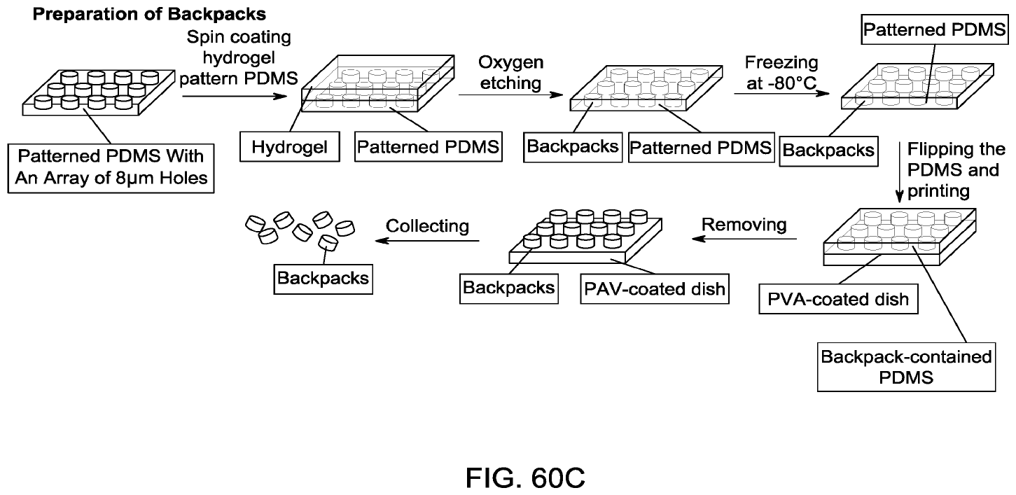

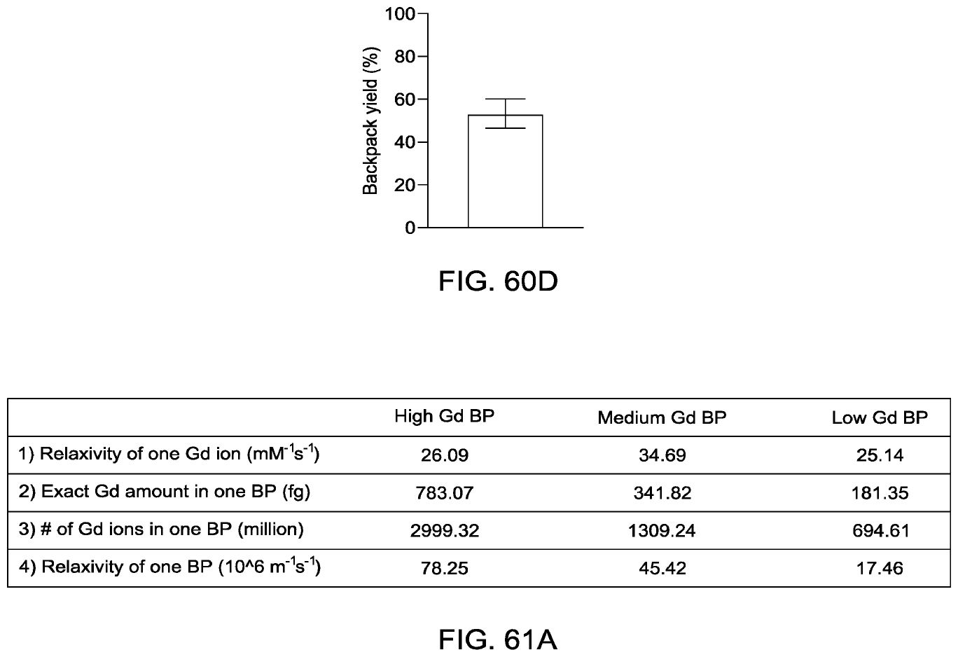

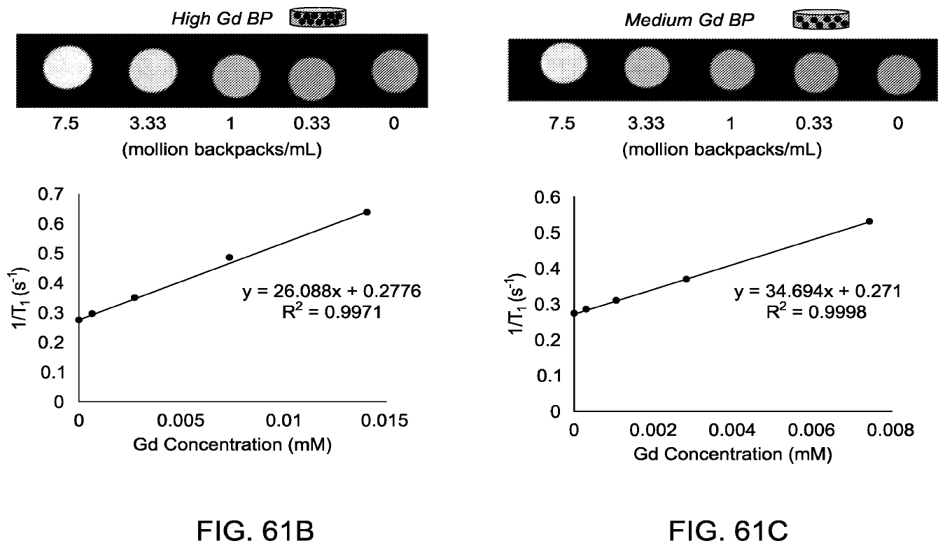

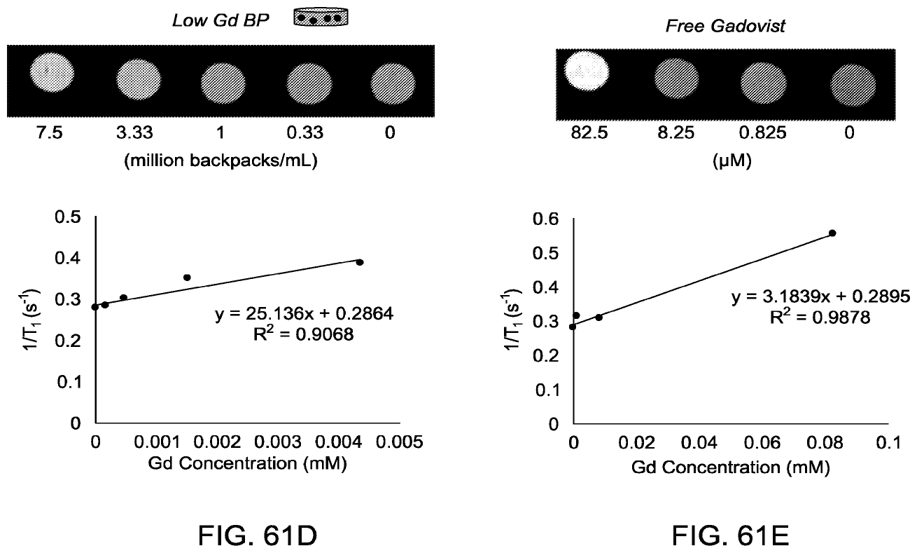

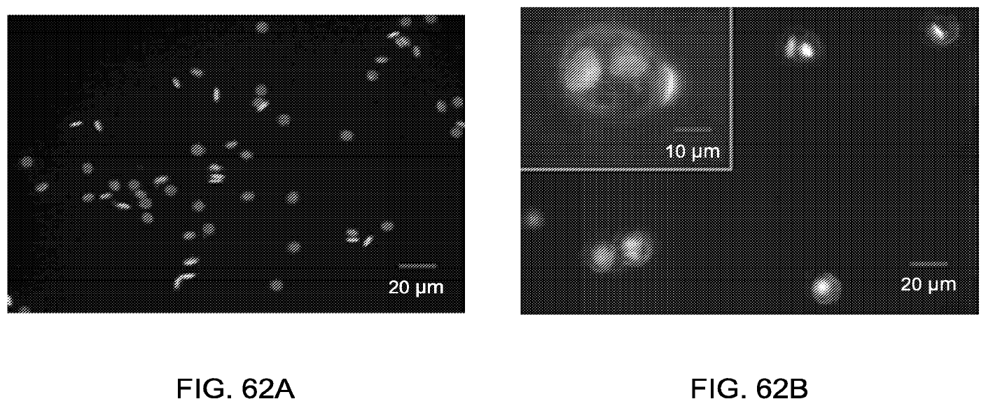

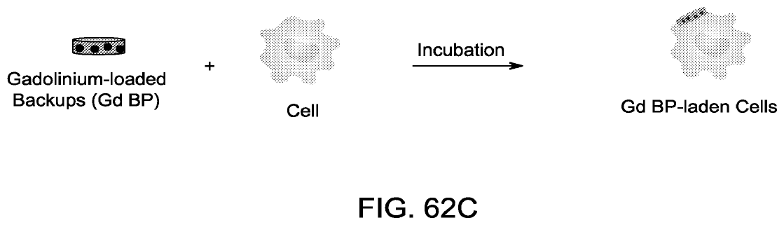

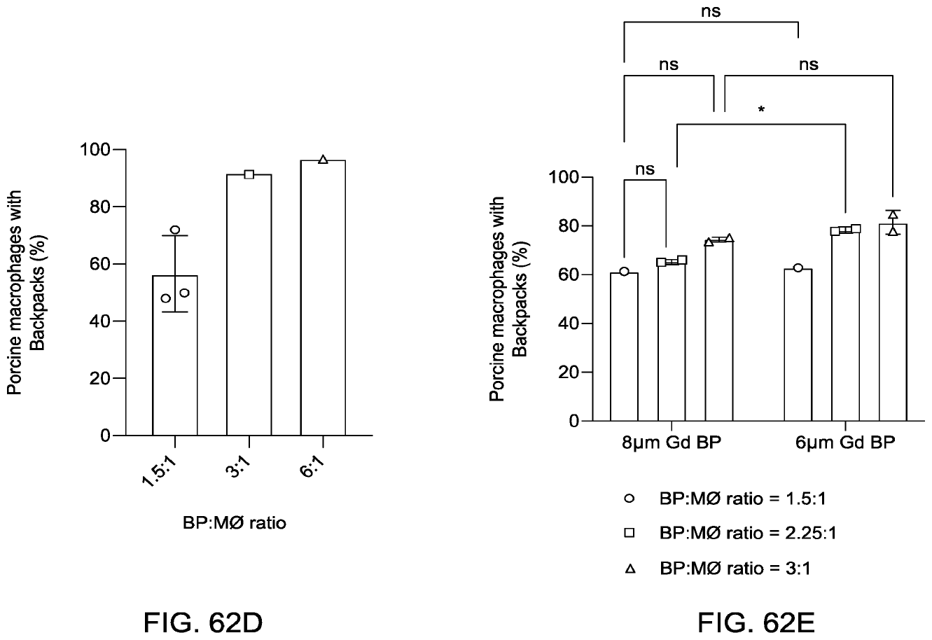

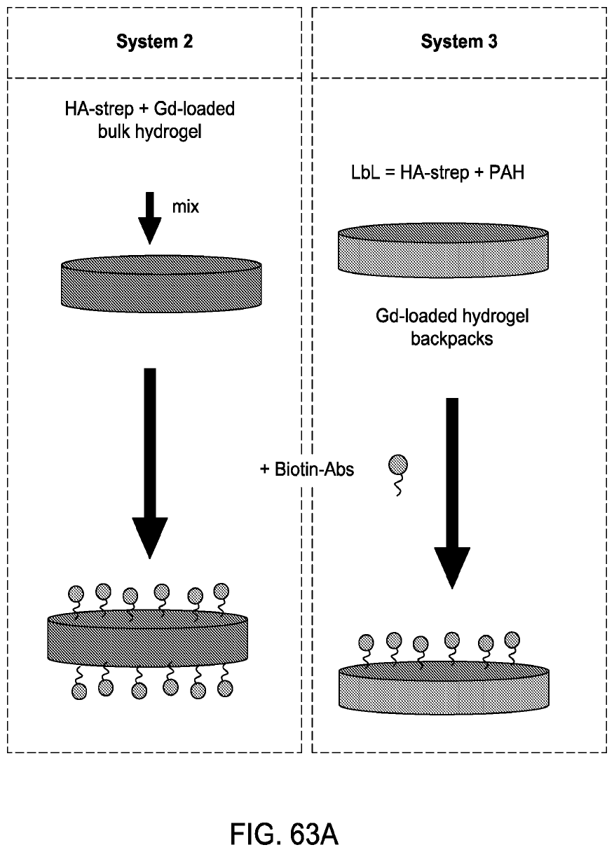

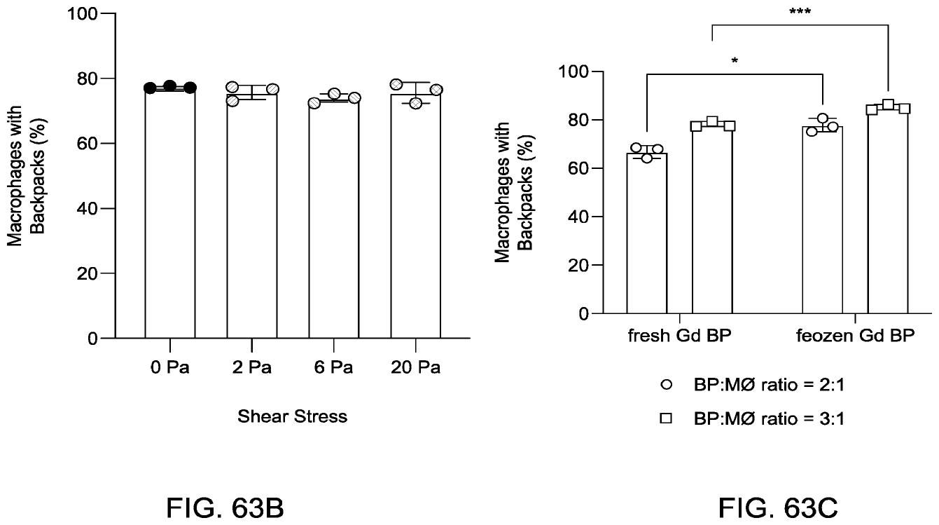

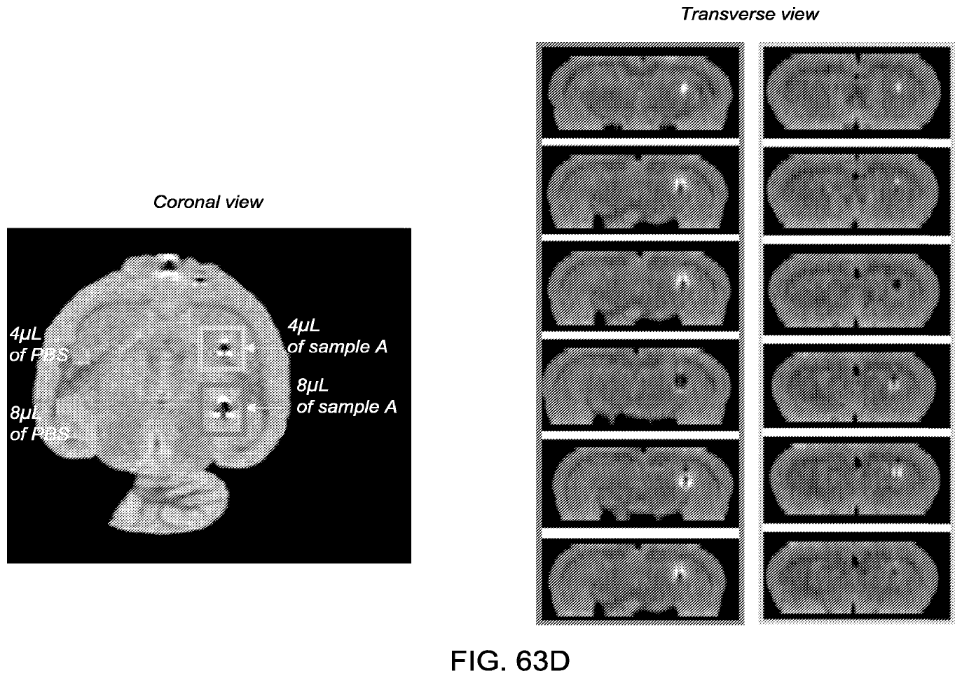

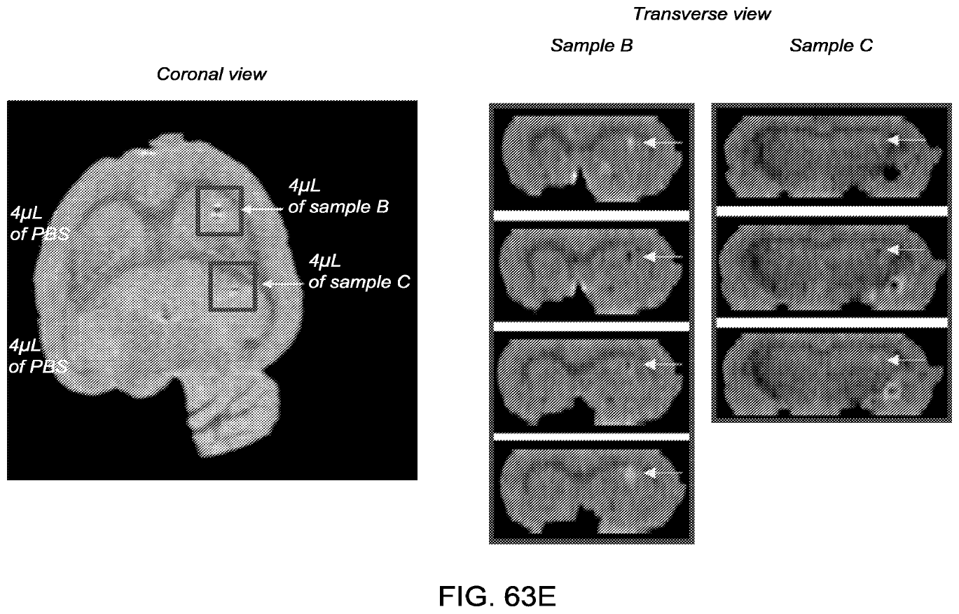

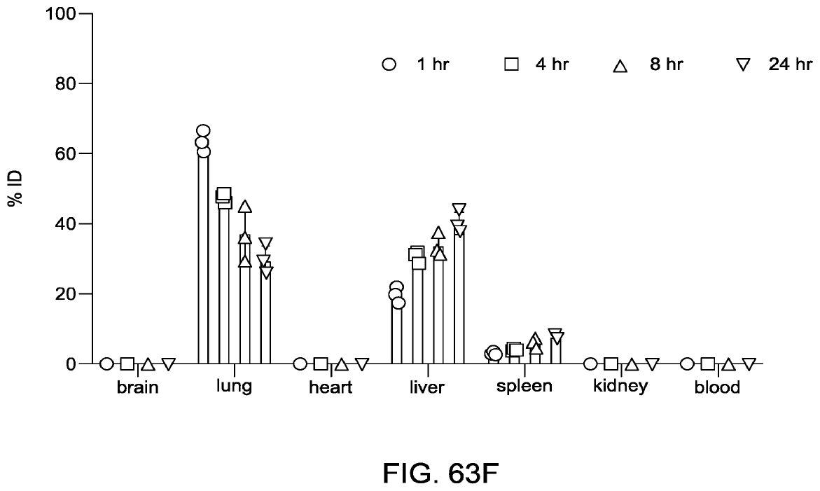

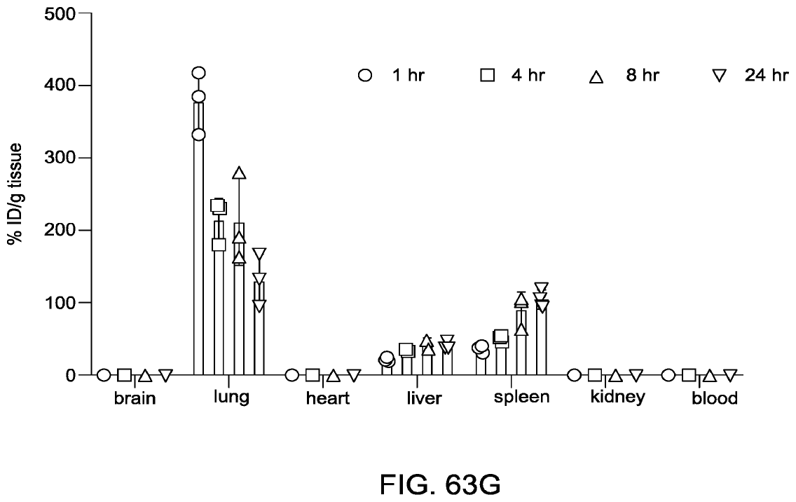

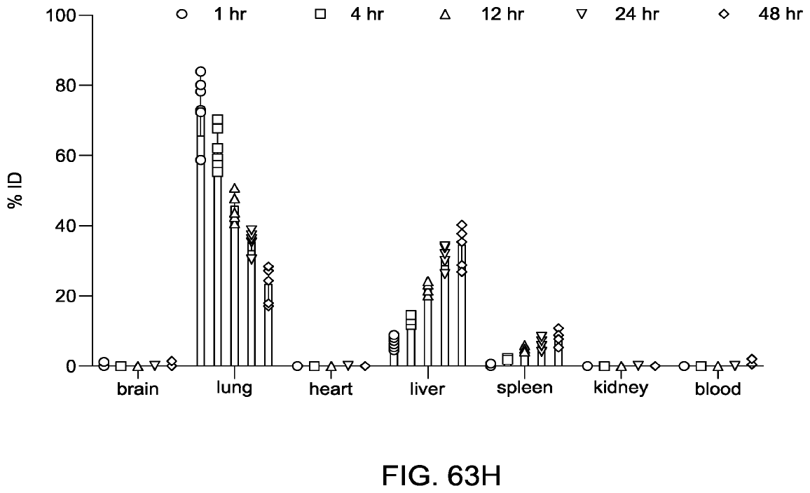

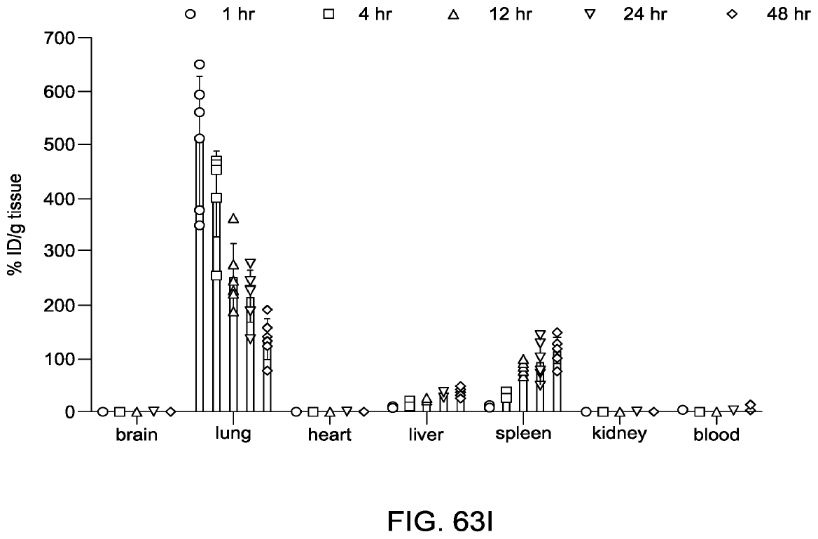

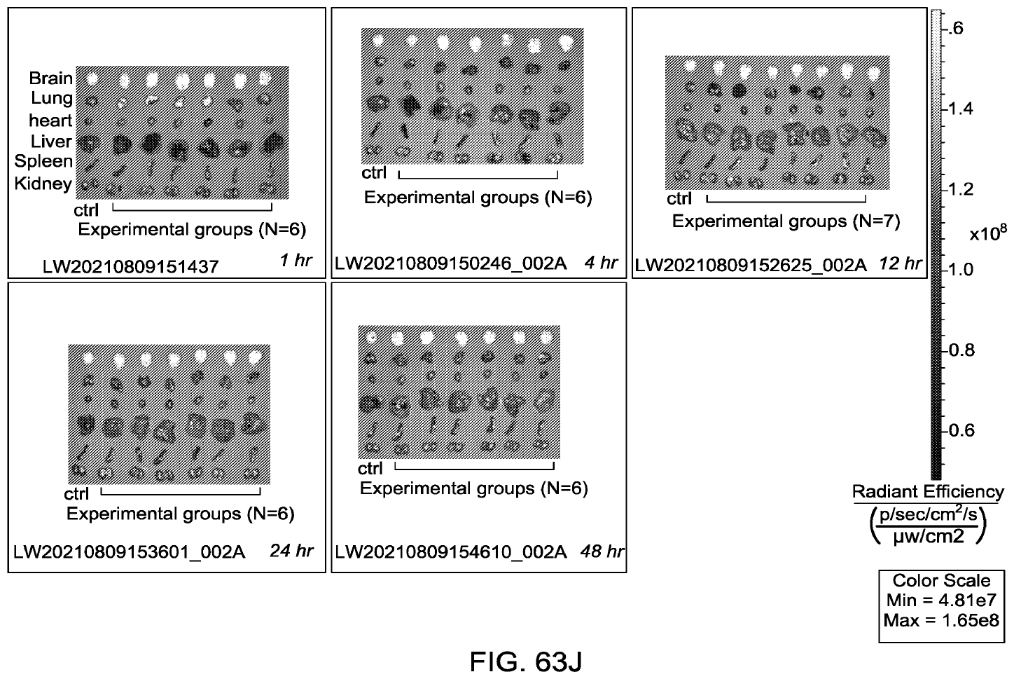

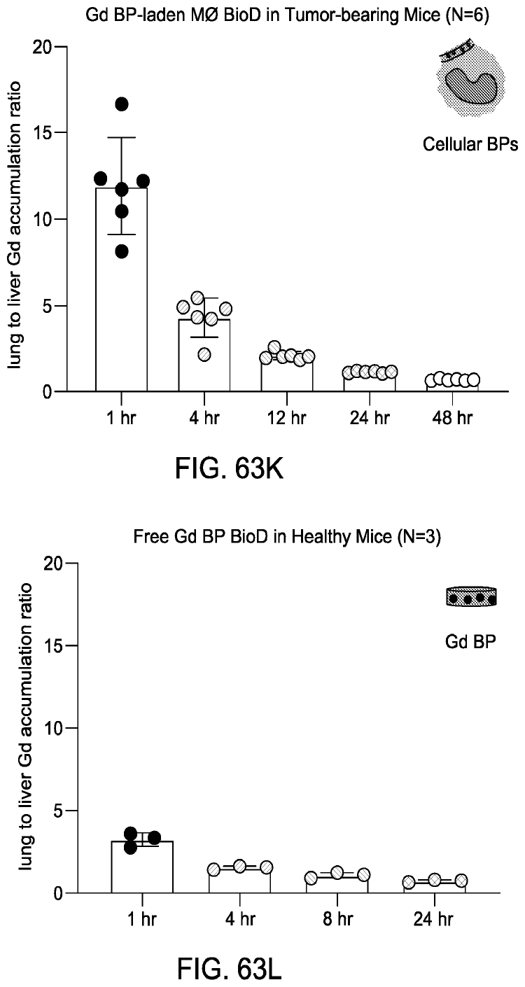

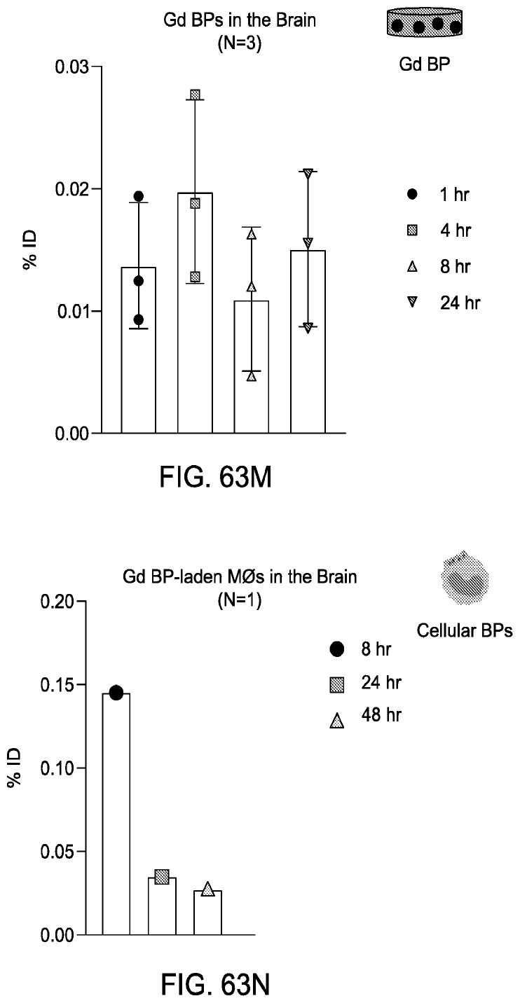

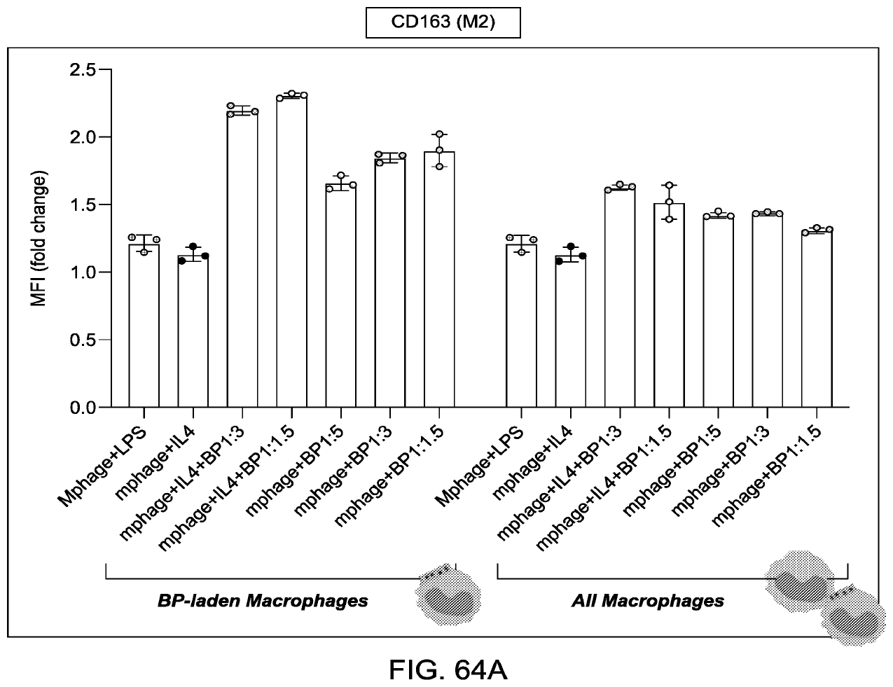

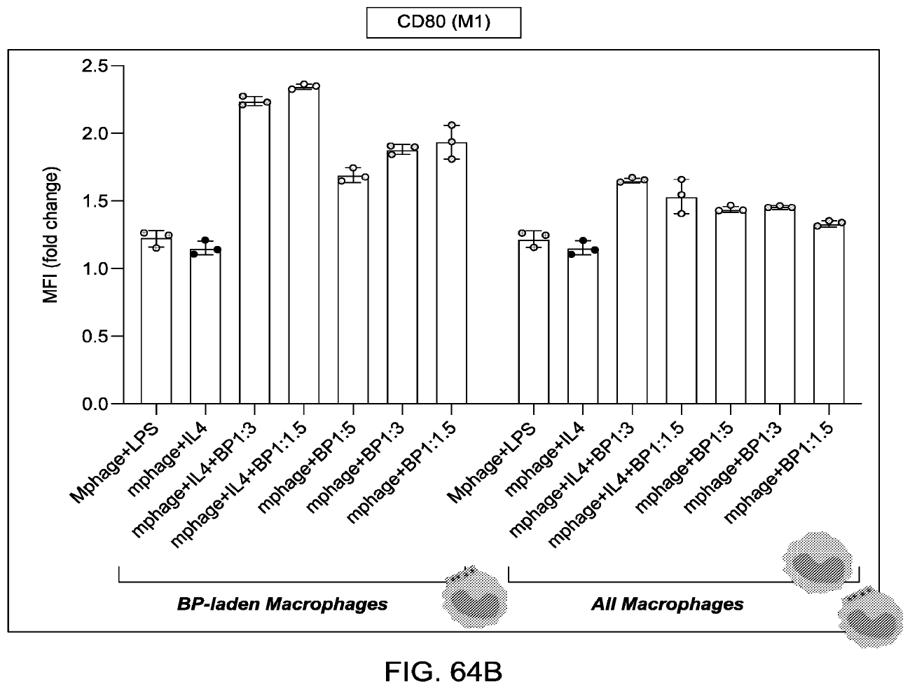

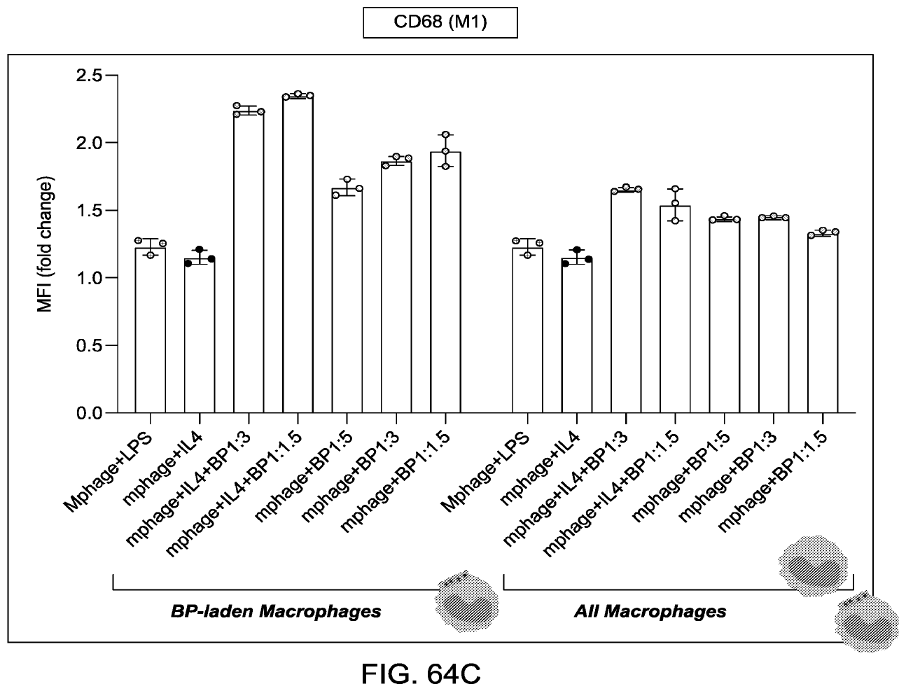

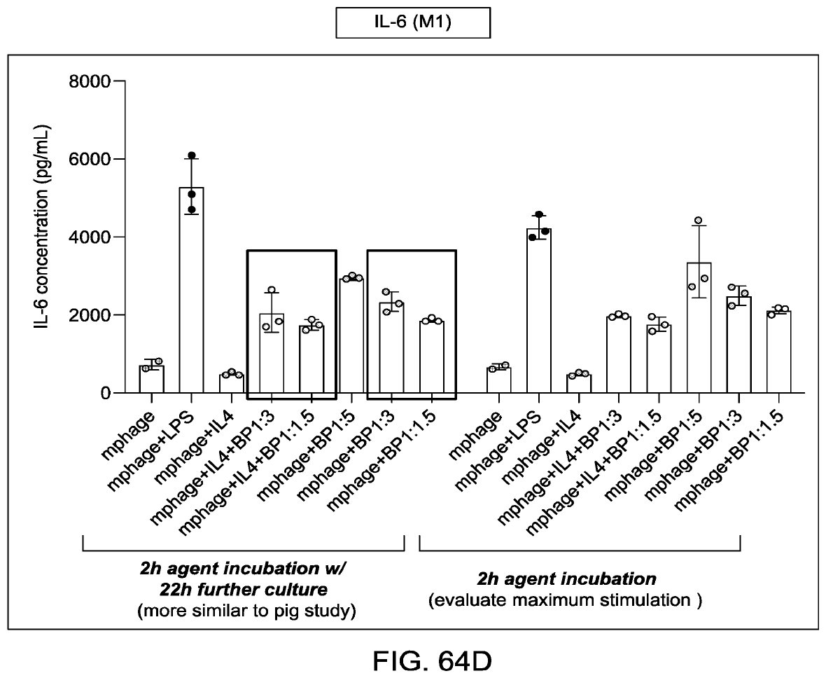

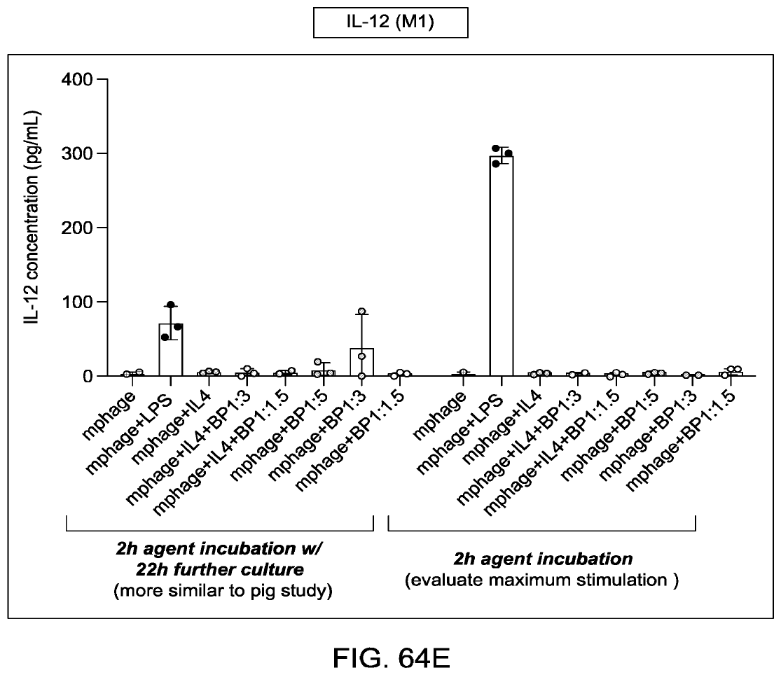

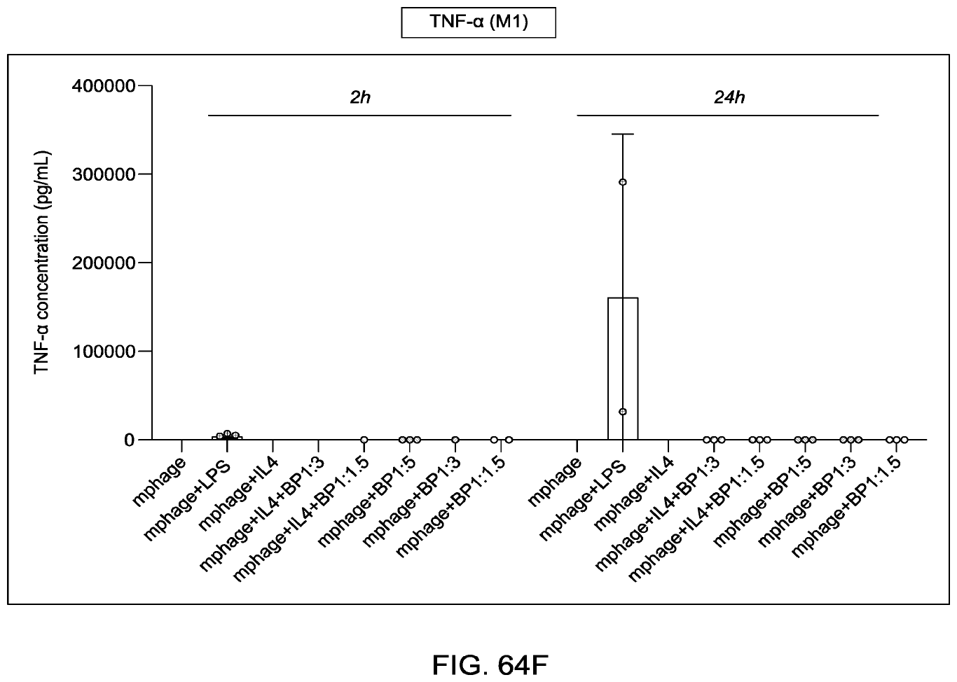

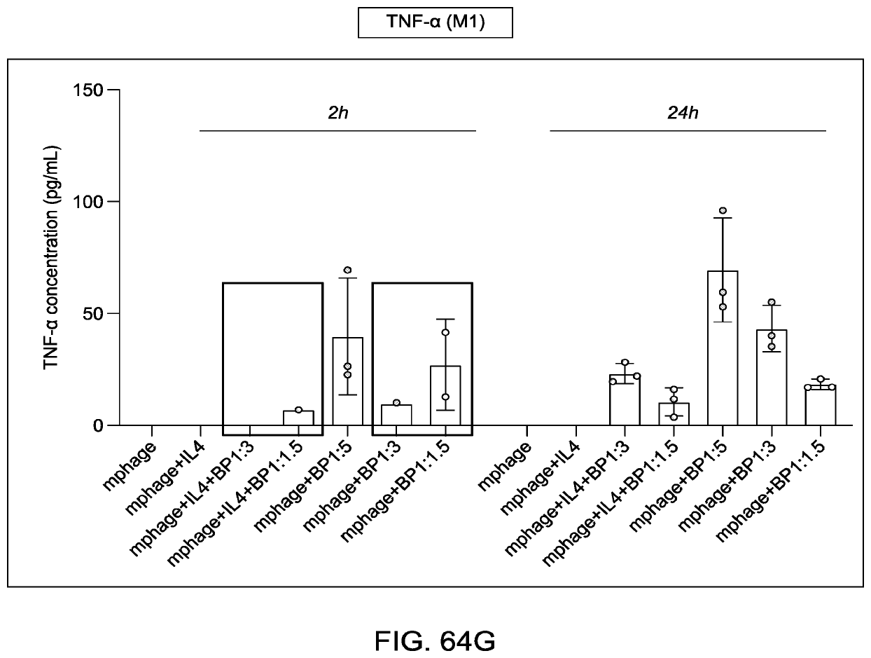

When backpacks loaded with IL-4 and Dex are adhered to monocytes, the cells display decreased M1 marker expression and increased M2 Marker expression (FIGS. 55A-55B). “Empty” or “blank” backpacks, on their own, also affect cell phenotype, as compared to monocyte-only controls (FIGS. 55C-55H).

The therapeutic efficacy of monocytes+backpacks was investigated using an Experimental autoimmune encephalomyelitis (EAE) chronic in vivo model, which is a widely-used animal model of multiple sclerosis (FIGS. 56A-56B). Injected monocytes accumulate in the CNS of EAE, even when BPs are adhered (FIGS. 56C-56D). In a 2 dose regimen treating acute EAE in vivo, the population of immune cells in the blood, brain, and spinal cord remained relatively similar across treatment groups (FIGS. 56E-56H). Cellular BPs modulate the phenotype of both infiltrating and resident myeloid cells in the brain after a 2 dose regimen treating acute EAE (FIGS. 56I-56K). Cellular BPs modulate the phenotype of both infiltrating and resident myeloid cells in the spinal cord after a 2 dose regimen treating acute EAE (FIGS. 56L-56M). Cellular BPs modulate the phenotype of T cells in the spinal cord after a 2 dose regimen treating acute EAE (FIG. 56N). Cellular backpacks decrease IFNg cytokine production in the CNS (FIGS. 56O-56P). Cellular BPs may decrease systemic inflammation, quantified by serum cytokine levels (FIG. 56Q).

### Example 9

A composition designed for treatment of traumatic brain injury (TBI) was designed, comprising macrophages with multiple-layer PLGA backpacks loaded with IL-4 and dexamethasone (FIG. 57A). These backpacks did not include a binding agent. These backpacks adhere to pig macrophages reproducibly (FIG. 57B).

### Example 10

Antibody functionalization of backpacks modulates specific cell-particle interactions (FIGS. 58A-58D). Binding of backpacks to circulating immune cells in vivo was measured (FIGS. 58E-581). Antibody functionalization does not alter BP biodistribution (FIGS. 58J-58N). Backpacks display some binding to organ-associated immune cells in vivo (FIGS. 580-58P). The content of a backpack can influence the organ biodistribution (FIGS. 59A-59H).

### Example 11

Backpacks comprising gadolinium were investigated. Herein, gadolinium (Gd)-loaded disk-shaped microparticles were used and referred to as “Gd-loaded hydrogel backpacks” and “Gd BPs”. Gd BP-laded monocytes and/or macrophages are referred to herein as a “cellular backpack system” or “cBP.” The structure of these backpacks is shown in FIG. 60A and the process of making the backpacks is depicted in FIGS. 60B-60C. Disk-shaped microparticles that incorporate the MR contrast agent gadolinium were made with this fabrication process with a fabrication yield of 50% (FIG. 60D).

BPs can be loaded with different amount of Gd (FIGS. 61B-61D) to obtain various relaxivities. The relaxivity of Gd within BPs (26.09/34.69/25.14 mM-is-1)(FIGS. 61B-61D) are much higher than that of gadovist (FIG. 61E; 3.18 mM⁻¹s⁻¹). Gd BPs are able to attach on the cell surface without being internalized (FIGS. 62A-62B). The binding efficiency of Gd BPs to murine macrophages varies from 50 to 90% depending on BP to cell ratios (FIG. 62D-62E). Both 6 μm and 8 um Gd BPs can attach on porcine macrophages with adhesion rates ranging from 60 to 80% (FIG. 62E).

Different backpack formulations, shown in Table 7, were tested. Anti-mouse IgG antibody was conjugated on single-layer Gd BPs with different methods (FIG. 63A), and these conjugations enhanced cell adhesion efficiencies (Table 7).

TABLE 7

| | Cell | BP:cell | |
|---|---|---|---|
| | source | ratio | Efficiency |
| Regular HAMA + 3x I2959 | commercial | 1.5 to 1 | 48 |
| | Balb/c Mø | | |
| Regular HAMA + 3x I2959 | commercial | 1.5 to 1 | 59 |
| with HA-strep mixed in | Balb/c Mø | | |
| bulk + anti-mouse IgG | | | |
| with biotin (system 2) | | | |
| Regular HAMA + 3x I2959 | commercial | 1.5 to 1 | 64 |
| with LbL (HA-strep/PAH) + | Balb/c Mø | | |
| anti-mouse IgG with | | | |
| biotin (system 3) | | | |
| Regular HAMA + 3x I2959 | self-collected | 1.5 to 1 | 50 |
| | Balb/c Mø | | |
| Regular HAMA + 3x I2959 | self-collected | 1.5 to 1 | 60 |
| with HA-strep mixed in | Balb/c Mø | | |
| bulk + anti-mouse IgG | | | |
| with biotin (system 2) | | | |
| Regular HAMA + 3x I2959 | self-collected | 1.5 to 1 | 66 |
| with LbL (HA-strep/PAH) + | Balb/c Mø | | |
| anti-mouse IgG with | | | |
| biotin (system 3) | | | |

cBPs are stable under various conditions. Gd BPs can stably attach to murine macrophages under various shear forces (2-20 Pa) (FIG. 63B). The binding efficiencies are similar in both fresh or frozen backpacks, indicating the freeze-thaw process does not affect adhesion of Gd BP to macrophages (FIG. 63C). Gd BPs can be injected into tissues and be observed by MRI.

An ex vivio MR imaging study was conducted in a rat brain. Sample A comprised 20 million BP/mL, Sample B comprised 3.33 million BP/mL, and Sample C comprised 1 million BP/mL. When Gd BPs were injected into tissues they could be observed by MRI (FIGS. 63D-63E).

Biodistribution of Gd BPs in healthy mice was examined (FIGS. 63F-63G). Gd BPs showed a >60% of accumulation in lung 1h post-injection with a decrease overtime, while the accumulation in liver and spleen increase with time. No overt accumulation was observed in other vital organs (brain, heart, and kidney). Biodistribution of cBP in mice with lung metastases was examined (FIGS. 63H-63J). 4T1-Fluc breast tumor cells were used to build lung metastatic model. Cellular backpacks showed a >70% of accumulation in lung 1h post-injection with a decrease over time, while the accumulation in liver and spleen increase with time. No overt accumulation was observed in other vital organs (brain, heart, and kidney). Cellular backpacks showed a significant accumulation in lung 1h post-injection with a decrease over time. A mild accumulation in liver can also be observed. No overt accumulation was observed in other vital organs (brain, heart, and kidney). Cellular backpacks showed a 3-fold higher lung to liver ratio than Gd BPs one hour post injection, indicating macrophages home to the inflammatory lung tissues (i.e., lung tissues with 4T1 breast cancer metastases) and enhanced Gd BP accumulation in the lung (FIGS. 63K-63L).

Gd BPs can induce both M1 and M2 biomarkers at the same time (FIGS. 64A-64C). Gd BPs induced a mild pro-inflammatory cytokine secretion, and the secretions increased with BP:cell ratios; however, compared to positive control —LPS, the levels were relatively low in IL-6 and extremely low in IL-12 and TNF-α (FIGS. 64D-64G) Moreover, with the addition of IL-4, the cytokine secretions mildly decreased.

1. A polymeric particle comprising at least one imaging reagent.

2. The polymeric particle of claim 1, wherein the at least one imaging reagent is at least one MRI contrast reagent.

3. The polymeric particle of claim 1, further comprising at least one binding reagent selected from the group consisting of: a CD11b binding reagent; a CD3 binding reagent; a CD19 binding reagent; a CD49b binding reagent; a CD56 binding reagent; a CD 11a binding reagent; a CD27 binding reagent; a CD44 binding reagent; a CD45 binding reagent; a NKG2D binding reagent; a NKp30 binding reagent; a NKp46 binding reagent; and an ICAM1 binding reagent.

4. A polymeric particle comprising at least one binding reagent selected from the group consisting of: a CD11b binding reagent; a CD3 binding reagent; a CD19 binding reagent; a CD49b binding reagent; a CD56 binding reagent; a CD11a binding reagent; a CD27 binding reagent; a CD44 binding reagent; a CD45 binding reagent; a NKG2D binding reagent; a NKp30 binding reagent; a NKp46 binding reagent; and an ICAM1 binding reagent.

5. The polymeric particle of claim 3, wherein the binding reagent is a CD11b binding reagent; a CD3 binding reagent; or a CD19 binding reagent.

6. The polymeric particle of claim 1, wherein the binding reagent is a CD11b binding reagent.

7. (canceled)

8. The polymeric particle of claim 3, wherein the binding reagent is an antibody or an antibody reagent.

9. (canceled)

10. (canceled)

11. (canceled)

12. (canceled)

13. The polymeric particle of claim 1, further comprising ICAM1, NKp30, and/or NKp46.

14. A polymeric particle comprising ICAM1, NKp30, and/or NKp46.

15. (canceled)

16. The polymeric particle of claim 1, wherein the polymeric particle further comprises one or more cell adhesive molecules (e.g., polyelectrolytes).

17.-44. (canceled)

45. The polymeric particle of claim 1, wherein the one or more imaging agents are selected from the group consisting of: one or more contrast agents, one or more MRI contrast agents, one or more microbubbles, one or more metal ions, one or more radioactive isotopes, one or more optical imaging agents, one or more SPECT imaging agents, and one or more PET imaging agents.

46. The polymeric particle of claim 45, wherein the one or more MRI contrast agents comprise one or more of: a gadoliunium-based contrast agent, superparamagnetic iron oxide, ultrasmall superparamagnetic iron oxide, superparamagnetic iron-platinum, manganese chelates, iron salts, and perflubron.

47. The polymeric particle of claim 46, wherein the gadoliunium-based contrast agent comprises one or more of: gadolinium, gadoxetate, gadobutrol, gadoterate, gadoteridol, gadopentetate, gadobenate, gadopentetic acid dimegulmine, gadoxentate, gadoversetamide, gadodiamide, gadofosveset, gadocoletic acid, gadomelitol, gadomer 17, and gadoxetic acid.

48.-62. (canceled)

63. The polymeric particle of claim 1, wherein the polymeric particle further comprises one or more payload reagents.

64. The polymeric particle of claim 63, wherein the payload reagent is a therapeutic molecule.

65.-71. (canceled)

72. The polymeric particle of claim 1, further comprising at least one priming agent.

73. The polymeric particle of claim 72, wherein the at least one priming agent is selected from the group consisting of: a caspase inhibitor; fMLF; C5A; LTB4; PAF; TNF-α; GCSF; GM-CSF; IFN-γ; IL-1β; IL8; IL-15; IL-18; IL-33; adiponectin; LPS; LAMs; lipopeptide; flagellin; ATP; substance P; CL097; and CL075.

74.-83. (canceled)

84. An engineered cellular composition comprising:
a. a cell; and
b. a polymeric particle of claim 1, wherein the particle is located on the cell surface of the cell.

85.-107. (canceled)

108. A method of treating cancer and/or a tumor in a subject in need thereof, the method comprising administering to the subject the engineered cellular composition of claim 84.

109. The method of claim 108, wherein the polymeric particle comprises a payload reagent that is a chemotherapeutic or the cell is a macrophage, NK cell, or T cell.

110.-135. (canceled)
